(19) United States
(12) Patent Application Publication
(10) Pub. No.: US 2025/0040427 A1
(43) Pub. Date: Jan. 30, 2025

(54) LIGHT-EMITTING ELEMENT, AMINE COMPOUND FOR THE LIGHT-EMITTING ELEMENT, AND ELECTRONIC DEVICE INCLUDING THE LIGHT-EMITTING ELEMENT

(71) Applicant: Samsung Display Co., Ltd., Yongin-si (KR)

(72) Inventors: MINJI KIM, Yongin-si (KR); BEOMJOON KIM, Yongin-si (KR); Chaeyeong KIM, Yongin-si (KR); BYEONGWOOK YOO, Yongin-si (KR); Jeong Min LEE, Yongin-si (KR); EUNJAE JEONG, Yongin-si (KR); SOHEE JO, Yongin-si (KR); HAN NA CHA, Yongin-si (KR); SANGHYUN HAN, Yongin-si (KR)

(21) Appl. No.: 18/626,185

(22) Filed: Apr. 3, 2024

(30) Foreign Application Priority Data
Jul. 12, 2023 (KR) 10-2023-0090609

(51) Int. Cl. H10K 85/60; C07C 211/54; C07C 211/61; C07D 307/91; C09K 11/06; H10K 50/15

(52) U.S. Cl. CPC H10K 85/633; C07C 211/54; C07C 211/61; C07D 307/91; C09K 11/06; H10K 85/636; C07C 2601/14; C07C 2603/18; C07C 2603/97; C09K 2211/1003; C09K 2211/1018; H10K 50/15; H10K 85/615; H10K 85/624; H10K 85/626; H10K 85/6574

(57) ABSTRACT

A light-emitting element includes a first electrode, a second electrode on the first electrode, and at least one functional layer between the first electrode and the second electrode and containing an amine compound represented by Formula 1.

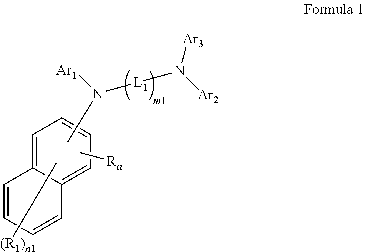

### CROSS-REFERENCE TO RELATED APPLICATIONS

The present application claims priority to and the benefit of Korean Patent Application No. 10-2023-0090609, filed on Jul. 12, 2023, in the Korean Intellectual Property Office, the entire content of which is incorporated herein by reference.

### BACKGROUND

### 1. Field

One or more embodiments of the present disclosure relates to a light-emitting element, an amine compound for the light-emitting element, and an electronic device including the light-emitting element.

### 2. Description of the Related Art

Recently, organic electroluminescence display devices and/or the like have been actively developed as image display devices. Organic electroluminescence display devices and/or the like are display devices including self-luminous type or kind light-emitting elements in which holes and electrons respectively (e.g., separately) injected from a first electrode and a second electrode recombine in a light emitting layer, thereby causing a light-emitting material of the light-emitting layer to emit light to implement display of images.

In applying a light-emitting element to display devices, improvements in luminous efficiency and lifespan are required and/or desired, and thus the material development for a light-emitting element capable of stably achieving such improvements, is continuously required and/or pursued.

For example, to achieve a light-emitting element having relatively high efficiency and relatively long-lifespan, materials having excellent or suitable hole transport ability and stability for a hole transport region of the light-emitting element are particularly under development and research.

### SUMMARY

One or more aspects of embodiments of the present disclosure are directed toward a light-emitting element having improved luminous efficiency and element lifespan.

One or more aspects of embodiments of the present disclosure are directed toward an amine compound capable of improving luminous efficiency and element lifespan of a light-emitting element.

On or more aspects of embodiments of the present disclosure are directed toward an electronic device having excellent or suitable display quality by including the light-emitting element having improved luminous efficiency and lifespan.

Additional aspects will be set forth in part in the description which follows and, in part, will be apparent from the description, or may be learned by practice of the presented embodiments.

According to one or more embodiments of the present disclosure, a light-emitting element includes a first electrode, a second electrode on the first electrode, and at least one functional layer between the first electrode and the second electrode and containing an amine compound represented by Formula 1.

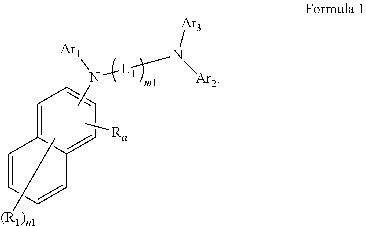

In Formula 1, Ar₁ to Ar₃ may each independently be a substituted or unsubstituted oxy group, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, an embodiment or casein which Ar₁ includes a fluorene group, a dibenzofuran group, or a dibenzothiophene group is excluded, L₁ may be a substituted or unsubstituted phenylene group, Rₐ may be a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms, R₁ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, n1 may be an integer of 0 to 6, m1 may be an integer of 1 to 4, and when m1 is 2, then an embodiment in which L₁ is represented by Formula A-1 is excluded.

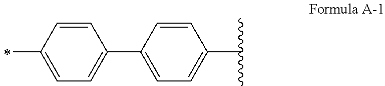

In Formula A-1, [symbol] is a position at which a nitrogen atom to which Ar₁ is connected in Formula 1, is connected, and

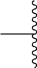

is a position at which a nitrogen atom, to which Ar₂ and Ar₃ are connected in Formula 1, is connected.

In one or more embodiments, the at least one functional layer may include a light-emitting layer, a hole transport region between the first electrode and the light-emitting layer, and an electron transport region between the light-emitting layer and the second electrode, and the hole transport region may include the amine compound represented by Formula 1.

In one or more embodiments, the hole transport region may include a hole injection layer on the first electrode and a hole transport layer on the hole injection layer, wherein the hole transport layer may contain the amine compound represented by Formula 1.

In one or more embodiments, a layer adjacent to the light-emitting layer among a plurality of layers included in the hole transport region may contain the amine compound represented by Formula 1.

In one or more embodiments, the amine compound represented by Formula may be a diamine compound.

In one or more embodiments, the amine compound represented by Formula may be represented by Formula 2.

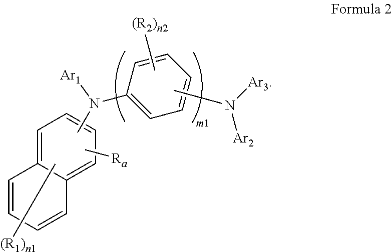

In Formula 2, R₂ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and n2 may be an integer of 0 to 4.

In Formula 2, the same descriptions as defined in Formula 1 may be applied to Ar₁ to Ar₃, Rₐ, R₁, n1, and m1.

In one or more embodiments, the amine compound represented by Formula may be represented by Formula 3.

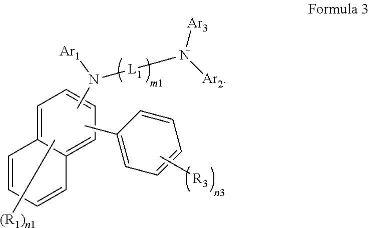

In Formula 3, R₃ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and n3 may be an integer of 0 to 5.

In Formula 3, the same descriptions as defined in Formula 1 may be applied to Ar₁ to Ar₃, L₁, R₁, n1, and m1.

In one or more embodiments, the amine compound represented by Formula may be represented by Formula 4-1 or Formula 4-2.

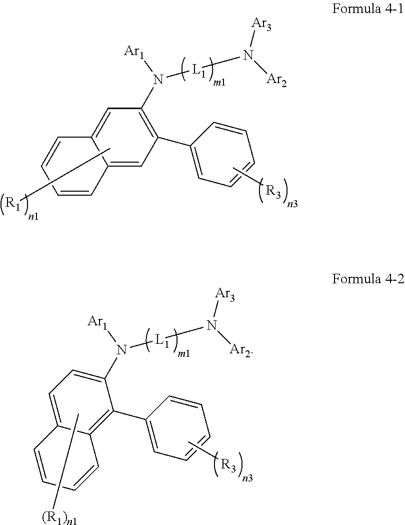

In Formulas 4-1 and 4-2, the same descriptions as defined in Formulas 1 and 3 may be applied to Ar₁ to Ar₃, L₁, R₁, R₃, n1, n3, and m1.

In one or more embodiments, the amine compound represented by Formula may be represented by any one selected from among Formulas 5-1 to 5-5.

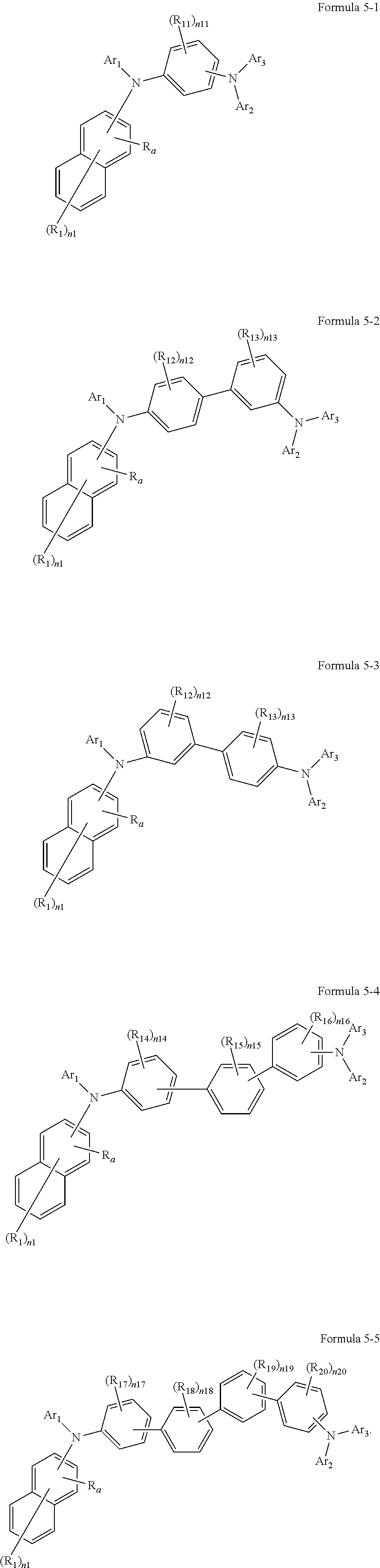

In Formulas 5-1 to 5-5, R₁₁ to R₂₀ may each independently be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and n11 to n20 may each independently be an integer of 0 to 4.

In Formulas 5-1 to 5-5, the same descriptions as defined in Formula 1 may be applied to Ar₁ to Ar₃, Rₐ, R₁, and n1.

In one or more embodiments, L₁ may be represented by any one selected from among Formulas L-1 to L-22.

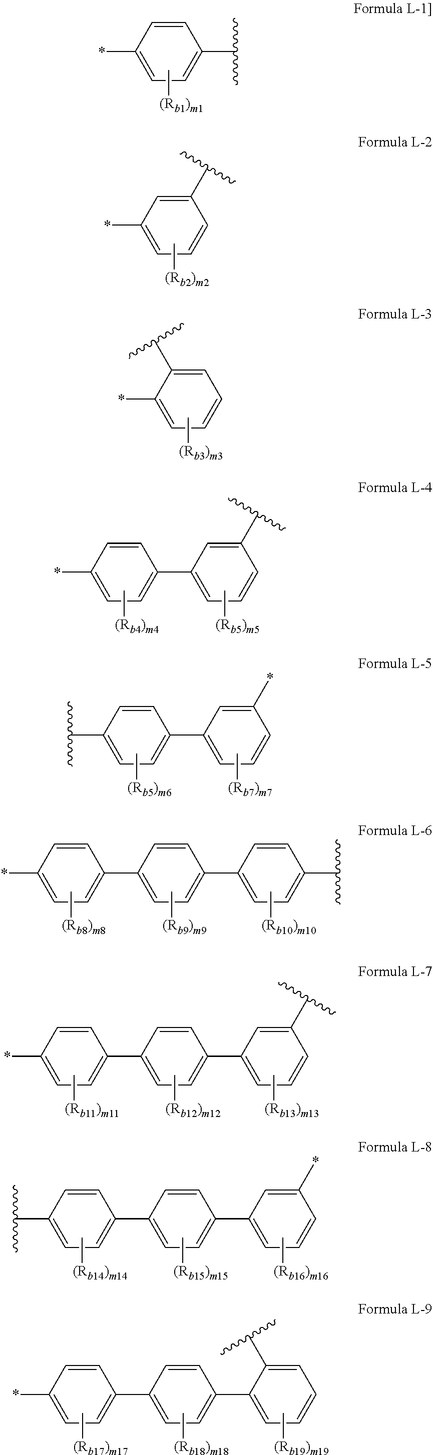

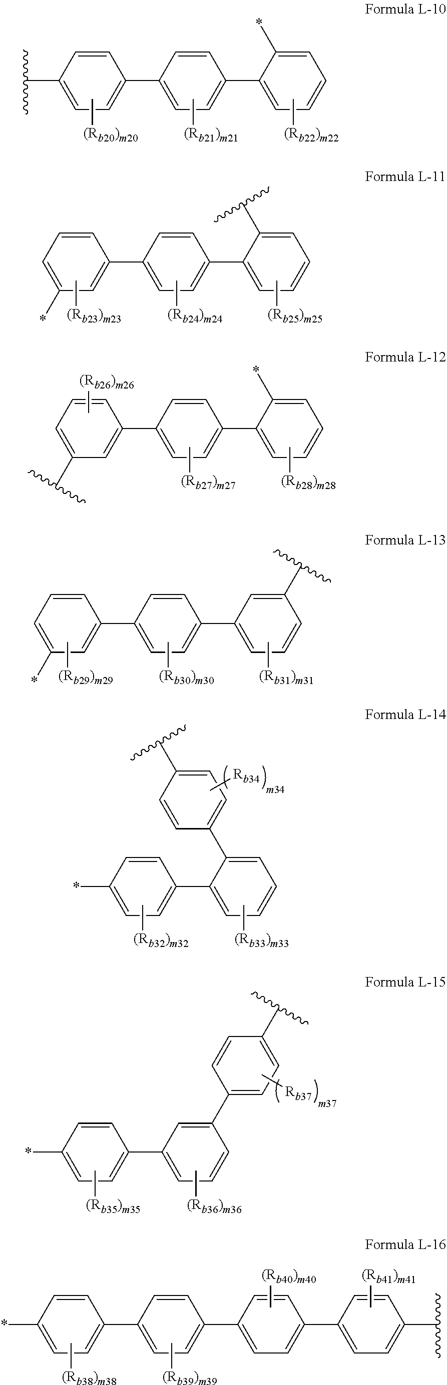

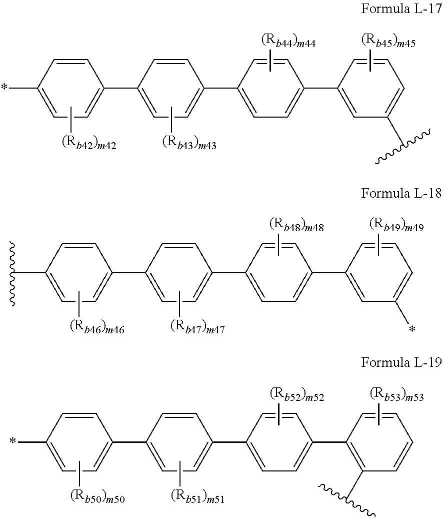

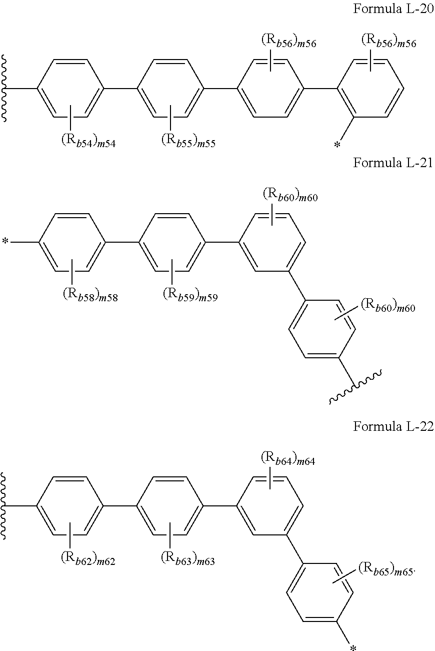

In Formulas L-1 to L-22, R_{b1} to R_{b65} may each independently be hydrogen, deuterium, a halogen, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and m1 to m65 may each independently be an integer of 0 to 4.

In one or more embodiments, Ar₁ may be a substituted or unsubstituted phenyl group, a substituted or unsubstituted biphenyl group, or a substituted or unsubstituted naphthyl group, and Ar₂ and Ar₃ may each independently be a substituted or unsubstituted phenyl group, a substituted or unsubstituted biphenyl group, a substituted or unsubstituted naphthyl group, or a substituted or unsubstituted fluorenyl group.

In one or more embodiments, Ar₁ may be represented by any one selected from among Formulas B-1 to B-4, and Ar₂ and Ar₃ may each independently be represented by any one selected from among Formulas B-1 to B-7.

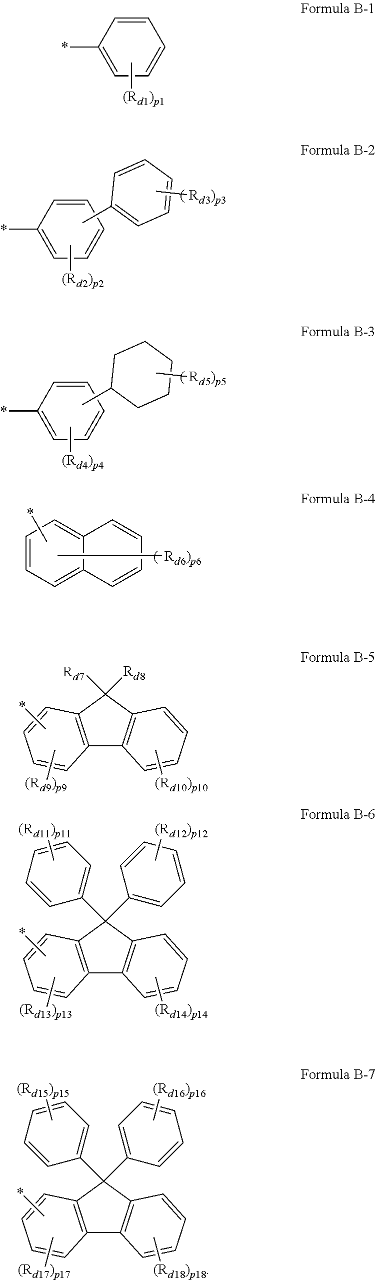

In Formulas B-1 to B-7, R_{d1} to R_{d18} may each independently be hydrogen, deuterium, a halogen, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, p1, p3, p11, and p12 may each independently be an integer of 0 to 5, p2, p4, p10, p14, p15, p16, and p18 may each independently be an integer of 0 to 4, p5 is an integer of 0 to 11, p6 is an integer of 0 to 7, and p9, p13, and p17 may each independently be an integer of 0 to 3.

In one or more embodiments, the amine compound represented by Formula may be represented by Formula 6-1 or Formula 6-2.

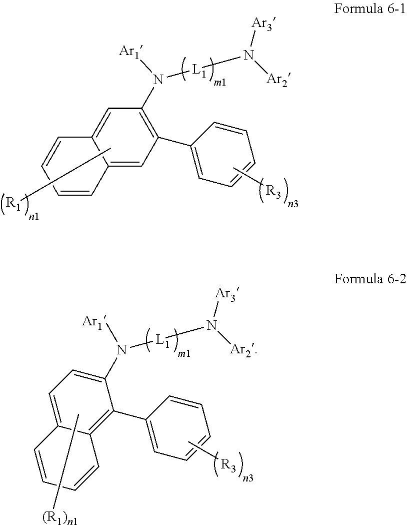

In Formulas 6-1 and 6-2, R₃ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, n3 may be an integer of 0 to 5, and Ar₁′ to Ar₃′ may each independently be selected from Substituent Group A, provided that Ar₁′ may not be any one selected from among a36 to a47.

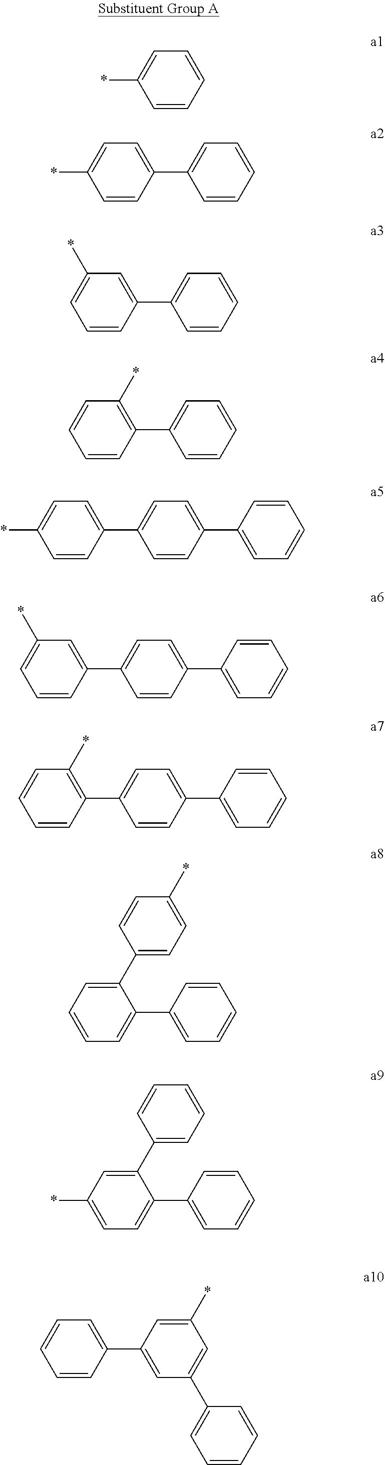

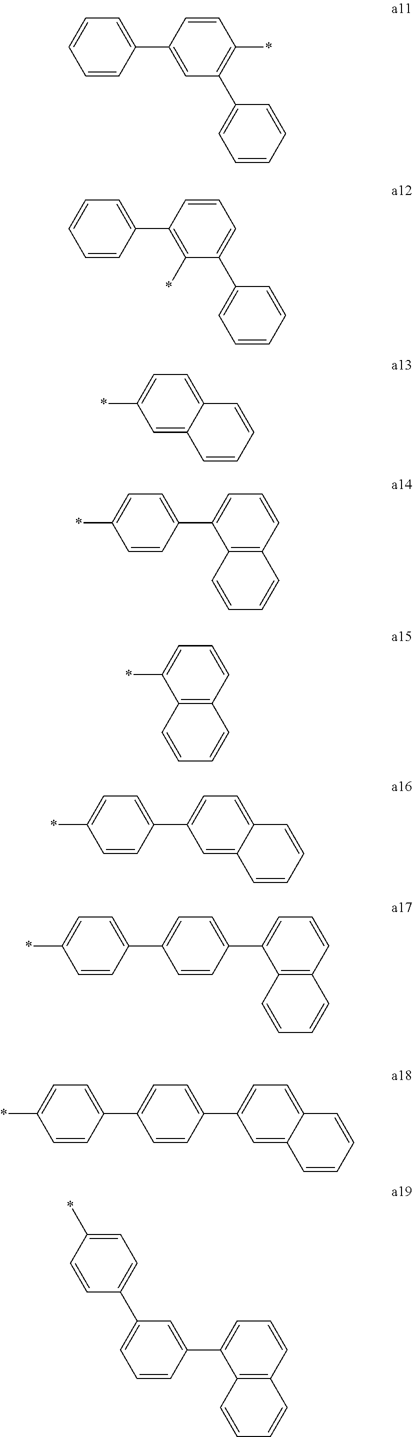

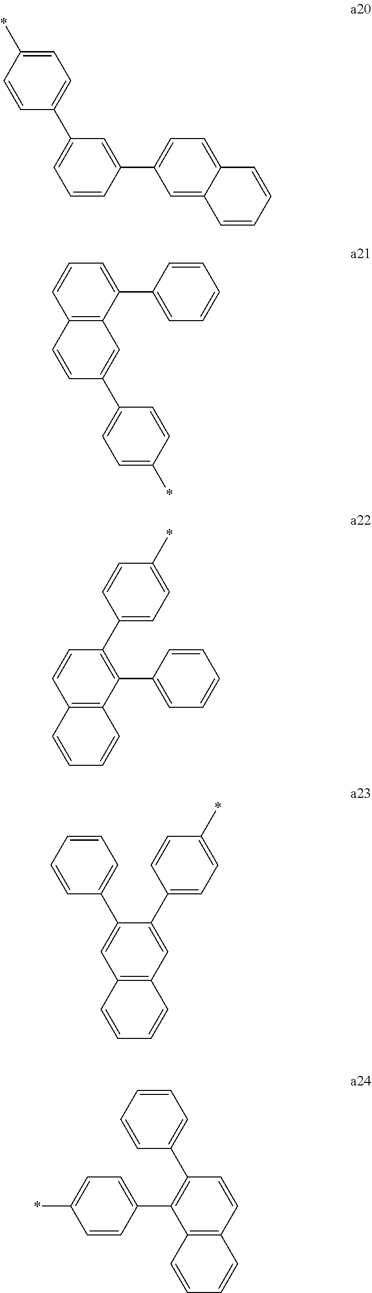

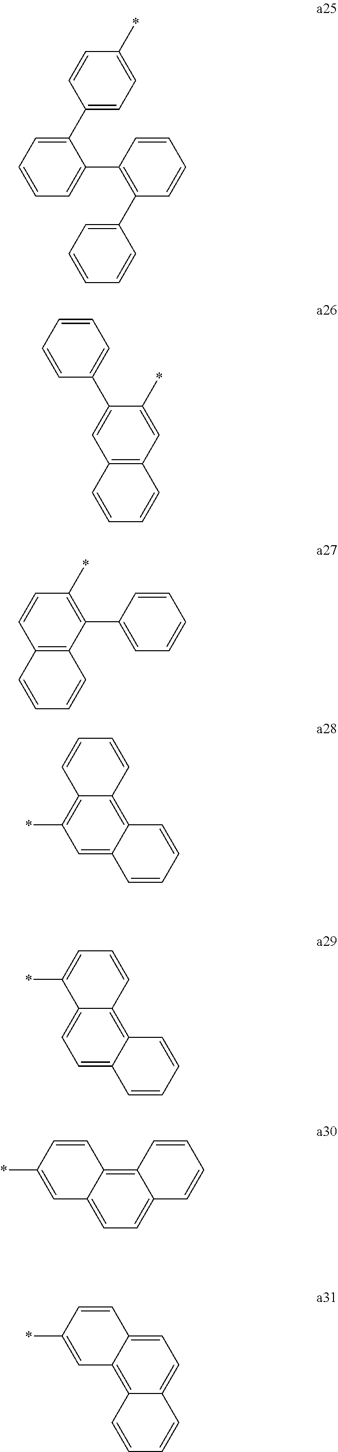

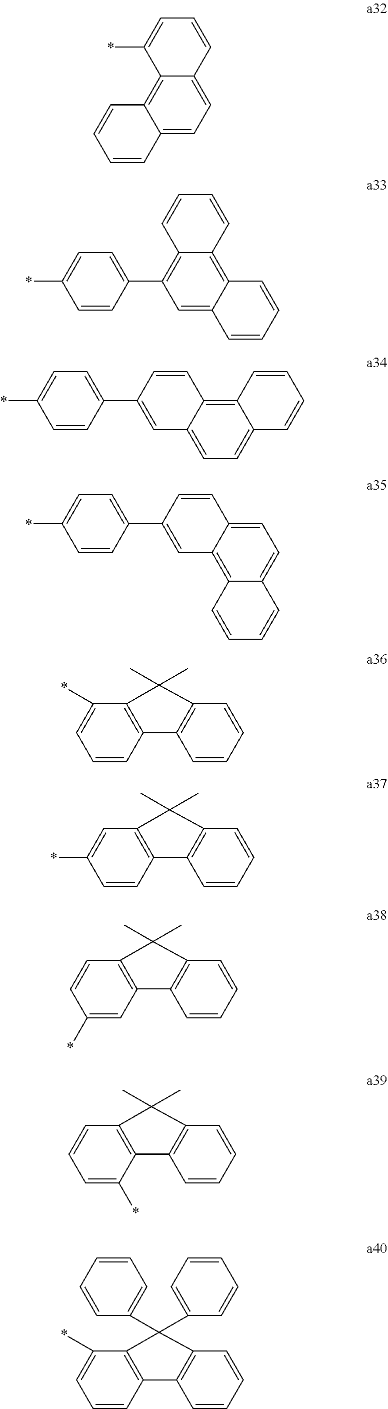

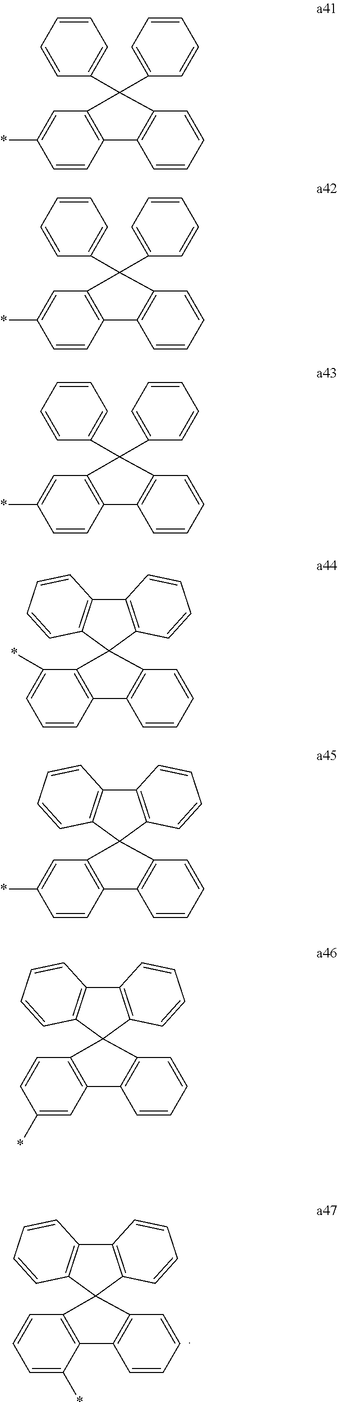

In Formulas 6-1 and 6-2, the same descriptions as defined in Formula 1 may be applied to L₁, R₁, n1, and m1.

In one or more embodiments of the present disclosure, for an electronic device selected from among a large sized display device such as a television, a monitor, and an outside billboard, and a small and medium sized display device such as a personal computer, a laptop computer, a personal digital assistant, a display device for a vehicle, a game console, a portable electronic device, and a camera, the electronic device may include at least one of the light-emitting elements, and the light-emitting element contains an amine compound represented by Formula 1.

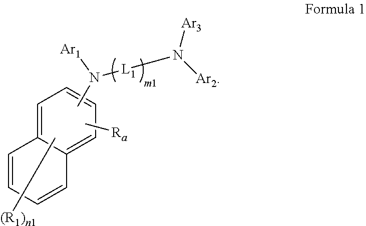

In Formula 1, Ar₁ to Ar₃ may each independently be a substituted or unsubstituted oxy group, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, an embodiment in which Ar₁ includes a fluorene group, a dibenzofuran group, or a dibenzothiophene group is excluded, L₁ may be a substituted or unsubstituted phenylene group, Rₐ may be a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms, R₁ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, n1 may be an integer of 0 to 6, m1 may be an integer of 1 to 4, and when m1 is 2, an embodiment in which L₁ is represented by Formula A-1 is excluded.

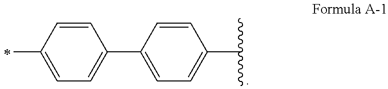

In Formula A-1, [symbol] is a position at which a nitrogen atom to which Ar₁ is connected in Formula 1 is connected, and

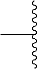

is a position at which a nitrogen atom to which Ar₂ and Ar₃ are connected in Formula 1, is connected.

In one or more embodiments, the electronic device may include a base layer, a circuit layer on the base layer, a display element layer on the circuit layer and including the light-emitting element, and an encapsulation layer disposed on the display element layer, wherein the light-emitting element may include a first electrode, a second electrode on the first electrode, and at least one functional layer between the first electrode and the second electrode and containing the amine compound represented by Formula 1.

In one or more embodiments, the light-emitting element may further include a capping layer on the second electrode, and the capping layer may have a refractive index of at least about 1.6 for light in a wavelength range of about 550 nm to about 660 nm.

In one or more embodiments, a light control layer on the encapsulation layer and containing quantum dots may be further included, the light-emitting element is to emit a first color light, wherein the light control layer may include a first light control part containing a first quantum dot that converts the first color light into a second color light with a longer wavelength than the first color light, a second light control part containing a second quantum dot that converts the first color light into a third color light with a longer wavelength than the first color light and the second color light, and a third light control part that transmits the first color light.

In one or more embodiments, a color filter layer on the light control layer may be further included, and the color filter layer may include a first filter that transmits the second color light, a second filter that transmits the third color light, and a third filter that transmits the first color light.

In one or more embodiments of the present disclosure, an amine compound represented by Formula 1 is provided.

### BRIEF DESCRIPTION OF THE FIGURES

The accompanying drawings are included to provide a further understanding of the present disclosure, and are incorporated in and constitute a part of this disclosure. The drawings illustrate example embodiments of the present disclosure and, together with the description, serve to explain principles of the present disclosure.

Above and/or other aspects of the present disclosure should become apparent and appreciated from the following description of embodiments taken in conjunction with the accompanying drawings. In the drawings:

FIG. 1 is a plan view illustrating a display apparatus according to one or more embodiments of the present disclosure;

FIG. 2 is a cross-sectional view of a display apparatus according to one or more embodiments of the present disclosure;

FIG. 3 is a cross-sectional view schematically illustrating a light-emitting element according to one or more embodiments of the present disclosure;

FIG. 4 is a cross-sectional view schematically illustrating a light-emitting element according to one or more embodiments of the present disclosure;

FIG. 5 is a cross-sectional view schematically illustrating a light-emitting element according to one or more embodiments of the present disclosure;

FIG. 6 is a cross-sectional view schematically illustrating a light-emitting element according to one or more embodiments of the present disclosure;

FIG. 7 is a cross-sectional view of a display device according to one or more embodiments of the present disclosure;

FIG. 8 is a cross-sectional view of a display device according to one or more embodiments of the present disclosure;

FIG. 9 is a cross-sectional view illustrating a display device according to one or more embodiments of the present disclosure;

FIG. 10 is a cross-sectional view illustrating a display device according to one or more embodiments of the present disclosure; and

FIG. 11 is a view illustrating a vehicle in which display devices according to one or more embodiments are disposed.

### DETAILED DESCRIPTION

The present disclosure may be modified in one or more suitable manners and have many forms, and thus specific embodiments will be exemplified in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements. In the accompanying drawings, the dimensions of each structure may be exaggeratingly illustrated for clarity of the present disclosure. It will be understood that, although the terms “first,” “second,” etc., may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” “one,” and “the” are intended to include the plural forms as well, unless the context clearly indicates otherwise. Further, the utilization of “may” when describing embodiments of the present disclosure refers to “one or more embodiments of the present disclosure”.

In the present disclosure, it will be understood that the terms “comprise(s)/comprising,” “include(s)/including,” “have/has/having” and/or the like specify the presence of features, numbers, steps, operations, component, parts, or combinations thereof disclosed in the disclosure, but do not exclude the possibility of presence or addition of one or more other features, numbers, steps, operations, component, parts, or combinations thereof. As used herein, the terms “and,” “or,” and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, when a layer, a film, a region, or a plate is referred to as being “on” or “in an upper portion of” another layer, film, region, or plate, it may be not only “directly on” the layer, film, region, or plate, but one or more intervening layers, films, regions, or plates may also be present. Opposite this, when a layer, a film, a region, or a plate is referred to as being “below”, “in a lower portion of” another layer, film, region, or plate, it can be not only directly under the layer, film, region, or plate, but one or more intervening layers, films, regions, or plates may also be present. In some embodiments, it will be understood that when a part is referred to as being “on” another part, it can be disposed above the other part, or disposed under the other part as well. In one or more embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, the term “substituted or unsubstituted” may refer to substituted or unsubstituted with at least one substituent selected from the group consisting of deuterium, a halogen, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified above may be substituted or unsubstituted. For example, a biphenyl group may be interpreted as an aryl group or a phenyl group substituted with a phenyl group.

In the present disclosure, the phrase “bonded to an adjacent group to form a ring” may refer to that a group is bonded to an adjacent group to form a substituted or unsubstituted hydrocarbon ring or a substituted or unsubstituted heterocycle. The hydrocarbon ring may include an aliphatic hydrocarbon ring and/or an aromatic hydrocarbon ring. The heterocycle includes an aliphatic heterocycle and an aromatic heterocycle. The hydrocarbon ring and the heterocycle may each be monocyclic or polycyclic. In some embodiments, the rings formed by adjacent groups being bonded to each other may be connected to another ring to form a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In one or more embodiments, two methyl groups in 4,5-dimethylphenanthrene may be interpreted as “adjacent groups” to each other.

In the present disclosure, examples of a halogen may include fluorine, chlorine, bromine, or iodine.

In the present disclosure, an alkyl group may be linear or branched. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments of the present disclosure are not limited thereto.

In the present disclosure, a cycloalkyl group may refer to a cyclic alkyl group. The number of carbons in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments of the present disclosure are not limited thereto.

In the present disclosure, an alkenyl group refers to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, etc., but embodiments of the present disclosure are not limited thereto.

In the present disclosure, an alkynyl group refers to a hydrocarbon group including at least one carbon-carbon triple bond in the middle or terminal of an alkyl group having 2 or more carbon atoms. The alkynyl group may be linear or branched. Although the number of carbon atoms is not specifically limited, it may be 2 to 30, 2 to 20, or 2 to 10. Examples of the alkynyl group may include an ethynyl group, a propynyl group, etc., but embodiments of the present disclosure are not limited thereto.

In the present disclosure, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the present disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 50, 6 to 40, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments of the present disclosure are not limited thereto.

In the present disclosure, a fluorenyl group may be substituted, and two substituents may be bonded to each other to form a spiro structure. Examples of the substituted fluorenyl group are as follows. However, embodiments of the present disclosure are not limited thereto.

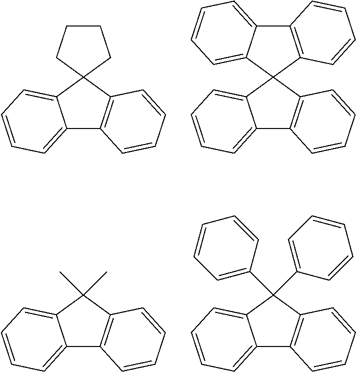

A heterocyclic group utilized herein may refer to any functional group or substituent derived from a ring containing at least one of B, O, N, P, Si, or Se as a heteroatom. The heterocyclic group may include an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, a heterocyclic group may contain at least one of B, O, N, P, Si or S as a heteroatom. When the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 60, 2 to 50, 2 to 40, 2 to 30, 2 to 20, or 2 to 10.

In the present disclosure, an aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments of the present disclosure are not limited thereto.

In the present disclosure, a heteroaryl group may contain at least one of B, O, N, P, Si, or S as a heteroatom. When the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 60, 2 to 50, 2 to 40, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, a silyl group may include an alkylsilyl group and/or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments of the present disclosure are not limited thereto.

In the present disclosure, the number of ring-forming carbon atoms in a carbonyl group is not specifically limited, for example, may be 1 to 40, 1 to 30, or 1 to 20. For example, the carbonyl group may have the following structures, but embodiments of the present disclosure are not limited thereto.

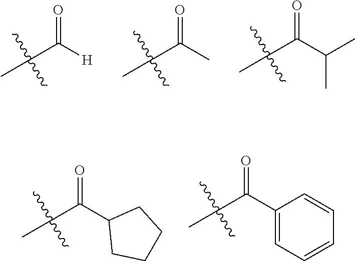

In the present disclosure, the number of carbon atoms in a sulfinyl group or a sulfonyl group is not particularly limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkylthio group and/or an arylthio group. The thio group may refer to that a sulfur atom is bonded to the alkyl group or the aryl group defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments of the present disclosure are not limited thereto.

In the present disclosure, an oxy group may refer to that an oxygen atom is bonded to the alkyl group or the aryl group defined above. The oxy group may include an alkoxy group and/or an aryl oxy group. The alkoxy group may be a linear chain, a branched chain, or a ring. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but embodiments of the present disclosure are not limited thereto.

A boron group herein may refer to that a boron atom is bonded to the alkyl group or the aryl group defined above. The boron group may include an alkyl boron group and/or an aryl boron group. Examples of the boron group may include a dimethylboron group, a trimethylboron group, a t-butyldimethylboron group, a diphenylboron group, a phenylboron group, etc., but embodiments of the present disclosure are not limited thereto.

In the present disclosure, the number of carbon atoms in an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, etc., but embodiments of the present disclosure are not limited thereto.

In the present disclosure, the alkyl group among an alkylthio group, an alkylsulfoxy group, an alkylaryl group, an alkylamino group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamino group, an arylboron group, an arylsilyl group, an arylamine group may be the same as the examples of the aryl group described above.

In the present disclosure, a direct linkage may refer to a single bond.

In the present disclosure,

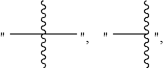

and [symbol] refer to a position to be connected.

Hereinafter, embodiments of the present disclosure will be described in more detail with reference to the accompanying drawings.

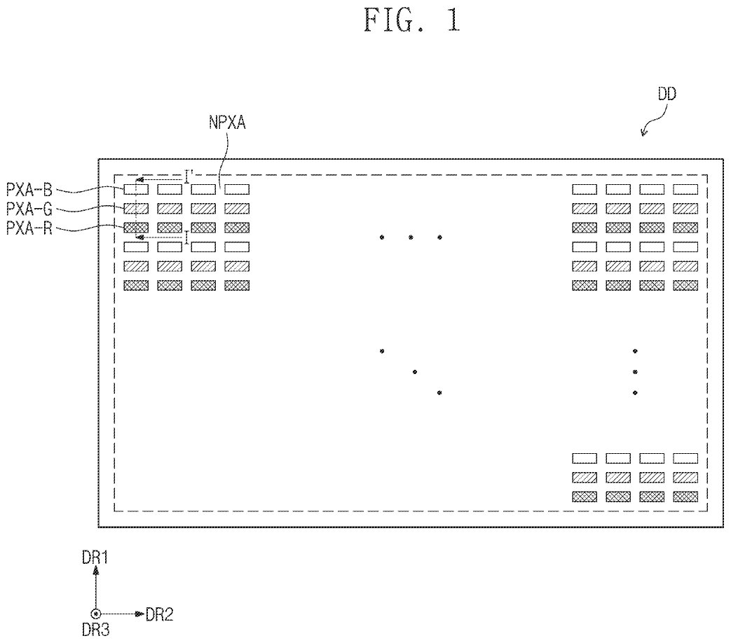

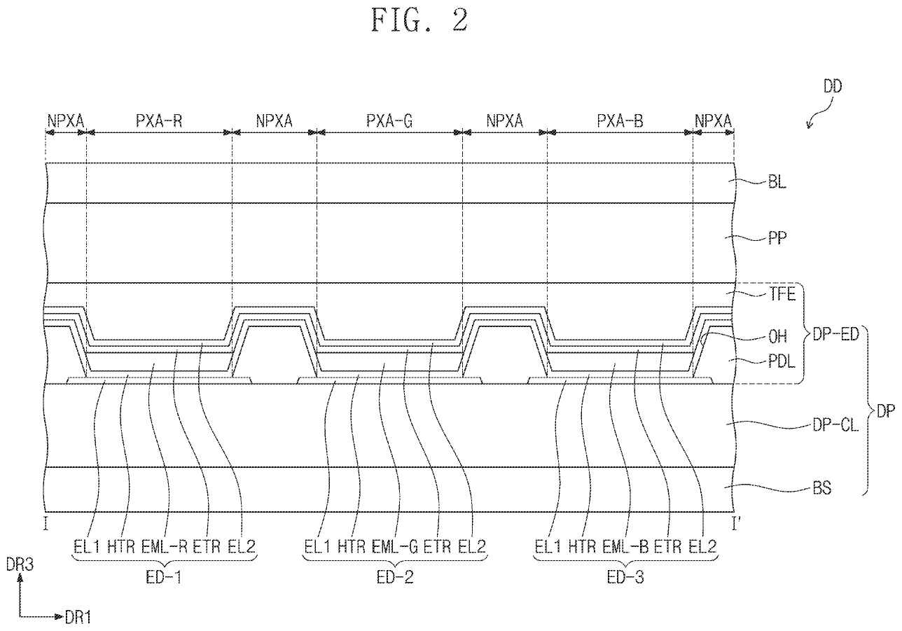

FIG. 1 is a plan view illustrating a display apparatus DD according to one or more embodiments of the present disclosure. FIG. 2 is a cross-sectional view of the display apparatus DD according to one or more embodiments. FIG. 2 is a cross-sectional view illustrating a part taken along line I-I′ of FIG. 1.

The display apparatus DD may include a display panel DP and an optical layer PP disposed on the display panel DP. The display panel DP may include light emitting elements ED-1, ED-2, and ED-3. The display apparatus DD may include a plurality of light emitting elements ED-1, ED-2, and ED-3. The optical layer PP may be disposed on the display panel DP to control reflected light in the display panel DP due to external light. The optical layer PP may include, for example, a polarization layer and/or a color filter layer. In some embodiments, the optical layer PP may not be provided from the display apparatus DD.

A base substrate BL may be disposed or provided on the optical layer PP. The base substrate BL may be a member which provides a base surface on which the optical layer PP disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may not be provided.

The display apparatus DD according to one or more embodiments may further include a filling layer. The filling layer may be disposed between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display device layer DP-ED. The display device layer DP-ED may include a pixel defining film PDL, the light emitting elements ED-1, ED-2, and ED-3 disposed between respective portions of the pixel defining film PDL, and an encapsulation layer TFE disposed on the light emitting elements ED-1, ED-2, and ED-3.

The base layer BS may be a member which provides a base surface on which the display device layer DP-ED is disposed. The base layer BS may be a glass substrate, a metal substrate, a plastic substrate, etc. However, embodiments of the present disclosure are not limited thereto, and the base layer BS may be an inorganic layer, an organic layer, or a composite material layer.

In one or more embodiments, the circuit layer DP-CL may be disposed on the base layer BS, and the circuit layer DP-CL may include a plurality of transistors. Each of the transistors may include a control electrode, an input electrode, and an output electrode. For example, in some embodiments, the circuit layer DP-CL may include switching transistor(s) and driving transistor(s) for driving the light emitting elements ED-1, ED-2, and ED-3 of the display device layer DP-ED.

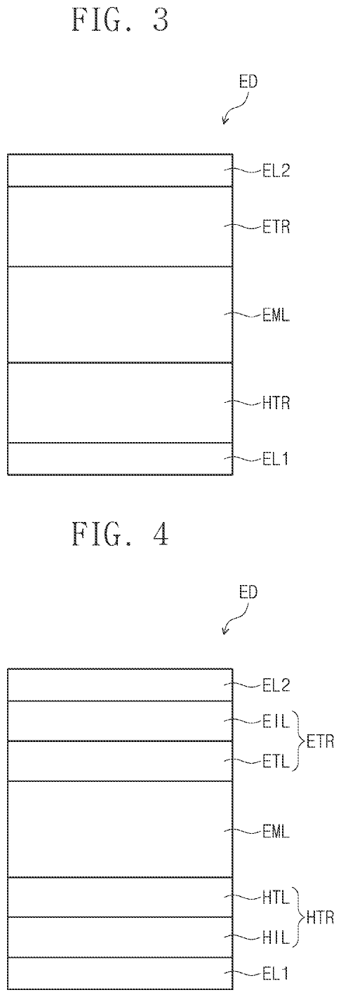

Each of the light emitting elements ED-1, ED-2, and ED-3 may have a structure of one of light emitting elements ED of embodiments according to FIGS. 3 to 6, which will be described later. Each of the light emitting elements ED-1, ED-2, and ED-3 may include a first electrode EL1, a hole transport region HTR, respective light-emitting layers EML-R, EML-G, and EML-B, an electron transport region ETR, and a second electrode EL2.

FIG. 2 illustrates an embodiment in which the respective light-emitting layers EML-R, EML-G, and EML-B of the light emitting elements ED-1, ED-2, and ED-3 are disposed in openings OH defined in the pixel defining film PDL, and the hole transport region HTR, the electron transport region ETR, and the second electrode EL2 are provided as a common layer in the entire light emitting elements ED-1, ED-2, and ED-3. However, embodiments of the present disclosure are not limited thereto, for example, in one or more embodiments, the hole transport region HTR and the electron transport region ETR may be provided by being patterned inside the openings OH defined in the pixel defining film PDL. For example, in some embodiments, the hole transport region HTR, the respective light-emitting layers EML-R, EML-G, and EML-B, and the electron transport region ETR of the light emitting elements ED-1, ED-2, and ED-3 may be provided by being patterned in an inkjet printing method.

The encapsulation layer TFE may cover the light emitting elements ED-1, ED-2, and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE may include at least one insulation layer. In some embodiments, the encapsulation layer TFE may include at least one inorganic film (hereinafter, an encapsulation-inorganic film). In some embodiments, the encapsulation layer TFE may include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. In some embodiments, the encapsulation-organic film may include an acrylic-based compound, an epoxy-based compound, and/or the like. In some embodiments, the encapsulation-organic film may include a photopolymerizable organic material, but embodiments of the present disclosure are not particularly limited thereto.

The encapsulation layer TFE may be disposed on the second electrode EL2 and may be disposed filling the opening OH.

Referring to FIG. 1 and FIG. 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced and/or apart (e.g., spaced) from each other on a plane.

Each of the light emitting regions PXA-R, PXA-G, and PXA-B may be a region divided by the pixel defining film PDL. The non-light emitting areas NPXA may be areas between adjacent light emitting areas PXA-R, PXA-G, and PXA-B, which correspond to the pixel defining film PDL. In one or more embodiments, the light emitting regions PXA-R, PXA-G, and PXA-B may respectively correspond to pixels. The pixel defining film PDL may divide the light emitting elements ED-1, ED-2, and ED-3. The respective light-emitting layers EML-R, EML-G, and EML-B of the light emitting elements ED-1, ED-2, and ED-3 may be disposed in openings OH defined in the pixel defining film PDL and separated from each other.

The light emitting regions PXA-R, PXA-G, and PXA-B may be divided into a plurality of groups according to the color of light generated from the light emitting elements ED-1, ED-2, and ED-3. In the display apparatus DD of one or more embodiments illustrated in FIG. 1 and FIG. 2, three light emitting regions PXA-R, PXA-G, and PXA-B, which emit red light, green light, and blue light, respectively, are exemplarily illustrated. For example, in one or more embodiments, the display device DD may include a red light emitting region PXA-R, a green light emitting region PXA-G, and a blue light emitting region PXA-B that are separated from each other.

In the display apparatus DD according to one or more embodiments, the plurality of light emitting elements ED-1, ED-2, and ED-3 may be to emit light beams having wavelengths different from each other. For example, in some embodiments, the display apparatus DD may include a first light emitting element ED-1 that emits red light, a second light emitting element ED-2 that emits green light, and a third light emitting element ED-3 that emits blue light. For example, in some embodiments, the red light emitting region PXA-R, the green light emitting region PXA-G, and the blue light emitting region PXA-B of the display apparatus DD may correspond to the first light emitting element ED-1, the second light emitting element ED-2, and the third light emitting element ED-3, respectively.

However, embodiments of the present disclosure are not limited thereto, and the first to third light emitting elements ED-1, ED-2, and ED-3 may be to emit light beams in substantially the same wavelength range or at least one light emitting element may be to emit a light beam in a wavelength range different from the others. For example, in some embodiments, the first to third light emitting elements ED-1, ED-2, and ED-3 may all emit blue light.

The light emitting regions PXA-R, PXA-G, and PXA-B in the display apparatus DD according to one or more embodiments may be arranged in a stripe form. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B each may be arranged with each other along the second direction axis DR2. In some embodiments, the red light emitting region PXA-R, the green light emitting region PXA-G, and the blue light emitting region PXA-B may be alternately arranged in the stated order along a first direction axis DR1.

FIG. 1 and FIG. 2 illustrate that all the light emitting regions PXA-R, PXA-G, and PXA-B have similar area, but embodiments of the present disclosure are not limited thereto. Thus, the light emitting regions PXA-R, PXA-G, and PXA-B may have different areas from each other according to the wavelength range of the emitted light. The areas of the light emitting regions PXA-R, PXA-G, and PXA-B may refer to areas when viewed on a plane defined by the first direction axis DR1 and the second direction axis DR2 (e.g., the areas in a plan view).

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display apparatus DD. For example, in one or more embodiments, the arrangement form of the light emitting regions PXA-R, PXA-G, and PXA-B may be a pentile (PENTILE®) arrangement form (for example, an RGBG matrix, an RGBG structure, or an RGBG matrix structure), or a diamond (Diamond Pixel™) arrangement form (e.g., a display (e.g., an OLED display) containing red, blue, and green (RGB) light-emitting regions arranged in the shape of diamonds). PENTILE® is a duly registered trademark of Samsung Display Co., Ltd. Diamond Pixel™ is a trademark of Samsung Display Co., Ltd.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but embodiments of the present disclosure are not limited thereto.

Hereinafter, FIG. 3 to FIG. 6 are each a cross-sectional view schematically showing light emitting elements according to one or more embodiments. The light emitting element ED according to one or more embodiments may include a first electrode EL1, a second electrode EL2 facing the first electrode EL1, and at least one functional layer between the first electrode EL1 and the second electrode EL2. The light emitting element ED of one or more embodiments may include an amine compound of one or more embodiments, which will be explained later, in the at least one functional layer.

The light emitting element ED may include a hole transport region HTR, a light-emitting layer EML, an electron transport region ETR, and/or the like, stacked in order (e.g., in the stated order), as the at least one functional layer. Referring to FIG. 3, in one or more embodiments, the light emitting element ED may include a first electrode EL1, a hole transport region HTR, a light-emitting layer EML, an electron transport region ETR, and a second electrode EL2, stacked in order (e.g., in the stated order).

The light emitting element ED according to one or more embodiments may include an amine compound of one or more embodiments, which will be explained later, in the hole transport region HTR between the first electrode EL1 and the second electrode EL2. However, embodiments of the present disclosure are not limited thereto. In addition to the hole transport region HTR, the light emitting element ED according to one or more embodiments may include an amine compound of one or more embodiments, which will be explained later, in the light-emitting layer EML or the electron transport region ETR which are a plurality of functional layer between the first electrode EL1 and the second electrode EL2, or may include an amine compound of one or more embodiments, which will be explained later, in a capping layer CPL on the second electrode EL2.

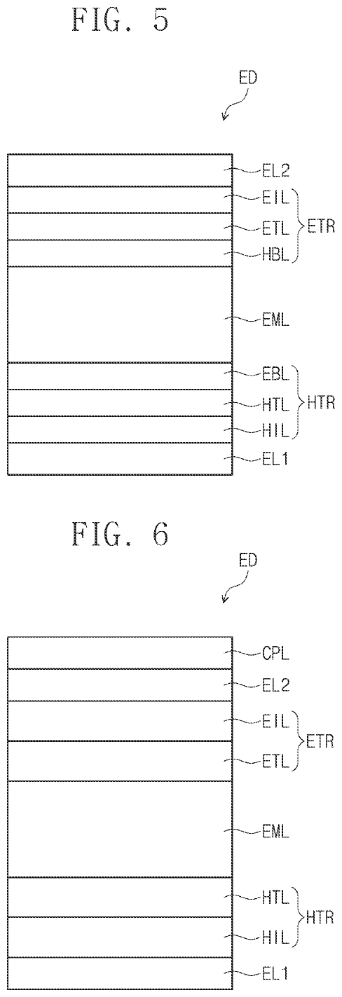

Compared with FIG. 3, FIG. 4 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. Compared with FIG. 3, FIG. 5 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of one or more embodiments including a capping layer CPL on a second electrode EL2.

The light emitting element ED according to one or more embodiments may include an amine compound of an embodiment, which will be explained later, in the hole transport region HTR. In the light emitting element ED according to one or more embodiments, at least one selected from among the hole injection layer HIL, the hole transport layer HTL, and the electron blocking layer EBL may include an amine compound of one or more embodiments. In one or more embodiments, a layer adjacent to the light-emitting layer among the plurality of layers included in the hole transport region HTR may contain an amine compound of one or more embodiments represented by Formula 1. For example, In the light emitting element ED according to one or more embodiments, the hole transport layer HTL may include an amine compound of one or more embodiments.

The first electrode EL1 has conductivity (e.g., is a conductor). The first electrode EL1 may be formed of a metal material, a metal alloy, and/or a conductive compound. The first electrode EL1 may be an anode or a cathode. However, embodiments of the present disclosure are not limited thereto. In one or more embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W), indium (In), tin (Sn), and zinc (Zn), any compound being of two or more selected therefrom, any mixture being of two or more selected therefrom among these, and/or any oxide thereof.

When the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, any compound thereof, or any mixture thereof (e.g., a mixture of Ag and Mg). In some embodiments, the first electrode EL1 may have a multilayer structure including a reflective film or a transflective film formed of one or more of the above-described materials, and a transparent conductive film formed of ITO, IZO, ZnO, ITZO, etc. For example, in some embodiments, the first electrode EL1 may have a three-layer structure of ITO/Ag/ITO, but embodiments of the present disclosure are not limited thereto. The first electrode EL1 may include one of the above-described metal materials, any combination of at least two metal materials of the above-described metal materials, any oxide of the above-described metal materials, and/or the like. A thickness of the first electrode EL1 may be from about 700 Å to about 10,000 Å. For example, in one or more embodiments, the thickness of the first electrode EL1 may be from about 1,000 Å to about 3,000 Å.

The hole transport region HTR may be provided on the first electrode EL1. The hole transport region HTR may include at least one of a hole injection layer HIL, a hole transport layer HTL, a buffer layer or an emission-auxiliary layer, or an electron blocking layer EBL. A thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, in one or more embodiments, the hole transport region HTR may have a single layer structure of a hole injection layer HIL or a hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order (e.g., in the stated order) from the first electrode EL1, but embodiments of the present disclosure are not limited thereto.

The hole transport region HTR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, The light-emitting element ED may contain an amine compound according to one or more embodiments in a hole transport region HTR. In the light-emitting element ED according to one or more embodiments, the hole transport region HTR may include a hole injection layer HIL and a hole transport layer HTL, and the hole transport layer HTL may contain the amine compound according to one or more embodiments. The amine compound according to one or more embodiments may be contained in an adjacent layer to a light-emitting layer EML among the layers included in the hole transport region HTR.

The amine compound according to one or more embodiments includes a structure in which two amine groups are connected via a linker. The amine compound according to one or more embodiments includes a first amine group, a second amine group, and a first linker connecting the first amine group and the second amine group. In one or more embodiments, the first linker may be a substituted or unsubstituted arylene group having 6 to 30 ring-forming carbon atoms.

The amine compound according to one or more embodiments may include a first substituent connected to the first amine group. The first substituent may include a naphthyl moiety and a first sub-substituent connected to the naphthyl moiety. The naphthyl moiety included in the first substituent may be connected to the first amine group. One among carbon atoms forming the naphthyl moiety may be connected to the nitrogen atom of the first amine group, and one among the remaining carbon atoms may be connected to the first sub-substituent. The first sub-substituent is directly connected to the naphthyl moiety. In one or more embodiments, the first sub-substituent may be a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to ring-forming carbon atoms. For example, in some embodiments, the first sub-substituent may be a substituted or unsubstituted phenyl group.

As utilized herein, the first substituent may be represented by Formula S1.

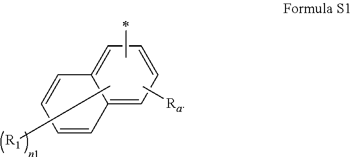

In Formula S1, Rₐ may correspond to the above-described first sub-substituent. Rₐ may be a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to ring-forming carbon atoms. In one or more embodiments, Rₐ may be a substituted or unsubstituted aryl group having 6 to 30 ring-forming carbon atoms or a substituted or unsubstituted heteroaryl group having 2 to 30 ring-forming carbon atoms.

In Formula S1, R₁ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₁ may be hydrogen.

In Formula S1, n1 is an integer of 0 to 6. In Formula 1, when n1 is 0, the amine compound according to one or more embodiments may be unsubstituted with R₁. In Formula 1, an embodiment in which n1 is 6 and R₁'s are all hydrogen may be the same as the embodiment in which n1 is 0. When n1 is an integer of 2 or more, R₁'s provided in plurality may be all the same or at least one selected from among the plurality of R₁'s may be different.

In Formula S1, [symbol] may be a position at which the above-described nitrogen atom of the first amine group is connected.

The amine compound according to one or more embodiments has a structure, as a basic structure, in which the first amine group and the second amine group are connected via the first linker, and the first linker may be substituted or unsubstituted phenylene, substituted or unsubstituted biphenylene, substituted or unsubstituted terphenylene, or substituted or unsubstituted quarterphenylene. However, an embodiment in which the first linker is substituted or unsubstituted p,p-biphenylene may be excluded due to steric reasons. For example, when the first linker is substituted or unsubstituted biphenylene, the first linker may be, for example, a substituted or unsubstituted m,p-biphenylene, a substituted or unsubstituted m,m-biphenylene, a substituted or unsubstituted o,p-biphenylene, a substituted or unsubstituted o,o-biphenylene, or a substituted or unsubstituted o,m-biphenylene.

As utilized herein, m,p-biphenylene may be represented by Formula B1-a.

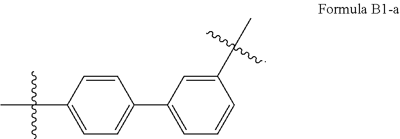 [SMILES: *c1ccc(-c2cccc(*)c2)cc1]

As utilized herein, m-m-biphenylene may be represented by Formula B1-b.

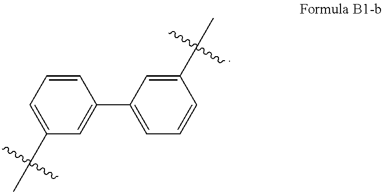 [SMILES: *c1cccc(-c2cccc(*)c2)c1]

As utilized herein, o,p biphenylene may be represented by Formula B11-c.

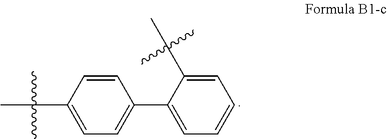 [SMILES: *c1ccc(-c2ccccc2*)cc1]

As utilized herein, o,o biphenylene may be represented by Formula B11-d.

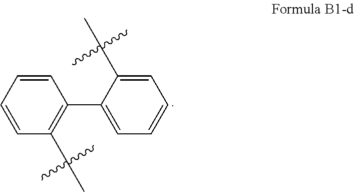 [SMILES: *c1ccccc1-c1ccccc1*]

As utilized herein, o,m biphenylene may be represented by Formula B11-e.

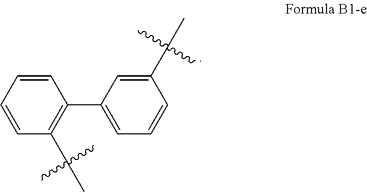 [SMILES: *c1cccc(-c2ccccc2*)c1]

As utilized herein, p,p biphenylene may be represented by Formula B1-f.

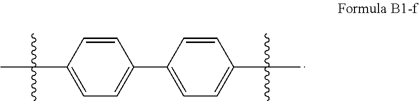 [SMILES: *c1ccc(-c2ccc(*)cc2)cc1]

In one or more embodiments, the nitrogen atom of the first amine group and the first sub-substituent, each connected to the naphthyl moiety of the first substituent, may be connected to the naphthyl moiety so as to be in an ortho-position relation. For example, in some embodiments, the first sub-substituent may be connected at a first carbon position of the naphthyl moiety, and the nitrogen atom of the first amine group may be connected to the second carbon position of the naphthyl moiety. In some embodiments, the nitrogen atom of the first amine group may be connected at the second or third carbon position of the naphthyl moiety, and the first sub-substituent may be connected to the remaining carbon atom selected from among the second and third carbon of the naphthyl moiety. However, embodiments of the present disclosure are not limited thereto. As utilized herein, carbons of the naphthyl moiety are numbered as represented in Formula N1. Meanwhile, for convenience of explanation, it is shown that the carbon number at a position where the two benzene rings are fused in Formula N1 is omitted.

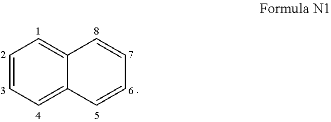

In one or more embodiments, the naphthyl moiety represented by Formula S1 may be represented by Formula S1-a or S1-b.

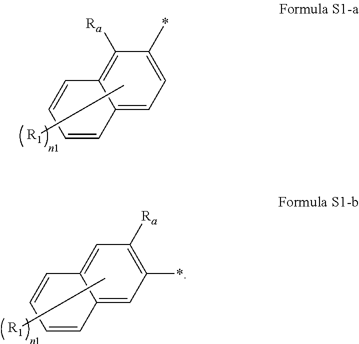

In Formulas S1-a and S1-b, the same descriptions as in Formula S1 may be applied to Rₐ, R₁ and n1.

In Formulas S1-a and S1-b, [symbol] may be a position at which the nitrogen atom of the first amine group is connected.

The amine compound according to one or more embodiments may be a diamine compound containing two amine groups. The amine compound according to one or more embodiments may be a diamine compound in which only two (exactly two) amine groups exist without forming a ring in a molecular structure.

In one or more embodiments, the amine compound may be represented by Formula 1.

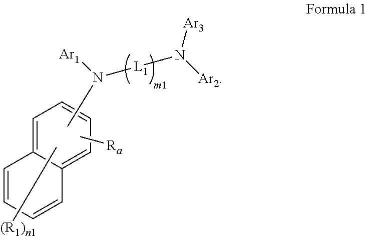

In Formula 1, the amine group to which Ar₁ is connected may correspond to the above-described first amine group, and the amine group to which Ar₂ and Ar₃ are connected may correspond to the above-described second amine group. In Formula 1, L₁ may correspond to the above-described first linker. The naphthyl group substituted with a substituent represented by R₁ in Formula 1 may correspond to the above-described naphthyl moiety, and a substituent represented by Rₐ may correspond to the above-described first sub-substituent.

In Formula 1, Ar₁ to Ar₃ may each independently be a substituted or unsubstituted oxy group, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms.

In one or more embodiments, Ar₁ may be a substituted or unsubstituted phenyl group, a substituted or unsubstituted biphenyl group, a substituted or unsubstituted cyclohexylphenyl group, or a substituted or unsubstituted naphthyl group. Ar₂ and Ar₃ may each independently be a substituted or unsubstituted phenyl group, a substituted or unsubstituted biphenyl group, a substituted or unsubstituted cyclohexylphenyl group, a substituted or unsubstituted naphthyl group, a substituted or unsubstituted fluorene group, or a substituted or unsubstituted dibenzofuran group.

In one or more embodiments, Ar₁ to Ar₃ may each independently be selected from Substituent Group A, which will be described later.

In the amine compound represented by Formula 1, according to some embodiments, an embodiment in which Ar₁ includes a fluorene group, a dibenzofuran group, or a dibenzothiophene group is excluded. In some embodiments, an embodiment in which Ar₁ is a fluorene group or a heteroaryl group having 2 to 30 ring-forming carbon atoms such as a dibenzofuran group or a dibenzothiophene group may be excluded. For example, in the amine compound represented by Formula 1, according to some embodiments, in addition to the first substituent, a substituent connected to the first amine group may include no fluorene group, dibenzofuran group, or dibenzothiophene group (e.g., may exclude a fluorene group, a dibenzofuran group, or a dibenzothiophene group). When Ar₁ in Formula 1 includes a fluorene group, a dibenzofuran group, or a dibenzothiophene group, a distance between molecules increases, and thus π-π stacking decreases. Therefore, hole transport ability may decrease, and hole mobility may be reduced. According to the present disclosure, because the embodiment in which Ar₁ included in the amine compound includes a fluorene group, a dibenzofuran group, or a dibenzothiophene group is excluded, hole transport characteristics may be improved. Therefore, improved luminous efficiency and element lifespan characteristics may be exhibited. In Formula 1, L₁ may be a substituted or unsubstituted phenylene group.

In Formula 1, Rₐ may be a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms. For example, in some embodiments, Rₐ may be a substituted or unsubstituted phenyl group.

In Formula 1, R₁ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₁ may be hydrogen.

In Formula 1, n1 is an integer of 0 to 6. In Formula 1, when n1 is 0, the amine compound according to one or more embodiments may be unsubstituted with R₁. An embodiment in which n1 is 6 and all R₁'s are hydrogens in Formula 1 may be the same as the embodiment where n1 is 0 in Formula 1. When n1 is an integer of 2 or more, R₁'s provided in plurality may be all the same, or at least one selected from among the plurality of R₁'s may be different.

In Formula 1, m1 is an integer of 1 to 4. For example, m1 may be 1, 3, or 4.

In the amine compound represented by Formula 1, according to one or more embodiments, when m1 is 2, an embodiment in which L₁ is represented by Formula A-1 is excluded. In the amine compound represented by Formula 1, according to one or more embodiments, an embodiment in which the first linker connecting the first amine group and the second amine group is p,p-biphenylene represented by Formula A-1 may be excluded. The linker represented by Formula A-1 have a structure in which two benzene rings are connected to the first amine group and the second amine group so as to be in a para-position relation, thus planarity within the molecule may increase, and may cause charge mobility to be lowered. According to the present disclosure, in the amine compound represented by Formula 1, according to one or more embodiments, because an embodiment in which the first linker connecting the first amine group and the second amine group is represented by Formula A-1 is excluded, the chare mobility may be improved. Therefore, luminous efficiency and element lifespan characteristics may be improved.

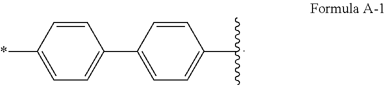

In Formula A-1, [symbol] is a position at which the nitrogen atom to which Ar₁ is connected in Formula 1 is connected, and

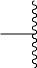

is a position at which the nitrogen atom to which Ar₂ and Ar₃ are connected in Formula 1 is connected.

In one or more embodiments, the amine compound represented by Formula may be represented by Formula 2.

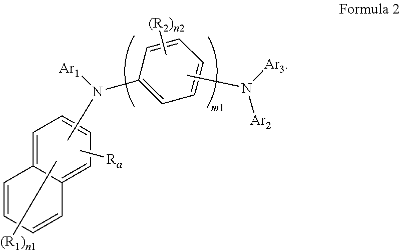

In Formula 2, R₂ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₂ may be hydrogen.

In Formula 2, n2 may be an integer of 0 to 4. In Formula 2, when n2 is 0, the amine compound according to one or more embodiments may be unsubstantiated with R₂. An embodiment in which n2 is 4 and all R₂'s are hydrogens in Formula 2 may be the same as the embodiment in which n2 is 0 in Formula 2. When n2 is an integer of 2 or more, R₂'s provided in plurality may be all the same, or at least one selected from among the plurality of R₂'s may be different.

In Formula 2, the same descriptions as in Formula 1 may be applied to Ar₁ to Ar₃, Rₐ, R₁, n1, and m1.

In one or more embodiments, the amine compound represented by Formula may be represented by Formula 3.

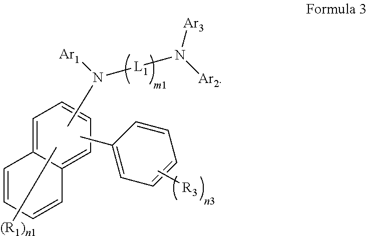

Formula 3 represents an embodiment in which a type or kind of Rₐ is specified in Formula 1. Formula 3 may correspond to an embodiment in which Rₐ is a substituted or unsubstituted phenyl group in Formula 1.

In Formula 3, R₃ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₃ may be hydrogen.

In Formula 3, n3 may be an integer of 0 to 5. In Formula 3, when n3 is 0, the amine compound according to one or more embodiments may be unsubstantiated with R₃. An embodiment in which n3 is 5 and all R₃'s are hydrogens in Formula 3 may be the same as the embodiment in which n3 is 0 in Formula 3. When n3 is an integer of 2 or more, R₃'s provided in plurality may be all the same, or at least one selected from among the plurality of R₃'s may be different.

In Formula 3, the same descriptions as in Formula 1 may be applied to Ar₁ to Ar₃, L₁, R₁, n1 and m1.

In one or more embodiments, the amine compound represented by Formula may be represented by Formula 4-1 or Formula 4-2.

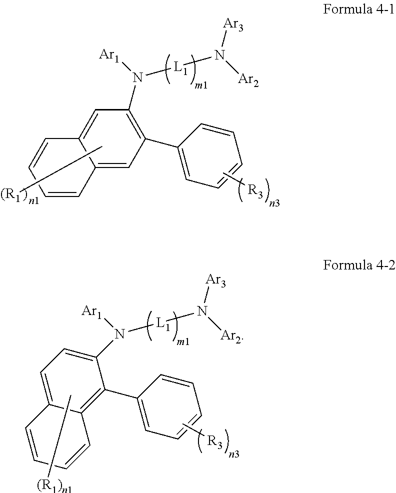

Formulas 4-1 and 4-2 represent embodiments in which substitution positions of the first amine group and Rₐ, each connected to the naphthyl moiety, are specified. In addition, Formulas 4-1 and 4-2 represent embodiments in which Rₐ is a substituted or unsubstantiated phenyl group in Formula 1.

In Formulas 4-1 and 4-2, the same descriptions as in Formulas 1 and 3 may be applied to Ar₁ to Ar₃, L₁, R₁, R₃, n1, n3, and m1.

In one or more embodiments, the amine compound represented by Formula may be represented by any one selected from among Formulas 5-1 to 5-5.

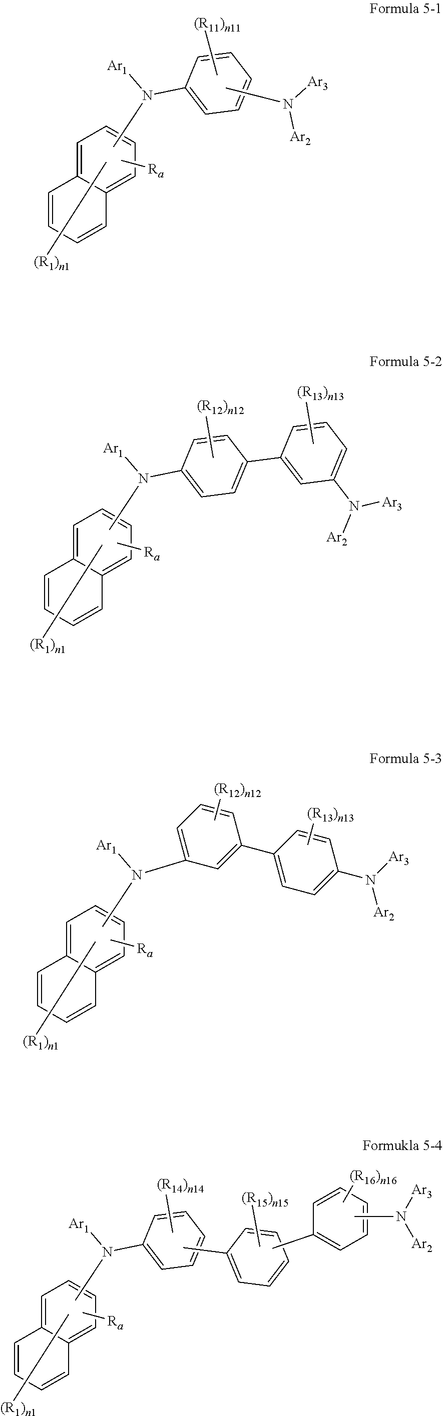

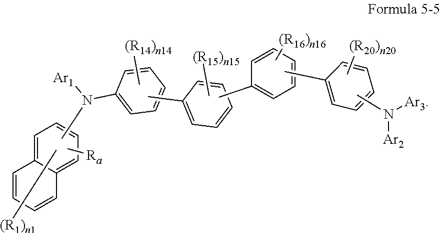

Formulas 5-1 to 5-5 represent embodiments in which types (kinds) of L₁ and the number of m1's are specified in Formula 1.

In Formulas 5-1 to 5-5, R₁₁ to R₂₀ may each independently be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₁₁ to R₂₀ may each independently be hydrogen.

In Formulas 5-1 to 5-5, n11 to n20 may each independently be an integer of to 4. In Formulas 5-1 to 5-5, when n11 to n20 are each 0, the amine compounds according to one or more embodiments may be unsubstituted with R₁₁ to R₂₀, respectively. Embodiments in which n11 to n20 are each 4 and all R₁₁'s to R₂₀'s are each hydrogen in Formulas 5-1 to 5-5 may be the same as embodiments in which n11 to n20 are each 0 in Formulas 5-1 to 5-5, respectively. When n11 to n20 each are an integer of 2 or more, R₁₁'s to R₂₀'s each provided in plurality may be all the same or at least one selected from among each of the plurality of R₁₁'s to R₂₀'s may be different.

In Formulas 5-1 to 5-5, the same descriptions as in Formula 1 may be applied to Ar₁ to Ar₃, Rₐ, R₁, and n1.

In one or more embodiments, L₁ may be represented by any one selected from among Formulas L-1 to L-22.

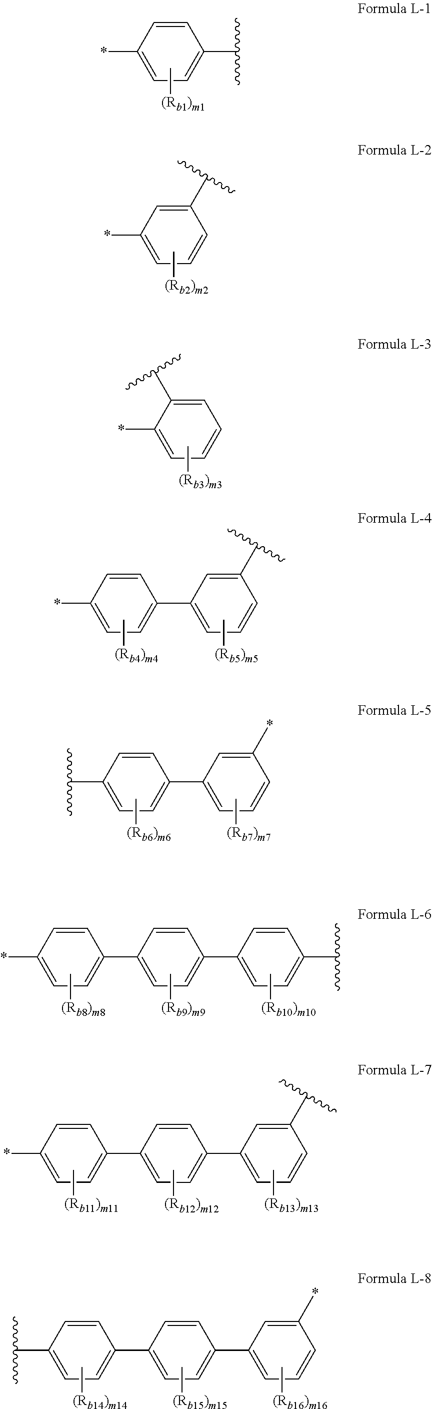

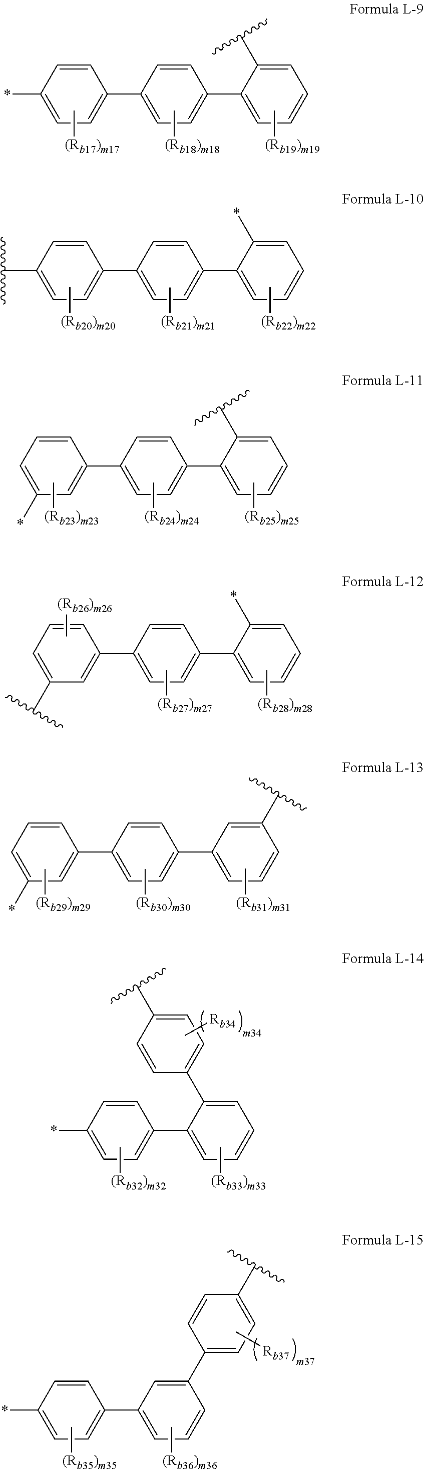

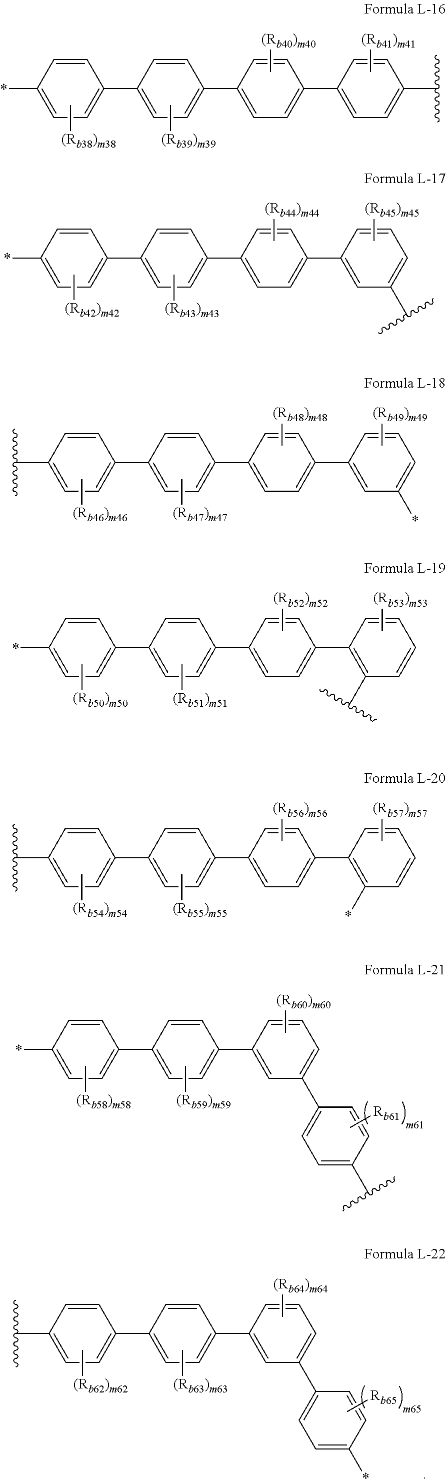

In Formulas L-1 to L-22, R_{b1} to R_{b65} may each independently be hydrogen, deuterium, a halogen, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R_{b1} to R_{b65} may each independently be hydrogen.

In Formulas L-1 to L-22, m1 to m65 may each independently be an integer of to 4. In Formulas L-1 to L-22, when m1 to m65 are each 0, the amine compounds according to one or more embodiments may be unsubstituted with R_{b1} to R_{b65}, respectively. Embodiments in which m1 to m65 are each 4 and all R_{b1} to R_{b65} are each hydrogen in Formulas L-1 to L-22 may be the same as embodiments in which m1 to m65 are each 0 in Formulas L-1 to L-22. When m1 to m65 are each an integer of 2 or more, each of R_{b1}'s to R_{b65}'s provided in plurality may be all the same, or at least one selected from among each of the plurality of R_{b1}'s to R_{b65}'s may be different.

In one or more embodiments, Ar₁ may be a substituted or unsubstituted phenyl group, a substituted or unsubstituted biphenyl group, or a substituted or unsubstituted naphthyl group, and Ar₂ and Ar₃ may each independently be a substituted or unsubstituted phenyl group, a substituted or unsubstituted biphenyl group, a substituted or unsubstituted naphthyl group, or a substituted or unsubstituted fluorenyl group.

In one or more embodiments, Ar₁ may be represented by any one selected from among Formulas B-1 to B-4, and Ar₂ and Ar₃ may each independently be represented by any one selected from among Formulas B-1 to B-7.

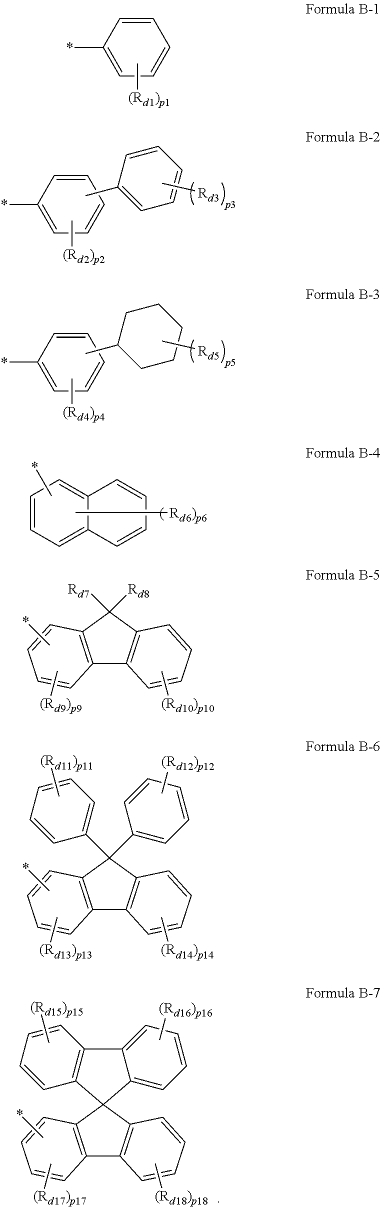

In Formulas B-1 to B-7, R_{d1} to R_{d18} may each independently be hydrogen, deuterium, a halogen, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R_{d1} to R_{d18} may each independently be hydrogen.

In Formulas B-1 to B-7, p1, p3, p11, and p12 may each independently be an integer of 0 to 5, p2, p4, p10, p14, p15, p16, and p18 may each independently be an integer of 0 to 4, p5 is an integer of 0 to 11, p6 is an integer of 0 to 7, and p9, p13, and p17 may each independently be an integer of 0 to 3.

When p1, p3, p11, and p12 are each 0, the amine compounds according to one or more embodiments may be unsubstituted with R_{d1}, R_{d3}, R_{d11}, and R_{d12}, respectively. Embodiments in which p1, p3, p11, and p12 are each 5 and R_{d1}, R_{d3}, R_{d11}, and R_{d12} are each hydrogen may be the same as the embodiments in which p1, p3, p11, and p12 are each 0.

When p2, p4, p10, p14, p15, p16 and p18 are each 0, the amine compounds according to one or more embodiments may be unsubstituted with R_{d2}, R_{d4}, R_{d10}, R_{d14}, R_{d15}, R_{d16}, and R_{d18}, respectively. Embodiments in which p2, p4, p10, p15, p16, and p18 are each 4 and R_{d2}, R_{d4}, R_{d10}, R_{d15}, R_{d16}, and R_{d18} are each hydrogen may be the same as the embodiments in which p2, p4, p10, p14, p15, p16, and p18 are each 0.

When p5 is 0, the amine compound according to one or more embodiments may be unsubstituted with R_{d5}. An embodiment in which p5 is 11 and all R_{d5}'s are each hydrogen may be the same as the embodiment in which p5 is 0. When p6 is 0, the amine compound according to one or more embodiments may be unsubstituted with R_{d6}. An embodiment in which p6 is 7 and R_{d6}'s are each hydrogen may be the same as the embodiment in which p6 is 0.

When p9, p13, and p17 are each 0, the amine compounds according to one or more embodiments may be unsubstituted with R_{d9}, R_{d13}, and R_{d17}, respectively. Embodiments in which p9, p13, and p17 are each 3 and R_{d9}, R_{d13}, and R_{d17} are each hydrogen may be the same as the embodiments in which p9, p13, and p17 are each 0.

When p1 to p6, and p9 to p18 are each an integer of 2 or more, each of R_{d1}'s to R_{d6}'s, and R_{d9}'s to R_{d18}'s provided in plurality may be the same, or at least one of the plurality of R_{d1}'s to R_{d6}'s and R_{d9}'s to R_{d18}'s may be different.

In one or more embodiments, the amine compound represented by Formula may be represented by Formula 6-1 or Formula 6-2.

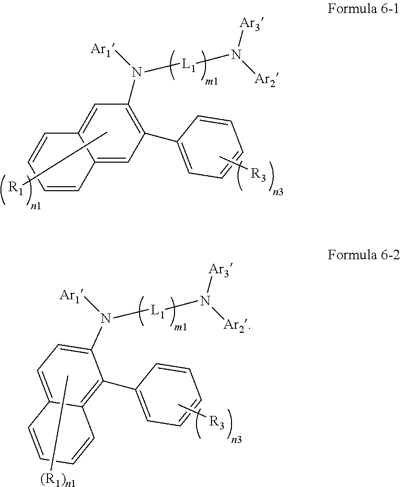

In Formulas 6-1 and 6-2, R₃ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₃ may be hydrogen.

In Formulas 6-1 and 6-2, n3 is an integer of 0 to 5. In Formulas 6-1 and 6-2, when n3 is 0, the amine compound according to one or more embodiments may be unsubstituted with R₃. An embodiment in which n3 is 5 and all R₃'s are each hydrogen in Formulas 6-1 and 6-2, may be the same as the embodiment in which n3 is 0 in Formulas 6-1 and 6-2. When n3 is an integer of 2 or more, R₃'s provided in plurality may be all the same or at least one selected from among the plurality of R₃'s may be different.

In Formulas 6-1 and 6-2, the same descriptions as in Formula 1 may be applied to L₁, R₁, n1, and m1.

In Formulas 6-1 and 6-2, Ar₁′ to Ar₃′ may each independently be selected from Substituent Group A, provided that Ar₁′ may not be any one selected from among a36 to a47.

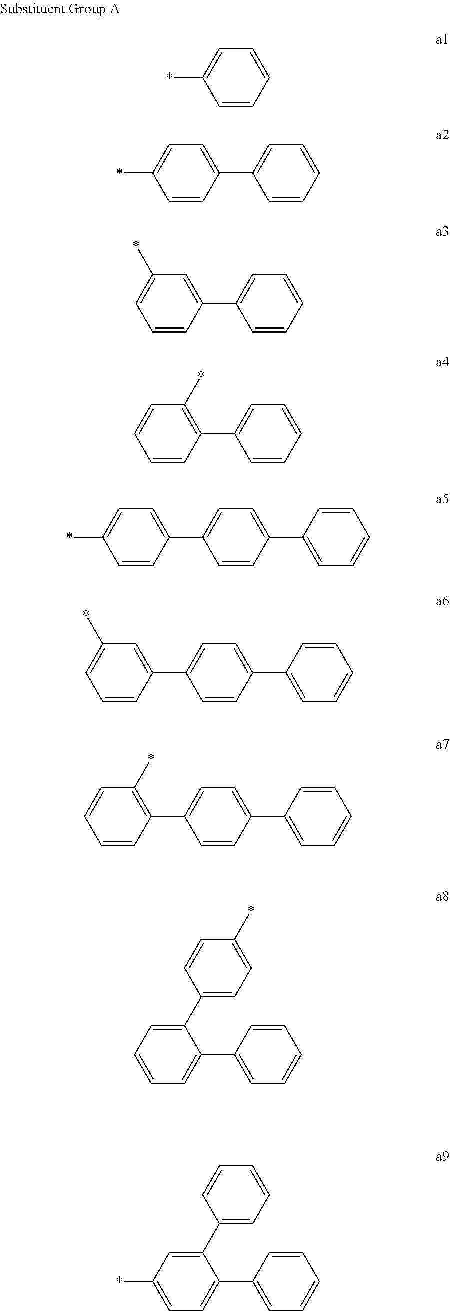

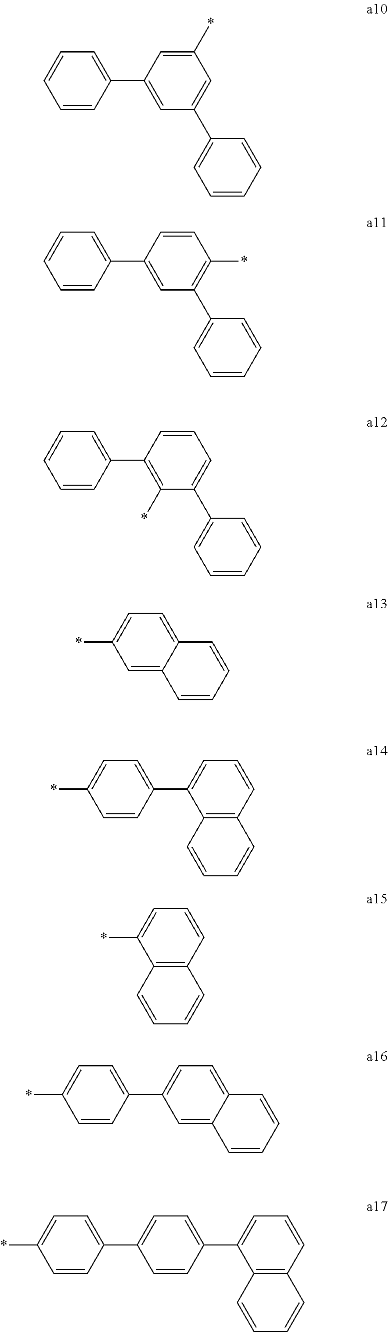

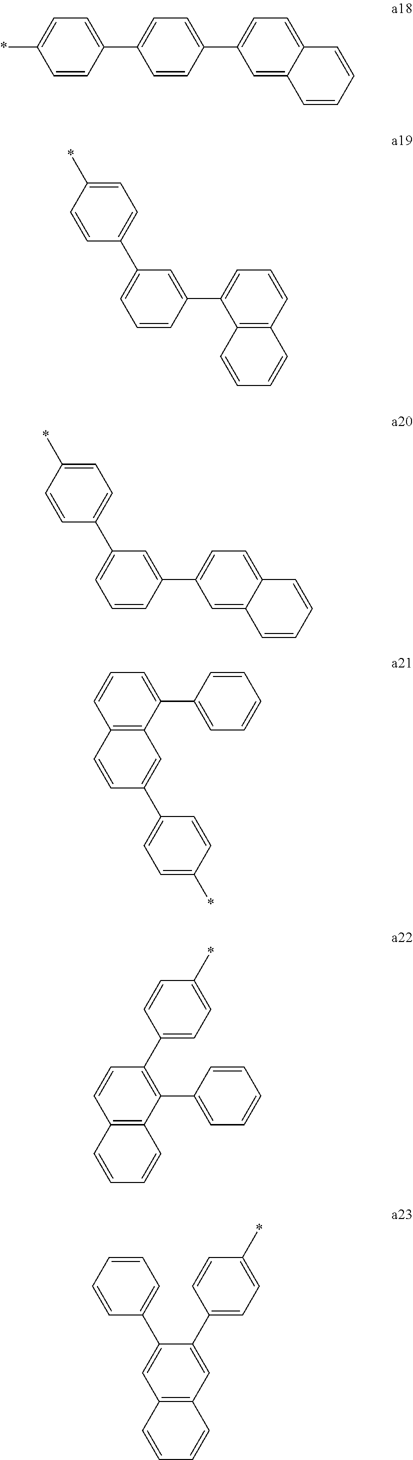

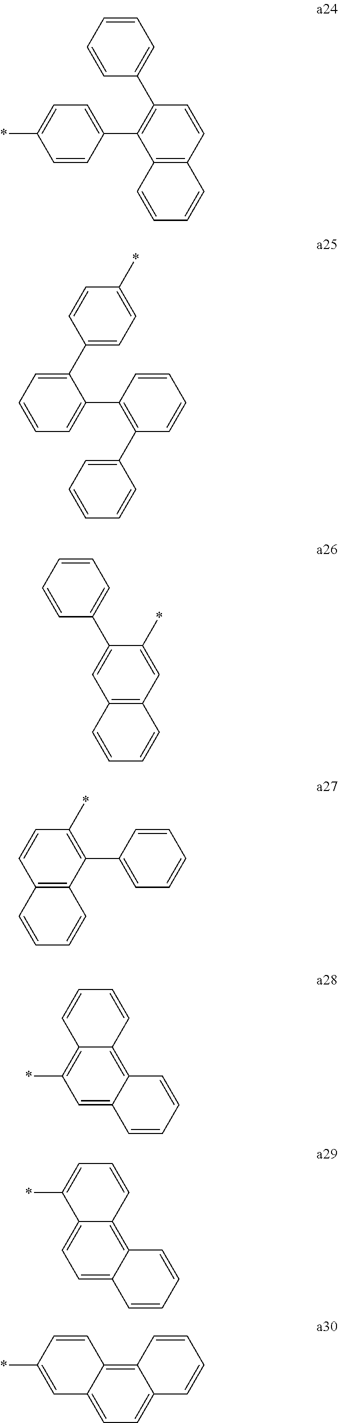

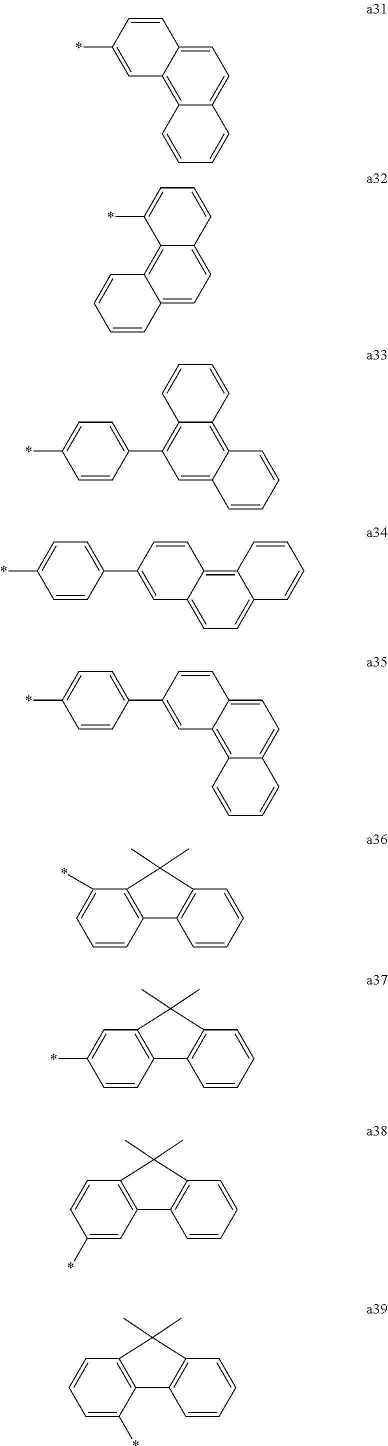

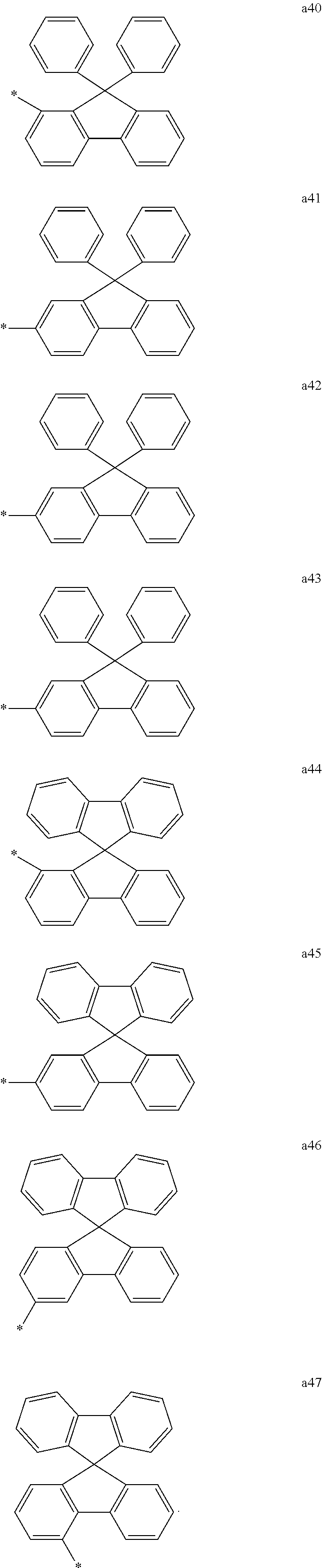

In one or more embodiments, the amine compound represented by Formula may be represented by Formula 7.

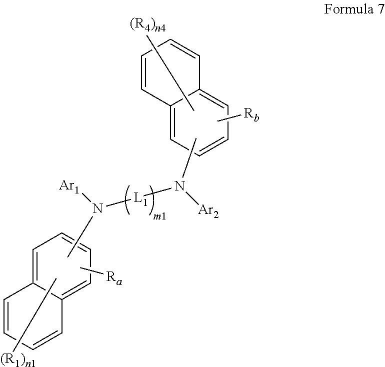

In Formula 7, R₄ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₄ may be hydrogen.

In Formula 7, n4 is an integer of 0 to 6. In Formula 7, when n4 is 0, the amine compound according to one or more embodiments may be unsubstituted with R₄. An embodiment in which n4 is 6 and all R₄'s are hydrogens in Formula 7 may be the same as the embodiment in which n4 is 0 in Formula 7. When n4 is an integer of 2 or more, R₄'s provided in plurality may be all the same, or at least one selected from among the plurality of R₄'s may be different.

In Formula 7, Rb may be a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms. For example, in some embodiments, Rb may be a substituted or unsubstituted phenyl group.

In Formula 7, the same descriptions as in Formula 1 may be applied to Ar₁, Ar₂, Rₐ, R₁, n1, L₁ and m1.

In one or more embodiments, the amine compound represented by Formula may be represented by Formula 8-1 or Formula 8-2.

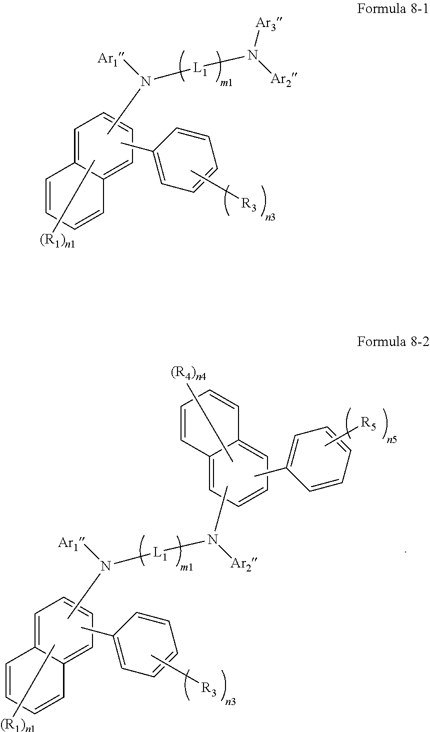

In Formula 8-2, R₅ may be hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in some embodiments, R₅ may be hydrogen.

In Formula 8-2, n5 is an integer of 0 to 5. In Formula 8-2, when n5 is 0, the amine compound according to one or more embodiments may be unsubstituted with R₅. An embodiment in which n5 is 5 and all R₅'s are hydrogens in Formula 8-2 may be the same as the embodiment in which n5 is 0 in Formula 8-2. When n5 is an integer of or more, R₅'s provided in plurality are all the same or at least one selected from among the plurality of R₅'s may be different.

In Formulas 8-1 and 8-2, Ar₁″ may be a substituted or unsubstituted aryl group having 6 to 30 ring-forming carbon atoms.

In Formulas 8-1 and 8-2, Ar₂″ and Ar₃″ may each independently be a substituted or unsubstituted aryl group having 6 to 30 ring-forming carbon atoms or a substituted or unsubstituted heteroaryl group having 2 to 30 ring-forming carbon atoms.

In one or more embodiments, Ar₁″ may be a substituted or unsubstituted phenyl group, a substituted or unsubstituted biphenyl group, a substituted or unsubstituted cyclohexylphenyl group, or a substituted or unsubstituted naphthyl group.

In one or more embodiments, Ar₂″ and Ar₃″ may each independently be a substituted or unsubstituted phenyl group, a substituted or unsubstituted biphenyl group, a substituted or unsubstituted cyclohexylphenyl group, a substituted or unsubstituted naphthyl group, a substituted or unsubstituted fluorene group, or a substituted or unsubstituted dibenzofuran group.

In one or more embodiments, Ar₁″, Ar₂″, and Ar₃″ may each independently be represented by any one selected from among Formulas B-1 to B-7, provided that Ar₁″ may not be represented by any one selected from among Formulas B-5 to B-7.

In one or more embodiments, Ar₁″, Ar₂″, and Ar₃″ may each independently be selected from Substituent Group A, provided that Ar₁″ may not be any one selected from among a36 to a47.

In one or more embodiments, at least one selected from among Ar₁″, Ar₂″, and Ar₃″ may be a substituted or unsubstituted aryl group having 6 to 15 carbon atoms. In some embodiments, at least one selected from among Ar₁″, Ar₂″, and Ar₃″ may be a substituted or unsubstituted phenyl group, a substituted or unsubstituted biphenyl group, a substituted or unsubstituted naphthyl group, or a substituted or unsubstituted fluorene group. However, embodiments of the present disclosure are not limited thereto.

In Formulas 8-1 and 8-2, the same descriptions as in Formula 1 may be applied to Rₐ, R₁, n1, L₁ and m1.

In Formulas 8-1 and 8-2, the same descriptions as in Formula 3 may be applied to R₃ and n3.

In Formulas 8-1 and 8-2, the same descriptions as in Formula 7 may be applied to R₄ and n4.

The amine compound according to one or more embodiments may be represented by one selected from among compounds in Compound Group 1. The hole transport region HTR of the light-emitting element ED according to one or more embodiments may contain at least one selected from among amine compounds disclosed in Compound Group 1. For example, in one or more embodiments, the hole transport layer HTL of the light-emitting element ED may include at least one selected from among the amine compounds disclosed in Compound Group 1.

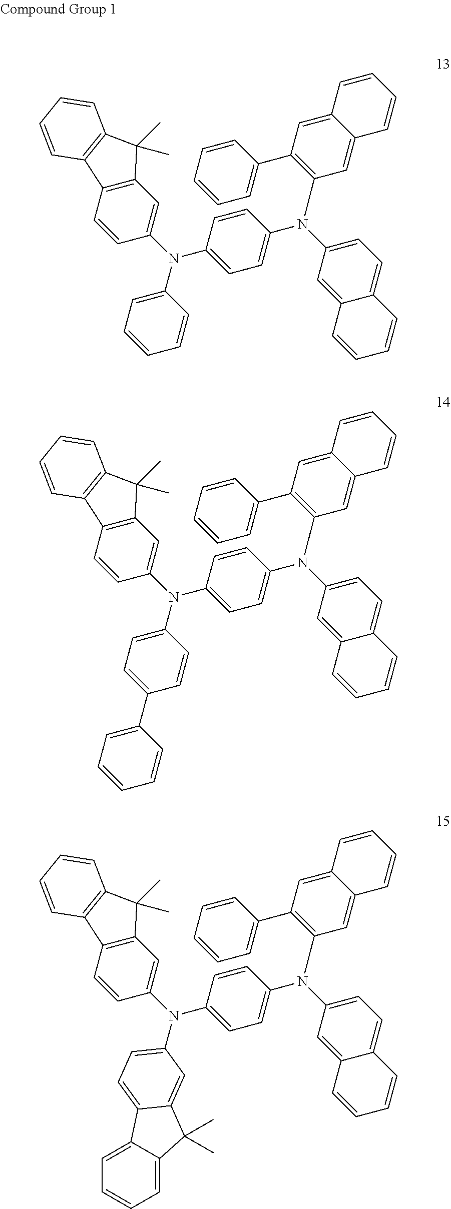 [SMILES: CC1(C)c2ccccc2-c2ccc(N(c3ccccc3)c3ccc(N(c4ccc5ccccc5c4)c4cc5ccccc5cc4-c4ccccc4)cc3)cc21 ; CC1(C)c2ccccc2-c2ccc(N(c3ccc(-c4ccccc4)cc3)c3ccc(N(c4ccc5ccccc5c4)c4cc5ccccc5cc4-c4ccccc4)cc3)cc21 ; CC1(C)c2ccccc2-c2ccc(N(c3ccc(N(c4ccc5ccccc5c4)c4cc5ccccc5cc4-c4ccccc4)cc3)c3ccc4c(c3)C(C)(C)c3ccccc3-4)cc21]

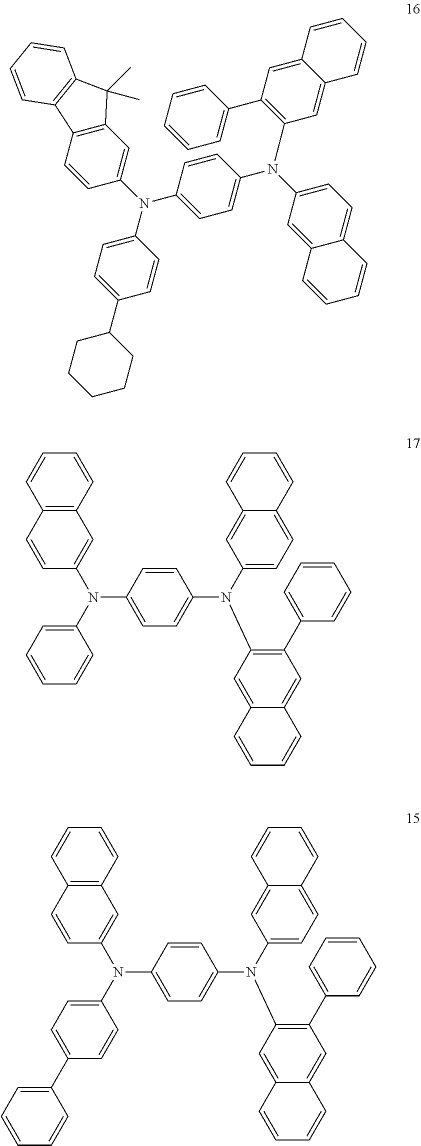 [SMILES: CC1(C)c2ccccc2-c2ccc(N(c3ccc(C4CCCCC4)cc3)c3ccc(N(c4ccc5ccccc5c4)c4cc5ccccc5cc4-c4ccccc4)cc3)cc21 ; c1ccc(-c2cc3ccccc3cc2N(c2ccc(N(c3ccccc3)c3ccc4ccccc4c3)cc2)c2ccc3ccccc3c2)cc1 ; c1ccc(-c2ccc(N(c3ccc(N(c4ccc5ccccc5c4)c4cc5ccccc5cc4-c4ccccc4)cc3)c3ccc4ccccc4c3)cc2)cc1]

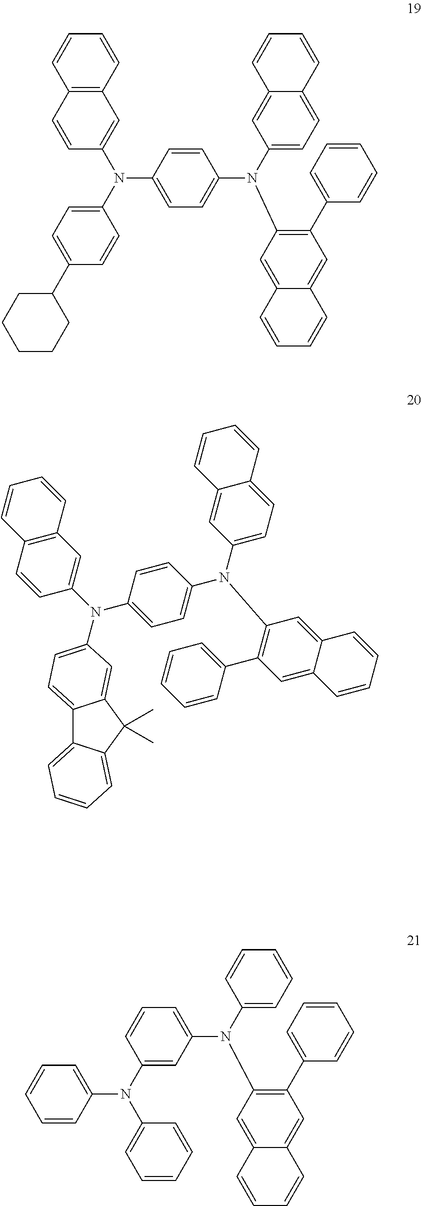 [SMILES: c1ccc(-c2cc3ccccc3cc2N(c2ccc(N(c3ccc(C4CCCCC4)cc3)c3ccc4ccccc4c3)cc2)c2ccc3ccccc3c2)cc1 ; CC1(C)c2ccccc2-c2ccc(N(c3ccc(N(c4ccc5ccccc5c4)c4cc5ccccc5cc4-c4ccccc4)cc3)c3ccc4ccccc4c3)cc21 ; c1ccc(-c2cc3ccccc3cc2N(c2ccccc2)c2cccc(N(c3ccccc3)c3ccccc3)c2)cc1]

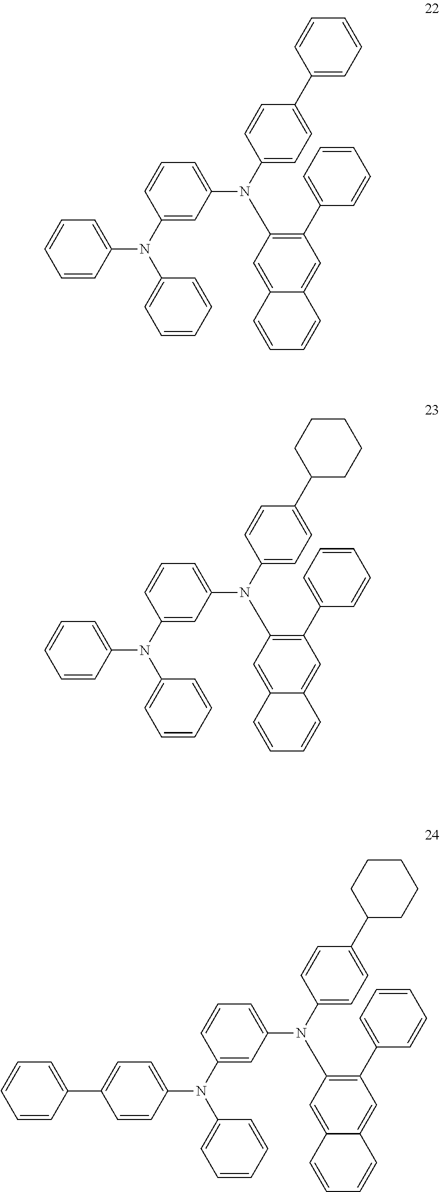 [SMILES: c1ccc(-c2ccc(N(c3cccc(N(c4ccccc4)c4ccccc4)c3)c3cc4ccccc4cc3-c3ccccc3)cc2)cc1 ; c1ccc(-c2cc3ccccc3cc2N(c2ccc(C3CCCCC3)cc2)c2cccc(N(c3ccccc3)c3ccccc3)c2)cc1 ; c1ccc(-c2ccc(N(c3ccccc3)c3cccc(N(c4ccc(C5CCCCC5)cc4)c4cc5ccccc5cc4-c4ccccc4)c3)cc2)cc1]

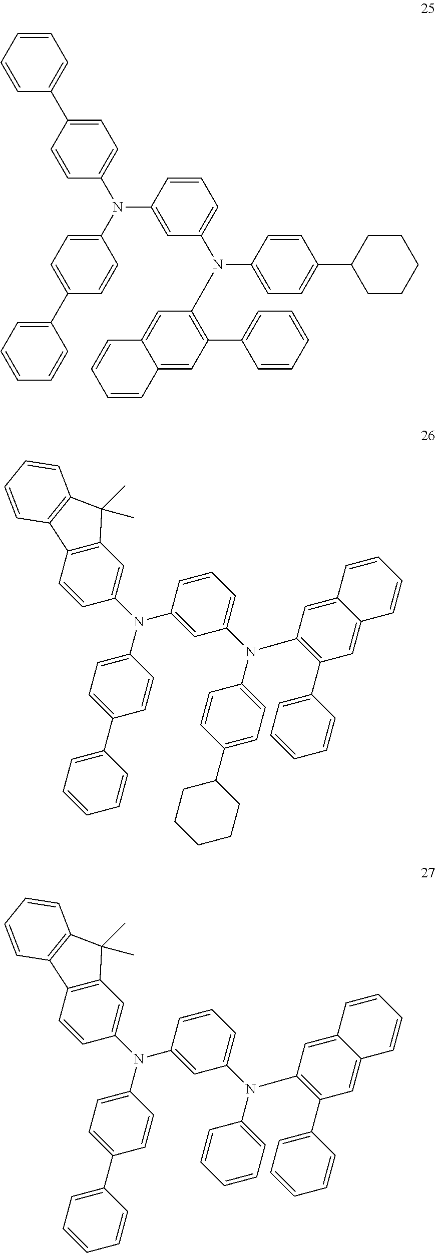 [SMILES: c1ccc(-c2ccc(N(c3ccc(-c4ccccc4)cc3)c3cccc(N(c4ccc(C5CCCCC5)cc4)c4cc5ccccc5cc4-c4ccccc4)c3)cc2)cc1 ; CC1(C)c2ccccc2-c2ccc(N(c3ccc(-c4ccccc4)cc3)c3cccc(N(c4ccc(C5CCCCC5)cc4)c4cc5ccccc5cc4-c4ccccc4)c3)cc21 ; CC1(C)c2ccccc2-c2ccc(N(c3ccc(-c4ccccc4)cc3)c3cccc(N(c4ccccc4)c4cc5ccccc5cc4-c4ccccc4)c3)cc21]

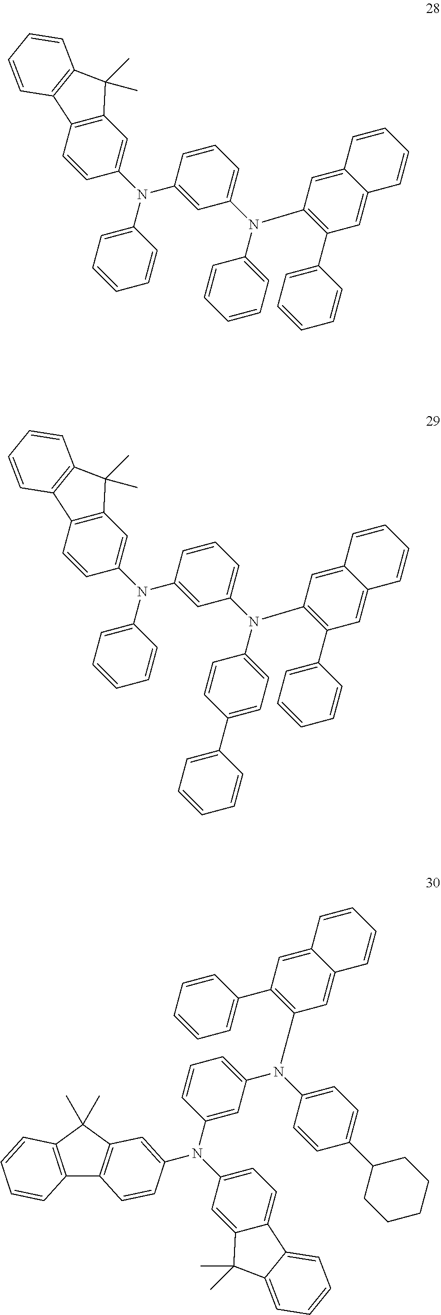 [SMILES: CC1(C)c2ccccc2-c2ccc(N(c3ccccc3)c3cccc(N(c4ccccc4)c4cc5ccccc5cc4-c4ccccc4)c3)cc21 ; CC1(C)c2ccccc2-c2ccc(N(c3ccccc3)c3cccc(N(c4ccc(-c5ccccc5)cc4)c4cc5ccccc5cc4-c4ccccc4)c3)cc21 ; CC1(C)c2ccccc2-c2ccc(N(c3cccc(N(c4ccc(C5CCCCC5)cc4)c4cc5ccccc5cc4-c4ccccc4)c3)c3ccc4c(c3)C(C)(C)c3ccccc3-4)cc21]

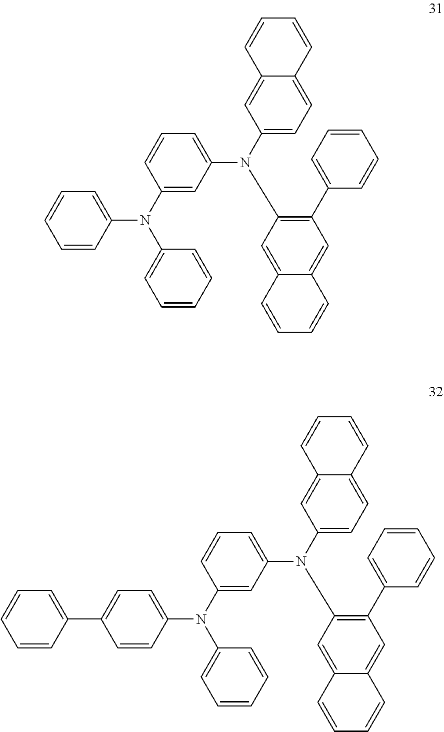 [SMILES: c1ccc(-c2cc3ccccc3cc2N(c2cccc(N(c3ccccc3)c3ccccc3)c2)c2ccc3ccccc3c2)cc1 ; c1ccc(-c2ccc(N(c3ccccc3)c3cccc(N(c4ccc5ccccc5c4)c4cc5ccccc5cc4-c4ccccc4)c3)cc2)cc1]

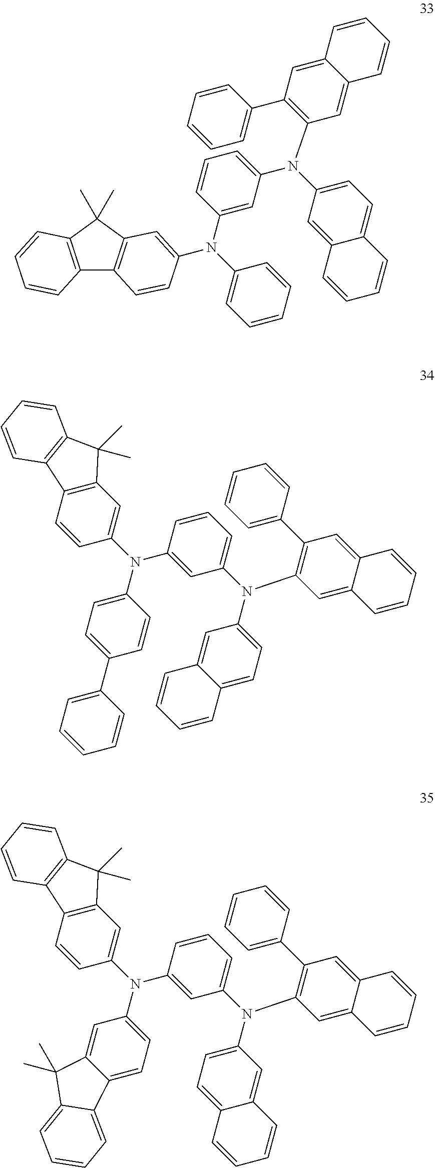 [SMILES: CC1(C)c2ccccc2-c2ccc(N(c3ccccc3)c3cccc(N(c4ccc5ccccc5c4)c4cc5ccccc5cc4-c4ccccc4)c3)cc21 ; CC1(C)c2ccccc2-c2ccc(N(c3ccc(-c4ccccc4)cc3)c3cccc(N(c4ccc5ccccc5c4)c4cc5ccccc5cc4-c4ccccc4)c3)cc21 ; CC1(C)c2ccccc2-c2ccc(N(c3cccc(N(c4ccc5ccccc5c4)c4cc5ccccc5cc4-c4ccccc4)c3)c3ccc4c(c3)C(C)(C)c3ccccc3-4)cc21]

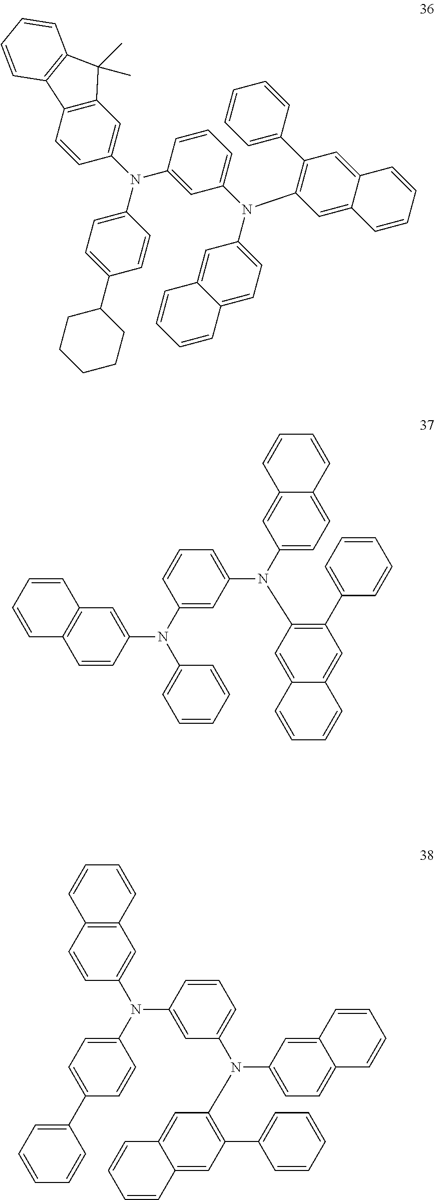 [SMILES: CC1(C)c2ccccc2-c2ccc(N(c3ccc(C4CCCCC4)cc3)c3cccc(N(c4ccc5ccccc5c4)c4cc5ccccc5cc4-c4ccccc4)c3)cc21 ; c1ccc(-c2cc3ccccc3cc2N(c2cccc(N(c3ccccc3)c3ccc4ccccc4c3)c2)c2ccc3ccccc3c2)cc1 ; c1ccc(-c2ccc(N(c3cccc(N(c4ccc5ccccc5c4)c4cc5ccccc5cc4-c4ccccc4)c3)c3ccc4ccccc4c3)cc2)cc1]

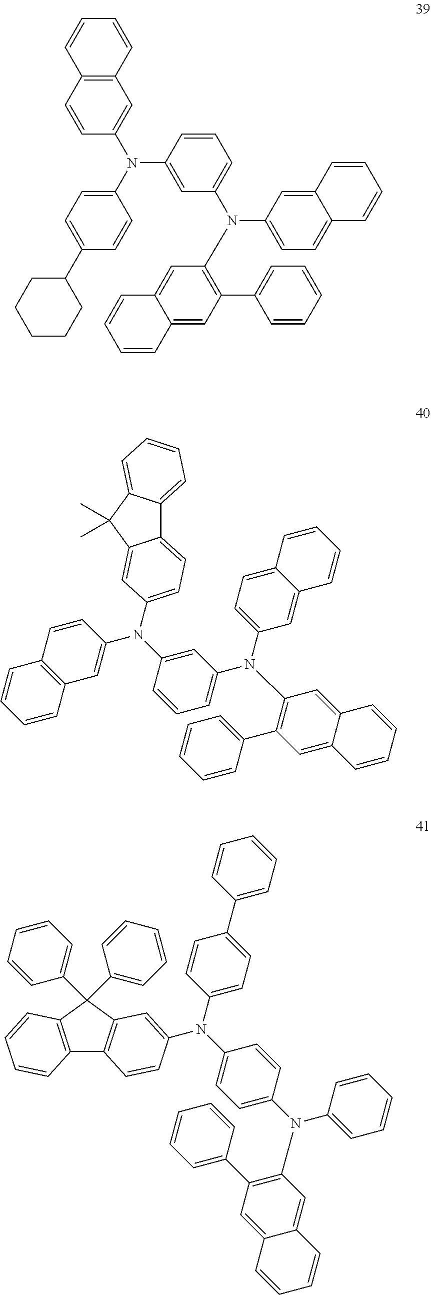 [SMILES: c1ccc(-c2cc3ccccc3cc2N(c2cccc(N(c3ccc(C4CCCCC4)cc3)c3ccc4ccccc4c3)c2)c2ccc3ccccc3c2)cc1 ; CC1(C)c2ccccc2-c2ccc(N(c3cccc(N(c4ccc5ccccc5c4)c4cc5ccccc5cc4-c4ccccc4)c3)c3ccc4ccccc4c3)cc21 ; c1ccc(-c2ccc(N(c3ccc(N(c4ccccc4)c4cc5ccccc5cc4-c4ccccc4)cc3)c3ccc4c(c3)C(c3ccccc3)(c3ccccc3)c3ccccc3-4)cc2)cc1]

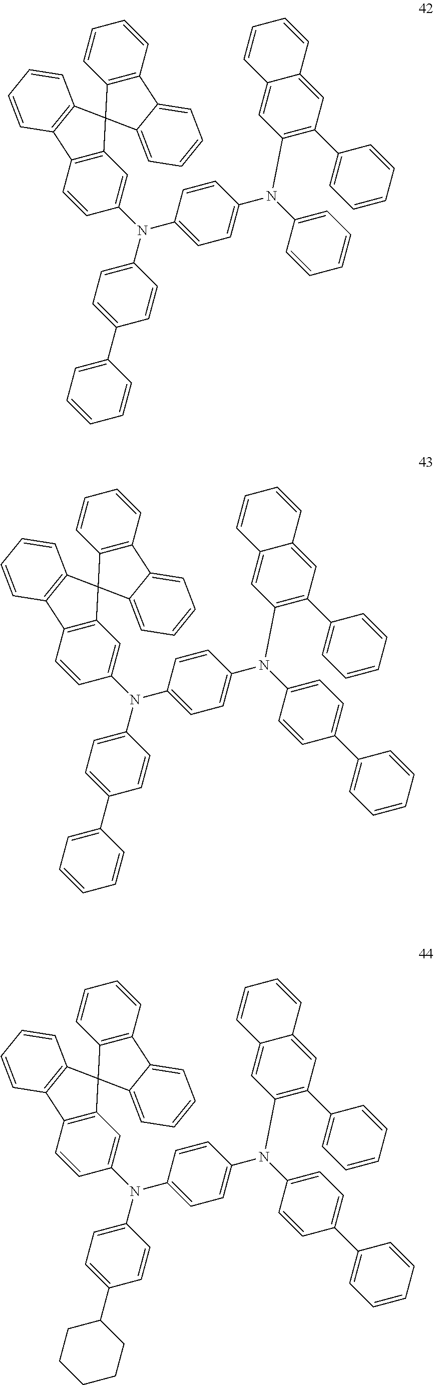 [SMILES: c1ccc(-c2ccc(N(c3ccc(N(c4ccccc4)c4cc5ccccc5cc4-c4ccccc4)cc3)c3ccc4c(c3)C3(c5ccccc5-c5ccccc53)c3ccccc3-4)cc2)cc1 ; c1ccc(-c2ccc(N(c3ccc(N(c4ccc(-c5ccccc5)cc4)c4cc5ccccc5cc4-c4ccccc4)cc3)c3ccc4c(c3)C3(c5ccccc5-c5ccccc53)c3ccccc3-4)cc2)cc1 ; c1ccc(-c2ccc(N(c3ccc(N(c4ccc(C5CCCCC5)cc4)c4ccc5c(c4)C4(c6ccccc6-c6ccccc64)c4ccccc4-5)cc3)c3cc4ccccc4cc3-c3ccccc3)cc2)cc1]

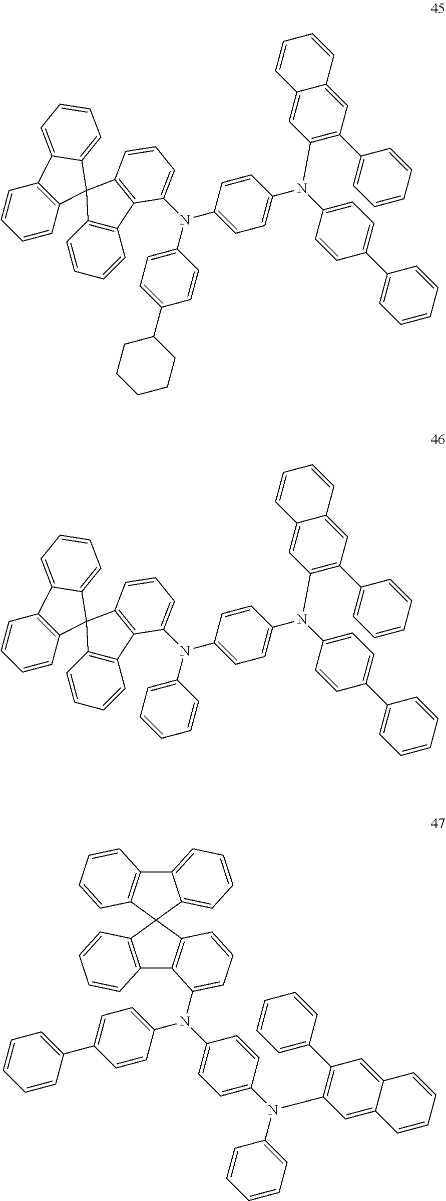 [SMILES: c1ccc(-c2ccc(N(c3ccc(N(c4ccc(C5CCCCC5)cc4)c4cccc5c4-c4ccccc4C54c5ccccc5-c5ccccc54)cc3)c3cc4ccccc4cc3-c3ccccc3)cc2)cc1 ; c1ccc(-c2ccc(N(c3ccc(N(c4ccccc4)c4cccc5c4-c4ccccc4C54c5ccccc5-c5ccccc54)cc3)c3cc4ccccc4cc3-c3ccccc3)cc2)cc1 ; c1ccc(-c2ccc(N(c3ccc(N(c4ccccc4)c4cc5ccccc5cc4-c4ccccc4)cc3)c3cccc4c3-c3ccccc3C43c4ccccc4-c4ccccc43)cc2)cc1]

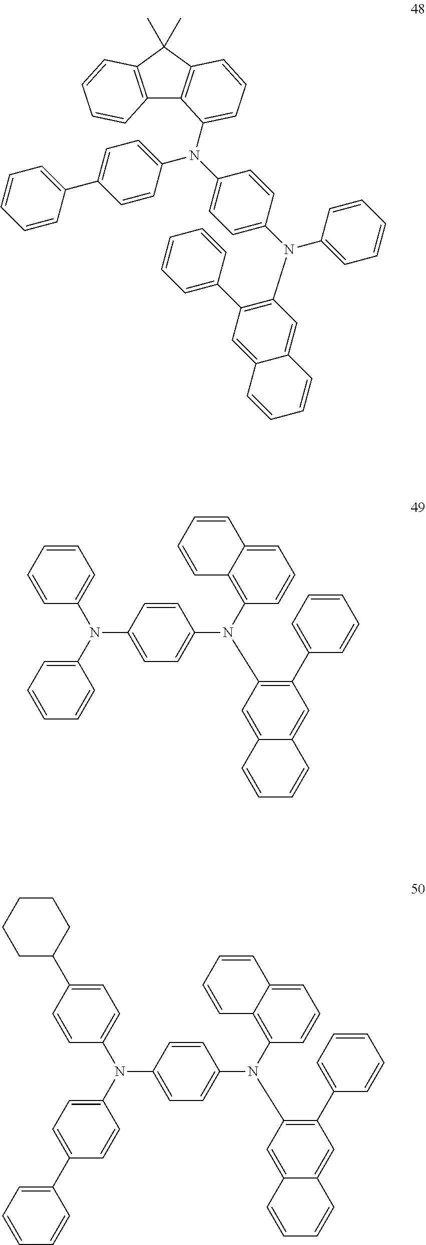 [SMILES: CC1(C)c2ccccc2-c2c(N(c3ccc(-c4ccccc4)cc3)c3ccc(N(c4ccccc4)c4cc5ccccc5cc4-c4ccccc4)cc3)cccc21 ; c1ccc(-c2cc3ccccc3cc2N(c2ccc(N(c3ccccc3)c3ccccc3)cc2)c2cccc3ccccc23)cc1 ; c1ccc(-c2ccc(N(c3ccc(C4CCCCC4)cc3)c3ccc(N(c4cc5ccccc5cc4-c4ccccc4)c4cccc5ccccc45)cc3)cc2)cc1]

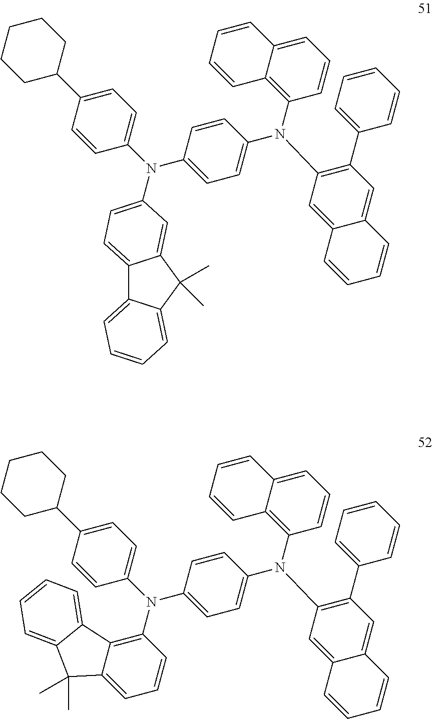 [SMILES: CC1(C)c2ccccc2-c2ccc(N(c3ccc(C4CCCCC4)cc3)c3ccc(N(c4cc5ccccc5cc4-c4ccccc4)c4cccc5ccccc45)cc3)cc21 ; CC1(C)c2ccccc2-c2c(N(c3ccc(C4CCCCC4)cc3)c3ccc(N(c4cc5ccccc5cc4-c4ccccc4)c4cccc5ccccc45)cc3)cccc21]

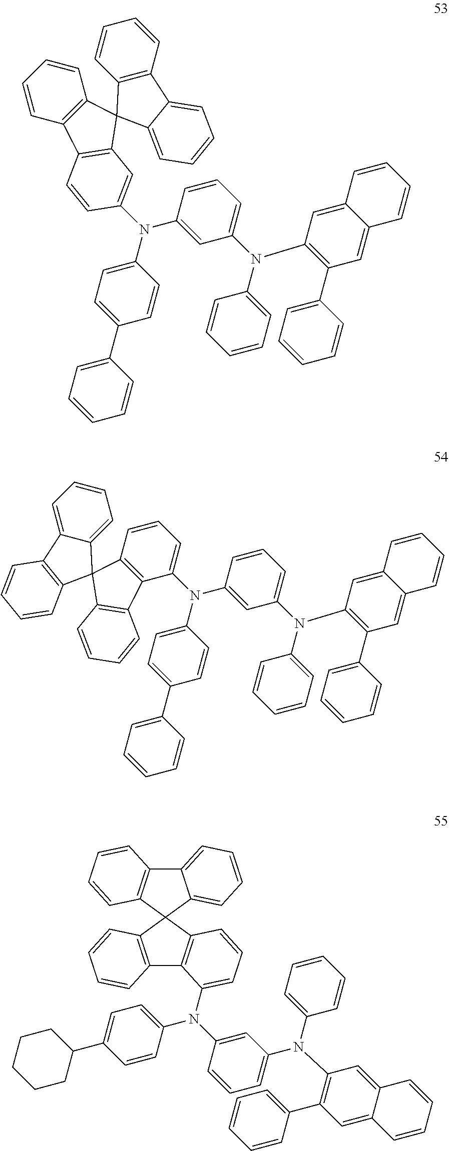 [SMILES: c1ccc(-c2ccc(N(c3cccc(N(c4ccccc4)c4cc5ccccc5cc4-c4ccccc4)c3)c3ccc4c(c3)C3(c5ccccc5-c5ccccc53)c3ccccc3-4)cc2)cc1 ; c1ccc(-c2ccc(N(c3cccc(N(c4ccccc4)c4cc5ccccc5cc4-c4ccccc4)c3)c3cccc4c3-c3ccccc3C43c4ccccc4-c4ccccc43)cc2)cc1 ; c1ccc(-c2cc3ccccc3cc2N(c2ccccc2)c2cccc(N(c3ccc(C4CCCCC4)cc3)c3cccc4c3-c3ccccc3C43c4ccccc4-c4ccccc43)c2)cc1]

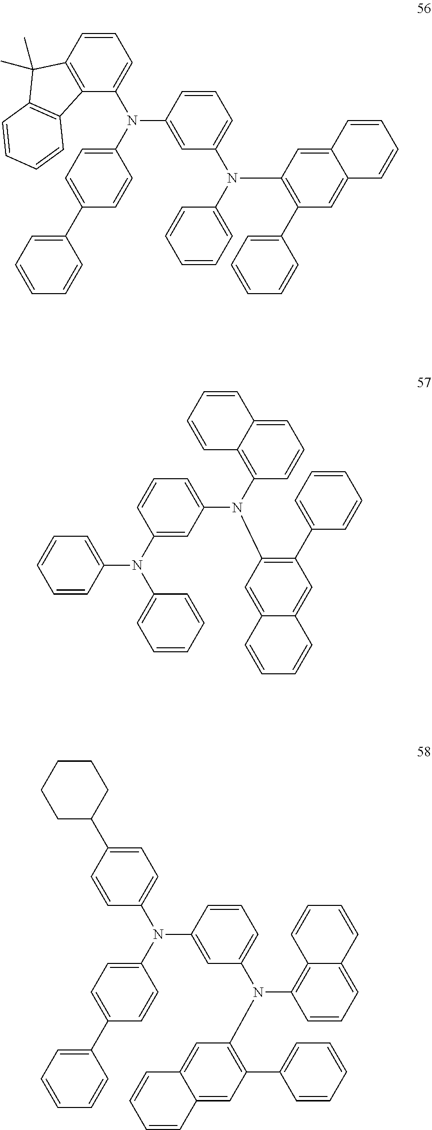 [SMILES: CC1(C)c2ccccc2-c2c(N(c3ccc(-c4ccccc4)cc3)c3cccc(N(c4ccccc4)c4cc5ccccc5cc4-c4ccccc4)c3)cccc21 ; c1ccc(-c2cc3ccccc3cc2N(c2cccc(N(c3ccccc3)c3ccccc3)c2)c2cccc3ccccc23)cc1 ; c1ccc(-c2ccc(N(c3ccc(C4CCCCC4)cc3)c3cccc(N(c4cc5ccccc5cc4-c4ccccc4)c4cccc5ccccc45)c3)cc2)cc1]

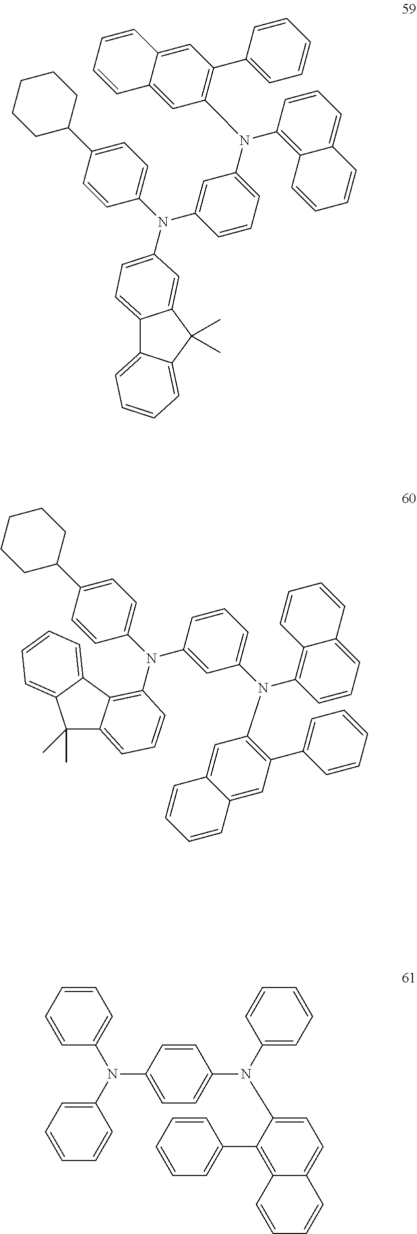 [SMILES: CC1(C)c2ccccc2-c2ccc(N(c3ccc(C4CCCCC4)cc3)c3cccc(N(c4cc5ccccc5cc4-c4ccccc4)c4cccc5ccccc45)c3)cc21 ; CC1(C)c2ccccc2-c2c(N(c3ccc(C4CCCCC4)cc3)c3cccc(N(c4cc5ccccc5cc4-c4ccccc4)c4cccc5ccccc45)c3)cccc21 ; c1ccc(-c2c(N(c3ccccc3)c3ccc(N(c4ccccc4)c4ccccc4)cc3)ccc3ccccc23)cc1]

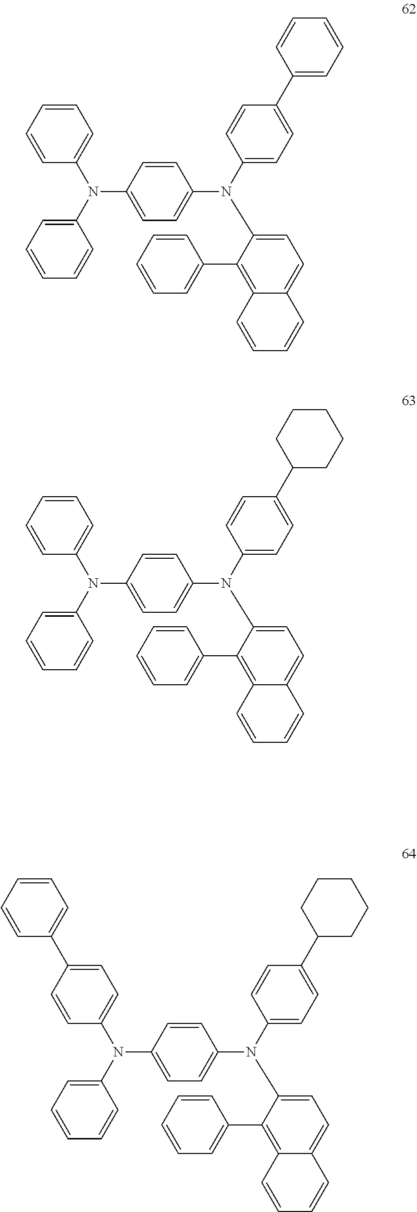 [SMILES: c1ccc(-c2ccc(N(c3ccc(N(c4ccccc4)c4ccccc4)cc3)c3ccc4ccccc4c3-c3ccccc3)cc2)cc1 ; c1ccc(-c2c(N(c3ccc(C4CCCCC4)cc3)c3ccc(N(c4ccccc4)c4ccccc4)cc3)ccc3ccccc23)cc1 ; c1ccc(-c2ccc(N(c3ccccc3)c3ccc(N(c4ccc(C5CCCCC5)cc4)c4ccc5ccccc5c4-c4ccccc4)cc3)cc2)cc1]

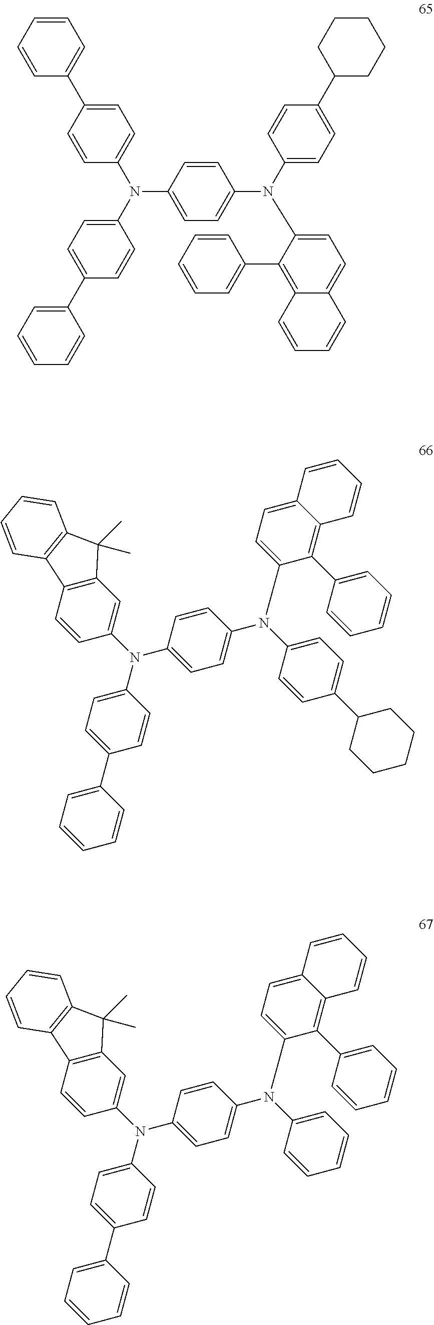 [SMILES: c1ccc(-c2ccc(N(c3ccc(-c4ccccc4)cc3)c3ccc(N(c4ccc(C5CCCCC5)cc4)c4ccc5ccccc5c4-c4ccccc4)cc3)cc2)cc1 ; CC1(C)c2ccccc2-c2ccc(N(c3ccc(-c4ccccc4)cc3)c3ccc(N(c4ccc(C5CCCCC5)cc4)c4ccc5ccccc5c4-c4ccccc4)cc3)cc21 ; CC1(C)c2ccccc2-c2ccc(N(c3ccc(-c4ccccc4)cc3)c3ccc(N(c4ccccc4)c4ccc5ccccc5c4-c4ccccc4)cc3)cc21]

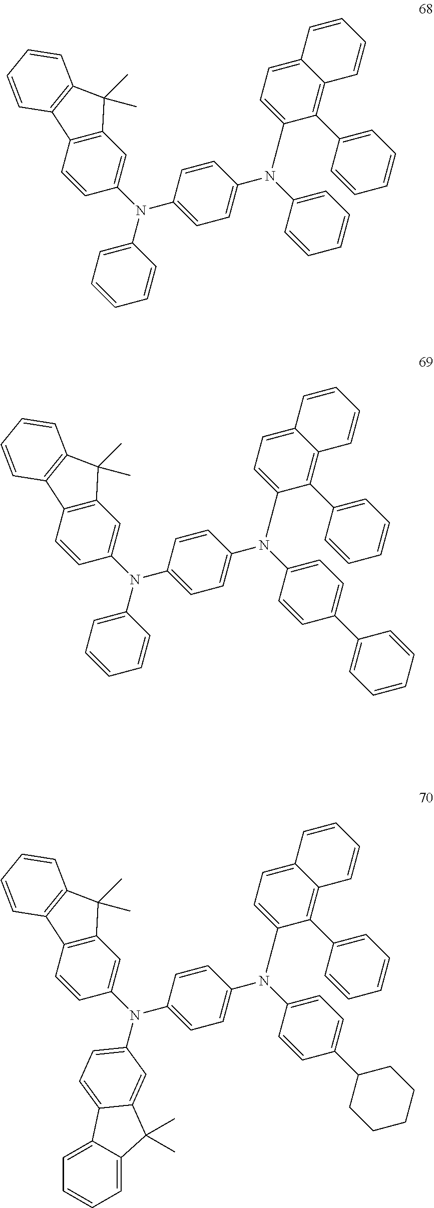 [SMILES: CC1(C)c2ccccc2-c2ccc(N(c3ccccc3)c3ccc(N(c4ccccc4)c4ccc5ccccc5c4-c4ccccc4)cc3)cc21 ; CC1(C)c2ccccc2-c2ccc(N(c3ccccc3)c3ccc(N(c4ccc(-c5ccccc5)cc4)c4ccc5ccccc5c4-c4ccccc4)cc3)cc21 ; CC1(C)c2ccccc2-c2ccc(N(c3ccc(N(c4ccc(C5CCCCC5)cc4)c4ccc5ccccc5c4-c4ccccc4)cc3)c3ccc4c(c3)C(C)(C)c3ccccc3-4)cc21]

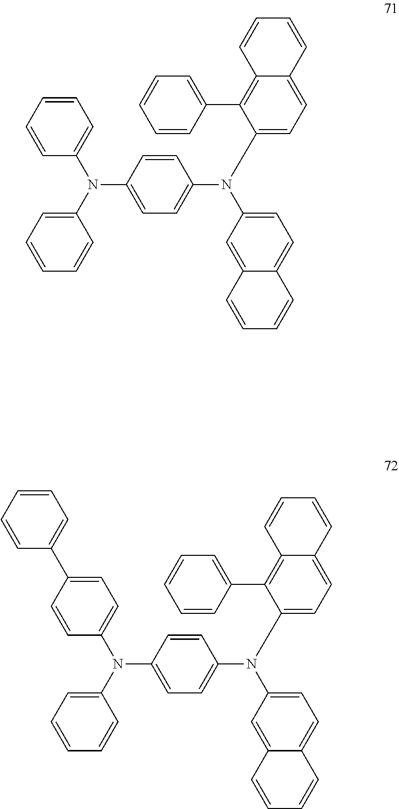 [SMILES: c1ccc(-c2c(N(c3ccc(N(c4ccccc4)c4ccccc4)cc3)c3ccc4ccccc4c3)ccc3ccccc23)cc1 ; c1ccc(-c2ccc(N(c3ccccc3)c3ccc(N(c4ccc5ccccc5c4)c4ccc5ccccc5c4-c4ccccc4)cc3)cc2)cc1]

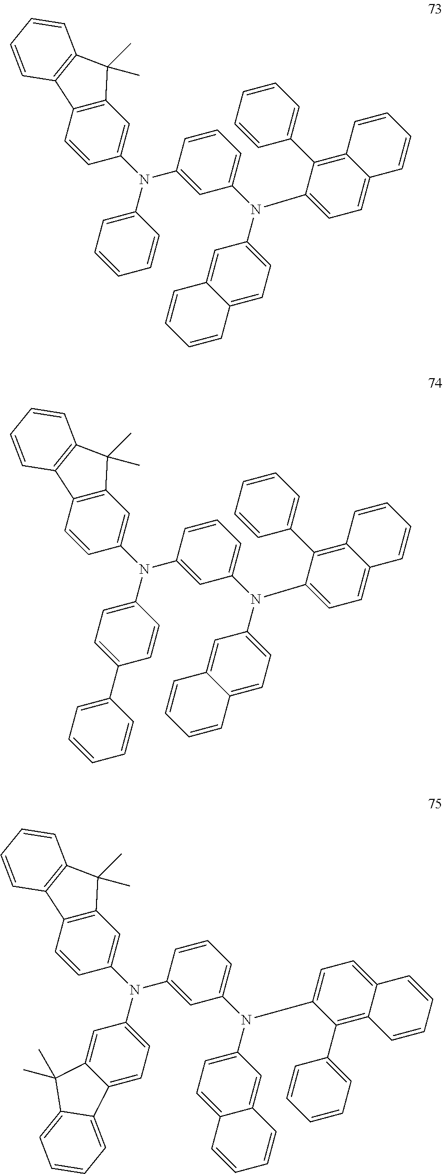 [SMILES: CC1(C)c2ccccc2-c2ccc(N(c3ccccc3)c3cccc(N(c4ccc5ccccc5c4)c4ccc5ccccc5c4-c4ccccc4)c3)cc21 ; CC1(C)c2ccccc2-c2ccc(N(c3ccc(-c4ccccc4)cc3)c3cccc(N(c4ccc5ccccc5c4)c4ccc5ccccc5c4-c4ccccc4)c3)cc21 ; CC1(C)c2ccccc2-c2ccc(N(c3cccc(N(c4ccc5ccccc5c4)c4ccc5ccccc5c4-c4ccccc4)c3)c3ccc4c(c3)C(C)(C)c3ccccc3-4)cc21]

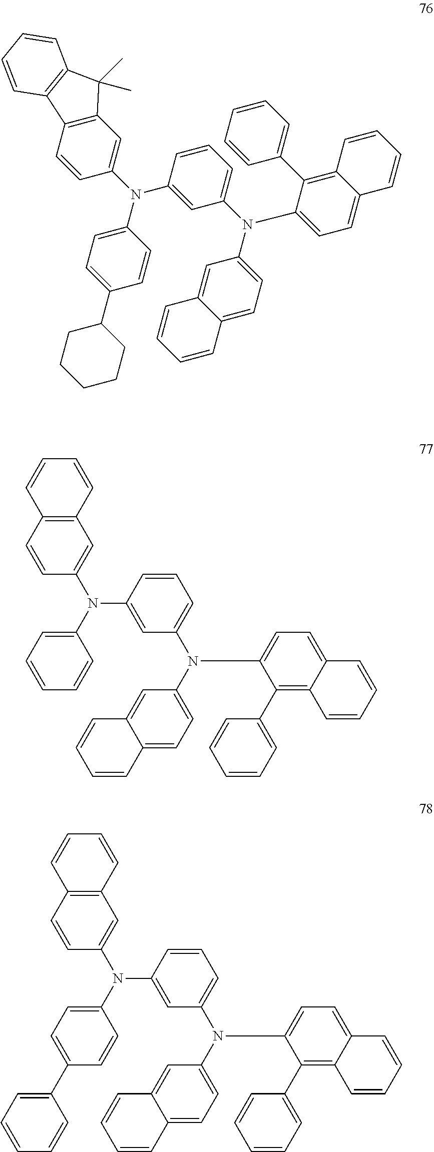 [SMILES: CC1(C)c2ccccc2-c2ccc(N(c3ccc(C4CCCCC4)cc3)c3cccc(N(c4ccc5ccccc5c4)c4ccc5ccccc5c4-c4ccccc4)c3)cc21 ; c1ccc(-c2c(N(c3cccc(N(c4ccccc4)c4ccc5ccccc5c4)c3)c3ccc4ccccc4c3)ccc3ccccc23)cc1 ; c1ccc(-c2ccc(N(c3cccc(N(c4ccc5ccccc5c4)c4ccc5ccccc5c4-c4ccccc4)c3)c3ccc4ccccc4c3)cc2)cc1]

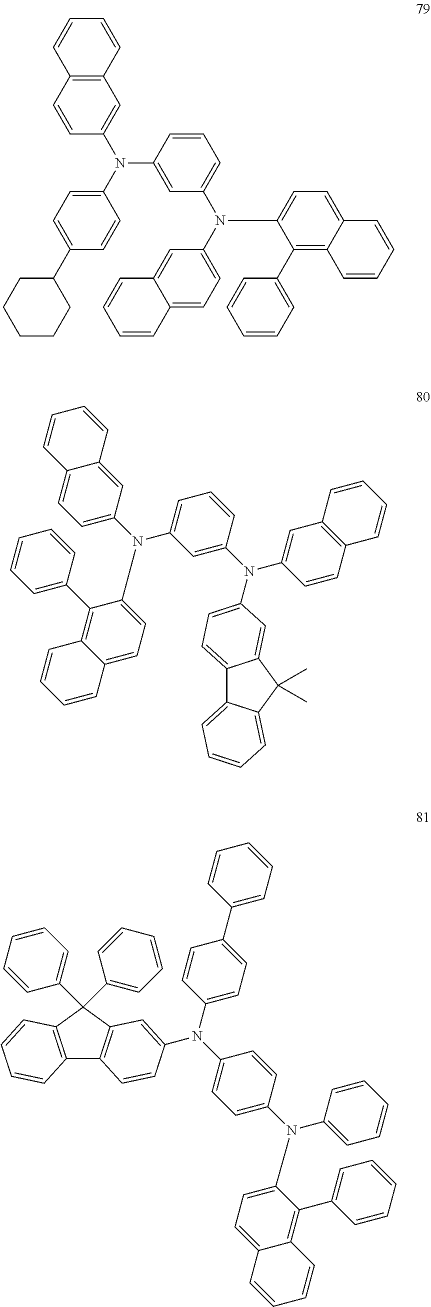 [SMILES: c1ccc(-c2c(N(c3cccc(N(c4ccc(C5CCCCC5)cc4)c4ccc5ccccc5c4)c3)c3ccc4ccccc4c3)ccc3ccccc23)cc1 ; CC1(C)c2ccccc2-c2ccc(N(c3cccc(N(c4ccc5ccccc5c4)c4ccc5ccccc5c4-c4ccccc4)c3)c3ccc4ccccc4c3)cc21 ; c1ccc(-c2ccc(N(c3ccc(N(c4ccccc4)c4ccc5ccccc5c4-c4ccccc4)cc3)c3ccc4c(c3)C(c3ccccc3)(c3ccccc3)c3ccccc3-4)cc2)cc1]

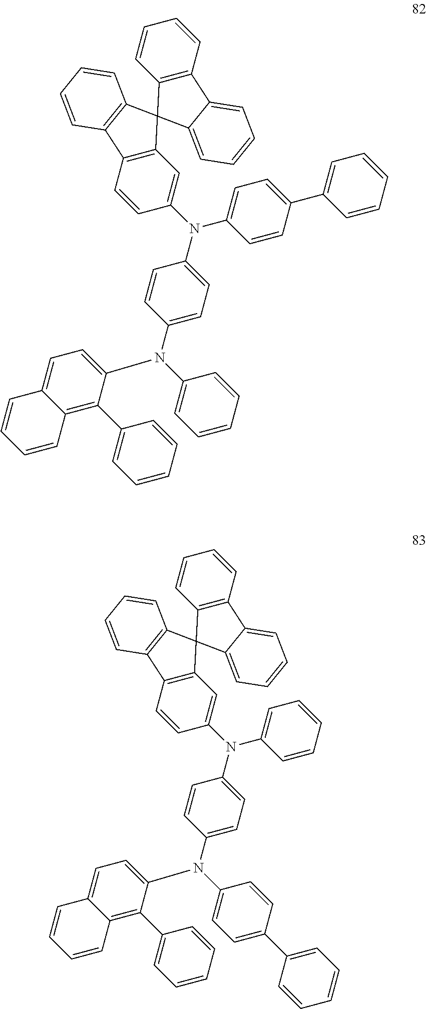 [SMILES: c1ccc(-c2ccc(N(c3ccc(N(c4ccccc4)c4ccc5ccccc5c4-c4ccccc4)cc3)c3ccc4c(c3)C3(c5ccccc5-c5ccccc53)c3ccccc3-4)cc2)cc1 ; c1ccc(-c2ccc(N(c3ccc(N(c4ccccc4)c4ccc5c(c4)C4(c6ccccc6-c6ccccc64)c4ccccc4-5)cc3)c3ccc4ccccc4c3-c3ccccc3)cc2)cc1]

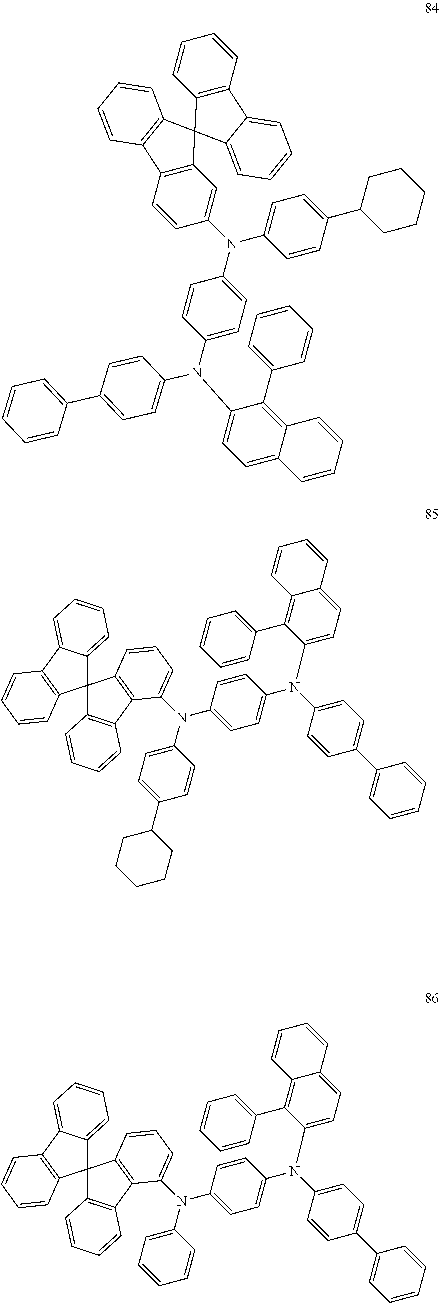 [SMILES: c1ccc(-c2ccc(N(c3ccc(N(c4ccc(C5CCCCC5)cc4)c4ccc5c(c4)C4(c6ccccc6-c6ccccc64)c4ccccc4-5)cc3)c3ccc4ccccc4c3-c3ccccc3)cc2)cc1 ; c1ccc(-c2ccc(N(c3ccc(N(c4ccc(C5CCCCC5)cc4)c4cccc5c4-c4ccccc4C54c5ccccc5-c5ccccc54)cc3)c3ccc4ccccc4c3-c3ccccc3)cc2)cc1 ; c1ccc(-c2ccc(N(c3ccc(N(c4ccccc4)c4cccc5c4-c4ccccc4C54c5ccccc5-c5ccccc54)cc3)c3ccc4ccccc4c3-c3ccccc3)cc2)cc1]

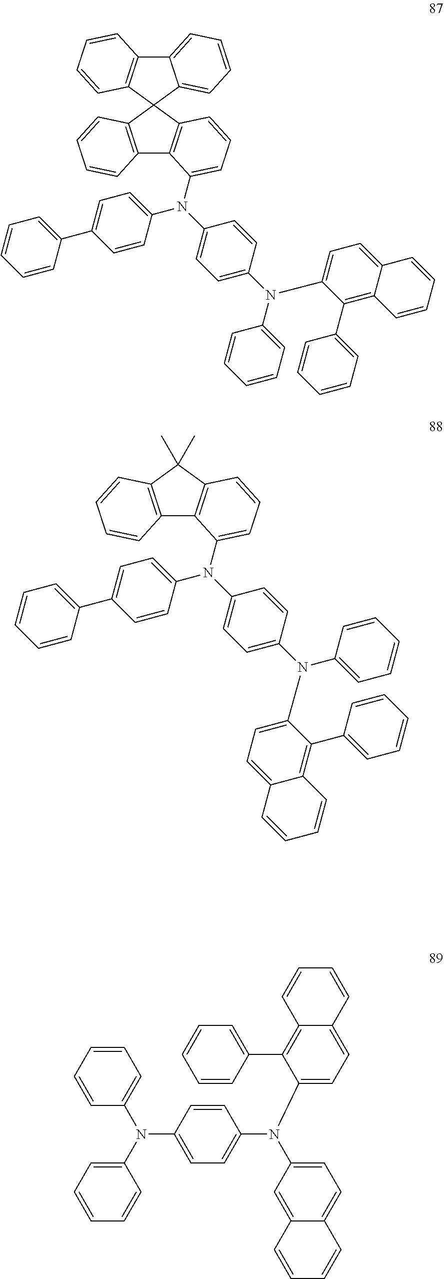 [SMILES: c1ccc(-c2ccc(N(c3ccc(N(c4ccccc4)c4ccc5ccccc5c4-c4ccccc4)cc3)c3cccc4c3-c3ccccc3C43c4ccccc4-c4ccccc43)cc2)cc1 ; CC1(C)c2ccccc2-c2c(N(c3ccc(-c4ccccc4)cc3)c3ccc(N(c4ccccc4)c4ccc5ccccc5c4-c4ccccc4)cc3)cccc21 ; c1ccc(-c2c(N(c3ccc(N(c4ccccc4)c4ccccc4)cc3)c3ccc4ccccc4c3)ccc3ccccc23)cc1]

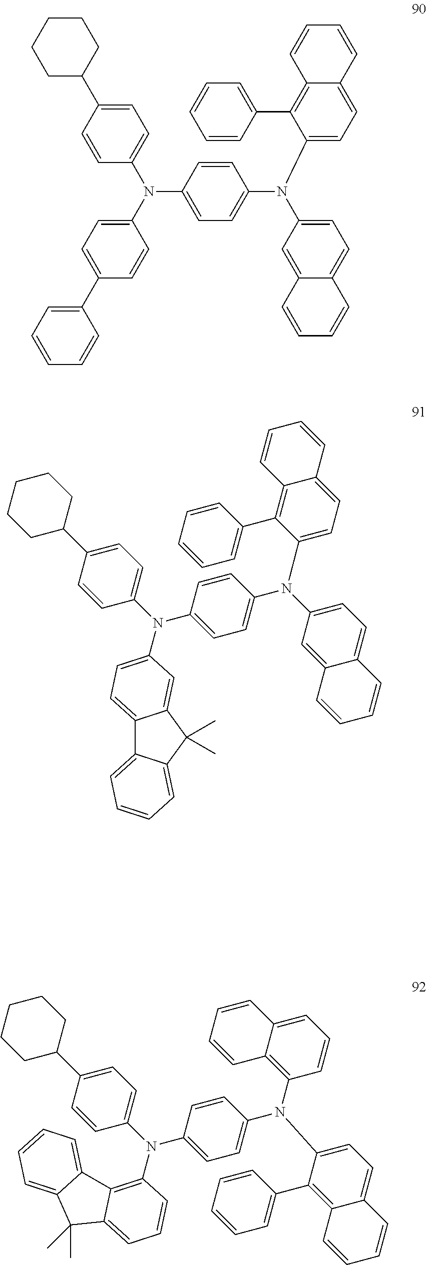 [SMILES: c1ccc(-c2ccc(N(c3ccc(C4CCCCC4)cc3)c3ccc(N(c4ccc5ccccc5c4)c4ccc5ccccc5c4-c4ccccc4)cc3)cc2)cc1 ; CC1(C)c2ccccc2-c2ccc(N(c3ccc(C4CCCCC4)cc3)c3ccc(N(c4ccc5ccccc5c4)c4ccc5ccccc5c4-c4ccccc4)cc3)cc21 ; CC1(C)c2ccccc2-c2c(N(c3ccc(C4CCCCC4)cc3)c3ccc(N(c4ccc5ccccc5c4-c4ccccc4)c4cccc5ccccc45)cc3)cccc21]

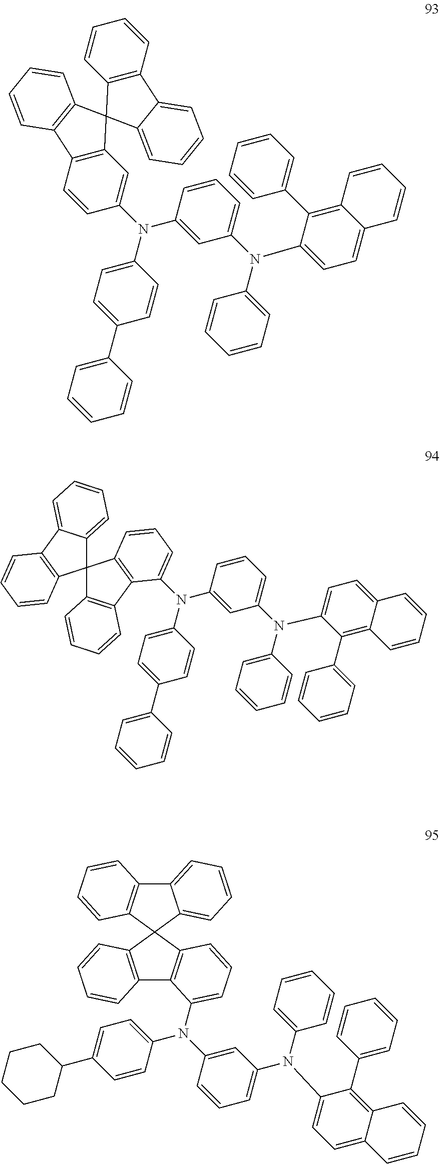 [SMILES: c1ccc(-c2ccc(N(c3cccc(N(c4ccccc4)c4ccc5ccccc5c4-c4ccccc4)c3)c3ccc4c(c3)C3(c5ccccc5-c5ccccc53)c3ccccc3-4)cc2)cc1 ; c1ccc(-c2ccc(N(c3cccc(N(c4ccccc4)c4ccc5ccccc5c4-c4ccccc4)c3)c3cccc4c3-c3ccccc3C43c4ccccc4-c4ccccc43)cc2)cc1 ; c1ccc(-c2c(N(c3ccccc3)c3cccc(N(c4ccc(C5CCCCC5)cc4)c4cccc5c4-c4ccccc4C54c5ccccc5-c5ccccc54)c3)ccc3ccccc23)cc1]

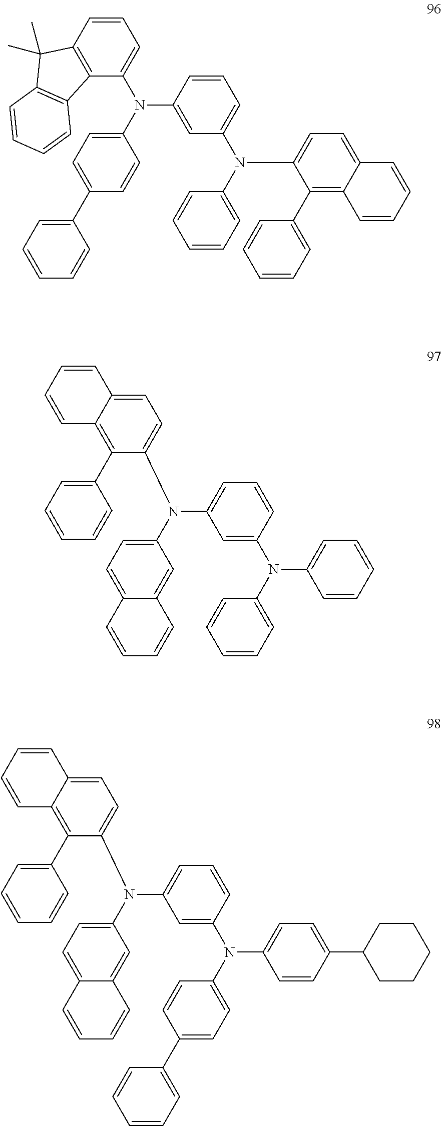 [SMILES: CC1(C)c2ccccc2-c2c(N(c3ccc(-c4ccccc4)cc3)c3cccc(N(c4ccccc4)c4ccc5ccccc5c4-c4ccccc4)c3)cccc21 ; c1ccc(-c2c(N(c3cccc(N(c4ccccc4)c4ccccc4)c3)c3ccc4ccccc4c3)ccc3ccccc23)cc1 ; c1ccc(-c2ccc(N(c3ccc(C4CCCCC4)cc3)c3cccc(N(c4ccc5ccccc5c4)c4ccc5ccccc5c4-c4ccccc4)c3)cc2)cc1]

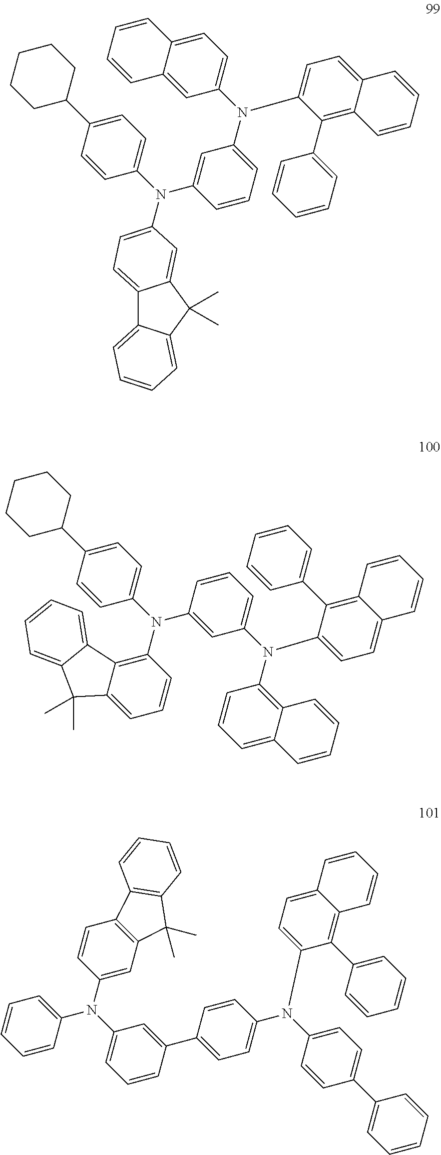 [SMILES: CC1(C)c2ccccc2-c2ccc(N(c3ccc(C4CCCCC4)cc3)c3cccc(N(c4ccc5ccccc5c4)c4ccc5ccccc5c4-c4ccccc4)c3)cc21 ; CC1(C)c2ccccc2-c2c(N(c3ccc(C4CCCCC4)cc3)c3cccc(N(c4ccc5ccccc5c4-c4ccccc4)c4cccc5ccccc45)c3)cccc21 ; CC1(C)c2ccccc2-c2ccc(N(c3ccccc3)c3cccc(-c4ccc(N(c5ccc(-c6ccccc6)cc5)c5ccc6ccccc6c5-c5ccccc5)cc4)c3)cc21]

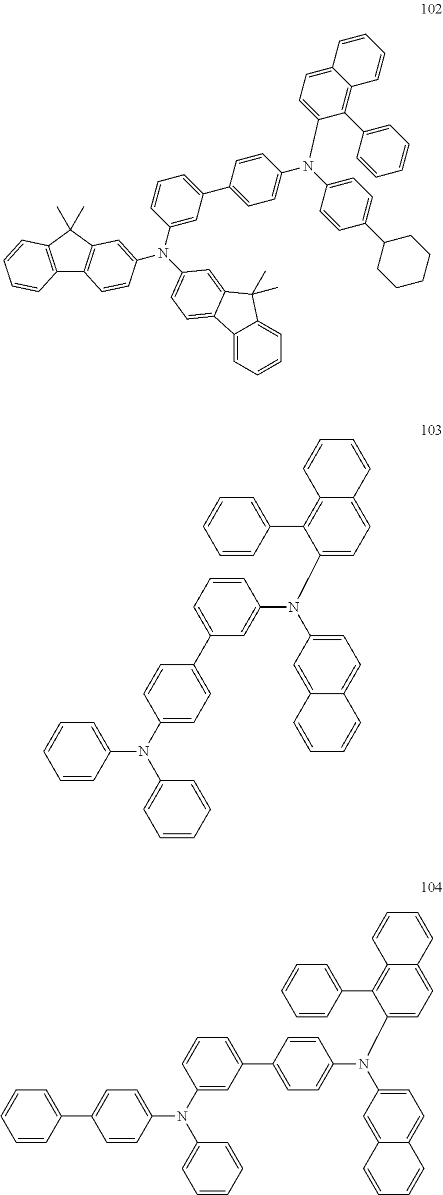 [SMILES: CC1(C)c2ccccc2-c2ccc(N(c3cccc(-c4ccc(N(c5ccc(C6CCCCC6)cc5)c5ccc6ccccc6c5-c5ccccc5)cc4)c3)c3ccc4c(c3)C(C)(C)c3ccccc3-4)cc21 ; c1ccc(-c2c(N(c3cccc(-c4ccc(N(c5ccccc5)c5ccccc5)cc4)c3)c3ccc4ccccc4c3)ccc3ccccc23)cc1 ; c1ccc(-c2ccc(N(c3ccccc3)c3cccc(-c4ccc(N(c5ccc6ccccc6c5)c5ccc6ccccc6c5-c5ccccc5)cc4)c3)cc2)cc1]

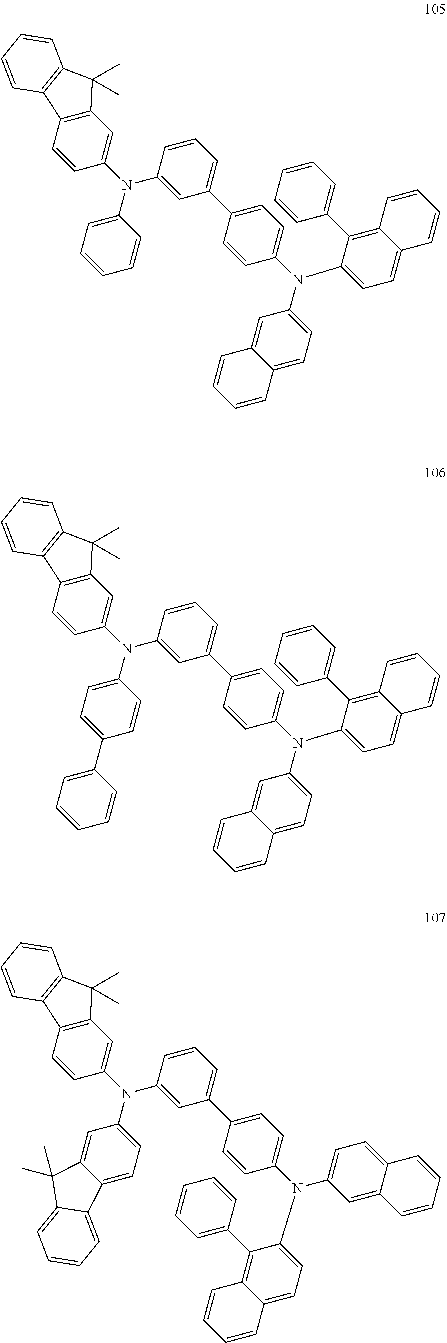 [SMILES: CC1(C)c2ccccc2-c2ccc(N(c3ccccc3)c3cccc(-c4ccc(N(c5ccc6ccccc6c5)c5ccc6ccccc6c5-c5ccccc5)cc4)c3)cc21 ; CC1(C)c2ccccc2-c2ccc(N(c3ccc(-c4ccccc4)cc3)c3cccc(-c4ccc(N(c5ccc6ccccc6c5)c5ccc6ccccc6c5-c5ccccc5)cc4)c3)cc21 ; CC1(C)c2ccccc2-c2ccc(N(c3cccc(-c4ccc(N(c5ccc6ccccc6c5)c5ccc6ccccc6c5-c5ccccc5)cc4)c3)c3ccc4c(c3)C(C)(C)c3ccccc3-4)cc21]

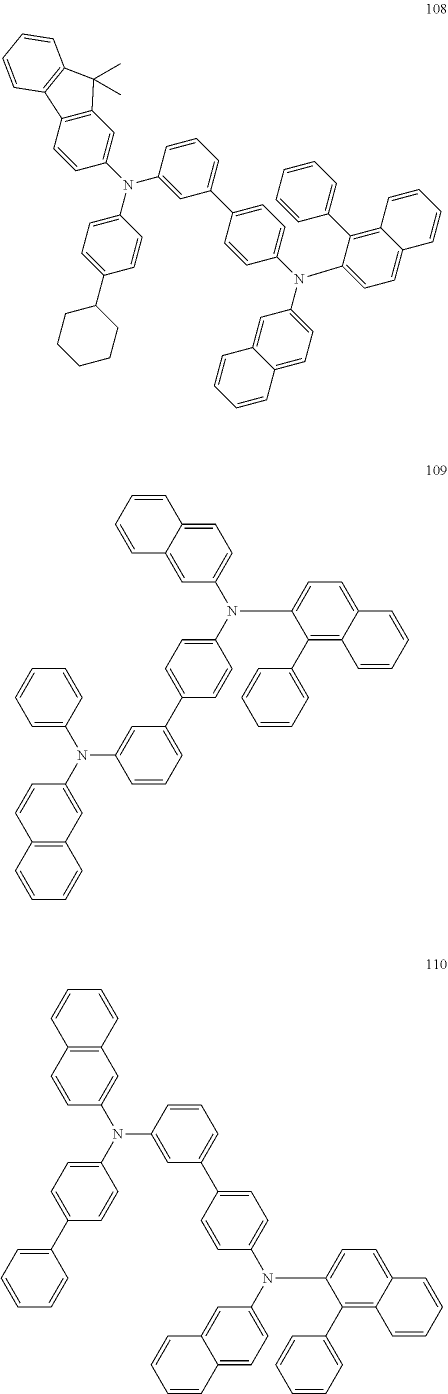 [SMILES: CC1(C)c2ccccc2-c2ccc(N(c3ccc(C4CCCCC4)cc3)c3cccc(-c4ccc(N(c5ccc6ccccc6c5)c5ccc6ccccc6c5-c5ccccc5)cc4)c3)cc21 ; c1ccc(-c2c(N(c3ccc(-c4cccc(N(c5ccccc5)c5ccc6ccccc6c5)c4)cc3)c3ccc4ccccc4c3)ccc3ccccc23)cc1 ; c1ccc(-c2ccc(N(c3cccc(-c4ccc(N(c5ccc6ccccc6c5)c5ccc6ccccc6c5-c5ccccc5)cc4)c3)c3ccc4ccccc4c3)cc2)cc1]

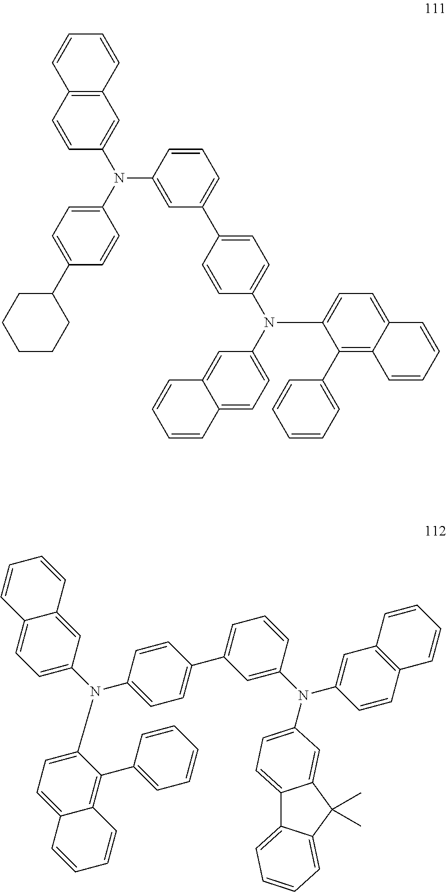 [SMILES: c1ccc(-c2c(N(c3ccc(-c4cccc(N(c5ccc(C6CCCCC6)cc5)c5ccc6ccccc6c5)c4)cc3)c3ccc4ccccc4c3)ccc3ccccc23)cc1 ; CC1(C)c2ccccc2-c2ccc(N(c3cccc(-c4ccc(N(c5ccc6ccccc6c5)c5ccc6ccccc6c5-c5ccccc5)cc4)c3)c3ccc4ccccc4c3)cc21]

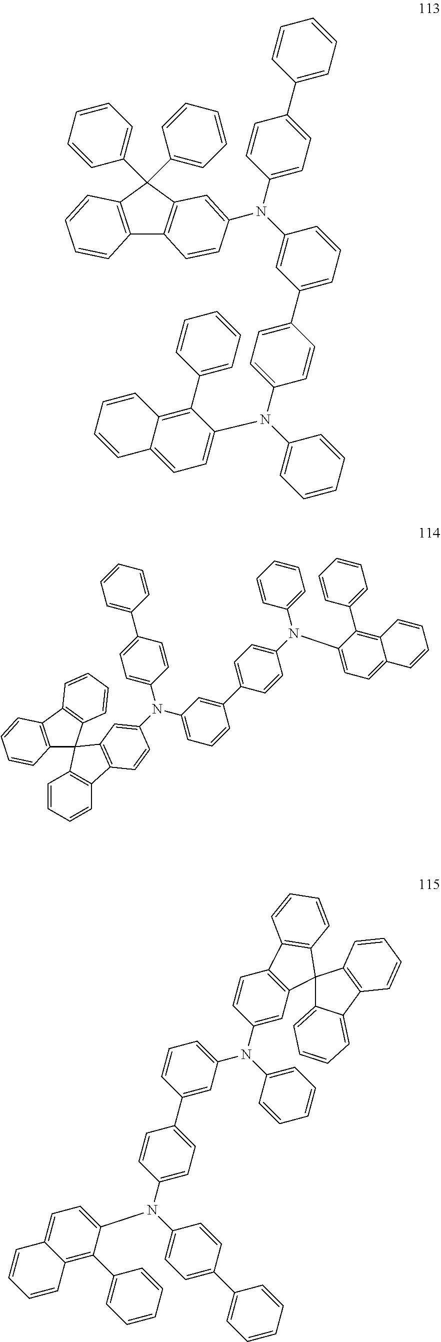 [SMILES: c1ccc(-c2ccc(N(c3cccc(-c4ccc(N(c5ccccc5)c5ccc6ccccc6c5-c5ccccc5)cc4)c3)c3ccc4c(c3)C(c3ccccc3)(c3ccccc3)c3ccccc3-4)cc2)cc1 ; c1ccc(-c2ccc(N(c3cccc(-c4ccc(N(c5ccccc5)c5ccc6ccccc6c5-c5ccccc5)cc4)c3)c3ccc4c(c3)C3(c5ccccc5-c5ccccc53)c3ccccc3-4)cc2)cc1 ; c1ccc(-c2ccc(N(c3ccc(-c4cccc(N(c5ccccc5)c5ccc6c(c5)C5(c7ccccc7-c7ccccc75)c5ccccc5-6)c4)cc3)c3ccc4ccccc4c3-c3ccccc3)cc2)cc1]

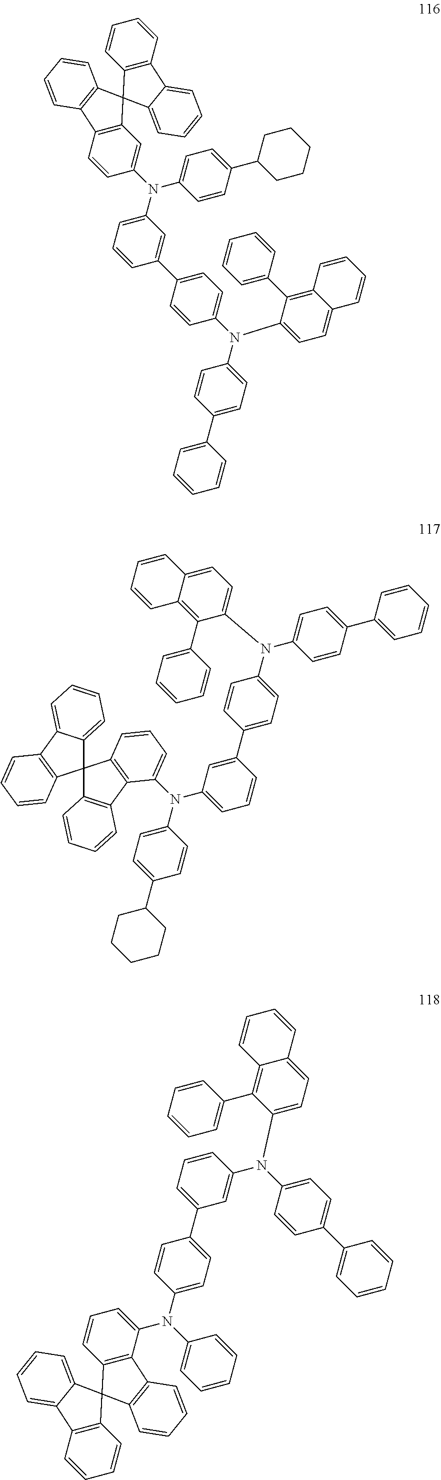 [SMILES: c1ccc(-c2ccc(N(c3ccc(-c4cccc(N(c5ccc(C6CCCCC6)cc5)c5ccc6c(c5)C5(c7ccccc7-c7ccccc75)c5ccccc5-6)c4)cc3)c3ccc4ccccc4c3-c3ccccc3)cc2)cc1 ; c1ccc(-c2ccc(N(c3ccc(-c4cccc(N(c5ccc(C6CCCCC6)cc5)c5cccc6c5-c5ccccc5C65c6ccccc6-c6ccccc65)c4)cc3)c3ccc4ccccc4c3-c3ccccc3)cc2)cc1 ; c1ccc(-c2ccc(N(c3cccc(-c4ccc(N(c5ccccc5)c5cccc6c5-c5ccccc5C65c6ccccc6-c6ccccc65)cc4)c3)c3ccc4ccccc4c3-c3ccccc3)cc2)cc1]

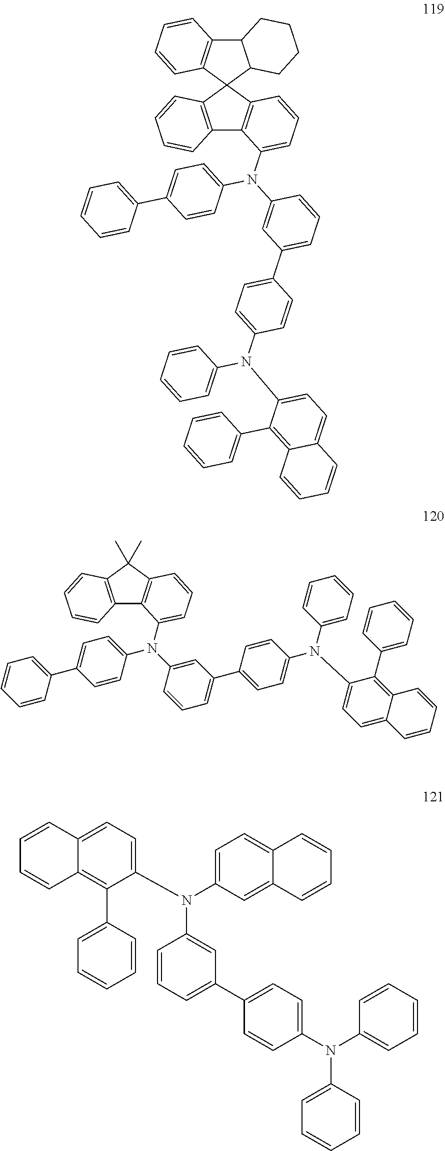 [SMILES: c1ccc(-c2ccc(N(c3cccc(-c4ccc(N(c5ccccc5)c5ccc6ccccc6c5-c5ccccc5)cc4)c3)c3cccc4c3-c3ccccc3C43c4ccccc4C4CCCCC43)cc2)cc1 ; CC1(C)c2ccccc2-c2c(N(c3ccc(-c4ccccc4)cc3)c3cccc(-c4ccc(N(c5ccccc5)c5ccc6ccccc6c5-c5ccccc5)cc4)c3)cccc21 ; c1ccc(-c2c(N(c3cccc(-c4ccc(N(c5ccccc5)c5ccccc5)cc4)c3)c3ccc4ccccc4c3)ccc3ccccc23)cc1]

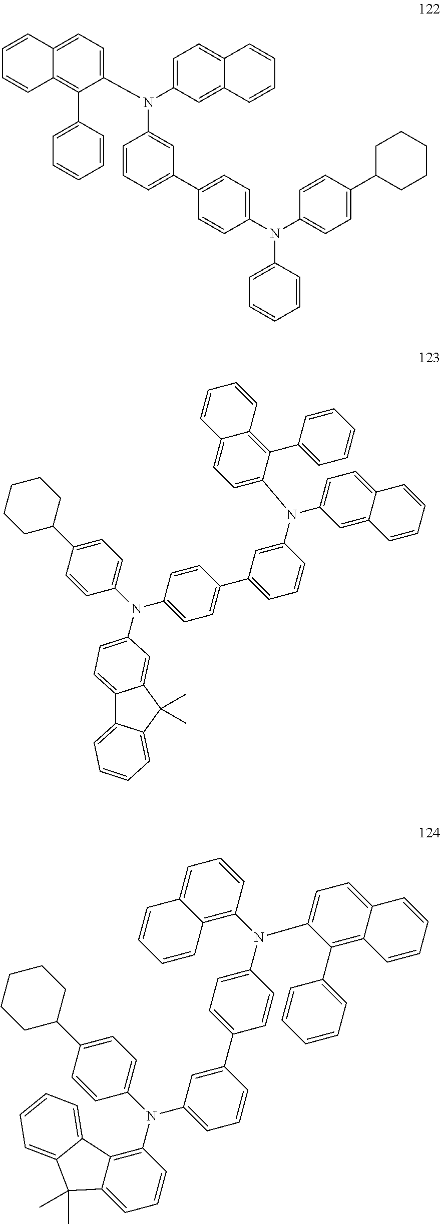 [SMILES: c1ccc(-c2c(N(c3cccc(-c4ccc(N(c5ccccc5)c5ccc(C6CCCCC6)cc5)cc4)c3)c3ccc4ccccc4c3)ccc3ccccc23)cc1 ; CC1(C)c2ccccc2-c2ccc(N(c3ccc(-c4cccc(N(c5ccc6ccccc6c5)c5ccc6ccccc6c5-c5ccccc5)c4)cc3)c3ccc(C4CCCCC4)cc3)cc21 ; CC1(C)c2ccccc2-c2c(N(c3ccc(C4CCCCC4)cc3)c3cccc(-c4ccc(N(c5ccc6ccccc6c5-c5ccccc5)c5cccc6ccccc56)cc4)c3)cccc21]

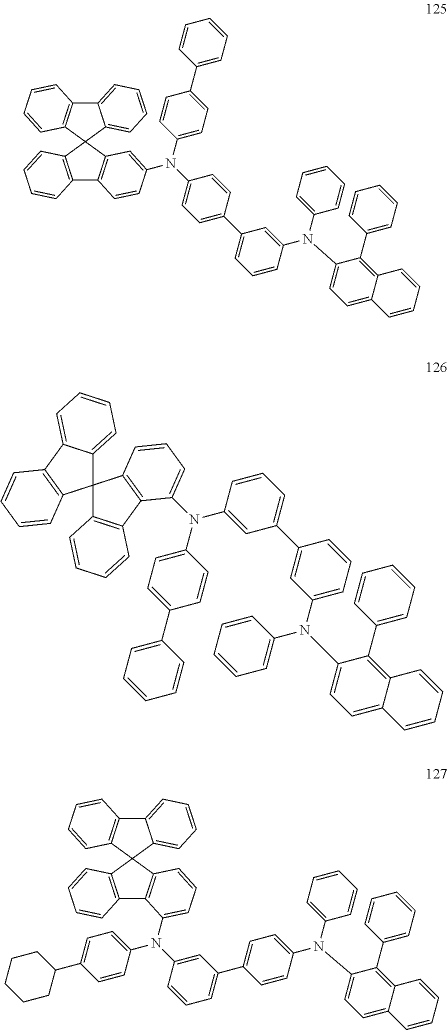 [SMILES: c1ccc(-c2ccc(N(c3ccc(-c4cccc(N(c5ccccc5)c5ccc6ccccc6c5-c5ccccc5)c4)cc3)c3ccc4c(c3)C3(c5ccccc5-c5ccccc53)c3ccccc3-4)cc2)cc1 ; c1ccc(-c2ccc(N(c3cccc(-c4cccc(N(c5ccccc5)c5ccc6ccccc6c5-c5ccccc5)c4)c3)c3cccc4c3-c3ccccc3C43c4ccccc4-c4ccccc43)cc2)cc1 ; c1ccc(-c2c(N(c3ccccc3)c3ccc(-c4cccc(N(c5ccc(C6CCCCC6)cc5)c5cccc6c5-c5ccccc5C65c6ccccc6-c6ccccc65)c4)cc3)ccc3ccccc23)cc1]

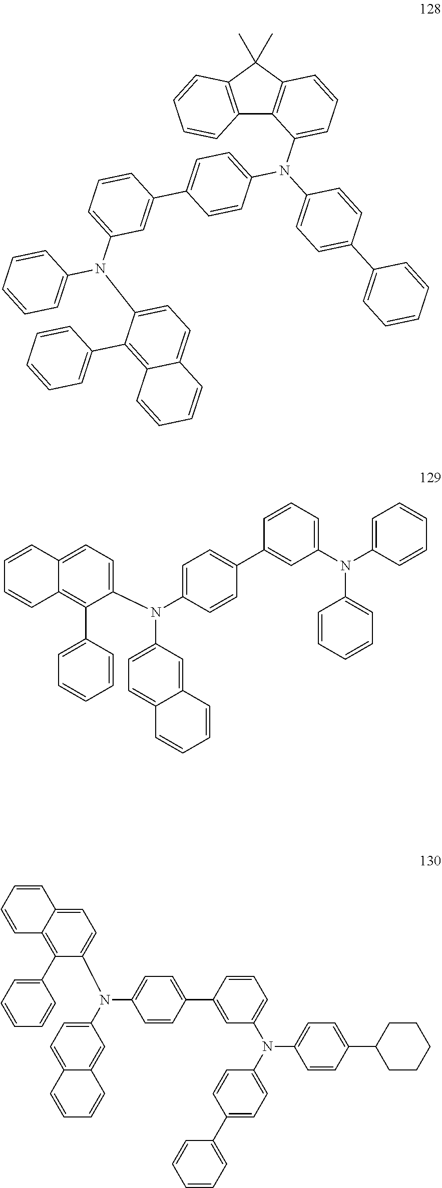 [SMILES: CC1(C)c2ccccc2-c2c(N(c3ccc(-c4ccccc4)cc3)c3ccc(-c4cccc(N(c5ccccc5)c5ccc6ccccc6c5-c5ccccc5)c4)cc3)cccc21 ; c1ccc(-c2c(N(c3ccc(-c4cccc(N(c5ccccc5)c5ccccc5)c4)cc3)c3ccc4ccccc4c3)ccc3ccccc23)cc1 ; c1ccc(-c2ccc(N(c3ccc(C4CCCCC4)cc3)c3cccc(-c4ccc(N(c5ccc6ccccc6c5)c5ccc6ccccc6c5-c5ccccc5)cc4)c3)cc2)cc1]

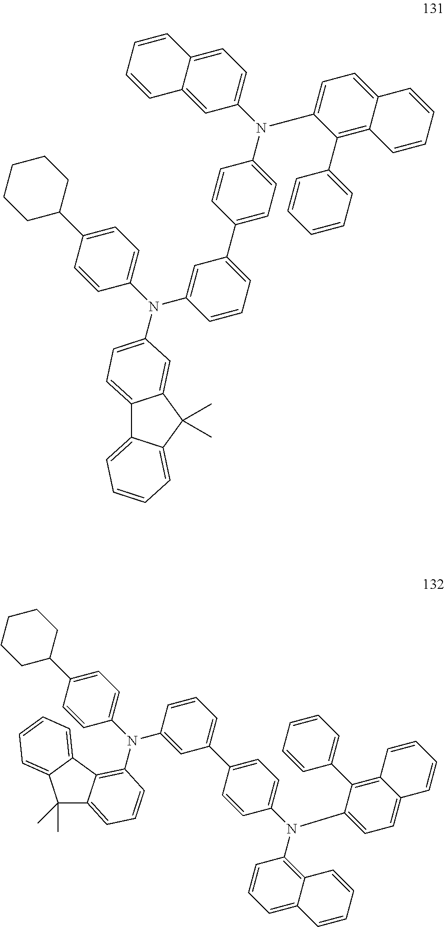 [SMILES: CC1(C)c2ccccc2-c2ccc(N(c3ccc(C4CCCCC4)cc3)c3cccc(-c4ccc(N(c5ccc6ccccc6c5)c5ccc6ccccc6c5-c5ccccc5)cc4)c3)cc21 ; CC1(C)c2ccccc2-c2c(N(c3ccc(C4CCCCC4)cc3)c3cccc(-c4ccc(N(c5ccc6ccccc6c5-c5ccccc5)c5cccc6ccccc56)cc4)c3)cccc21]

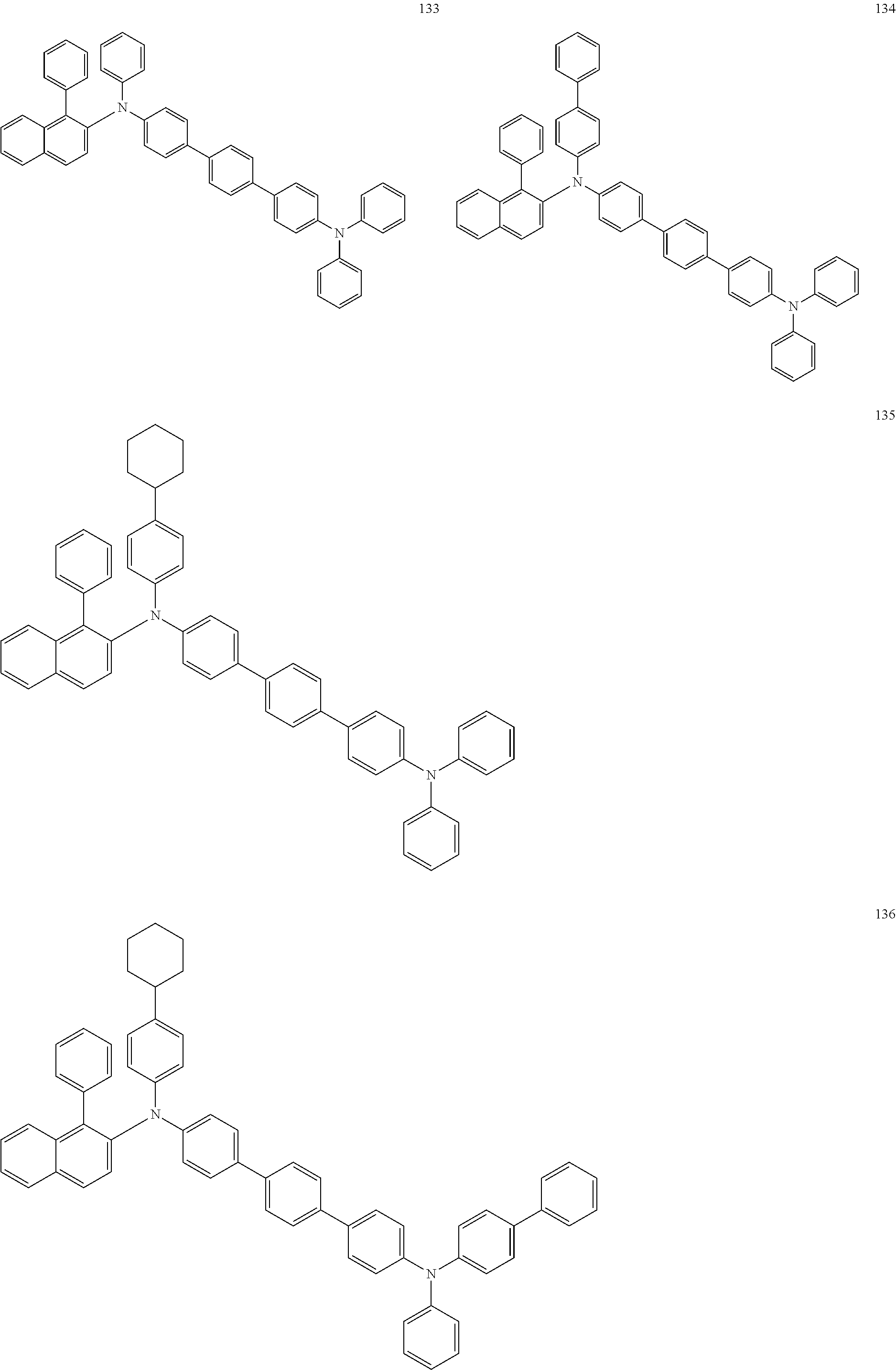 [SMILES: c1ccc(-c2c(N(c3ccccc3)c3ccc(-c4ccc(-c5ccc(N(c6ccccc6)c6ccccc6)cc5)cc4)cc3)ccc3ccccc23)cc1 ; c1ccc(-c2ccc(N(c3ccc(-c4ccc(-c5ccc(N(c6ccccc6)c6ccccc6)cc5)cc4)cc3)c3ccc4ccccc4c3-c3ccccc3)cc2)cc1 ; c1ccc(-c2c(N(c3ccc(-c4ccc(-c5ccc(N(c6ccccc6)c6ccccc6)cc5)cc4)cc3)c3ccc(C4CCCCC4)cc3)ccc3ccccc23)cc1 ; c1ccc(-c2ccc(N(c3ccccc3)c3ccc(-c4ccc(-c5ccc(N(c6ccc(C7CCCCC7)cc6)c6ccc7ccccc7c6-c6ccccc6)cc5)cc4)cc3)cc2)cc1]

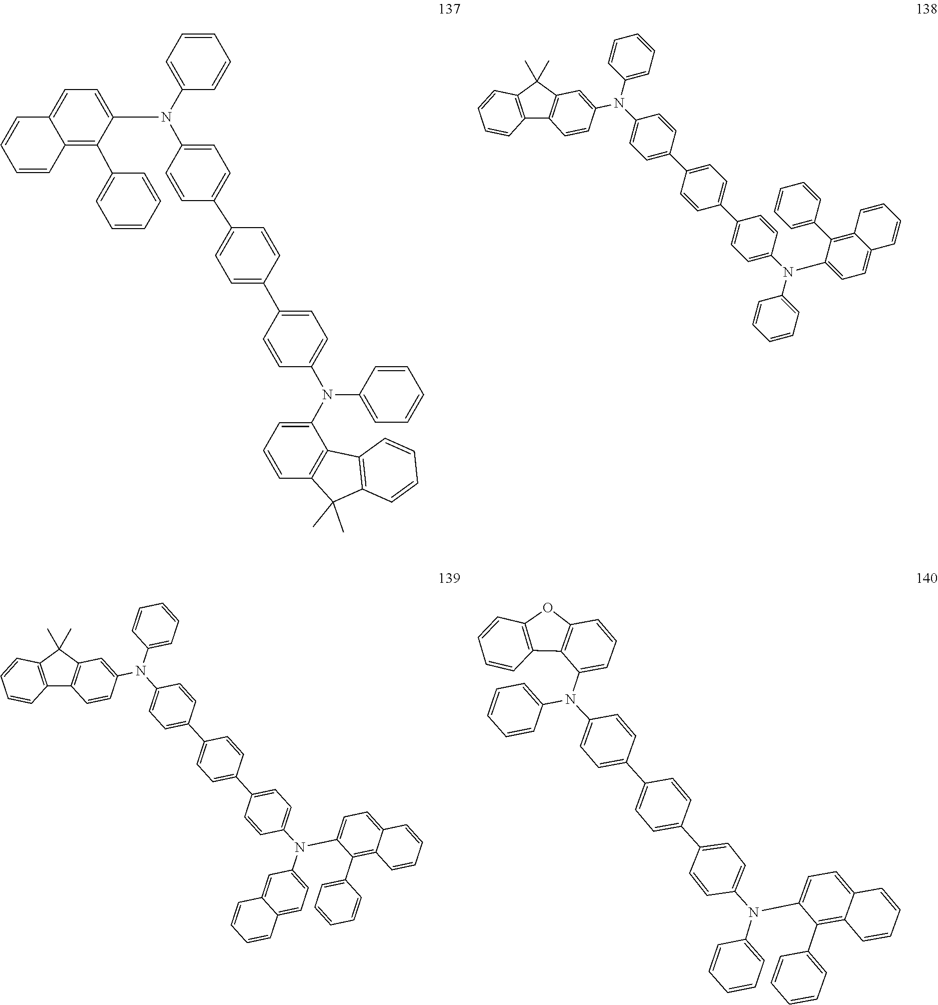 [SMILES: CC1(C)c2ccccc2-c2c(N(c3ccccc3)c3ccc(-c4ccc(-c5ccc(N(c6ccccc6)c6ccc7ccccc7c6-c6ccccc6)cc5)cc4)cc3)cccc21 ; CC1(C)c2ccccc2-c2ccc(N(c3ccccc3)c3ccc(-c4ccc(-c5ccc(N(c6ccccc6)c6ccc7ccccc7c6-c6ccccc6)cc5)cc4)cc3)cc21 ; CC1(C)c2ccccc2-c2ccc(N(c3ccccc3)c3ccc(-c4ccc(-c5ccc(N(c6ccc7ccccc7c6)c6ccc7ccccc7c6-c6ccccc6)cc5)cc4)cc3)cc21 ; c1ccc(-c2c(N(c3ccccc3)c3ccc(-c4ccc(-c5ccc(N(c6ccccc6)c6cccc7oc8ccccc8c67)cc5)cc4)cc3)ccc3ccccc23)cc1]

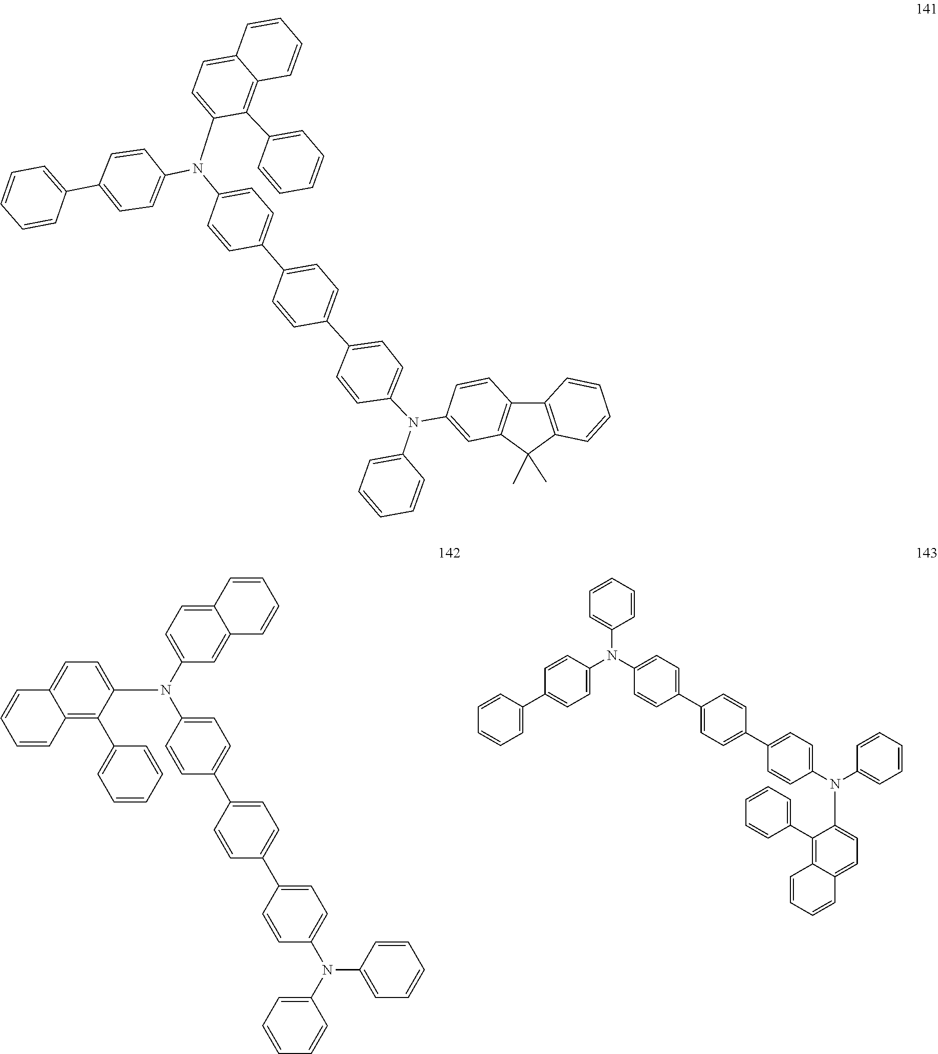 [SMILES: CC1(C)c2ccccc2-c2ccc(N(c3ccccc3)c3ccc(-c4ccc(-c5ccc(N(c6ccc(-c7ccccc7)cc6)c6ccc7ccccc7c6-c6ccccc6)cc5)cc4)cc3)cc21 ; c1ccc(-c2c(N(c3ccc(-c4ccc(-c5ccc(N(c6ccccc6)c6ccccc6)cc5)cc4)cc3)c3ccc4ccccc4c3)ccc3ccccc23)cc1 ; c1ccc(-c2ccc(N(c3ccccc3)c3ccc(-c4ccc(-c5ccc(N(c6ccccc6)c6ccc7ccccc7c6-c6ccccc6)cc5)cc4)cc3)cc2)cc1]

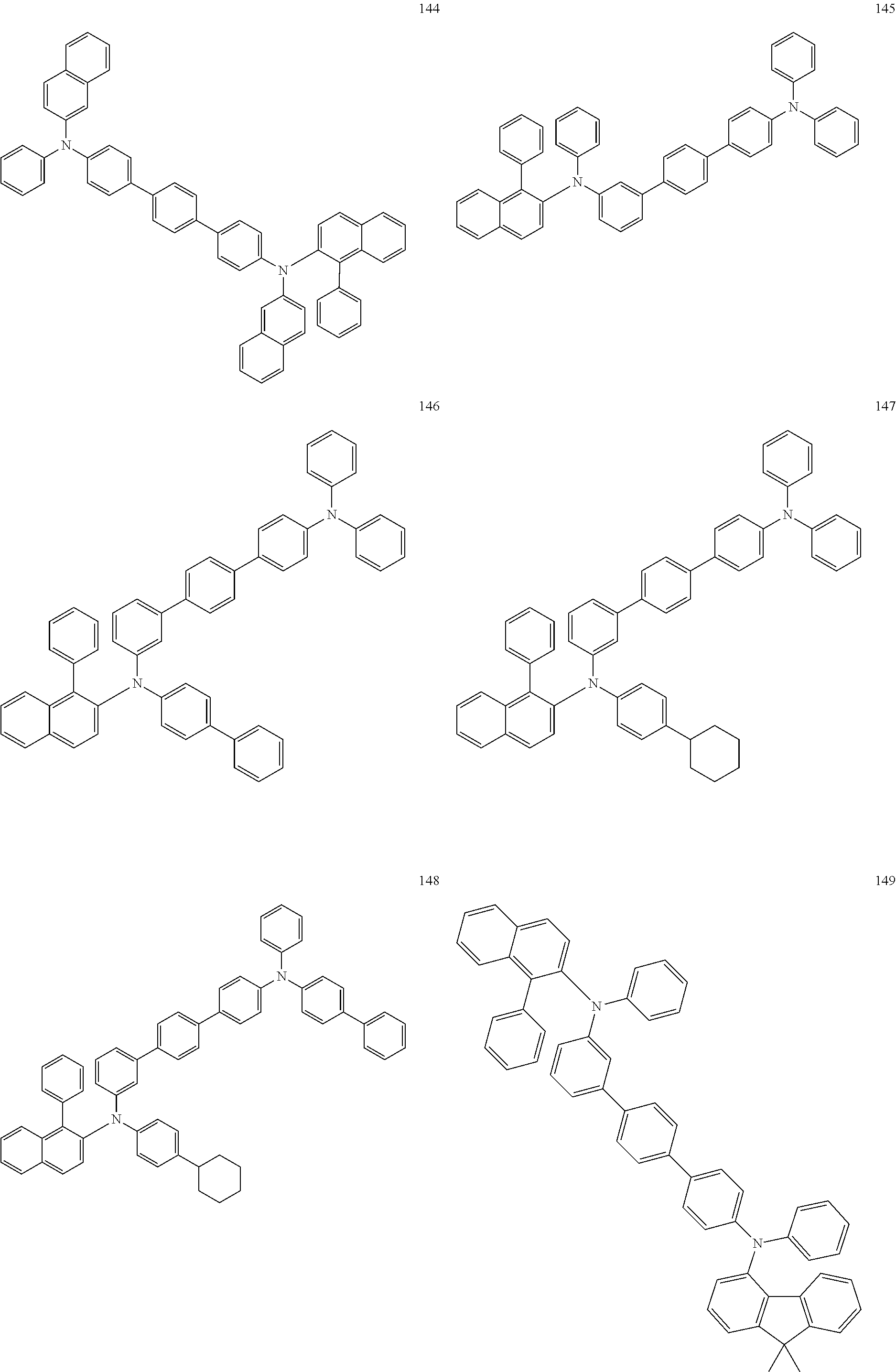

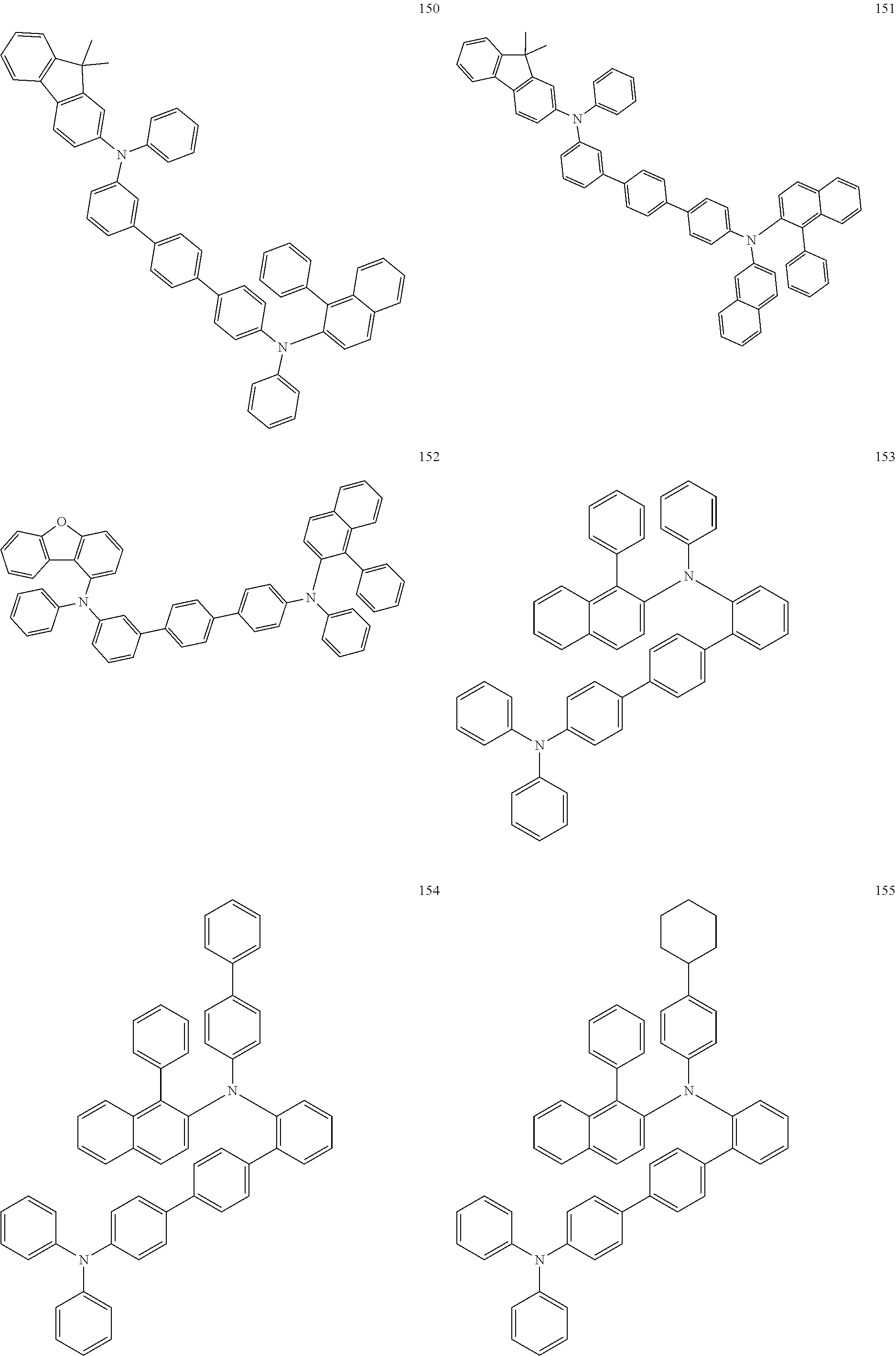

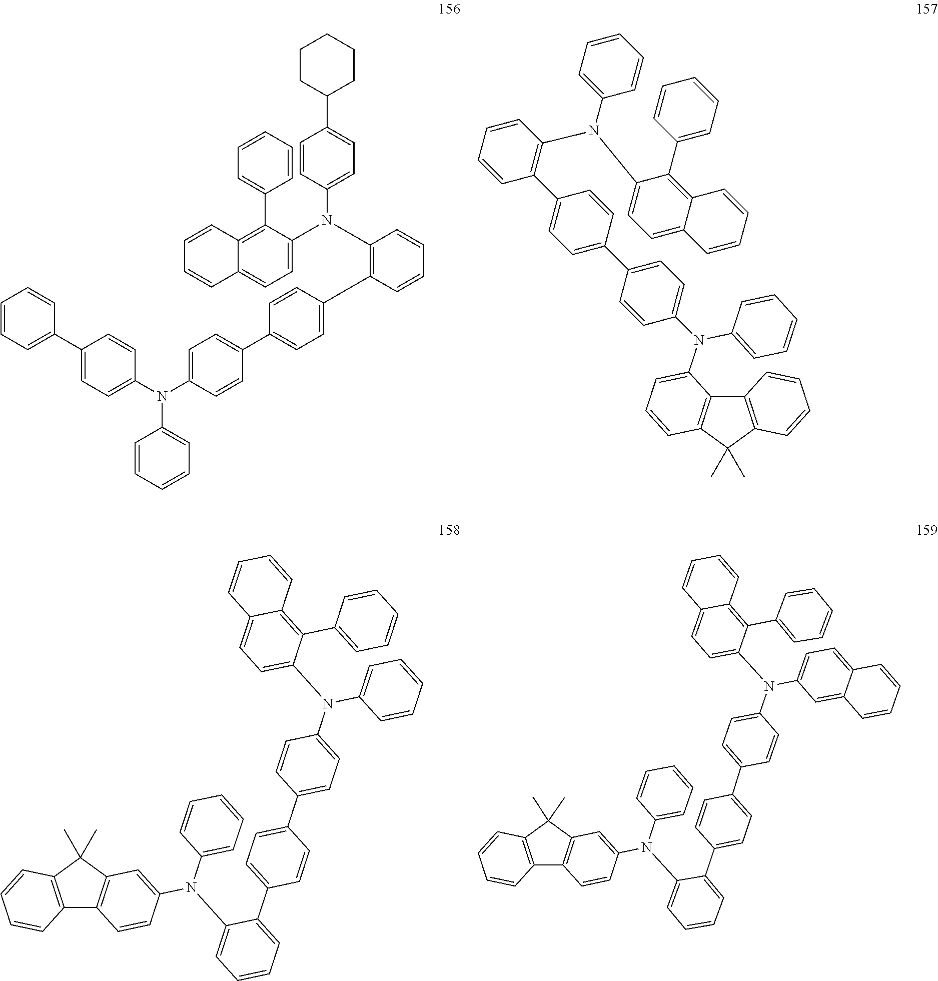 [SMILES: c1ccc(-c2ccc(N(c3ccccc3)c3ccc(-c4ccc(-c5ccccc5N(c5ccc(C6CCCCC6)cc5)c5ccc6ccccc6c5-c5ccccc5)cc4)cc3)cc2)cc1 ; CC1(C)c2ccccc2-c2c(N(c3ccccc3)c3ccc(-c4ccc(-c5ccccc5N(c5ccccc5)c5ccc6ccccc6c5-c5ccccc5)cc4)cc3)cccc21 ; CC1(C)c2ccccc2-c2ccc(N(c3ccccc3)c3ccccc3-c3ccc(-c4ccc(N(c5ccccc5)c5ccc6ccccc6c5-c5ccccc5)cc4)cc3)cc21 ; CC1(C)c2ccccc2-c2ccc(N(c3ccccc3)c3ccccc3-c3ccc(-c4ccc(N(c5ccc6ccccc6c5)c5ccc6ccccc6c5-c5ccccc5)cc4)cc3)cc21]

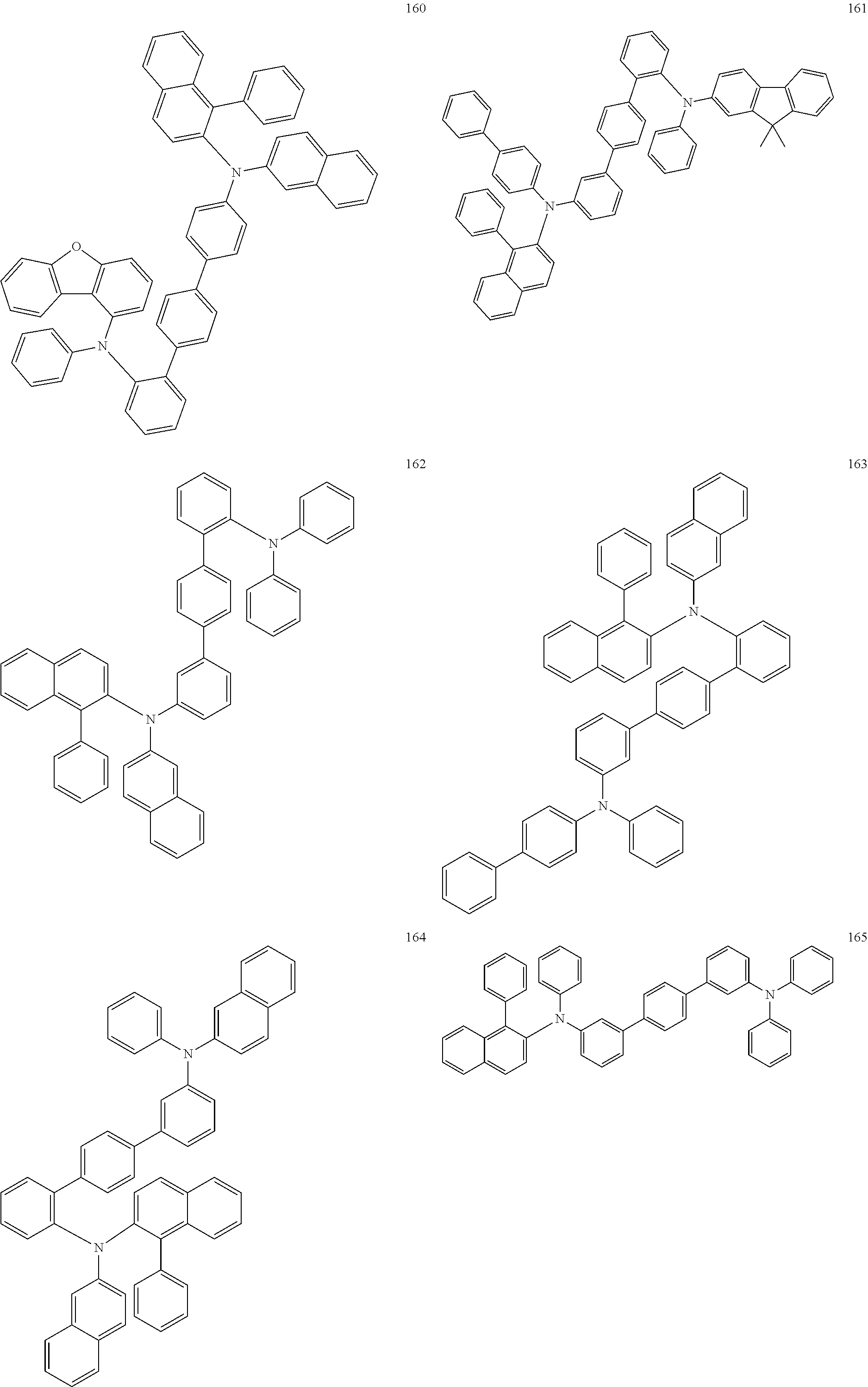

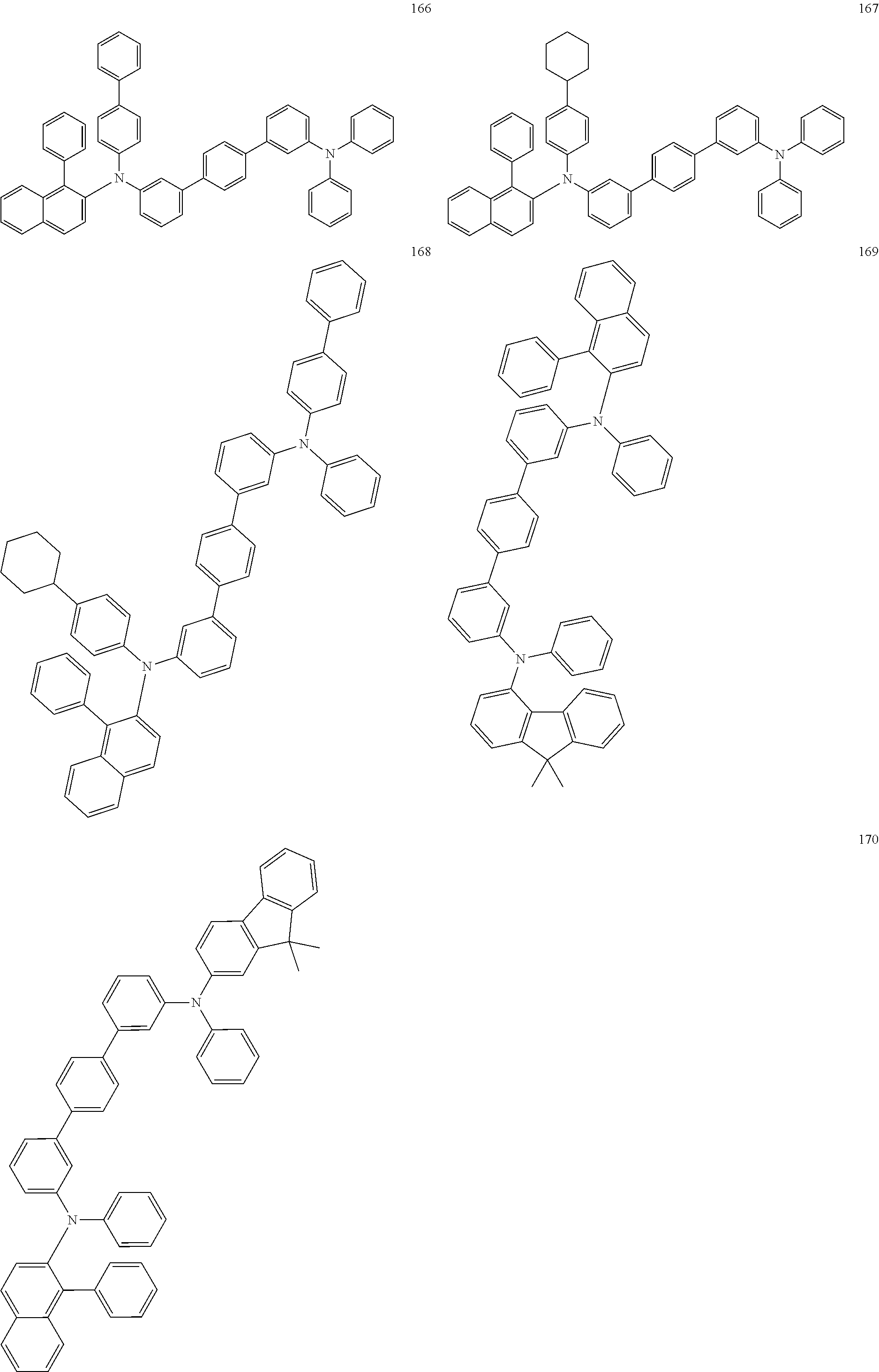

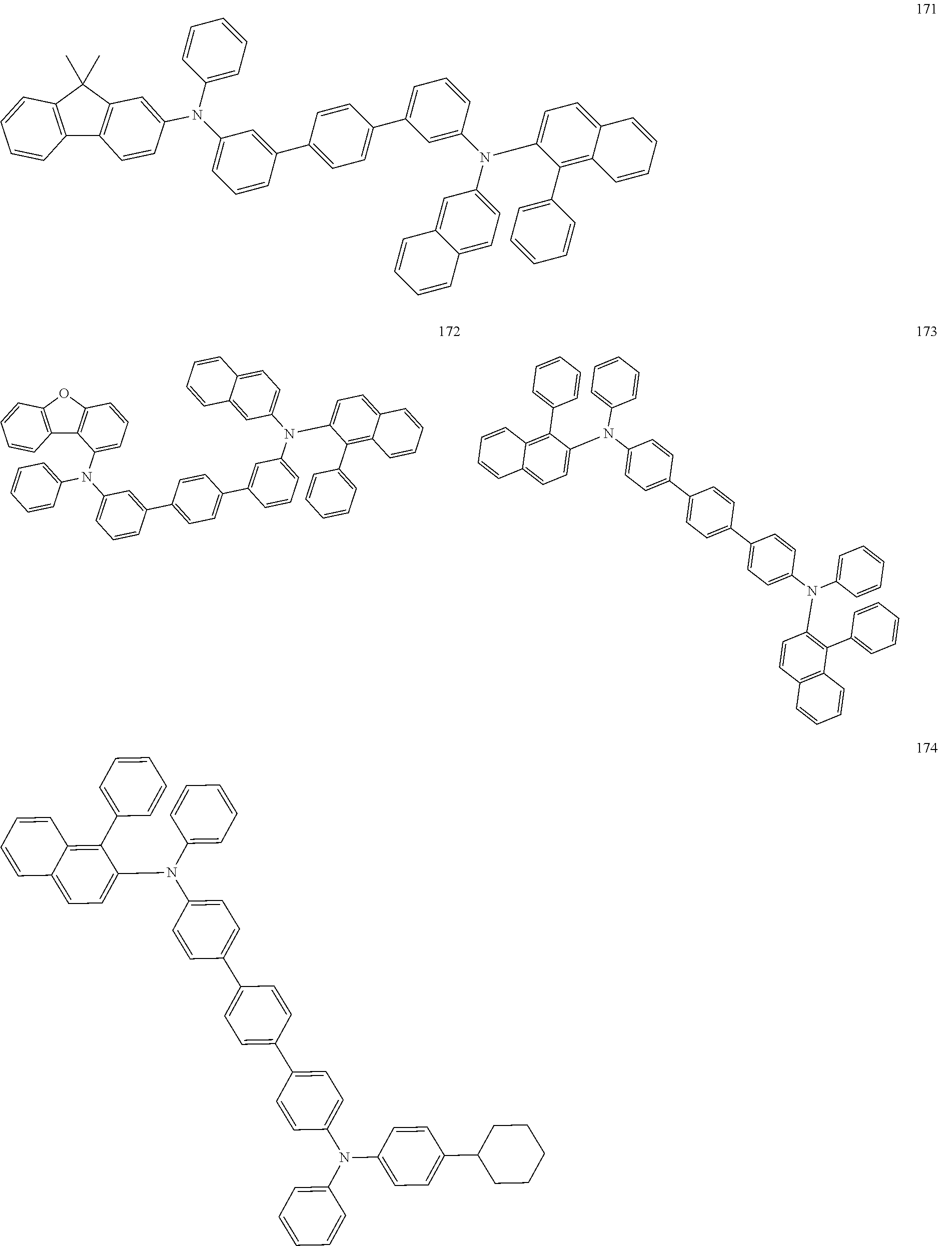 [SMILES: CC1(C)c2ccccc2-c2ccc(N(c3ccccc3)c3cccc(-c4ccc(-c5cccc(N(c6ccc7ccccc7c6)c6ccc7ccccc7c6-c6ccccc6)c5)cc4)c3)cc21 ; c1ccc(-c2c(N(c3cccc(-c4ccc(-c5cccc(N(c6ccccc6)c6cccc7oc8ccccc8c67)c5)cc4)c3)c3ccc4ccccc4c3)ccc3ccccc23)cc1 ; c1ccc(-c2c(N(c3ccccc3)c3ccc(-c4ccc(-c5ccc(N(c6ccccc6)c6ccc7ccccc7c6-c6ccccc6)cc5)cc4)cc3)ccc3ccccc23)cc1 ; c1ccc(-c2c(N(c3ccccc3)c3ccc(-c4ccc(-c5ccc(N(c6ccccc6)c6ccc(C7CCCCC7)cc6)cc5)cc4)cc3)ccc3ccccc23)cc1]

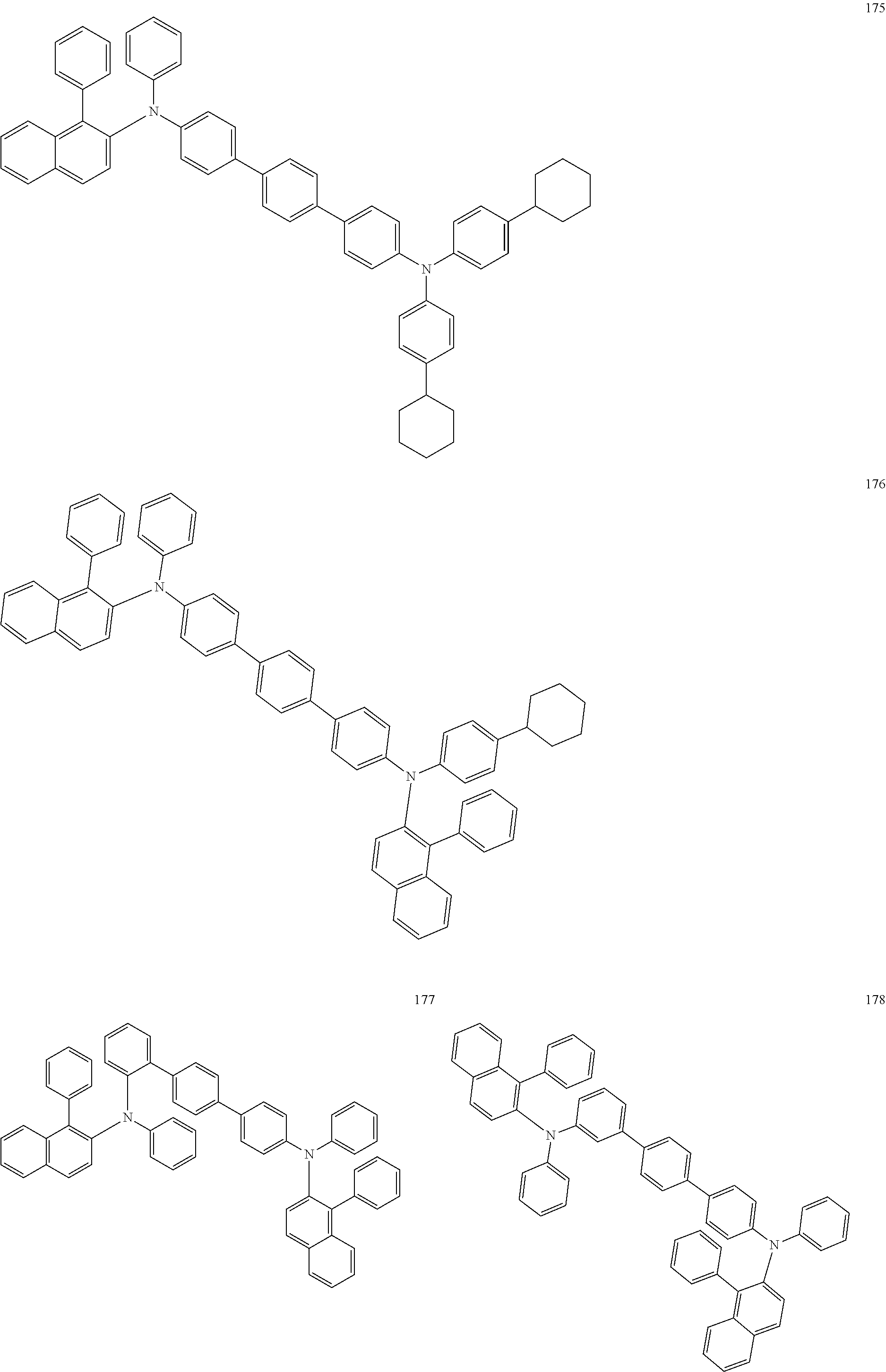 [SMILES: c1ccc(-c2c(N(c3ccccc3)c3ccc(-c4ccc(-c5ccc(N(c6ccc(C7CCCCC7)cc6)c6ccc(C7CCCCC7)cc6)cc5)cc4)cc3)ccc3ccccc23)cc1 ; c1ccc(-c2c(N(c3ccccc3)c3ccc(-c4ccc(-c5ccc(N(c6ccc(C7CCCCC7)cc6)c6ccc7ccccc7c6-c6ccccc6)cc5)cc4)cc3)ccc3ccccc23)cc1 ; c1ccc(-c2c(N(c3ccccc3)c3ccc(-c4ccc(-c5ccccc5N(c5ccccc5)c5ccc6ccccc6c5-c5ccccc5)cc4)cc3)ccc3ccccc23)cc1 ; c1ccc(-c2c(N(c3ccccc3)c3ccc(-c4ccc(-c5cccc(N(c6ccccc6)c6ccc7ccccc7c6-c6ccccc6)c5)cc4)cc3)ccc3ccccc23)cc1]

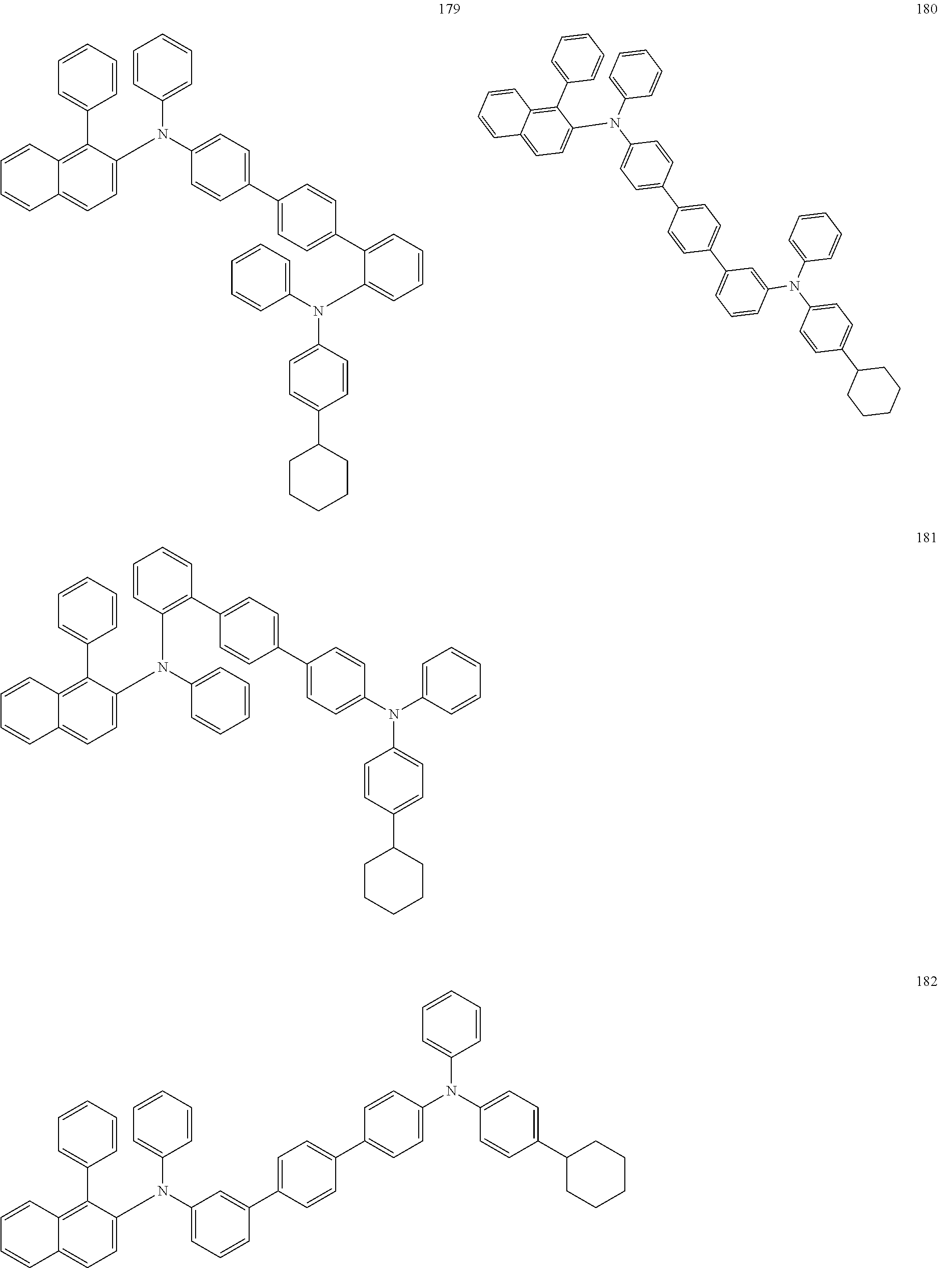 [SMILES: c1ccc(-c2c(N(c3ccccc3)c3ccc(-c4ccc(-c5ccccc5N(c5ccccc5)c5ccc(C6CCCCC6)cc5)cc4)cc3)ccc3ccccc23)cc1 ; c1ccc(-c2c(N(c3ccccc3)c3ccc(-c4ccc(-c5cccc(N(c6ccccc6)c6ccc(C7CCCCC7)cc6)c5)cc4)cc3)ccc3ccccc23)cc1 ; c1ccc(-c2c(N(c3ccccc3)c3ccccc3-c3ccc(-c4ccc(N(c5ccccc5)c5ccc(C6CCCCC6)cc5)cc4)cc3)ccc3ccccc23)cc1 ; c1ccc(-c2c(N(c3ccccc3)c3cccc(-c4ccc(-c5ccc(N(c6ccccc6)c6ccc(C7CCCCC7)cc6)cc5)cc4)c3)ccc3ccccc23)cc1]

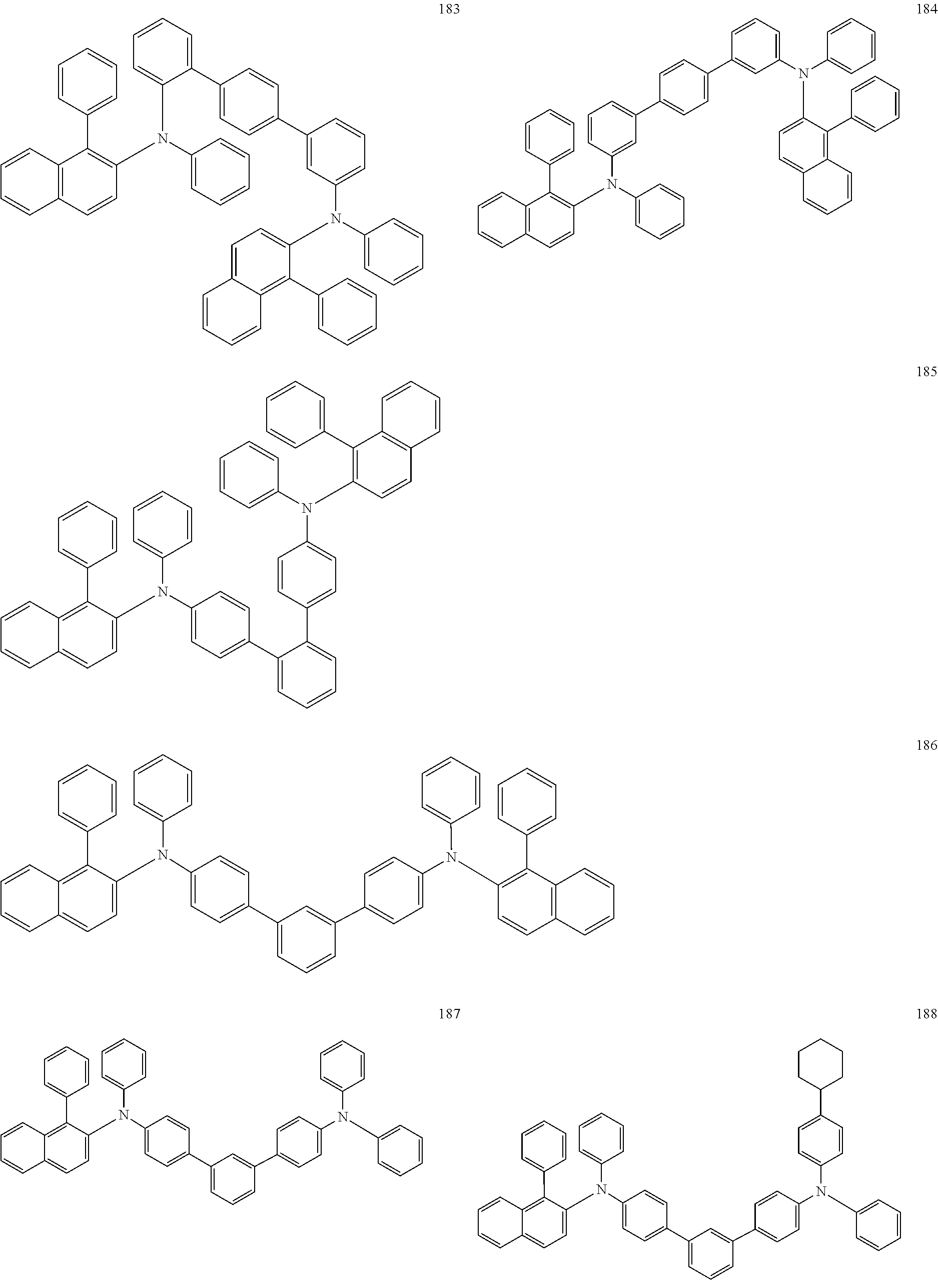

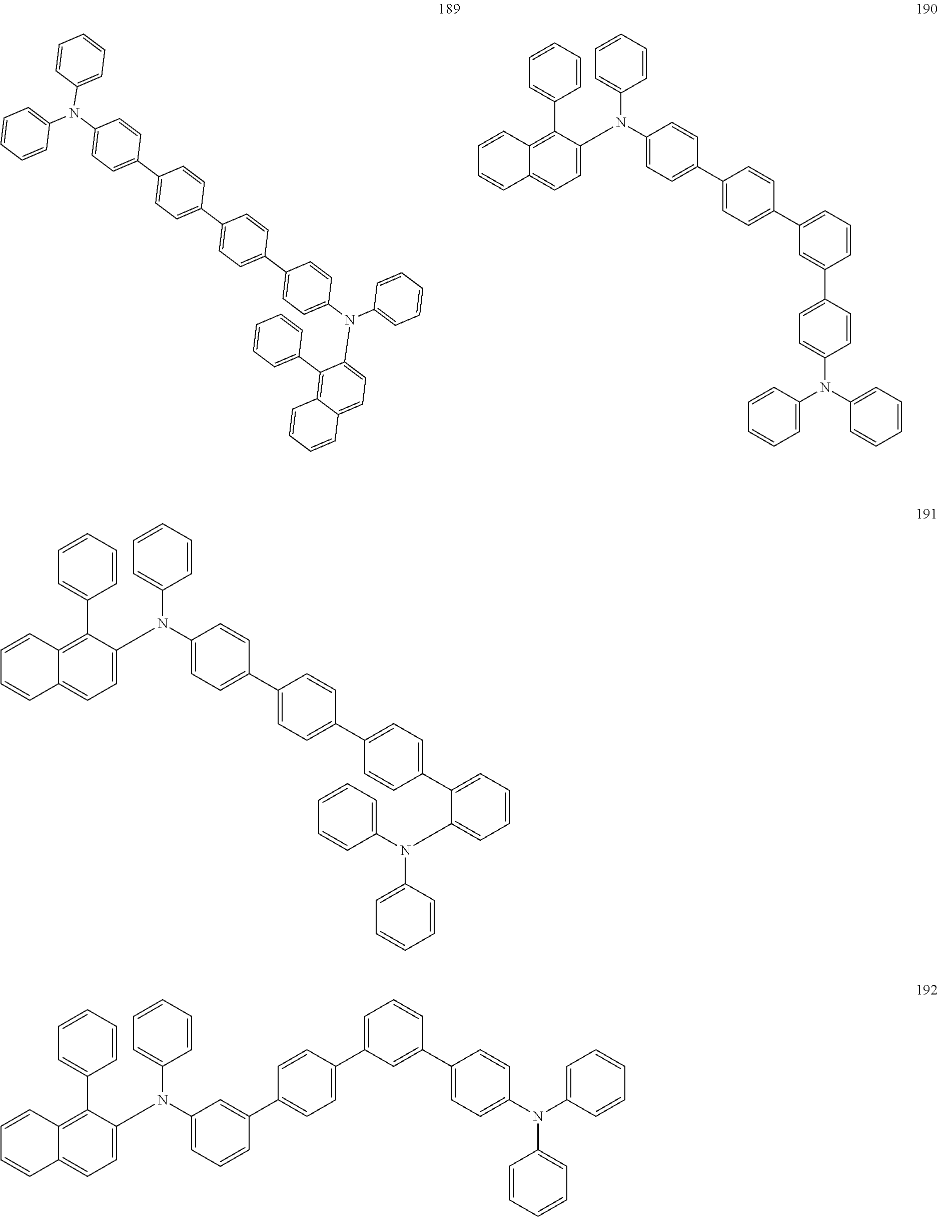 [SMILES: c1ccc(-c2c(N(c3ccccc3)c3ccc(-c4ccc(-c5ccc(-c6ccc(N(c7ccccc7)c7ccccc7)cc6)cc5)cc4)cc3)ccc3ccccc23)cc1 ; c1ccc(-c2c(N(c3ccccc3)c3ccc(-c4ccc(-c5cccc(-c6ccc(N(c7ccccc7)c7ccccc7)cc6)c5)cc4)cc3)ccc3ccccc23)cc1 ; c1ccc(-c2c(N(c3ccccc3)c3ccc(-c4ccc(-c5ccc(-c6ccccc6N(c6ccccc6)c6ccccc6)cc5)cc4)cc3)ccc3ccccc23)cc1 ; c1ccc(-c2c(N(c3ccccc3)c3cccc(-c4ccc(-c5cccc(-c6ccc(N(c7ccccc7)c7ccccc7)cc6)c5)cc4)c3)ccc3ccccc23)cc1]

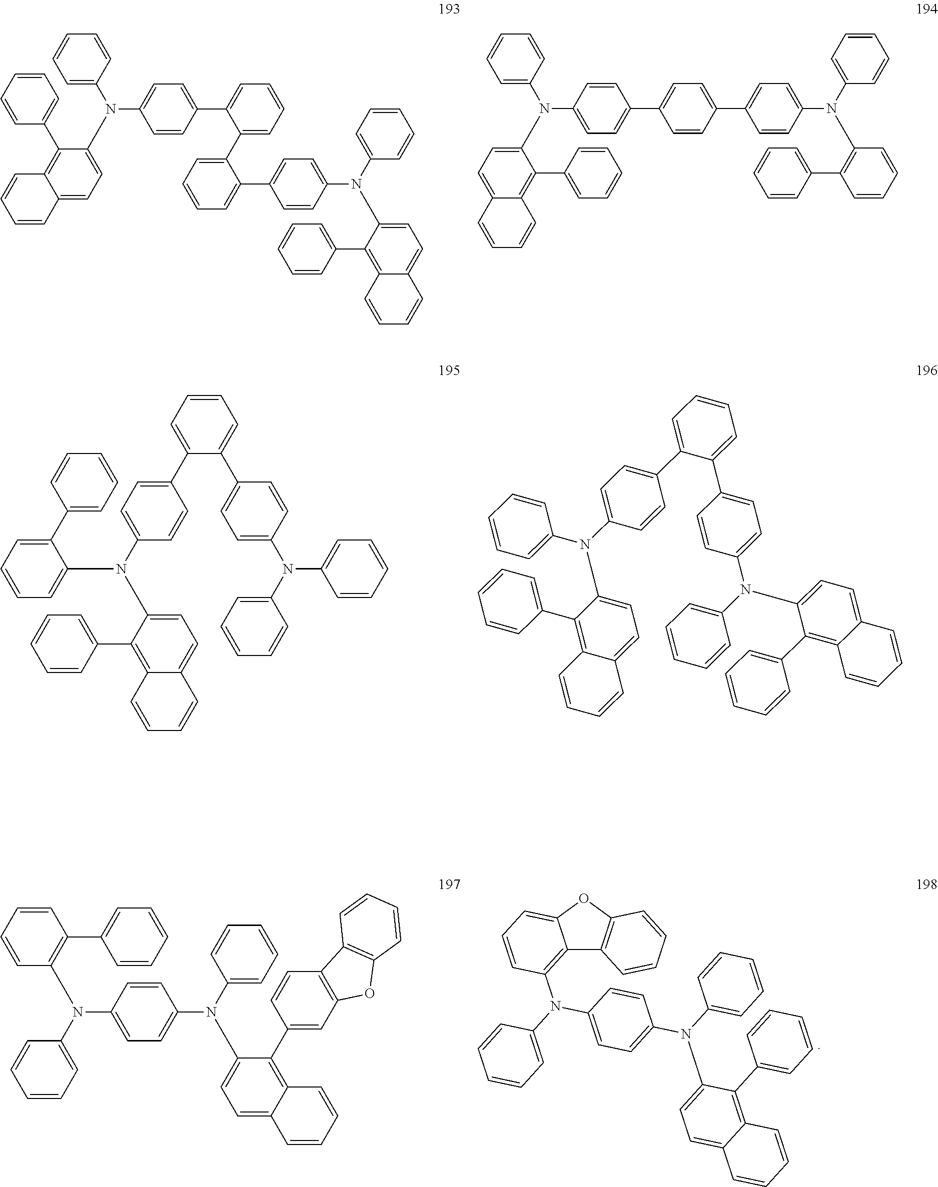

The amine compound according to one or more embodiments includes a structure in which two amine groups are connected via a linker. The amine compound according to one or more embodiments includes a first amine group, a second amine group, and a first linker connecting the first amine group and the second amine group. The amine compound according to one or more embodiments may include a first substituent connected to the first amine group. The first substituent may include a naphthyl moiety and a first sub-substituent connected to the naphthyl moiety. The naphthyl moiety included in the first substituent may be connected to the first amine group. One among carbon atoms forming the naphthyl moiety may be connected to a nitrogen atom of the first amine group, and one among the remaining carbon atoms may be connected to the first sub-substituent. The first sub-substituent may be directly connected to the naphthyl moiety. The amine compound having such a structure, according to one or more embodiments, may exhibit excellent or suitable electrical stability and high charge transport ability, and as a result, when the amine compound according to one or more embodiments is applied to the light-emitting element, luminous efficiency and element lifespan may be improved. Therefore, when the amine compound according to one or more embodiments of the present disclosure is applied to the hole transport region HTR of the light-emitting element ED, the light-emitting element having high efficiency, low voltage, and long-lifespan may be achieved.

With reference to FIGS. 3 to 6 again, the light-emitting element according to one or more embodiments of the present disclosure will be then further described.

As described above, the hole transport region HTR may contain the above-described amine compound according to one or more embodiments of the present disclosure. For example, the hole transport region HTR may contain the amine compound represented by Formula 1.

When the hole transport region HTR has a multilayer structure having a plurality of layers, then any one layer among the plurality of layers may contain the amine compound represented by Formula 1. For example, in one or more embodiments, the hole transport region HTR may include a hole injection layer HIL on the first electrode EL1 and a hole transport layer HTL on the hole injection layer HIL, wherein the hole transport layer HTL may contain the amine compound represented by Formula 1. However, embodiments of the present disclosure are not limited thereto, and, for example, in some embodiments, the hole injection layer HIL may contain the amine compound represented by Formula 1.

The hole transport region HTR may contain one, or two or more types (kinds) of the amine compounds represented by Formula 1. For example, in one or more embodiments, the hole transport region HTR may contain at least one selected from among compounds present in the above-described Compound Group 1.

In one or more embodiments, the hole transport region HTR may further include a compound represented by Formula H-1:

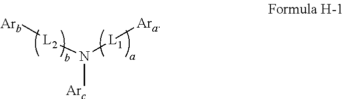

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In some embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms or a substituted or unsubstituted heteroaryl group having 2 to 30 ring-forming carbon atoms. In one or more embodiments, in Formula H-1, Ar_{c} may be a substituted or unsubstituted aryl group having 6 to 30 ring-forming carbon atoms or a substituted or unsubstituted heteroaryl group having 2 to 30 ring-forming carbon atoms.

The compound represented by Formula H-1 may be a monoamine compound. In some embodiments, the compound represented by Formula H-1 may be a diamine compound in which at least one selected from among Arₐ to Ar_{c} includes an amine group as a substituent. In some embodiments, the compound represented by Formula H-1 may be a carbazole-based compound including a substituted or unsubstituted carbazole group in at least one of Arₐ or Ar_{b}, or a fluorene-based compound including a substituted or unsubstituted fluorene group in at least one of Arₐ or Ar_{b}.

The compound represented by Formula H-1 may be any one selected from among compounds in Compound Group H. However, the compounds listed in Compound Group H are mere examples, and the compounds represented by Formula H-1 are not limited to those represented in Compound Group H:

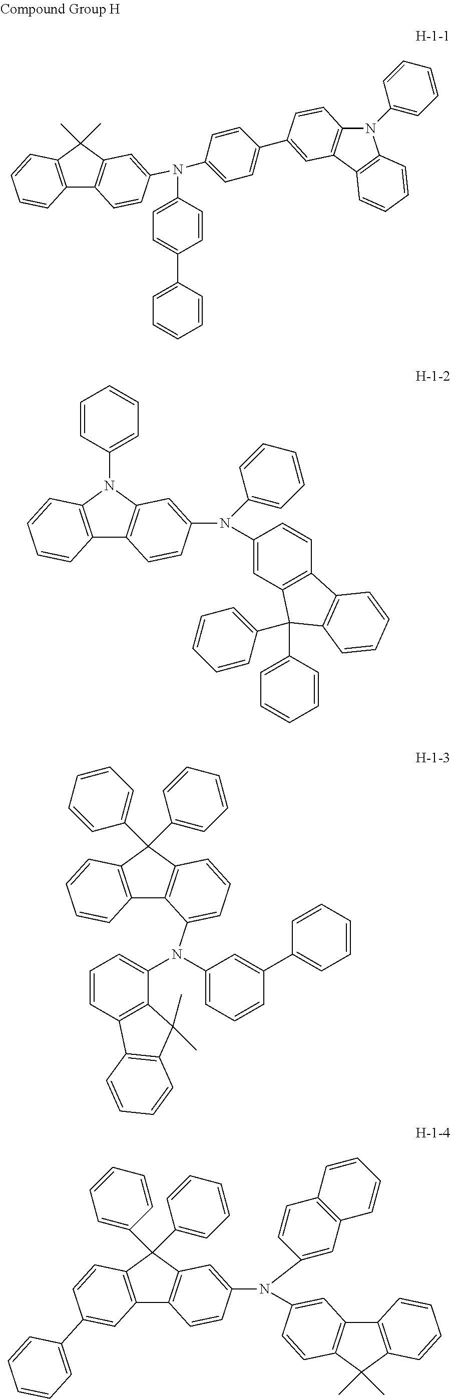 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

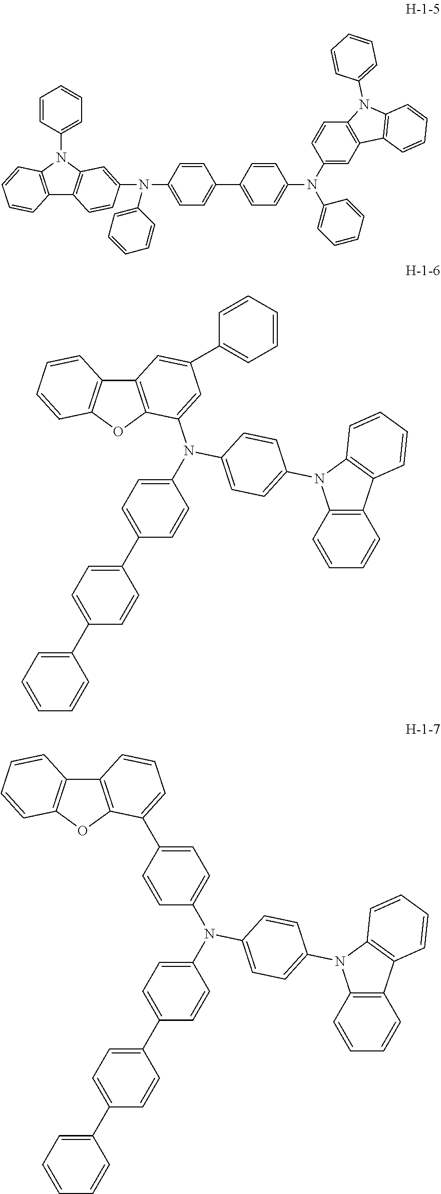

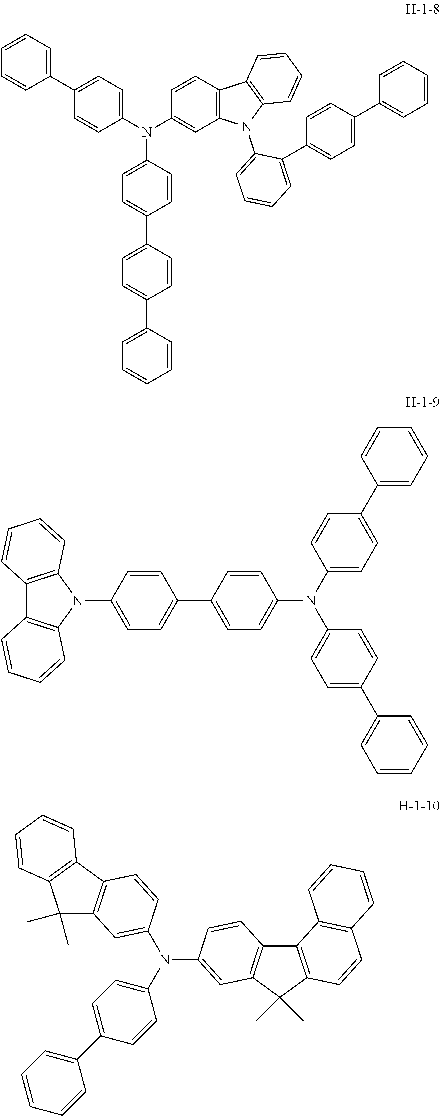 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

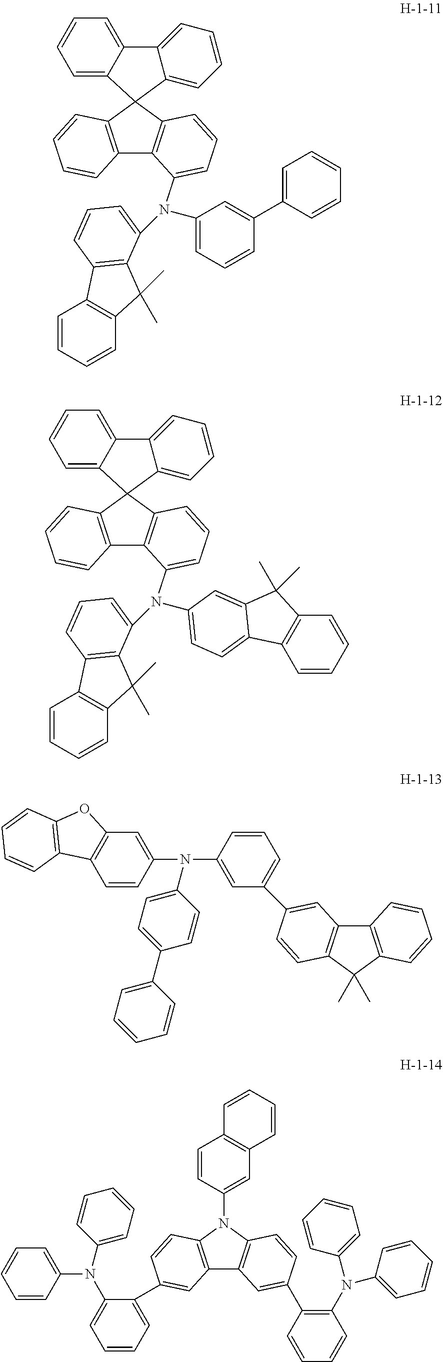

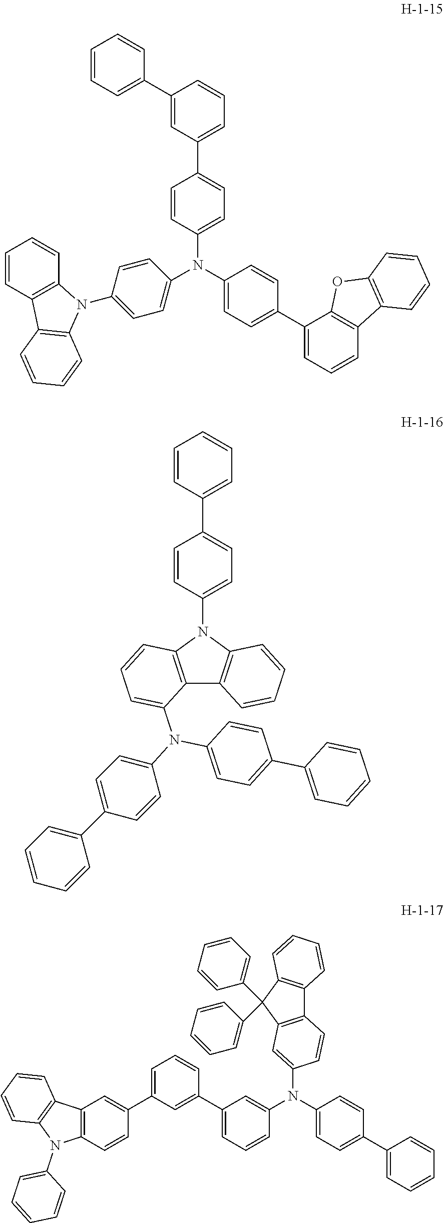 [SMILES: c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

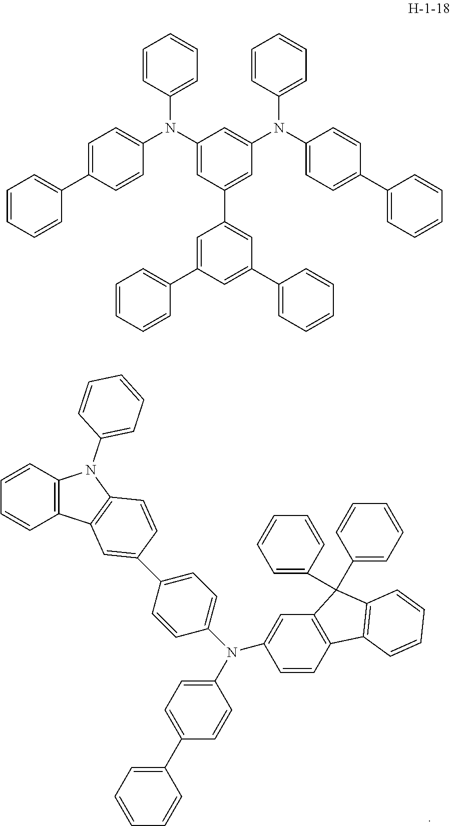 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In some embodiments, the hole transport region HTR may further include any suitable material generally available in the art.

For example, in some embodiments, the hole transport region HTR may include at least one selected from a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methyl phenyl)phenylamino] triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

In one or more embodiments, the hole transport region HTR may include at least one selected from a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl]benzenamine](TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may include at least one selected from 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include one or more of the above-described compounds of the hole transport region in at least one of a hole injection layer HIL, a hole transport layer HTL, or an electron blocking layer EBL.

A thickness of the hole transport region HTR may be from about 100 Å to about 10,000 Å, for example, from about 100 Å to about 5,000 Å. When the hole transport region HTR includes a hole injection layer HIL, the hole injection layer HIL may have, for example, a thickness of about 30 Å to about 1,000 Å. When the hole transport region HTR includes a hole transport layer HTL, the hole transport layer HTL may have a thickness of about 250 Å to about 1,000 Å. For example, when the hole transport region HTR includes an electron blocking layer EBL, the electron blocking layer EBL may have a thickness of about 10 Å to about 1,000 Å. When the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

In one or more embodiments, the hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments of the present disclosure are not limited thereto. For example, in some embodiments, the p-dopant may include a metal halide compound such as CuI and/or RbI, a quinone derivative such as tetracyanoquinodimethane (TCNQ) and/or 2,3,5,6-tetrafluoro-7,7,8,8-tetracyanoquinodimethane (F4-TCNQ), a metal oxide such as tungsten oxide and/or molybdenum oxide, a cyano group-containing compound such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments of the present disclosure are not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the light-emitting layer EML and may thus increase light emission efficiency. A material that may be included in the hole transport region HTR may be utilized as a material to be included in the buffer layer. The electron blocking layer EBL is a layer that serves to prevent or reduce the electron injection from the electron transport region ETR to the hole transport region HTR.

The light-emitting layer EML may be provided on the hole transport region HTR. The light-emitting layer EML may have, for example, a thickness of about 100 Å to about 1000 Å or about 100 Å to about 300 Å. The light-emitting layer EML may have a single layer formed of a single material, a single layer formed of a plurality of different materials, or a multilayer structure having a plurality of layers formed of a plurality of different materials.

In the light emitting element ED according to one or more embodiments, the light-emitting layer EML may be to emit blue light. The light emitting element ED according to one or more embodiments may include the above-described amine compound according to one or more embodiments in the hole transport region HTR to exhibit high luminous efficiency and long lifetime characteristics in the blue light-emitting region. However, embodiments of the present disclosure are not limited thereto.

In the light emitting element ED of one or more embodiments, the light-emitting layer EML may include at least one of anthracene derivatives, pyrene derivatives, fluoranthene derivatives, chrysene derivatives, dihydrobenzanthracene derivatives, or triphenylene derivatives. For example, in some embodiments, the light-emitting layer EML may include one or more anthracene derivatives and/or one or more pyrene derivatives.

In the light emitting elements ED of one or more embodiments, shown in FIG. 3 to FIG. 6, the light-emitting layer EML may include a host and a dopant. For example, in some embodiments, the light-emitting layer EML may include a compound represented by Formula E-1. The compound represented by Formula E-1 may be utilized as a fluorescence host material.

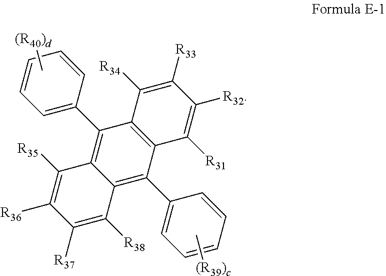

In Formula E-1, R₃₁ to R₄₀ may each independently be hydrogen, deuterium, a halogen, a substituted or unsubstituted silyl group, a substituted or unsubstituted thiol group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined (e.g., bonded) with an adjacent group to form a ring. In some embodiments, one or more selected from among R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

The compound represented by Formula E-1 may be any one selected from among compound E1 to E19.

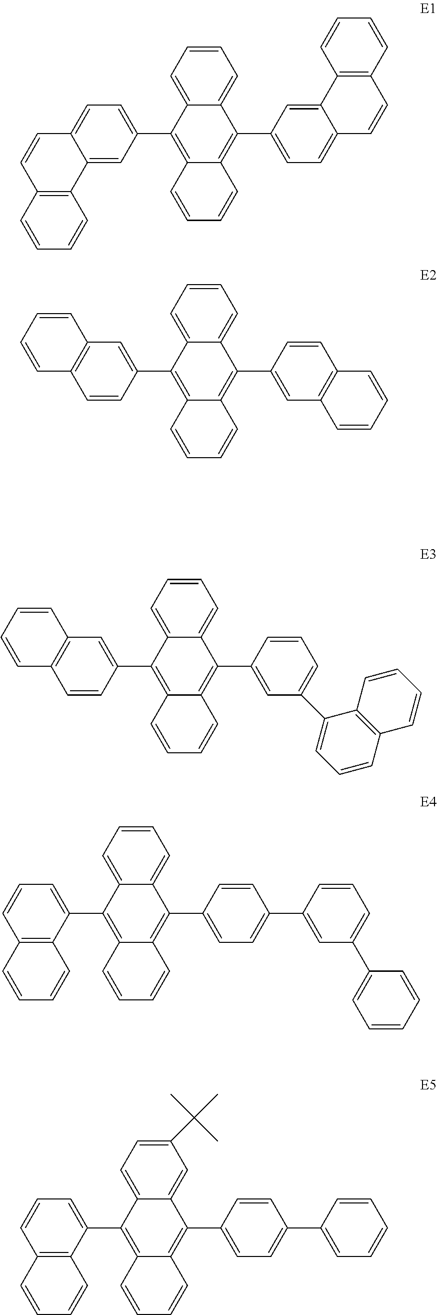

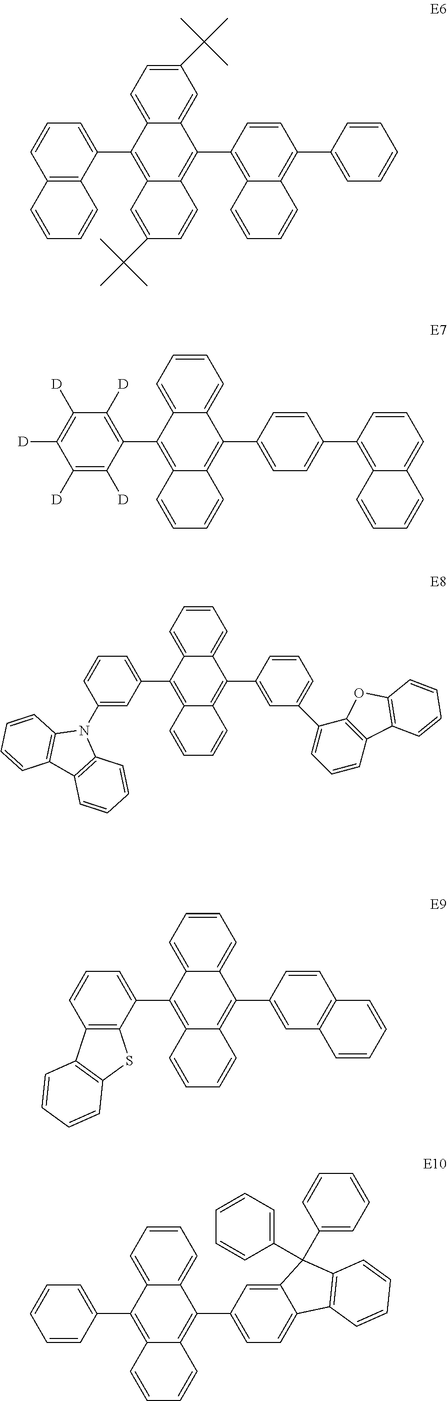

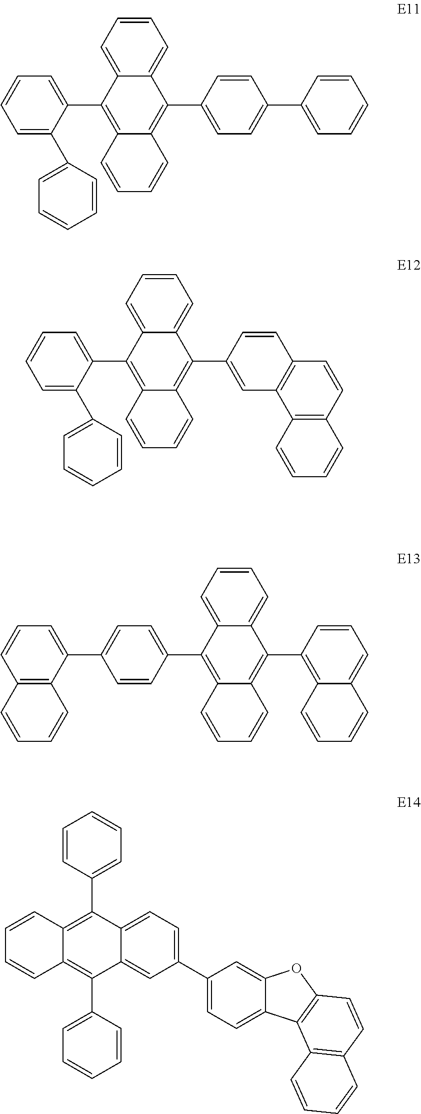

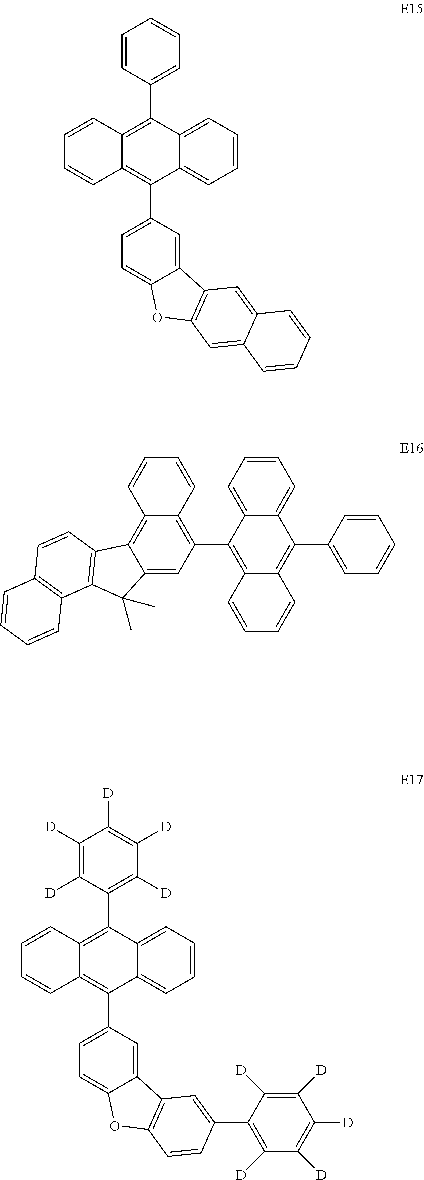

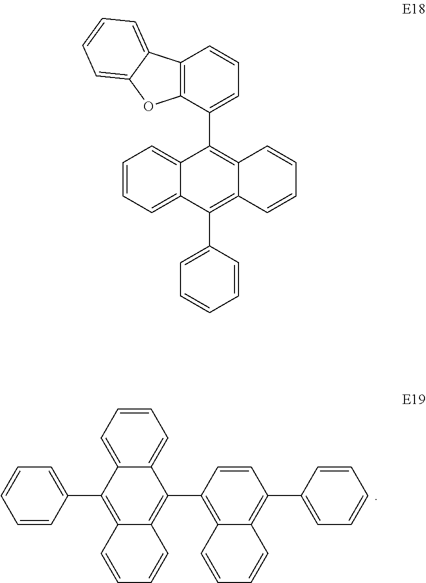

In one or more embodiments, the light-emitting layer EML may include a compound represented by Formula E-2a or Formula E-2b. The compound represented by Formula E-2a or Formula E-2b may be utilized as a phosphorescence host material.

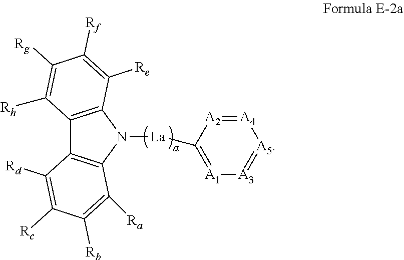

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” is an integer of 2 or more, multiple La's may each independently be a substituted or unsubstituted arylene group of 6 to 30 ring-forming carbon atoms or a substituted or unsubstituted heteroarylene group of 2 to 30 ring-forming carbon atoms.

In some embodiments, in Formula E-2a, A₁ to A₅ may each independently be N or CRᵢ. Rₐ to Rᵢ may each independently be hydrogen, deuterium, a substituted or unsubstituted amine group, a substituted or unsubstituted thiol group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In some embodiments, one or more selected from among Rₐ to Rᵢ may be combined with an adjacent group to form a hydrocarbon ring or a heterocycle including N, O, S, etc. as a ring-forming atom.

In some embodiments, in Formula E-2a, two or three selected from A₁ to A₅ may be N, and the remainder may be CRᵢ.

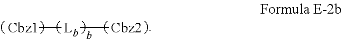

In Formula E-2b, Cbz1 and Cbz2 may each independently be an unsubstituted carbazole group or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and when “b” is an integer of 2 or more, multiple L_{b}'s may each independently be a substituted or unsubstituted arylene group of 6 to 30 ring-forming carbon atoms or a substituted or unsubstituted heteroarylene group of 2 to 30 ring-forming carbon atoms.

The compound represented by Formula E-2a or Formula E-2b may be any one selected from among compounds in Compound Group E-2. However, the compounds shown in Compound Group E-2 are only examples, and the compound represented by Formula E-2a or Formula E-2b is not limited to the compounds represented in Compound Group E-2.

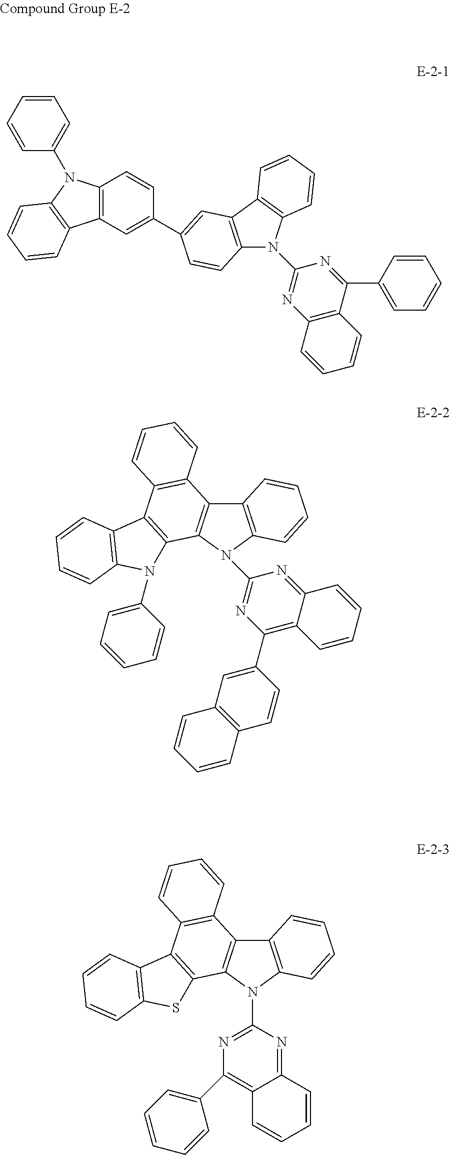 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

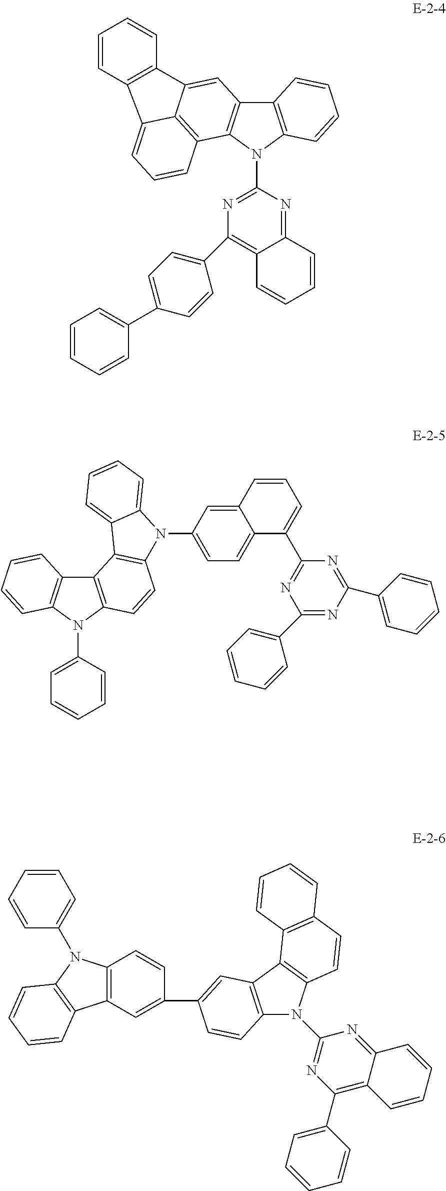 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

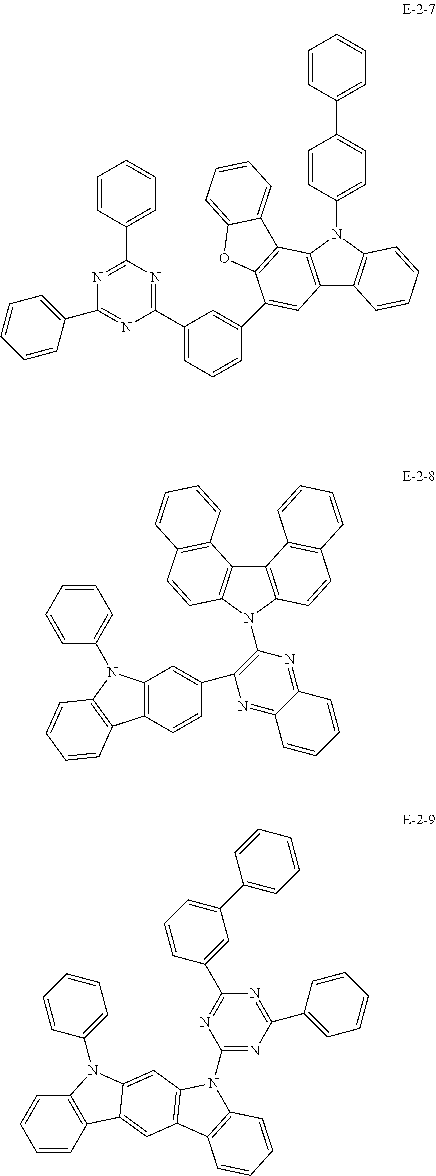 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

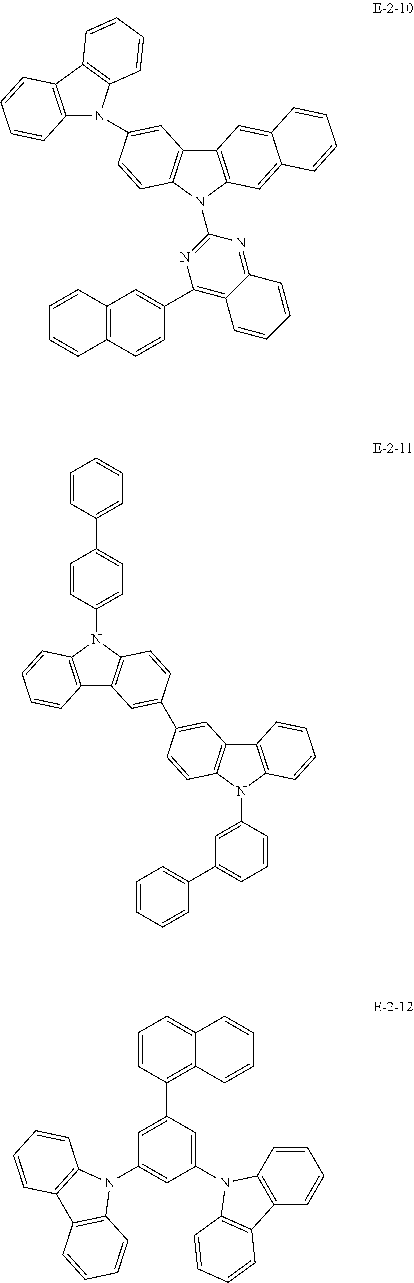 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

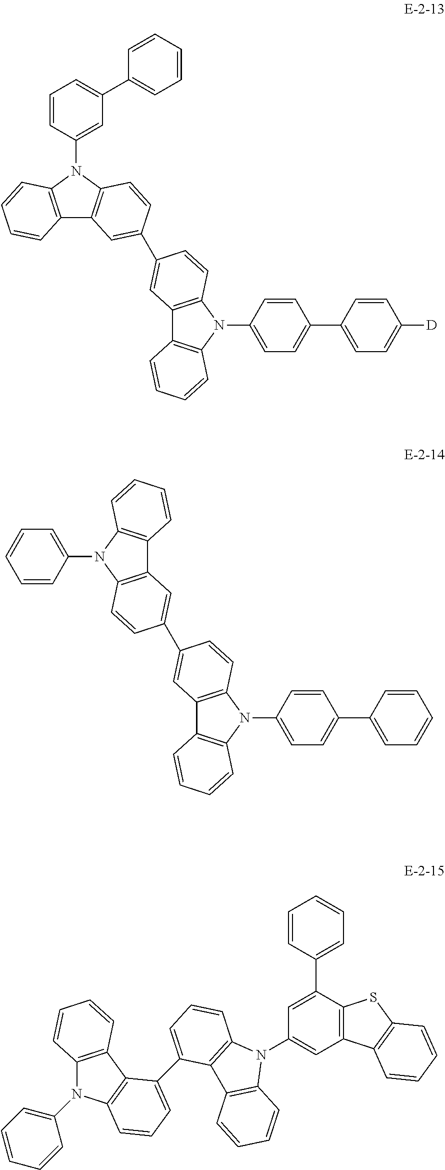 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

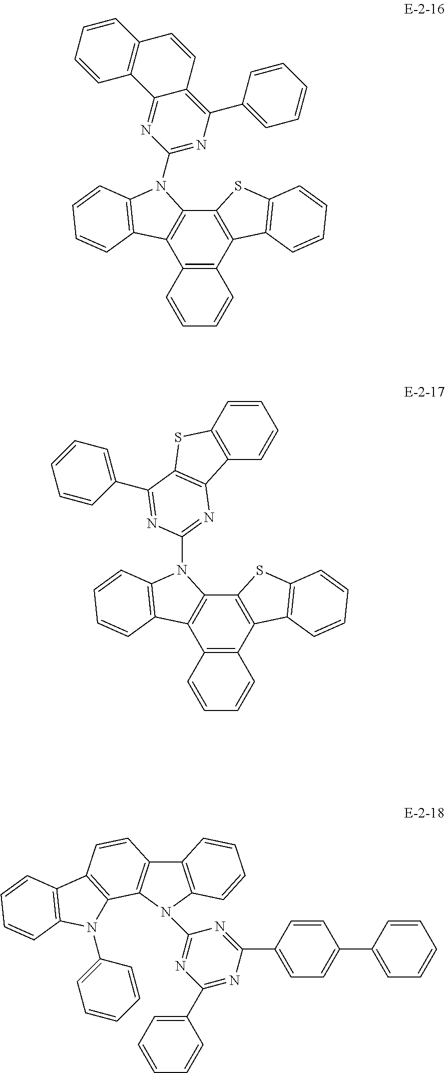 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

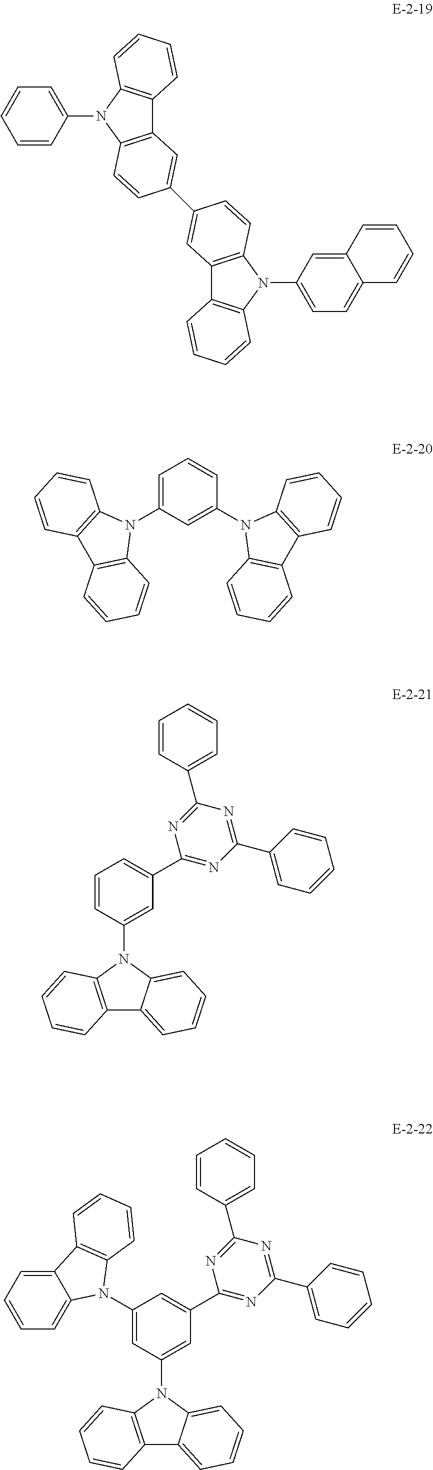 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

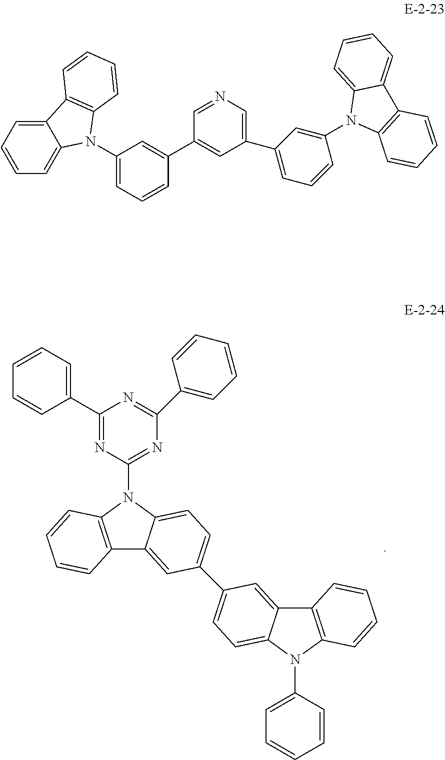 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, the light-emitting layer EML may further include a material well-suitable in the art as a host material. For example, the light-emitting layer EML may include, as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazol-2-yl)benzene (TPBi). However, embodiments of the present disclosure are not limited thereto. For example, tris(8-hydroxyquinolinato)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as the host material.

In one or more embodiments, the emission layer EML may include a compound represented by Formula M-a or Formula M-b. The compound represented by Formula M-a or Formula M-b may be utilized as a phosphorescence dopant material. In some embodiments, the compound represented by Formula M-a or Formula M-b may be utilized as an auxiliary dopant material.

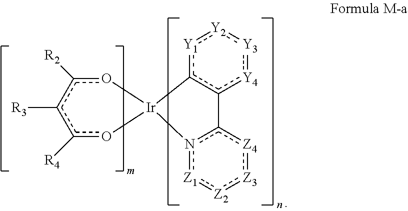

In Formula M-a, Y₁ to Y₄, and Z₁ to Z₄ may each independently be CR₁ or N, and R₁ to R₄ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” is or 1, and “n” is 2 or 3. In Formula M-a, when “m” is 0, “n” is 3, and when “m” is 1, “n” is 2.

The compound represented by Formula M-a may be utilized as a phosphorescence dopant.

The compound represented by Formula M-a may be any one selected from among Compounds M-a1 to M-a25. However, Compounds M-a1 to M-a25 are mere examples, and the compound represented by Formula M-a is not limited to the compounds represented by Compounds M-a1 to M-a25.

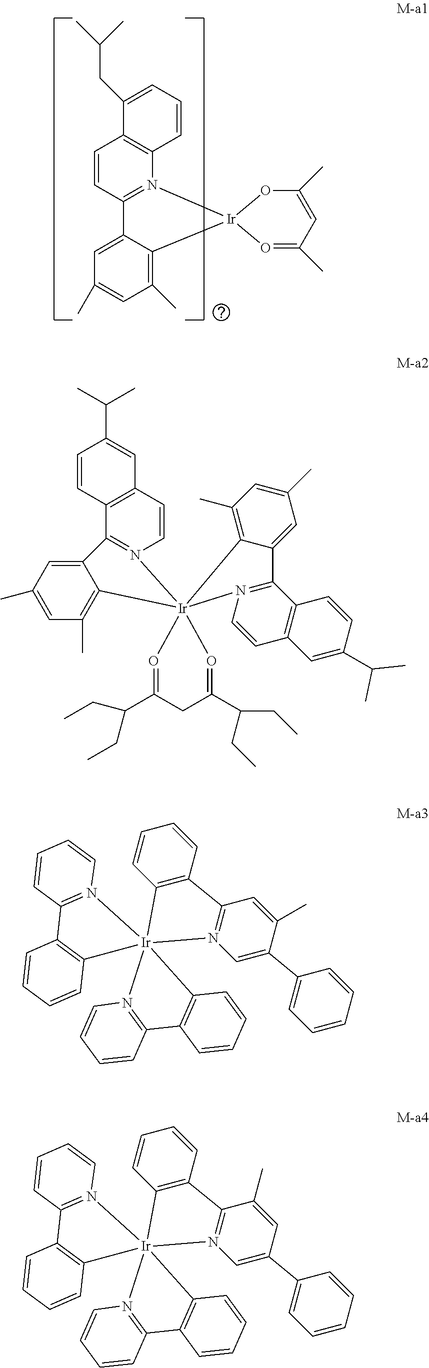

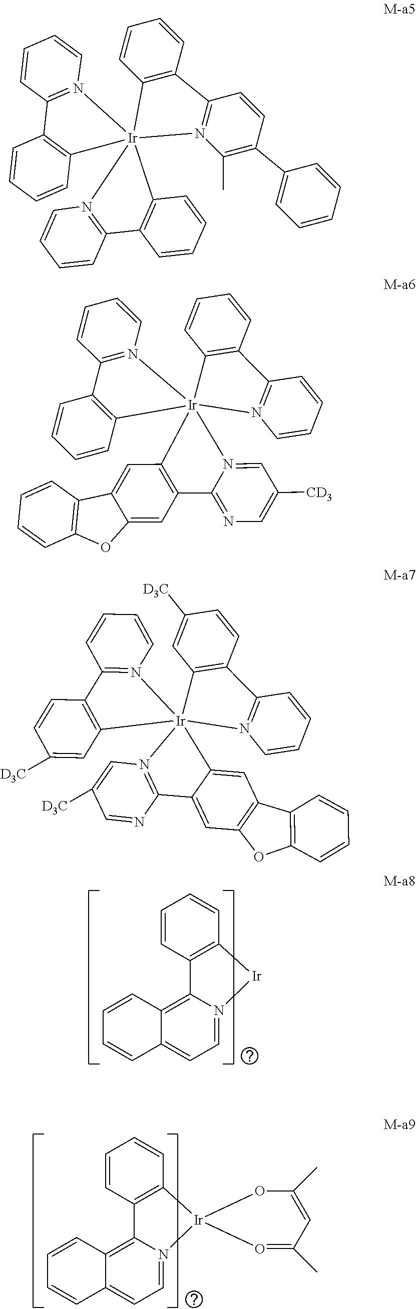

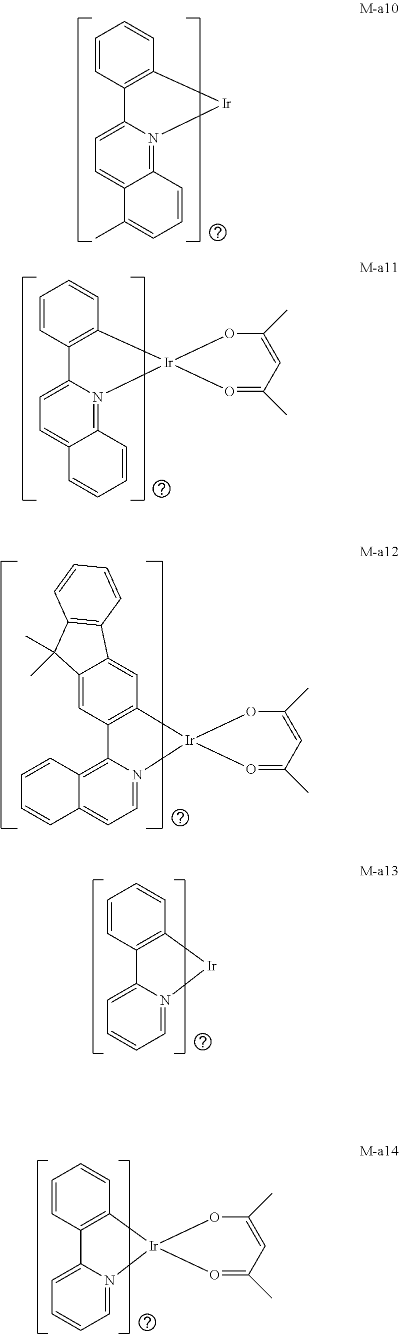

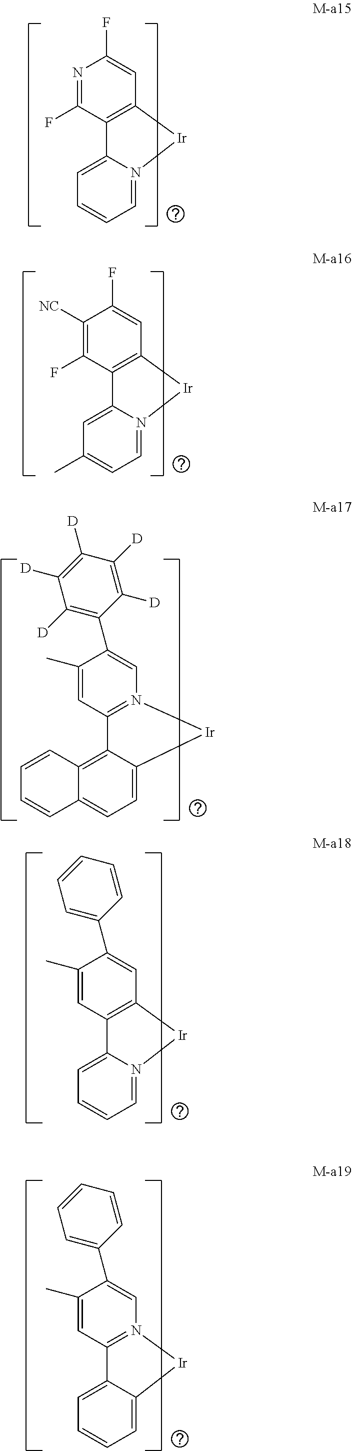

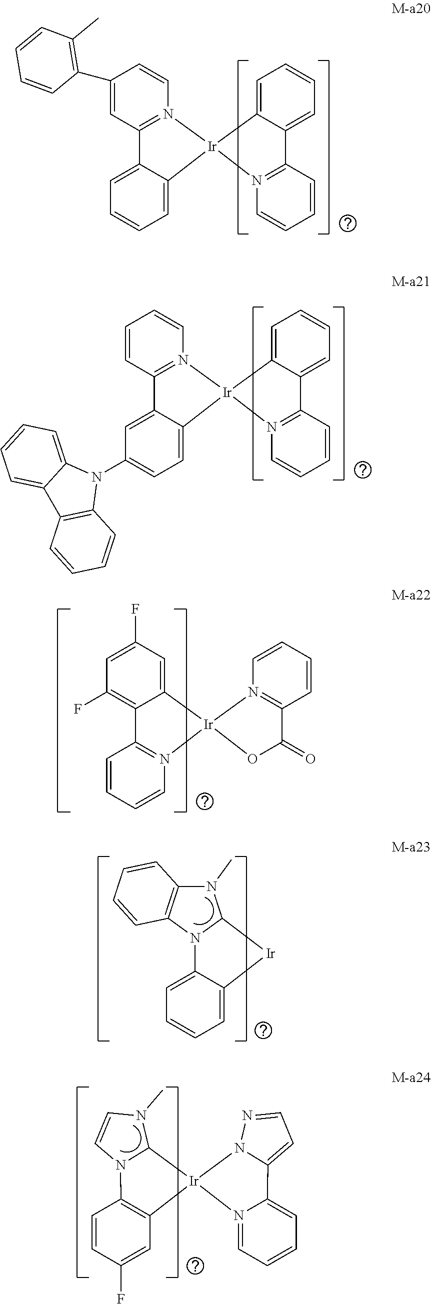

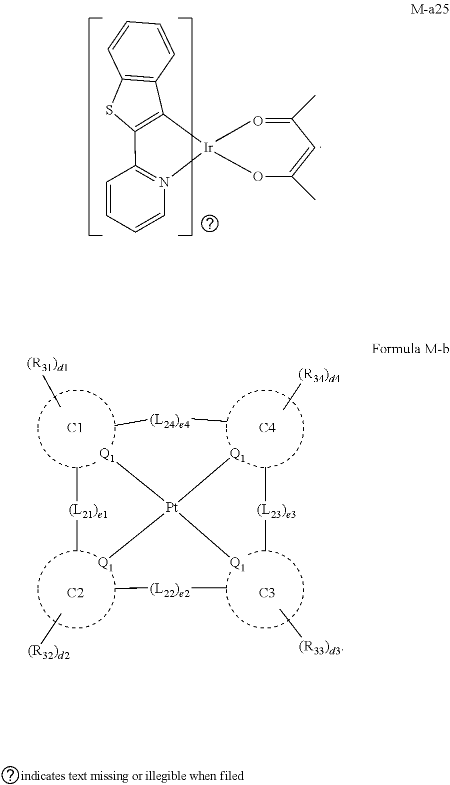

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

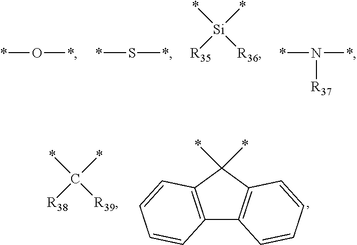

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may each independently be an integer of 0 to 4.

The compound represented by Formula M-b may be utilized as a blue phosphorescence dopant or a green phosphorescence dopant. In some embodiments, the compound represented by Formula M-b may be an auxiliary dopant and may be further included in the light-emitting layer EML.

The compound represented by Formula M-b may be any one selected from among Compound M-b-1 to Compound M-b-11. However, the compounds are mere examples, and the compound represented by Formula M-b is not limited to Compound M-b-1 to Compound M-b-11.

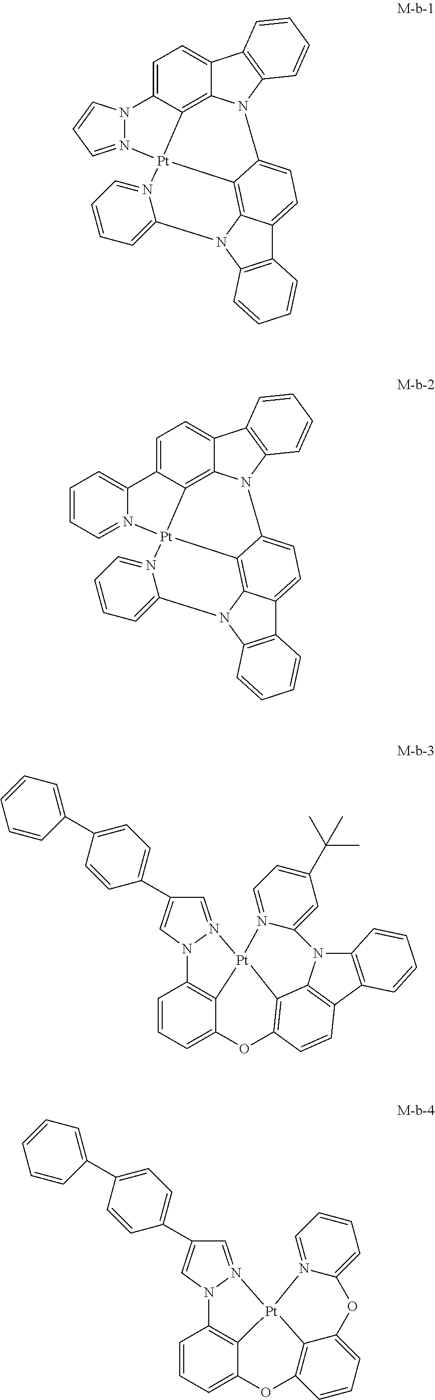

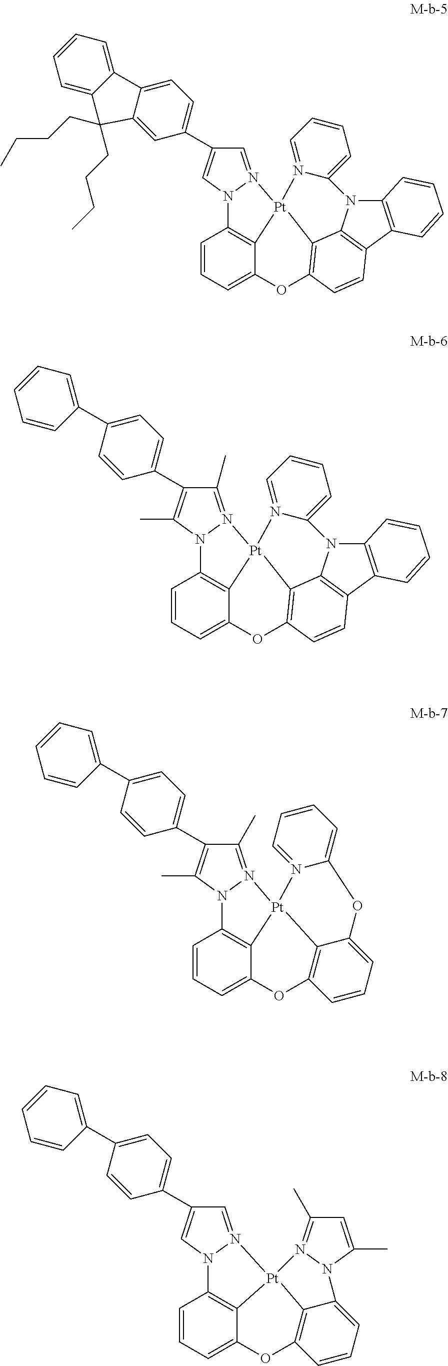

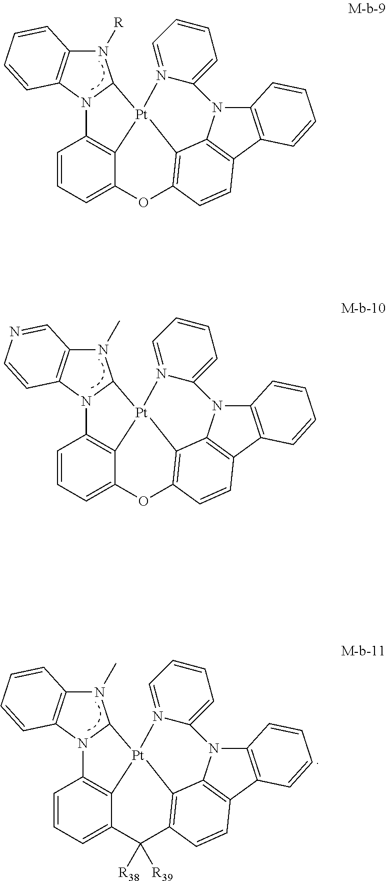

In the compounds above, R, R₃₈, and R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In one or more embodiments, the light-emitting layer EML may contain a first compound represented by any one selected from among Formulas F-a to F-c, a second compound represented by Formula HT-1, a third compound represented by Formula ET-1, and a fourth compound represented by Formula D-1.

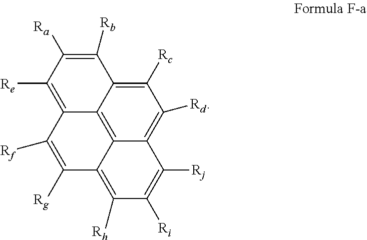

In Formula F-a, two selected from Rₐ to Rⱼ may each independently be substituted with •—NAr₁Ar₂. The remainder not substituted with •—NAr₁Ar₂ among Rₐ to Rⱼ may each independently be hydrogen, deuterium, a halogen, a cyano group, a substituted or unsubstituted amine group, a substituted or unsubstituted alkyl group of to 20 carbon atoms, a substituted or unsubstituted aryl group of 6 to 30 ring-forming carbon atoms, or a substituted or unsubstituted heteroaryl group of 2 to 30 ring-forming carbon atoms. In •—NAr₁Ar₂, Ar₁ and Ar₂ may each independently be a substituted or unsubstituted aryl group of 6 to 30 ring-forming carbon atoms or a substituted or unsubstituted heteroaryl group of 2 to 30 ring-forming carbon atoms. For example, in some embodiments, at least one selected from among Ar₁ and Ar₂ may be a heteroaryl group including O or S as a ring-forming atom.

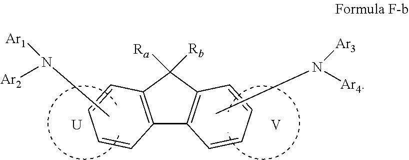

In Formula F-b, Rₐ and Rb may each independently be hydrogen, deuterium, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Ar₁ to Ar₄ may each independently be a substituted or unsubstituted aryl group of 6 to 30 ring-forming carbon atoms or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms. In some embodiments, at least one selected from among Ar₁ to Ar₄ may be a heteroaryl group including O or S as a ring-forming atom.

In Formula F-b, the number of rings represented by U and V may each independently be 0 or 1. For example, in Formula F-b, when the number of U or V is 1, one ring forms a fused ring at the designated part by U or V, and when the number of U or V is 0, a ring is not present at the designated part by U or V. For example, when the number of U is 0, and the number of V is 1, or when the number of U is 1, and the number of V is 0, a fused ring having the fluorene core of Formula F-b may be a ring compound with four rings. In some embodiments, when the number of both (e.g., simultaneously) U and V is 0, the fused ring of Formula F-b may be a ring compound with three rings. In some embodiments, when the number of both (e.g., simultaneously) U and V is 1, a fused ring having the fluorene core of Formula F-b may be a ring compound with five rings.

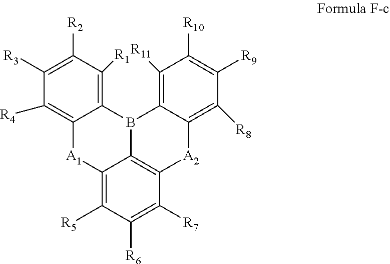

In Formula F-c, A₁ and A₂ may each independently be O, S, Se, or NRₘ, and Rₘ may be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thiol group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A₂ may each independently be combined with the substituents of an adjacent ring to form a fused ring. For example, when A₁ and A₂ may each independently be NRₘ, A₁ may be combined with R₄ or R₅ to form a ring. In some embodiments, A₂ may be combined with R₇ or R₈ to form a ring.

In one or more embodiments, the second compound may be utilized as a hole transporting host material of the light-emitting layer EML.

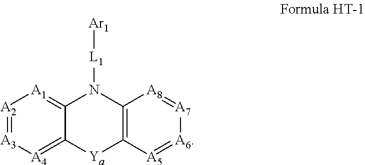

In Formula HT-1, A₁ to A₈ may each independently be N or CR₅₁. For example, in some embodiments, all A₁ to A₈ may be CR₅₁. In some embodiments, any one selected from among A₁ to A₈ may be N, and the remainder may be CR₅₁.

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, in some embodiments, L₁ may be a direct linkage, a substituted or unsubstituted phenylene group, a substituted or unsubstituted divalent biphenyl group, a substituted or unsubstituted divalent carbazole group, and/or the like, but embodiments of the present disclosure are not limited thereto.

In Formula HT-1, Yₐ may be a direct linkage, CR₅₂R₅₃, or SiR₅₄R₅₅. For example, it may refer to that two benzene rings connected with the nitrogen atom of Formula HT-1 may be connected via a direct linkage,

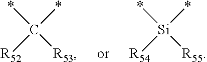

In Formula HT-1, when Yₐ is a direct linkage, the substituent represented by Formula HT-1 may include a carbazole moiety.

In Formula HT-1, Ar₁ may be a substituted or unsubstituted aryl group of 6 to ring-forming carbon atoms or a substituted or unsubstituted heteroaryl group of 2 to ring-forming carbon atoms. For example, in some embodiments, Ar₁ may be a substituted or unsubstituted carbazole group, a substituted or unsubstituted dibenzofuran group, a substituted or unsubstituted dibenzothiophene group, a substituted or unsubstituted biphenyl group, and/or the like, but embodiments of the present disclosure are not limited thereto.

In Formula HT-1, R₅₁ to R₅₅ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In some embodiments, one or more of R₅₁ to R₅₅ may be combined with an adjacent group to form a ring. For example, in some embodiments, R₅₁ to R₅₅ may each independently be hydrogen or deuterium. In some embodiments, R₅₁ to R₅₅ may each independently be an unsubstituted methyl group or an unsubstituted phenyl group.

In one or more embodiments, the second compound represented by Formula HT-1 may be any one selected from among compounds represented in Compound Group 2. The light-emitting layer EML may include at least one selected from among the compounds represented in Compound Group 2 as a hole transport host material.

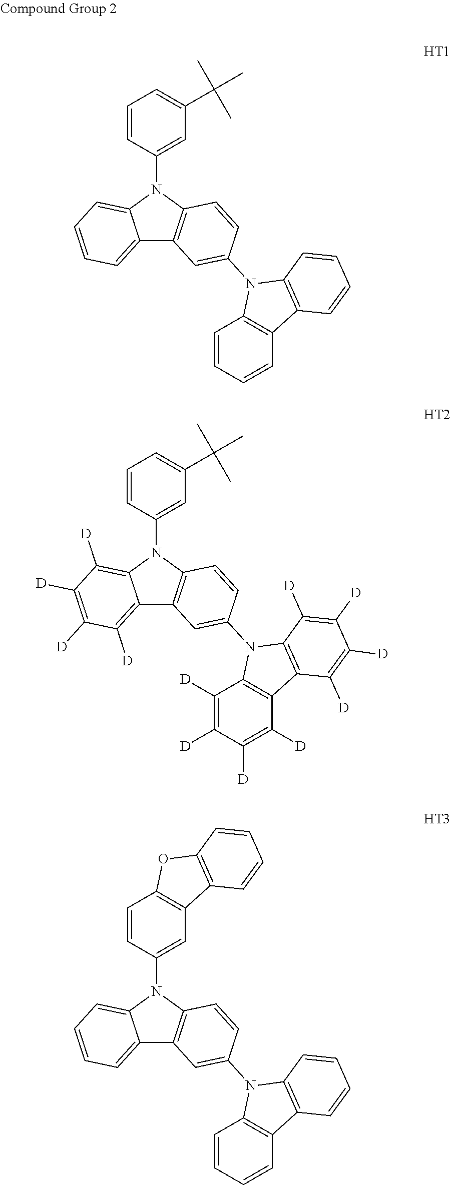 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

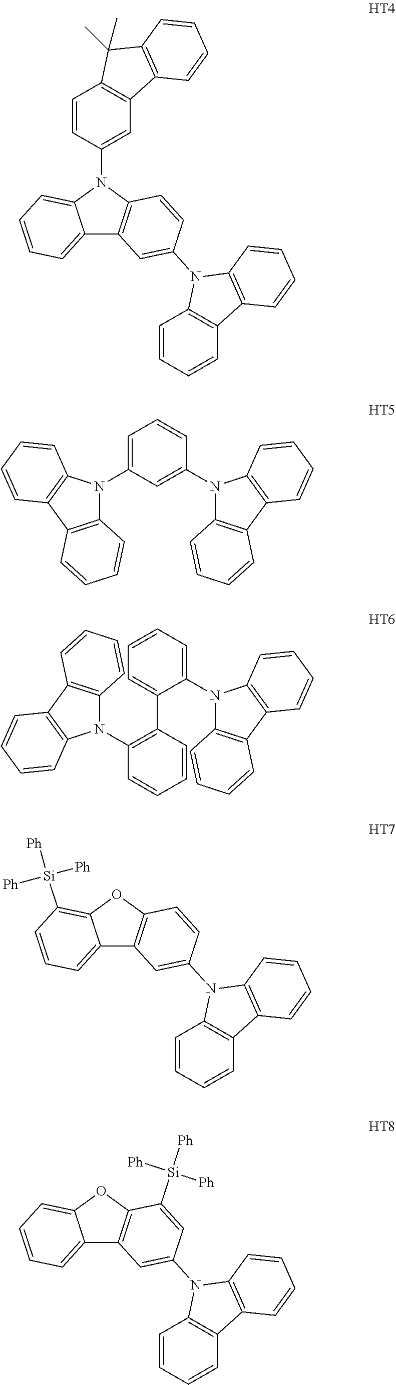

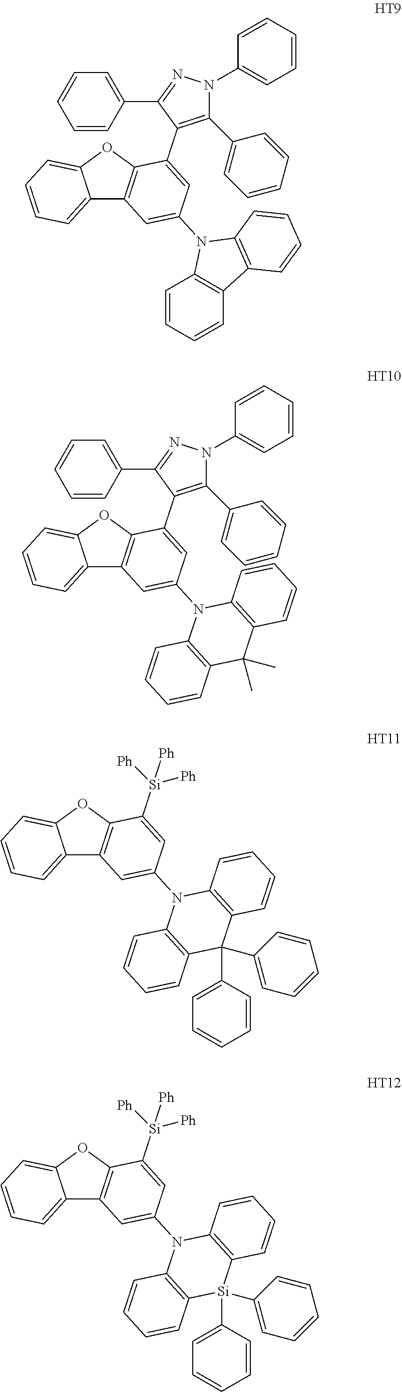 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

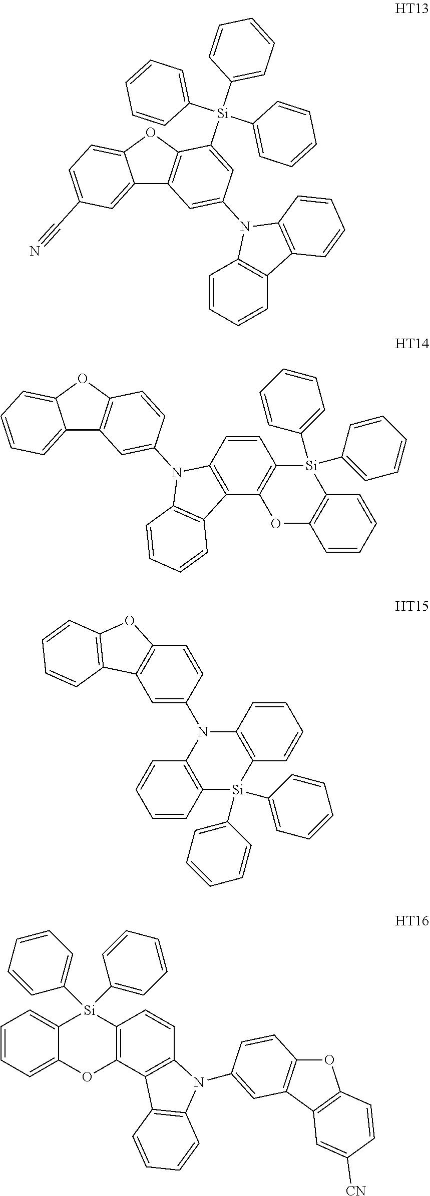 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1]

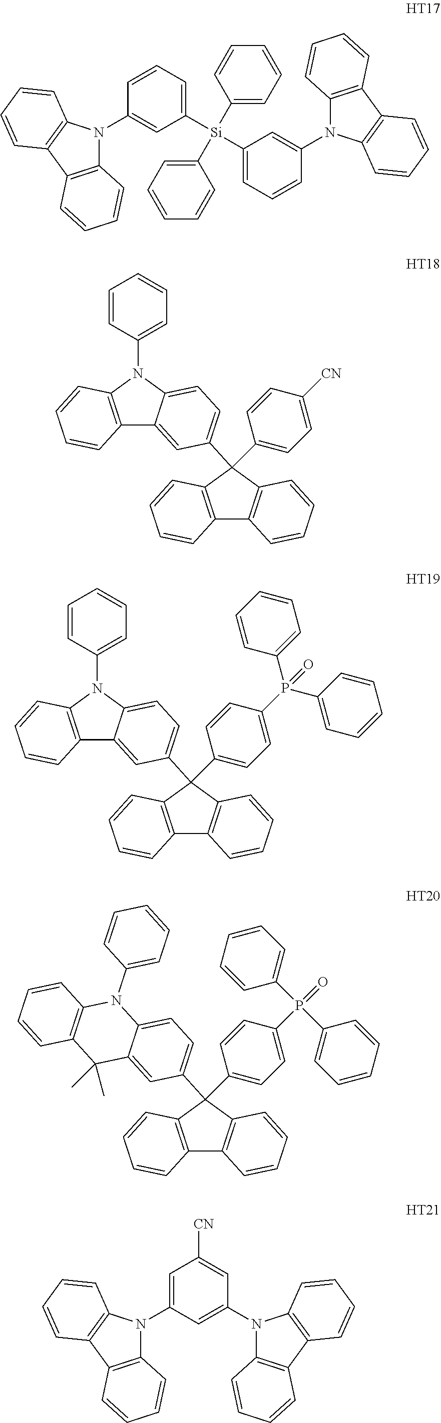 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1]

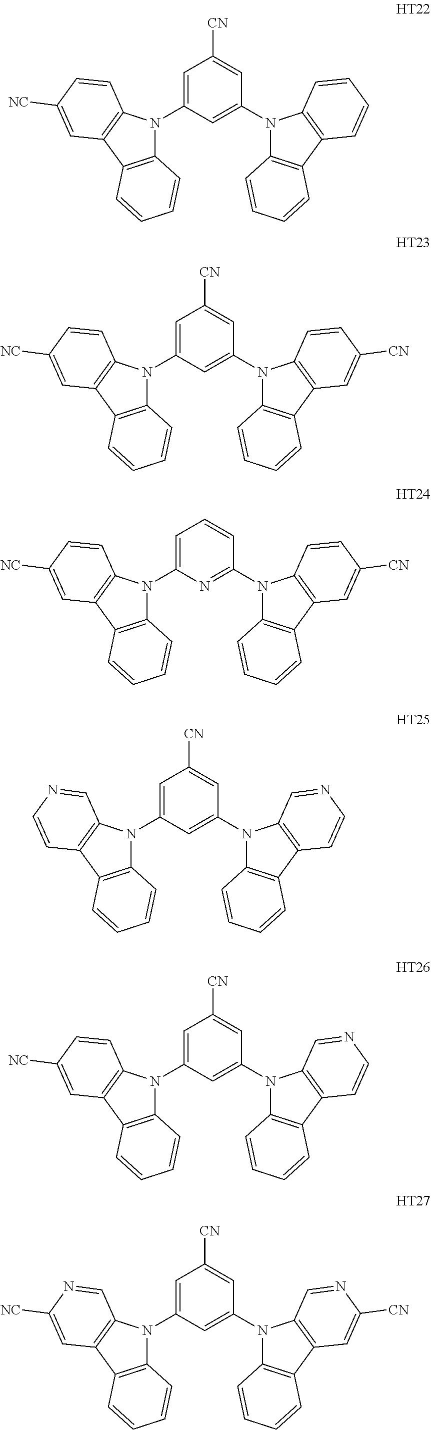 [SMILES: N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1]

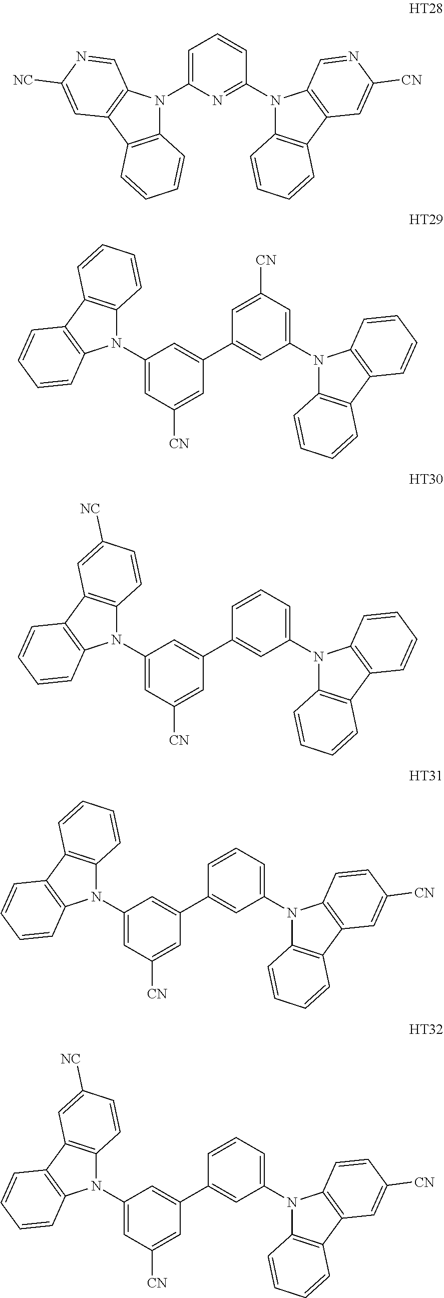 [SMILES: N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

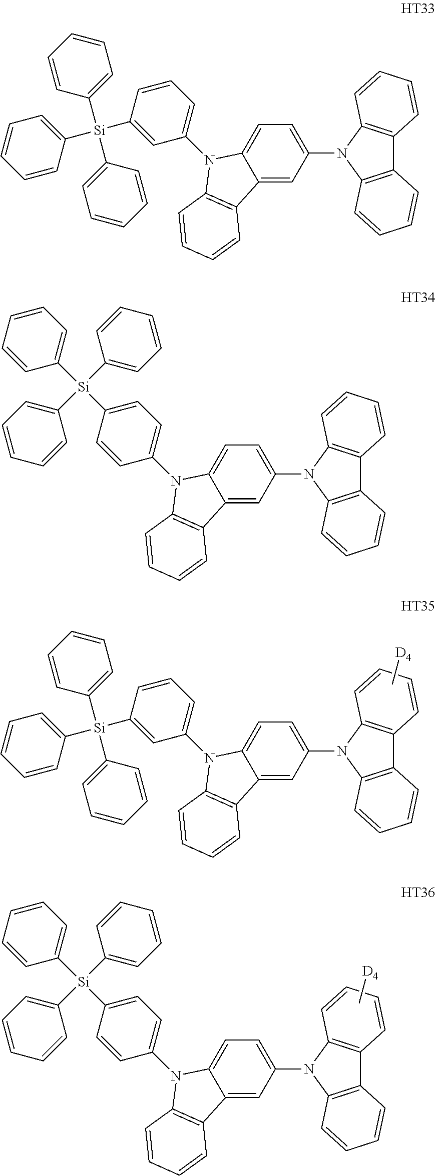

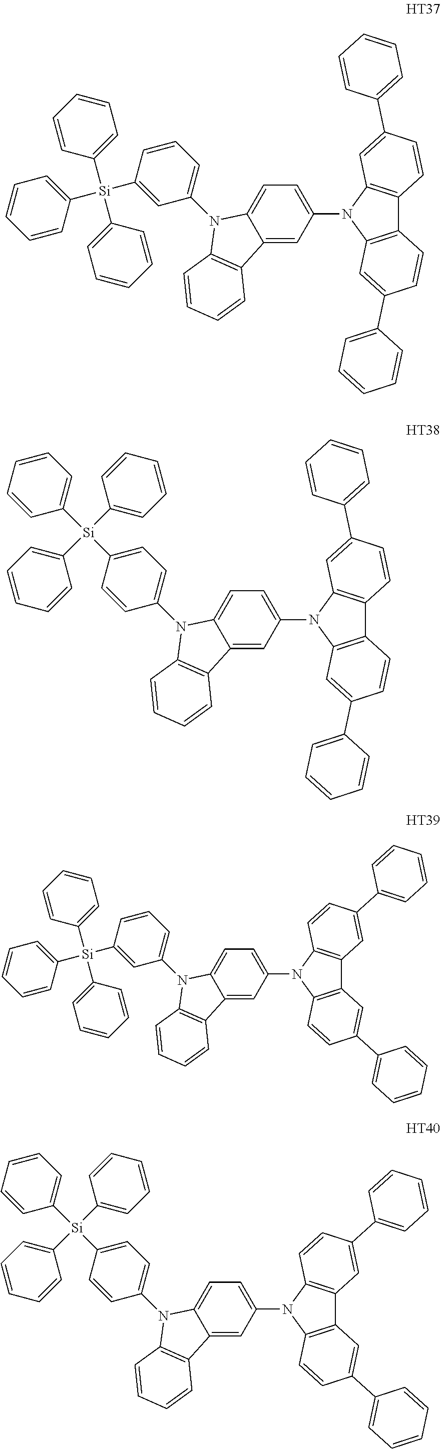 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1]

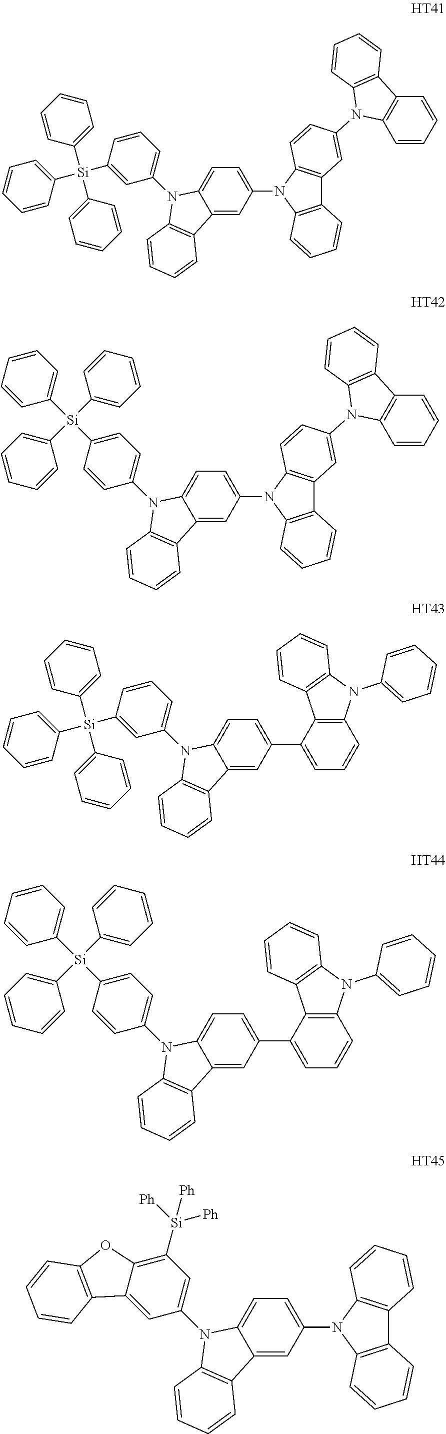

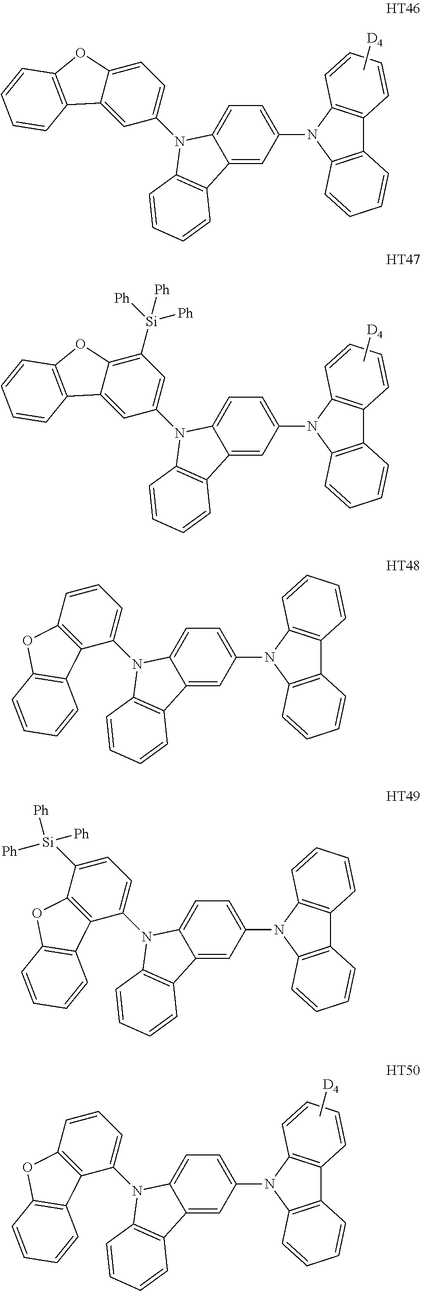

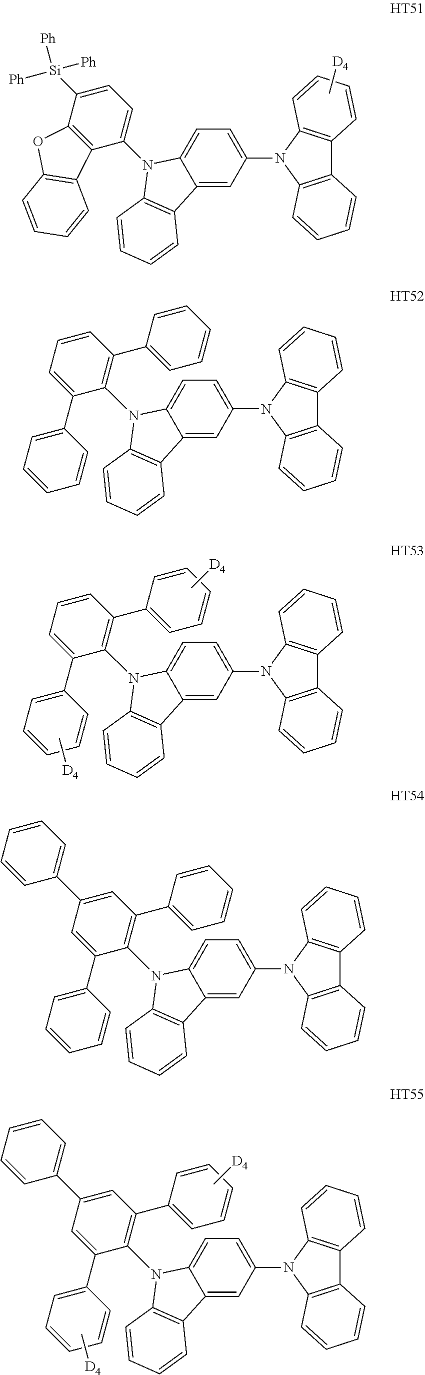

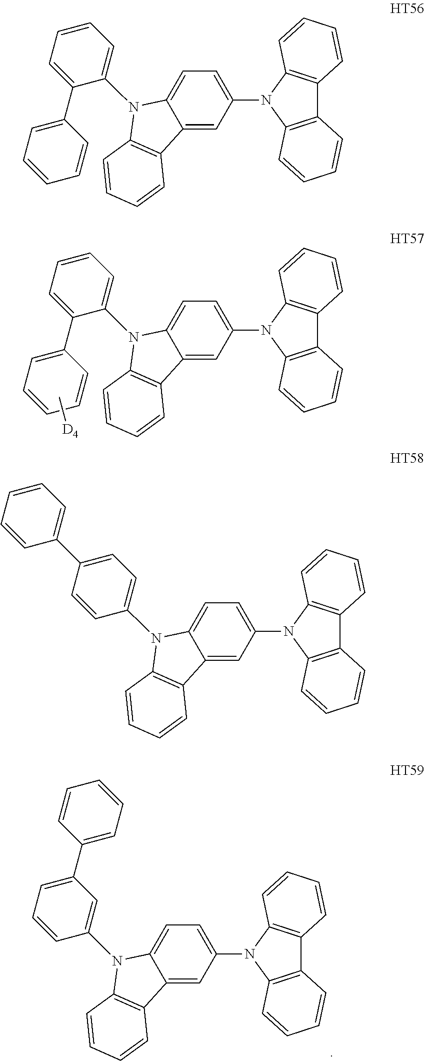

In the example compounds suggested in Compound Group 2, “D” refers to deuterium, and “Ph” may refer to a substituted or unsubstituted phenyl group. For example, in some embodiments, in the example compounds suggested in Compound Group 2, “Ph” may be an unsubstituted phenyl group.

In one or more embodiments, the light-emitting layer EML may contain the third compound represented by Formula ET-1. For example, the third compound may be utilized as an electron transporting host material of the light-emitting layer EML.

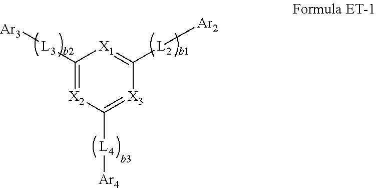

In Formula ET-1, at least one selected from among X₁ to X₃ may be N, and the remainder may be CR₅₆. For example, in some embodiments, only one selected from among X₁ to X₃ may be N, and the remainder two may each independently be CR₅₆. In these embodiments, the third compound represented by Formula ET-1 may include a pyridine moiety. In some embodiments, two selected from among X₁ to X₃ may be N, and the remainder may be CR₅₆. In these embodiments, the third compound represented by Formula ET-1 may include a pyrimidine moiety. In some embodiments, X₁ to X₃ may each be N. In these embodiments, the third compound represented by Formula ET-1 may include a triazine moiety.

In Formula ET-1, R₅₆ may be hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

In Formula ET-1, b1 to b3 may each independently be an integer of 0 to 10.

In Formula ET-1, Ar₂ to Ar₄ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in some embodiments, Ar₂ to Ar₄ may be substituted or unsubstituted phenyl groups or substituted or unsubstituted carbazole groups.

In Formula ET-1, L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when each of b1 to b3 is an integer of 2 or more, L₂ to L₄ may each independently be a substituted or unsubstituted arylene group of 6 to 30 ring-forming carbon atoms or a substituted or unsubstituted heteroarylene group of 2 to 30 ring-forming carbon atoms.

In one or more embodiments, the third compound may be any one selected from among compounds in Compound Group 3. The light emitting element ED of one or more embodiments may include any one selected from among the compounds in Compound Group 3.

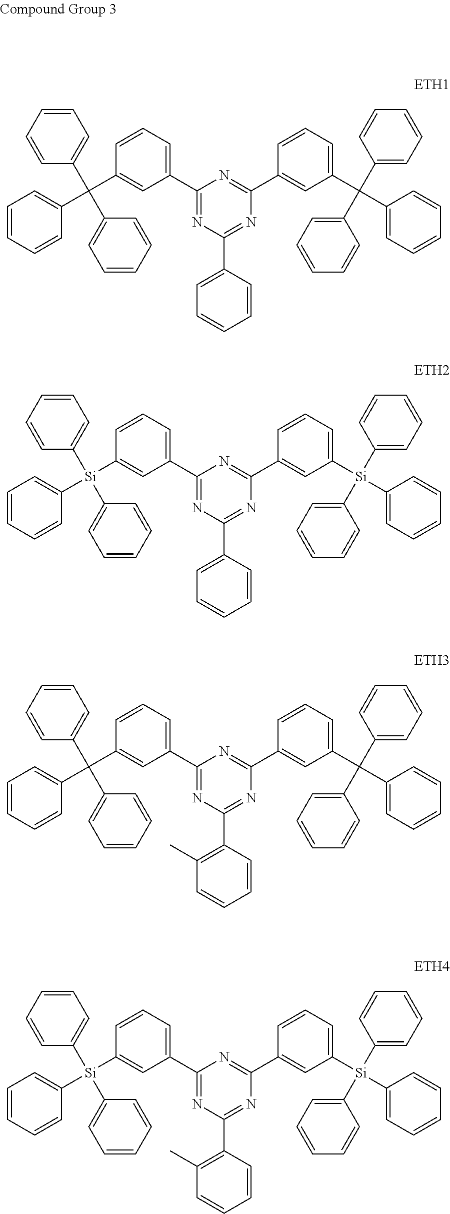 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

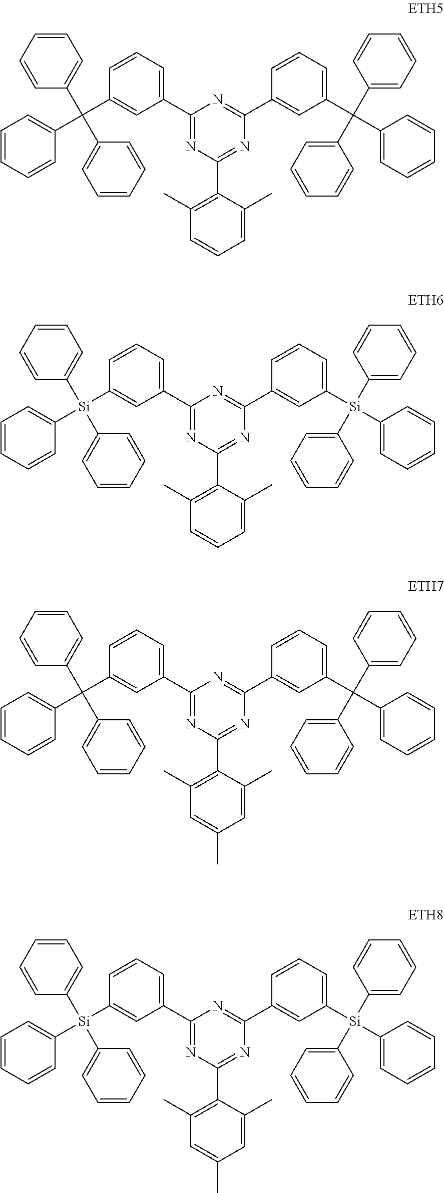 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

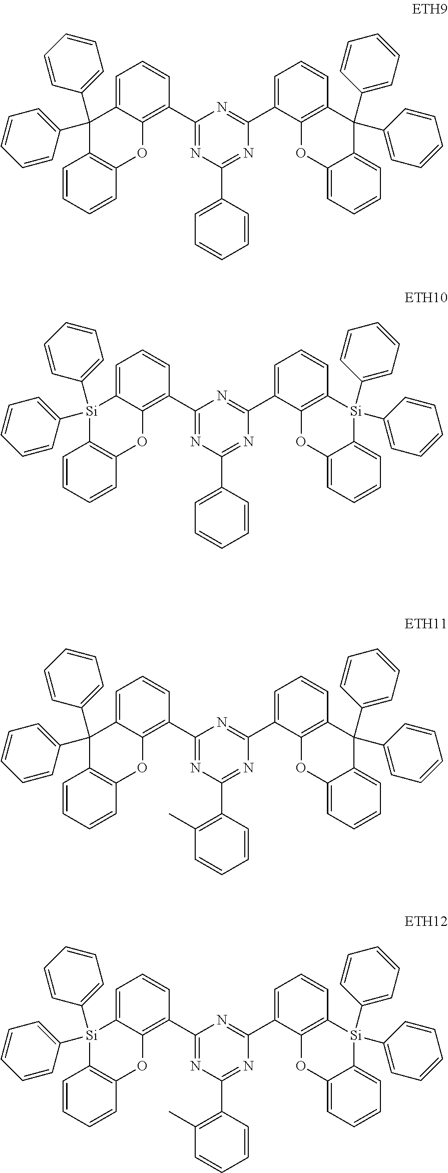 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

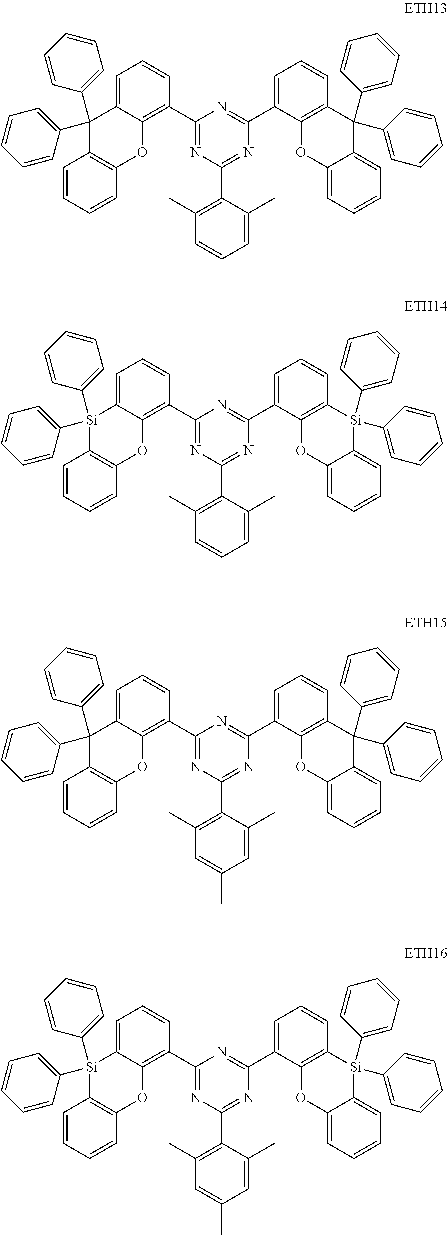 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

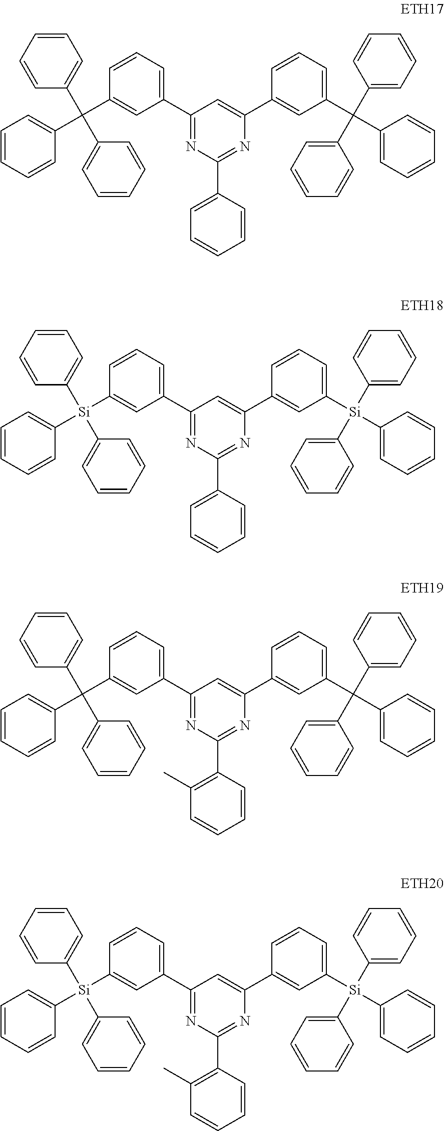 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

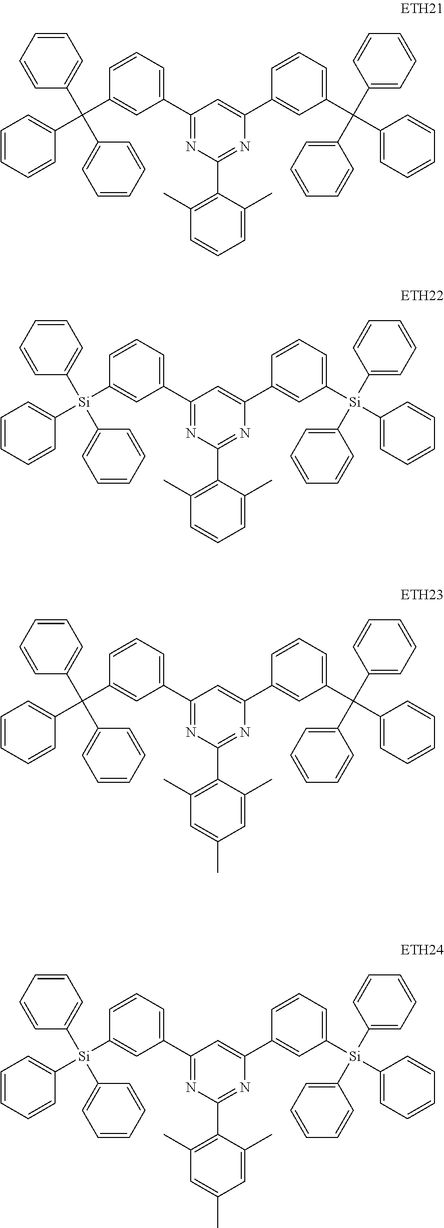 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

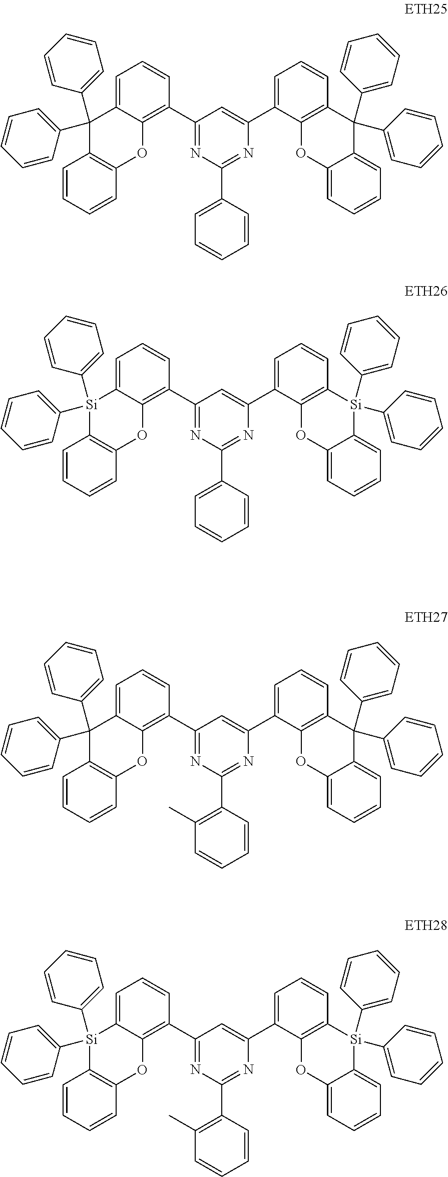 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

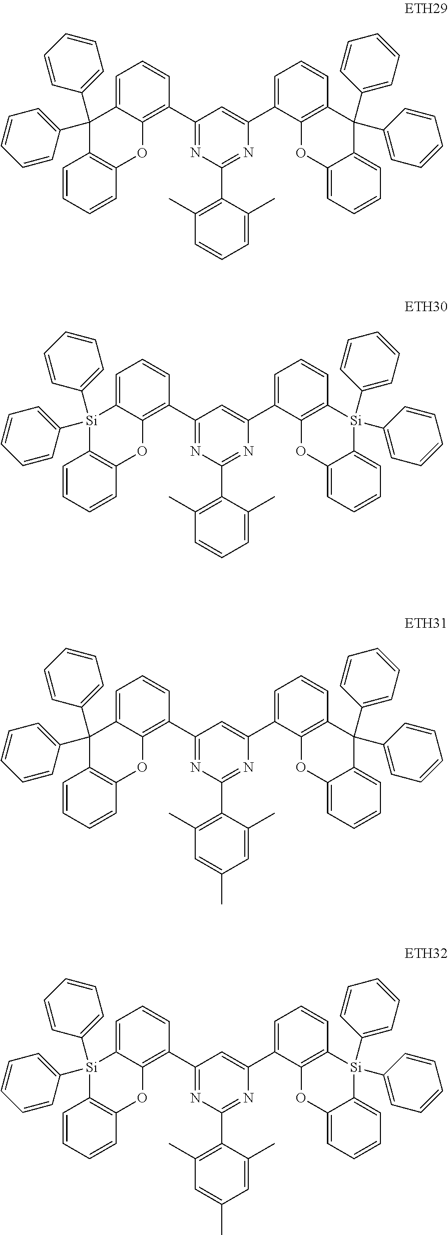 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

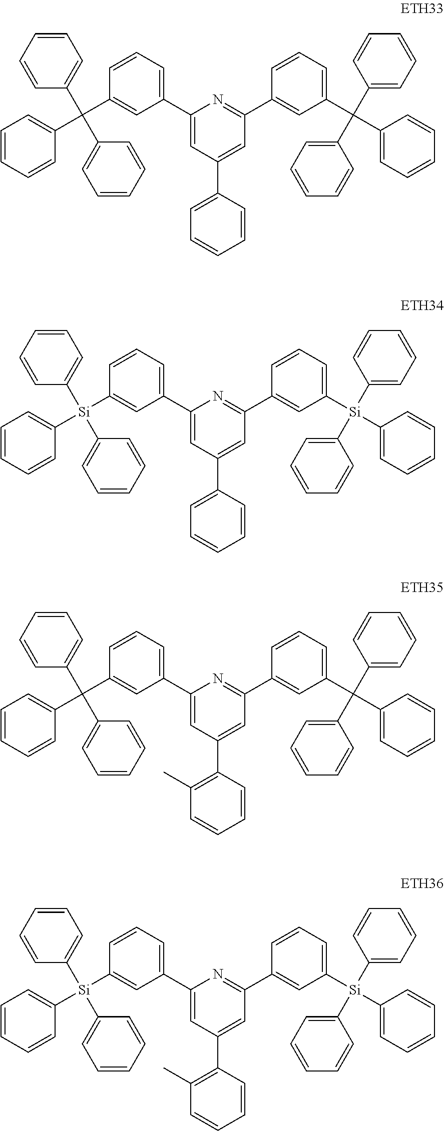 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

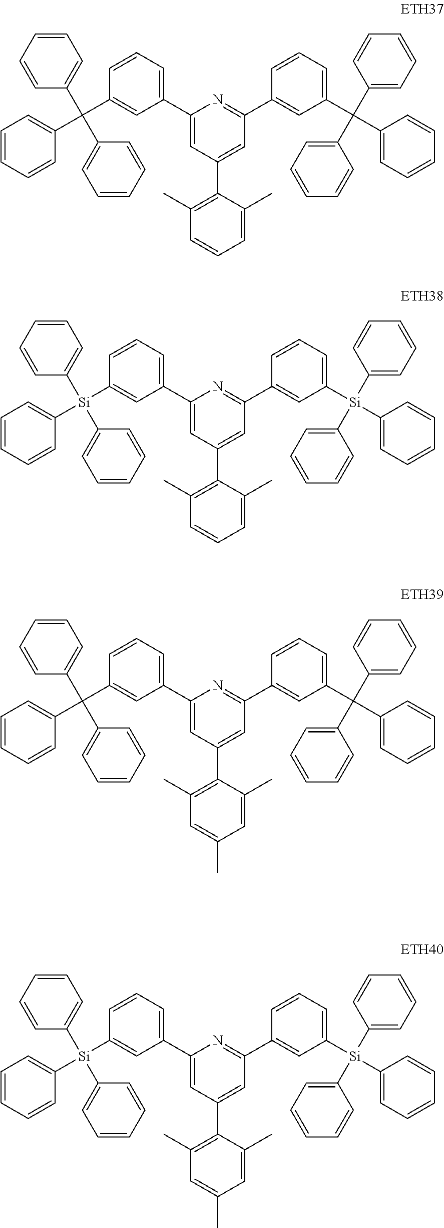 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

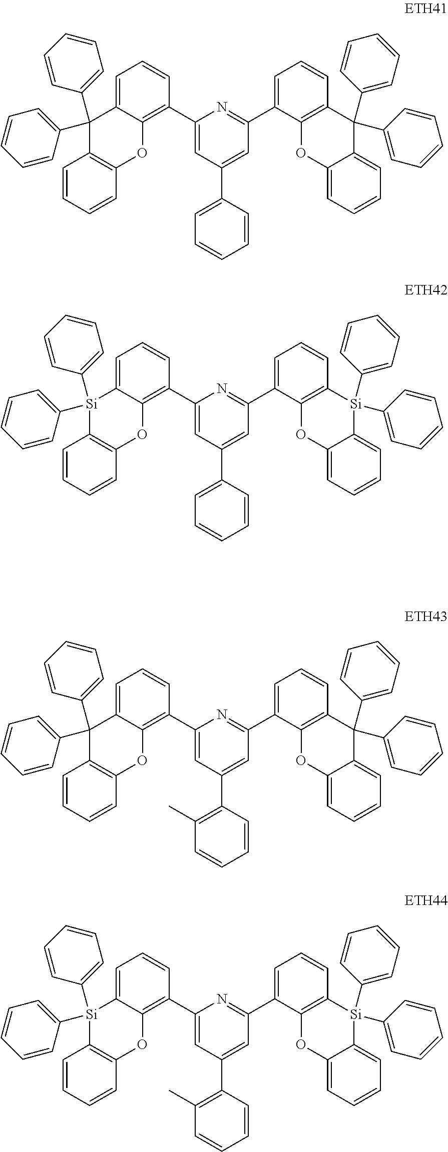 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

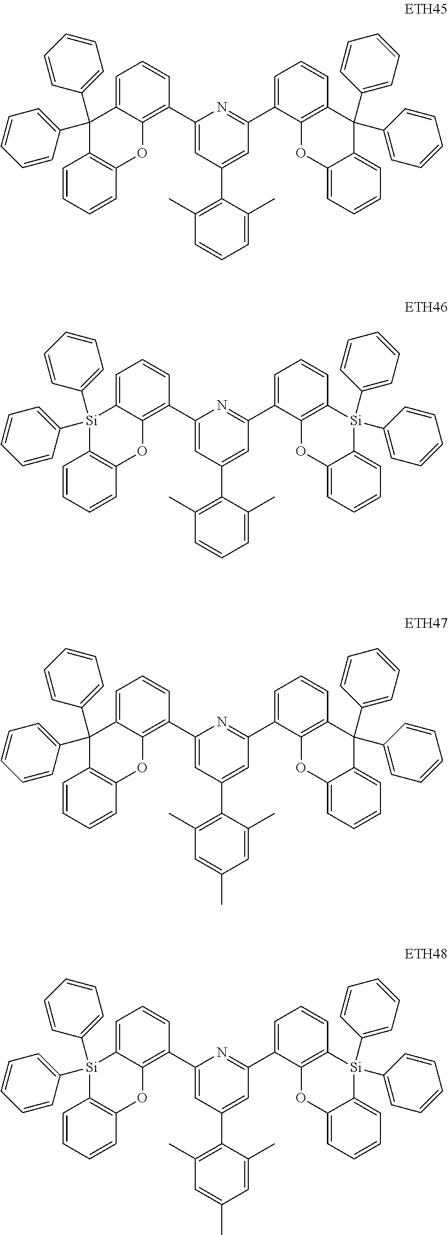 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

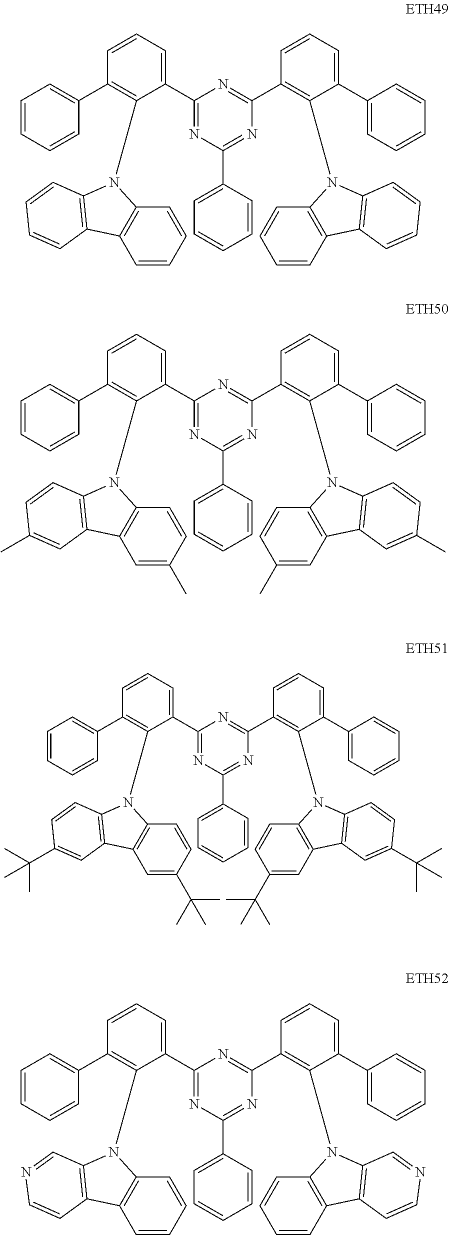

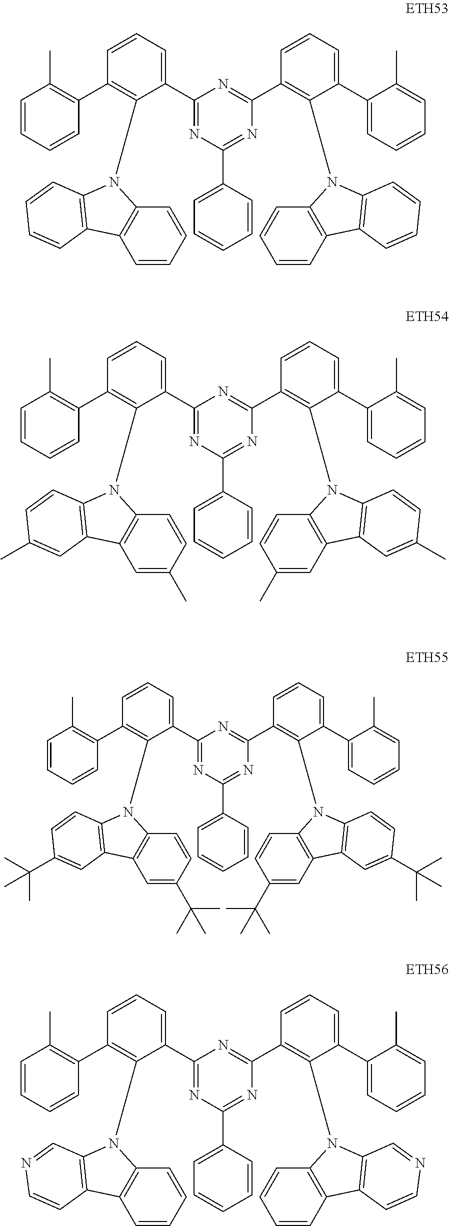

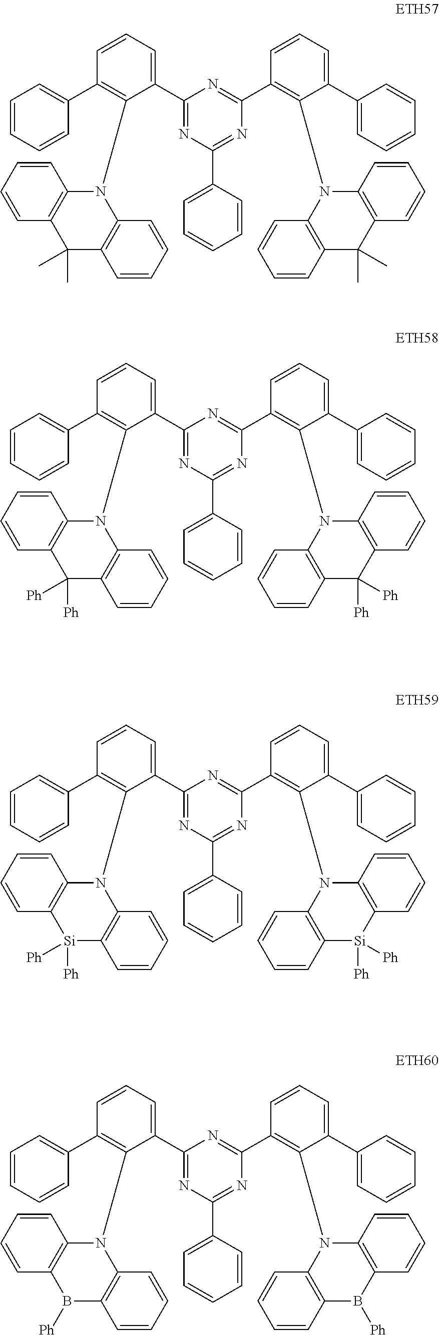

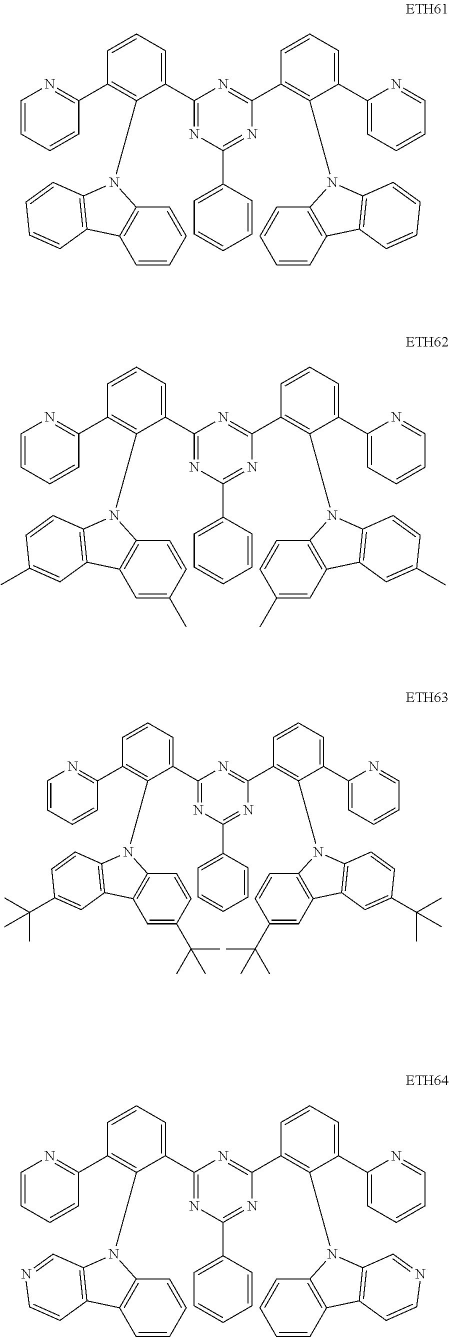

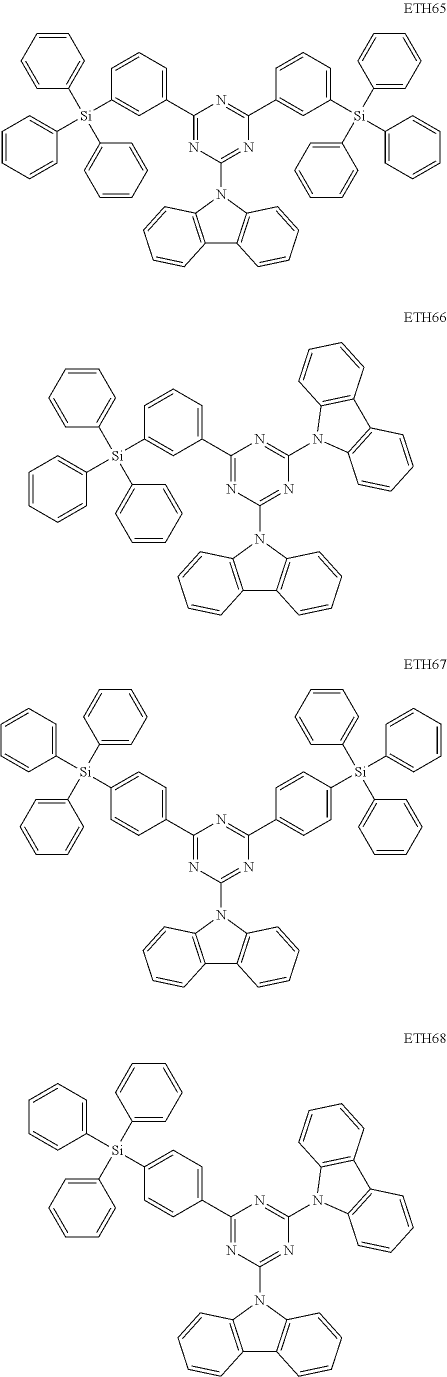 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

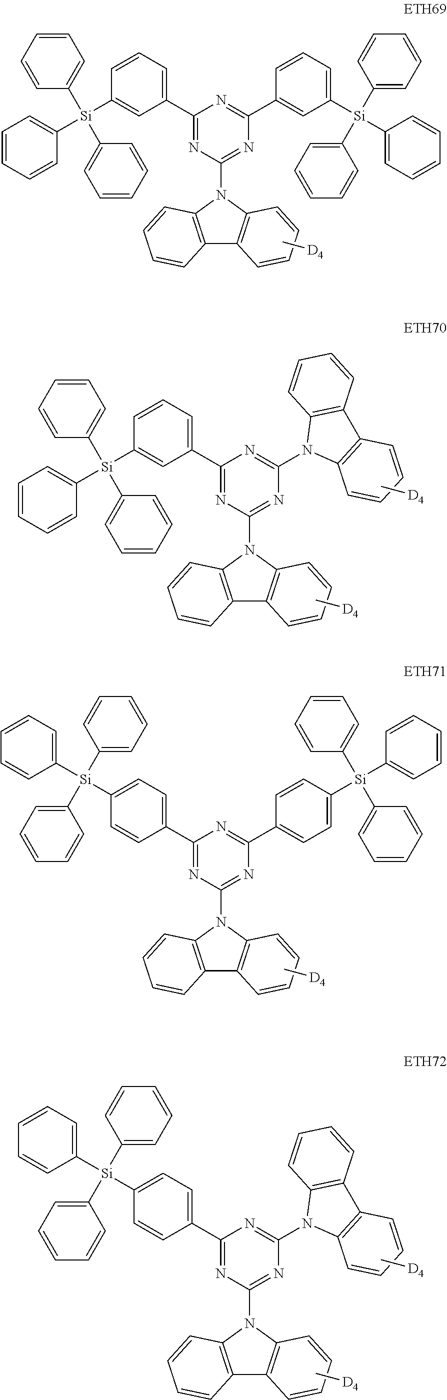

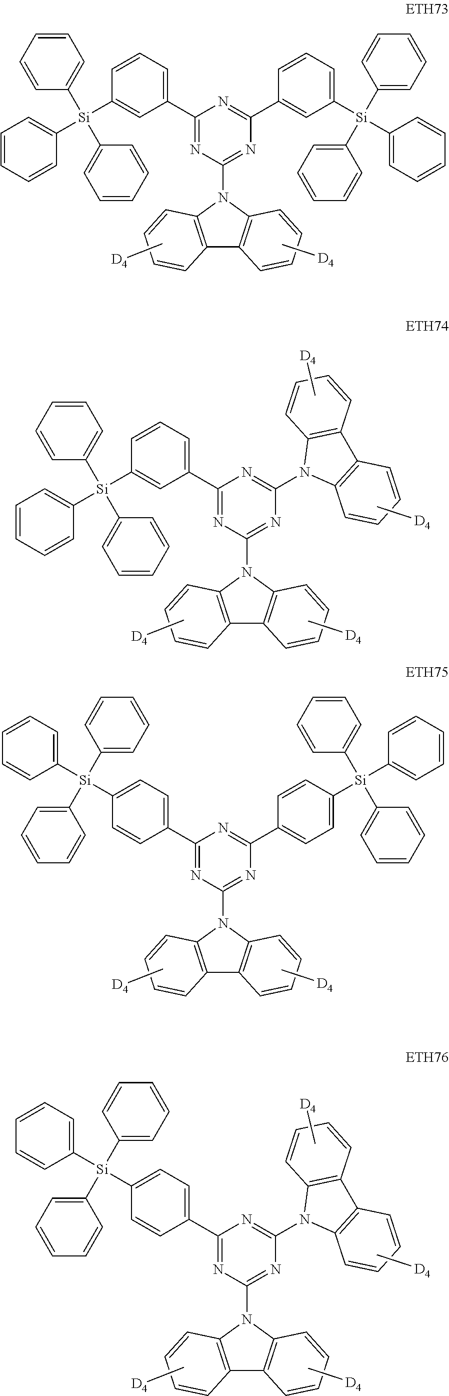

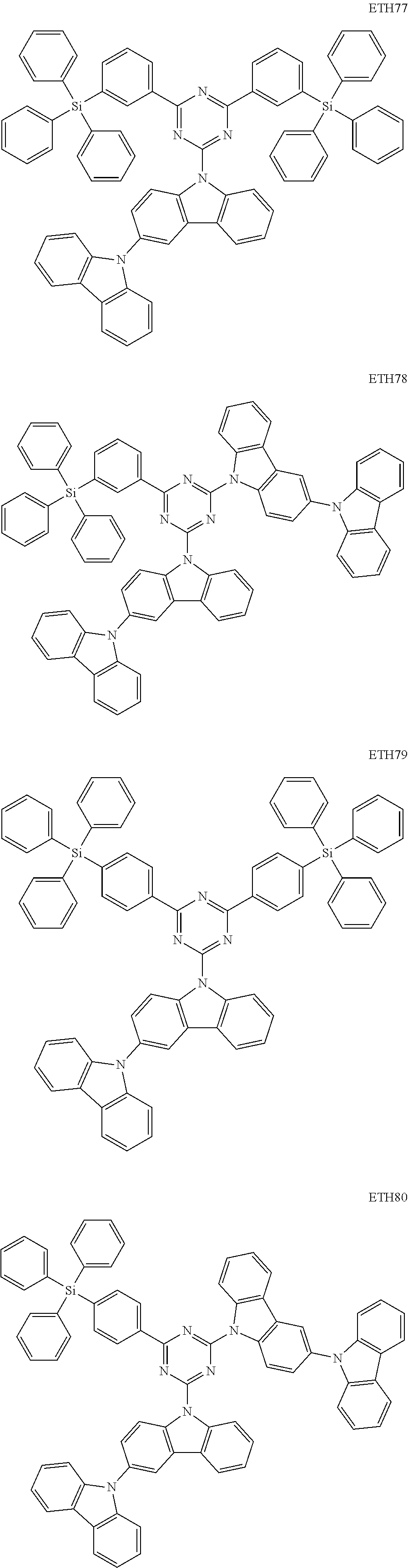

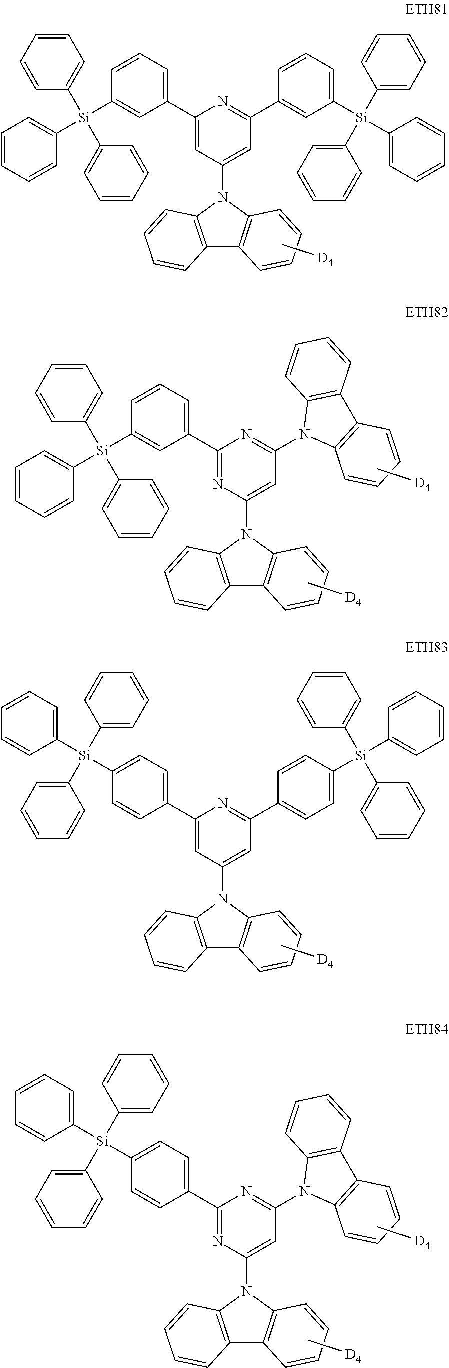

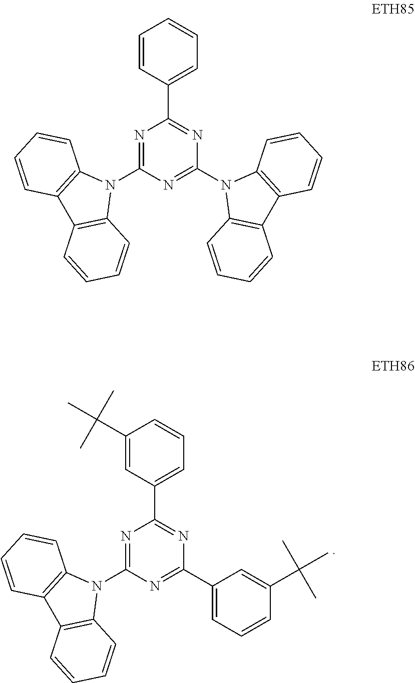 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In the example compounds suggested in Compound Group 3, “D” refers to deuterium, and “Ph” refers to an unsubstituted phenyl group.

In one or more embodiments, the light-emitting layer EML may include the second compound and the third compound, and the second compound and the third compound may form exciplex. In the light-emitting layer EML, exciplex may be formed by a hole transport host and an electron transport host. The triplet energy of the exciplex formed by the hole transport host and the electron transport host may correspond to a difference between a lowest unoccupied molecular orbital (LUMO) energy level of the electron transport host and a highest occupied molecular orbital (HOMO) energy level of the hole transport host.

For example, an absolute value of the triplet energy level (T1) of the exciplex formed by the hole transport host and the electron transport host may be about 2.4 eV to about 3.0 eV. In some embodiments, the triplet energy of the exciplex may be a smaller value than the energy gap of each host material. The exciplex may have a triplet energy of about 3.0 eV or less, that is the energy gap between the hole transport host and the electron transport host.

In one or more embodiments, the light-emitting layer EML may include a fourth compound in addition to the first compound to the third compound. The fourth compound may be utilized as a phosphorescence sensitizer of the light-emitting layer EML. Because energy may transfer from the fourth compound to the first compound, light emission may arise.

For example, in one or more embodiments, the light-emitting layer EML may include an organometallic complex which contains platinum (Pt) as a center metal atom and contain ligands bonded to the center metal atom, as the fourth compound. In the light-emitting element ED according to one or more embodiments, the light-emitting layer EML may contain a compound represented by Formula D-1 as the fourth compound.

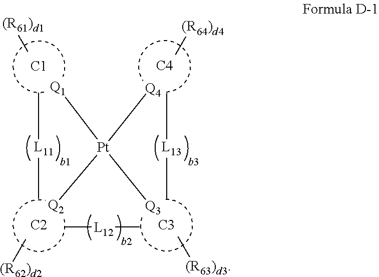

In Formula D-1, Q₁ to Q₄ may each independently be C or N.

In Formula D-1, C1 to C4 may each independently be a substituted or unsubstituted hydrocarbon ring of 5 to 30 ring-forming carbon atoms or a substituted or unsubstituted heterocycle of 2 to 30 ring-forming carbon atoms.

In Formula D-1, L₁₁ to L₁₃ may each independently be a direct linkage,

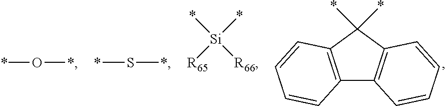

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, “ [symbol] ” refers to a part connected with C1 to C4.

In Formula D-1, b1 to b3 may each independently be 0 or 1. When b1 is 0, C1 and C2 may be unconnected. When b2 is 0, C2 and C3 may be unconnected.

When b3 is 0, C3 and C4 may be unconnected.

In Formula D-1, R₆₁ to R₆₆ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to ring-forming carbon atoms, or a substituted or unsubstituted heteroaryl group of 2 to ring-forming carbon atoms. In some embodiments, one or more of R₆₁ to R₆₆ may be combined with an adjacent group to form a ring. In some embodiments, R₆₁ to R₆₆ may each independently be a substituted or unsubstituted methyl group or a substituted or unsubstituted t-butyl group.

In Formula D-1, d1 to d4 may each independently be an integer of 0 to 4. In Formula D-1, when d1 to d4 are each 0, the fourth compound may be unsubstituted with R₆₁ to R₆₄, respectively. An embodiment in which d1 to d4 are each 4, and R₆₁ to R₆₄ are each hydrogen, may be the same as an embodiment in which d1 to d4 are each 0. When d1 to d4 are integers of 2 or more, each of multiple R₆₁'s to R₆₄'s may be all the same, or at least one selected from among multiple R₆₁'s to R₆₄'s may be different.

In Formula D-1, C1 to C4 may each independently be a substituted or unsubstituted hydrocarbon ring or a substituted or unsubstituted heterocycle, represented by any one selected from among C-1 to C-4.

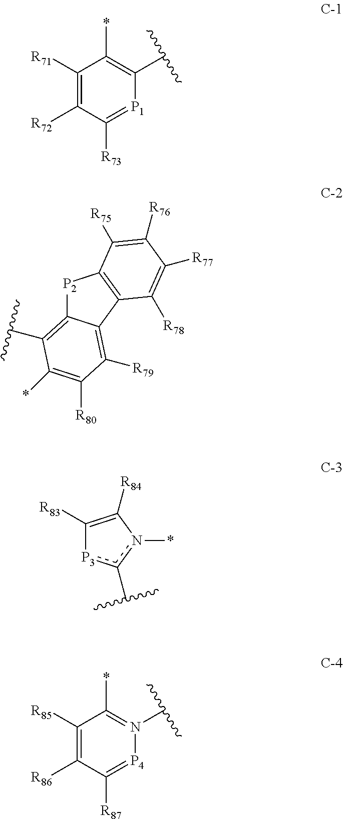

In C-1 to C-4, P₁ may be C—• or CR₇₄, P₂ may be N—• or NR₈₁, P₃ may be N—• or NR₈₂, and P₄ may be C—• or CR₈₈. R₇₁ to R₈₈ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In addition, in C-1 to C-4,

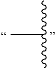

is a part connected with a central metal atom of Pt, and “ [symbol] ” corresponds to a part connected with an adjacent ring group (C1 to C4) or a linker (L₁₁ to L₁₃).

The light-emitting layer EML of one or more embodiments may include the first compound that is a fused polycyclic compound, and at least one selected from among the second to fourth compounds. For example, in some embodiments, the light-emitting layer EML may include the first compound, the second compound, and the third compound. In the light-emitting layer EML, the second compound and the third compound may form exciplex, and via the exciplex, energy transfer to the first compound may arise, and light emission may arise.

In some embodiments, the light-emitting layer EML may include the first compound, the second compound, the third compound, and the fourth compound. In the light-emitting layer EML, the second compound and the third compound may form exciplex, and via the exciplex, energy transfer to the fourth compound and the first compound may arise, and light emission may arise. In some embodiments, the fourth compound may be a sensitizer. In the light emitting element ED of one or more embodiments, the fourth compound included in the light-emitting layer EML may act as a sensitizer and may play the role of transferring energy from a host to the first compound that is a light-emitting dopant. For example, the fourth compound that plays the role of an auxiliary dopant may accelerate energy transfer to the first compound that is a light emitting dopant and increase the light emitting ratio of the first compound. Accordingly, the emission efficiency of the light-emitting layer EML of one or more embodiments may be improved. In some embodiments, when the energy transfer to the first compound increases, excitons formed in the light-emitting layer EML may not be accumulated but rapidly emit light, and thus the deterioration of the light-emitting element may be reduced. Accordingly, the lifetime of the light emitting element ED of one or more embodiments may increase.

The light emitting element ED of one or more embodiments includes all of the first compound, the second compound, the third compound, and the fourth compound, and the light-emitting layer EML may include the combination of two host materials and two dopant materials. In the light emitting element ED of one or more embodiments, the light-emitting layer EML may include the second compound and the third compound, which are two different hosts, the first compound which emits delayed fluorescence, and the fourth compound including an organometallic complex, concurrently (e.g., simultaneously), and may show excellent or suitable emission efficiency properties.

In one or more embodiments, the fourth compound represented by Formula D-1 may be at least one selected from among compounds represented in Compound Group 4. The light-emitting layer EML may include at least one selected from among the compounds represented in Compound Group 4 as a sensitizer material.

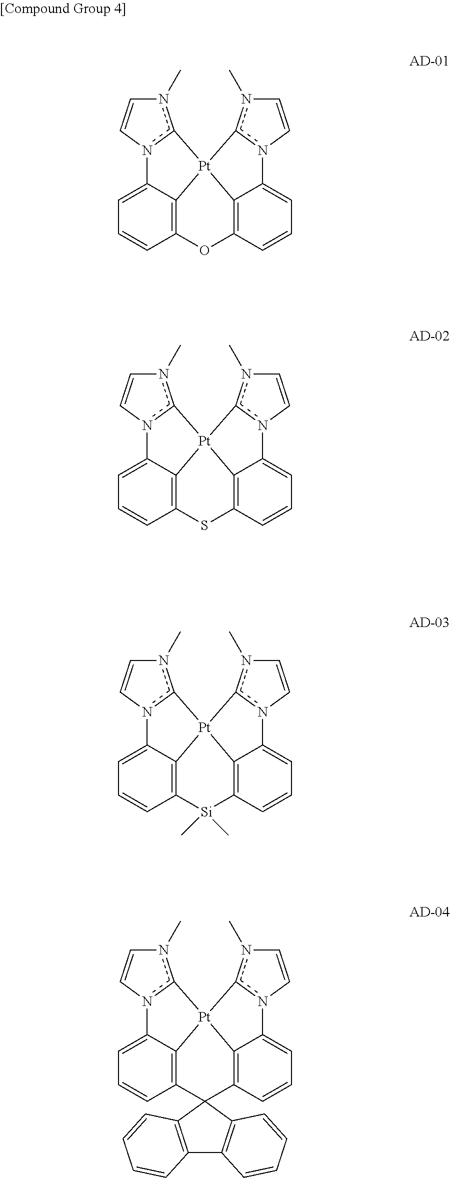

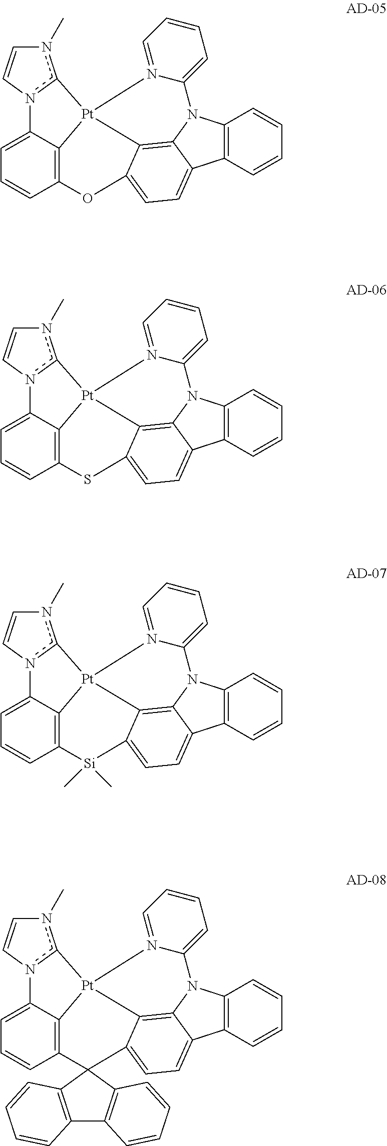

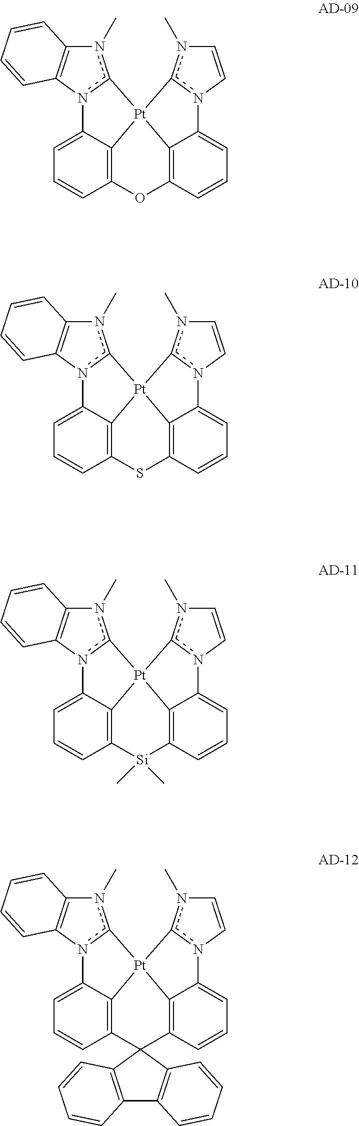

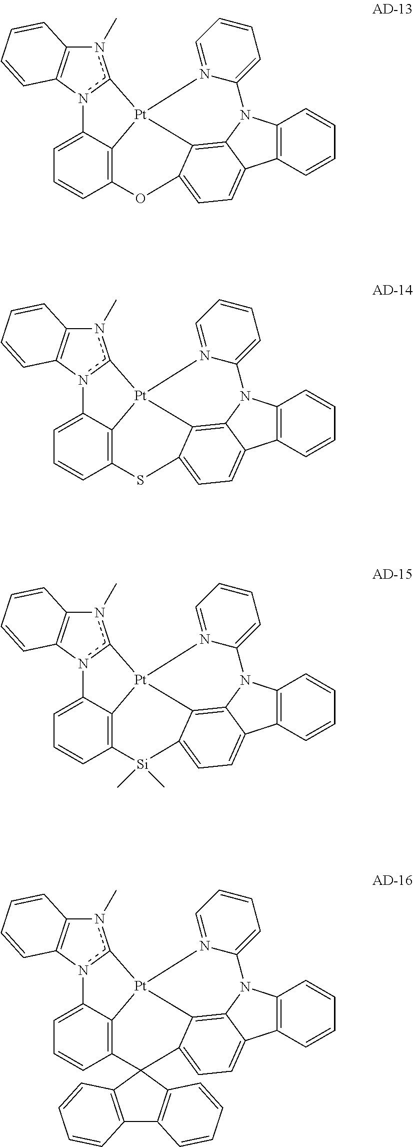

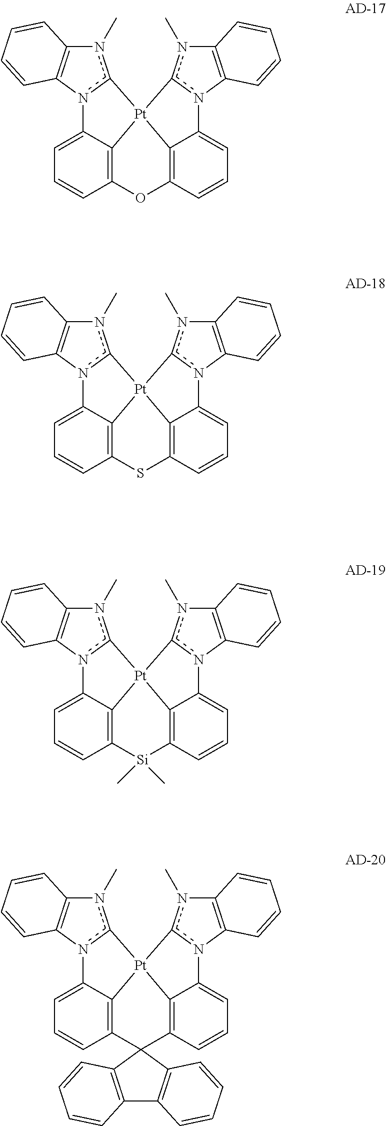

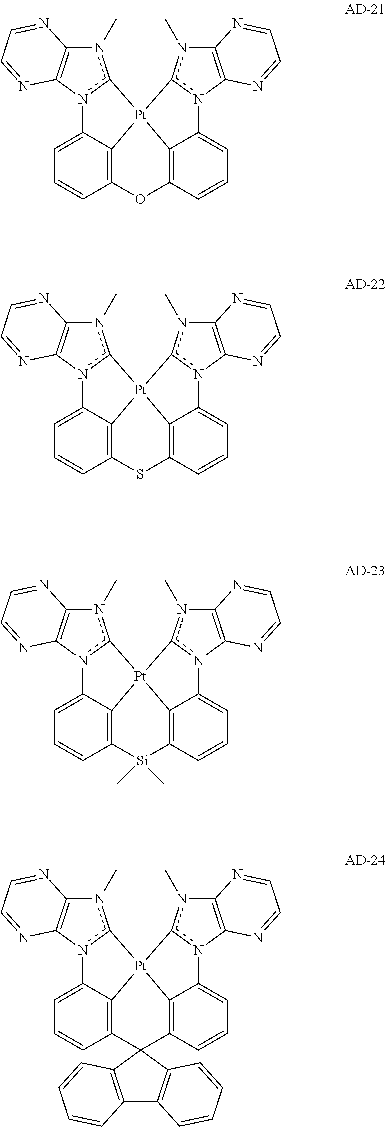

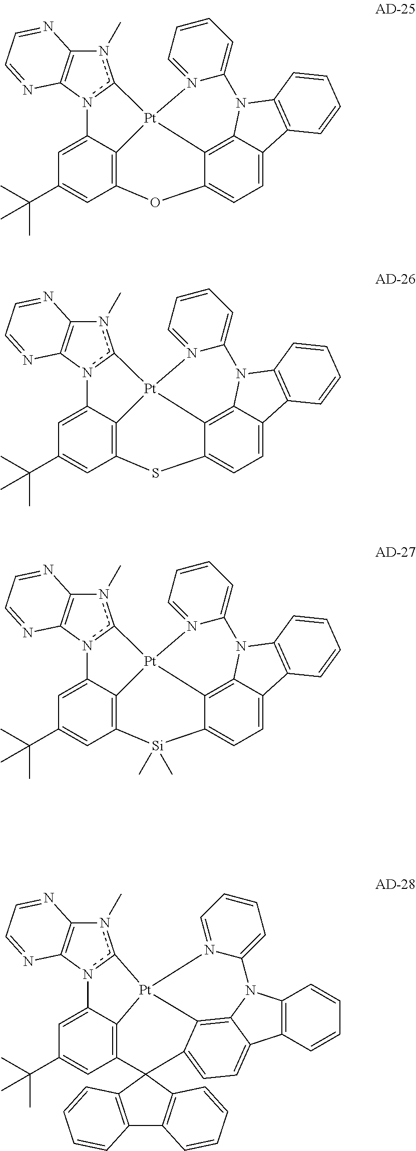

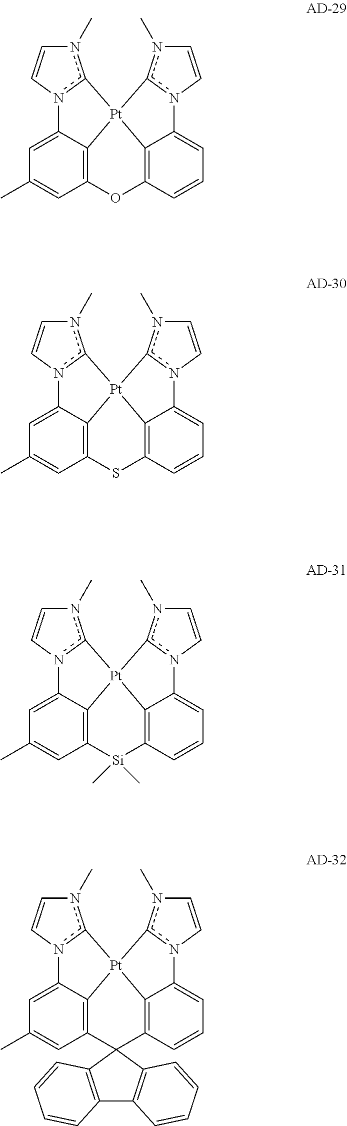

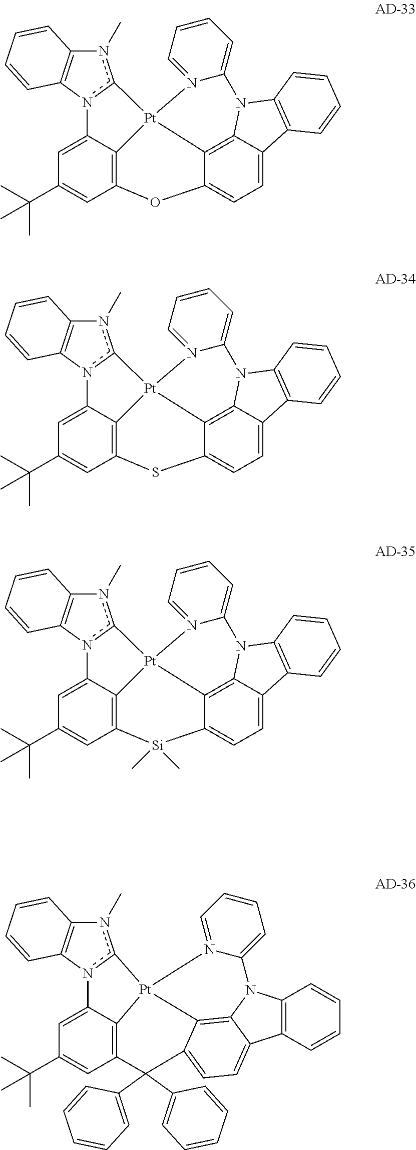

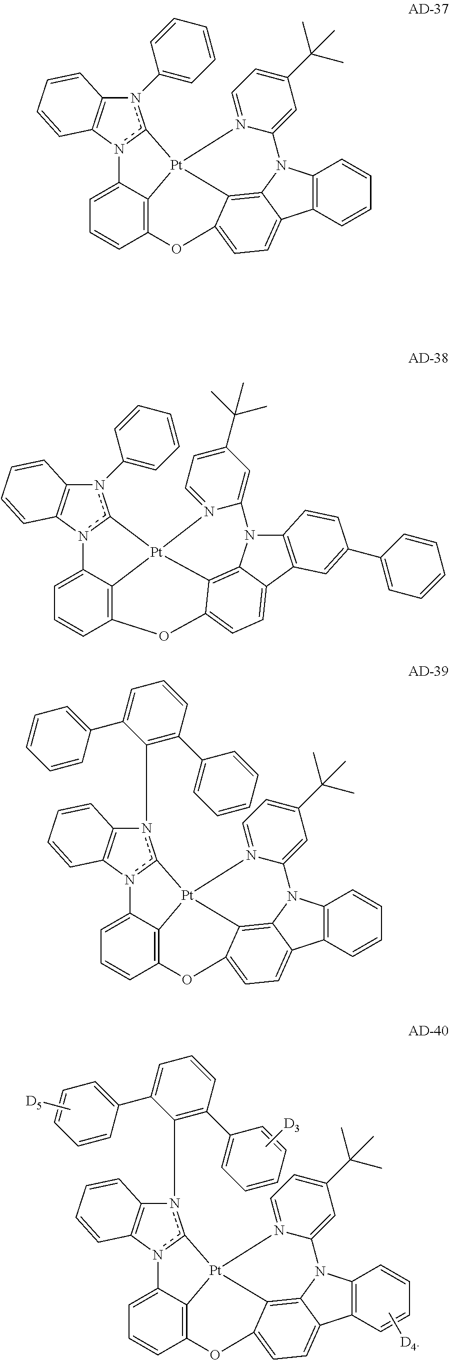

In the example compounds suggested in Compound Group 4, “D” refers to deuterium.

In the light emitting element ED of one or more embodiments, when the light-emitting layer EML includes all of the first compound, the second compound, the third compound, and the fourth compound, an amount of the first compound may be about 0.1 wt % to about 5 wt % based on the total weight of the first compound, the second compound, the third compound, and the fourth compound. However, embodiments of the present disclosure are not limited thereto. When the amount of the first compound satisfies the above-described ratio, energy transfer from the second compound and the third compound to the first compound may increase, and accordingly, the emission efficiency and device lifetime may increase.

In the light-emitting layer EML, a total amount of the second compound and the third compound may be the remaining amount excluding the amount of the first compound and the fourth compound. For example, the total amount of the second compound and the third compound may be about 65 wt % to about 95 wt % based on the total weight of the first compound, the second compound, the third compound, and the fourth compound.

In the total amount of the second compound and the third compound, a weight ratio of the second compound and the third compound may be about 3:7 to about 7:3.

When the total amount of the second compound and the third compound satisfies the above-described ratio, charge balance properties in the light-emitting layer EML may be improved, and emission efficiency and device lifetime may be improved and/or increased. When the total amount of the second compound and the third compound deviates from the above-described ratio range, charge balance in the light-emitting layer EML may be broken, emission efficiency may be degraded and/or decreased, and the device may be easily deteriorated.

When the light-emitting layer EML includes the fourth compound, an amount of the fourth compound may be about 4 wt % to 30 wt % based on the total weight of the first compound, the second compound, the third compound, and the fourth compound in the light-emitting layer EML. However, embodiments of the present disclosure are not limited thereto. When the amount of the fourth compound satisfies the above-described amount, energy transfer from a host to the first compound that is a light emitting dopant may increase, and emission ratio may be improved. Accordingly, the emission efficiency of the light-emitting layer EML may be improved. When the amount ratio of the first compound, the second compound, the third compound, and the fourth compound, included in the light-emitting layer EML satisfies the above-described amount ratio, excellent or suitable emission efficiency and long lifetime of the light emitting element may be achieved.

In one or more embodiments, the light-emitting layer EML may include, as a suitable dopant material, one or more selected from styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and derivatives thereof (for example, 2,5,8,11-tetra-t-butylperylene (TBP)), pyrene and derivatives thereof (for example, 1,1-dipyrene, 1,4-dipyrenylbenzene, and 1,4-bis(N,N-diphenylamino)pyrene), etc.

In one or more embodiments, the light-emitting layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb), or thulium (Tm). For example, in some embodiments, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), or platinum octaethyl porphyrin (PtOEP) may be utilized as the phosphorescence dopant. However, embodiments of the present disclosure are not limited thereto.

In some embodiments, the emission layer EML may include a hole transport host and an electron transport host. In some embodiments, the emission layer EML may include an auxiliary dopant and a light emitting dopant. In some embodiments, the auxiliary dopant may include a phosphorescence dopant material and/or a thermally activated delayed fluorescence dopant. For example, in some embodiments, the emission layer EML may include a hole transport host, an electron transport host, an auxiliary dopant, and a light emitting dopant.

In one or more embodiments, the light-emitting layer may include a quantum dot.

In the present disclosure, the quantum dot refers to the crystal of a semiconductor compound. The quantum dot may be to emit light in one or more suitable emission wavelengths according to the size of the crystal. The quantum dot may be to emit light in one or more suitable emission wavelengths by controlling an element ratio in the quantum dot compound.

The diameter of the quantum dot may be, for example, about 1 nm to about nm.

The quantum dot may be synthesized by a chemical bath deposition, a metal organic chemical vapor deposition, a molecular beam epitaxy, or a similar process therewith.

The chemical bath deposition is a method of mixing an organic solvent and a precursor material of a quantum dot and then, growing a quantum dot particle crystal.

During growing the crystal, the organic solvent may naturally play the role of a dispersant which is coordinated on the surface of the quantum dot crystal and may control the growth of the crystal. Accordingly, the chemical bath deposition is more advantageous when compared to a vapor deposition method including a metal organic chemical vapor deposition (MOCVD) and/or a molecular beam epitaxy (MBE), and the growth of the quantum dot particle may be controlled or selected through a low-cost process.

In one or more embodiments, the light-emitting layer EML may include a quantum dot material. In one or more embodiments, the quantum dot material may have a core/shell structure. The core of the quantum dot may be selected from a II-VI group compound, a III—VI group compound, a 1-III-VI group compound, a III—V group compound, a Ill-II-V group compound, a IV—VI group compound, a IV group element, a IV group compound, and combinations thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof. In some embodiments, the Group II-VI compound may further include a Group I metal and/or a Group IV element. The Group I—II-VI compound may be selected from CuSnS and/or CuZnS, and the Group II—IV-VI compound may be selected from ZnSnS and/or the like. The Group I—II-IV-VI compound may be selected from quaternary compounds selected from the group consisting of Cu₂ZnSnS₂, Cu₂ZnSnS₄, Cu₂ZnSnSe₄, Ag₂ZnSnS₂, and any mixture thereof.

The III-VI group compound may include a binary compound such as In₂S₃ and/or In₂Se₃, a ternary compound such as InGaS₃ and/or InGaSes, or any combination thereof.

The I—III—VI group compound may be selected from a ternary compound selected from the group consisting of AgInS, AgInS₂, CuInS, CulnS₂, AgGaS₂, CuGaS₂, CuGaO₂, AgGaO₂, AgAIO₂, and mixtures thereof, and/or a quaternary compound such as AgInGaS₂ and/or CulnGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof; a ternary compound selected from the group consisting of GaNP, GaNAs, GaNSb, GaPAs, GaPSb, AlNP, AlNAs, AlNSb, AlPAs, AlPSb, InGaP, InAIP, InNP, InNAs, InNSb, InPAs, InPSb, and mixtures thereof; and a quaternary compound selected from the group consisting of GaAINP, GaAINAs, GaAINSb, GaAIPAs, GaAIPSb, GaInNP, GaInNAs, GalnNSb, GaInPAs, GalnPSb, InAINP, InAINAs, InAINSb, InAIPAs, InAIPSb, and mixtures thereof. In some embodiments, the Ill-V group compound may further include a II group metal. For example, InZnP, etc. may be selected as a III—II—V group compound.

The Group IV-VI compound may be selected from the group consisting of a binary compound selected from the group consisting of SnS, SnSe, SnTe, PbS, PbSe, PbTe, and any mixture thereof; a ternary compound selected from the group consisting of SnSeS, SnSeTe, SnSTe, PbSeS, PbSeTe, PbSTe, SnPbS, SnPbSe, SnPbTe, and any mixture thereof; and a quaternary compound selected from the group consisting of SnPbSSe, SnPbSeTe, SnPbSTe, and any mixture thereof.

The Group II—IV-V compound may be selected from a ternary compound selected from the group consisting of ZnSnP, ZnSnP₂, ZnSnAs₂, ZnGeP₂, ZnGeAs₂, CdSnP₂, CdGeP₂, and any mixture thereof.

The Group IV element may be selected from the group consisting of Si, Ge, and a mixture thereof. The Group IV compound may be a binary compound selected from the group consisting of SiC, SiGe, and any mixture thereof.

Each element included in the multi-element compound such as the binary compound, ternary compound, and quaternary compound may be present in particles at a substantially uniform concentration or a non-substantially uniform concentration.

For example, Formula above indicates the types (kinds) of elements included in a compound, and element ratios in the compound may be different. For example, AgInGaS₂ may indicate AgInₓGa₁₋ₓS₂ (x is a real number between 0 and 1).

In one or more embodiments, constituting elements of the binary compound, the ternary compound, or the quaternary compound may be present at substantially uniform concentration in a particle or may be present at a partially different concentration distribution within substantially the same particle. In some embodiments, a core/shell structure in which one quantum dot wraps another quantum dot may be desirable. The interface of the core and the shell may have a concentration gradient in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, or combinations thereof.

For example, the metal or non-metal oxide for the shell may include a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄, and/or NiO, and/or a ternary compound such as MgAl₂O₄, CoFe₂O₄, NiFe₂O₄, and/or CoMn₂O₄, but embodiments of the present disclosure are not limited thereto.

Also, the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

The quantum dot may have a full width of half maximum (FWHM) of emission spectrum of about 45 nm or less, about 40 nm or less, or about 30 nm or less. Within this range, color purity or color reproducibility may be improved. In some embodiments, light emitted via such a quantum dot is emitted in all directions, and light view angle properties may be improved.

In addition, the shape of the quantum dot may be generally utilized shapes in the art, without specific limitation. For example, the shape of spherical nanoparticle, pyramidal nanoparticle, multi-arm nanoparticle, or cubic nanoparticle, nanotube, nanowire, nanofiber, nanoplate particle, etc. may be utilized.

As the size of the quantum dot or the ratio of elements in the quantum dot compound is regulated, the energy band gap of the quantum dot may be accordingly controlled or selected to obtain light of one or more suitable wavelengths from a quantum dot emission layer. Therefore, by utilizing the quantum dots as described above (e.g., utilizing quantum dots of different sizes and/or having different element ratios in the quantum dot compound), a light emitting element emitting light of one or more suitable wavelengths may be obtained. For example, the size of the quantum dots and/or the ratio of elements in the quantum dot compound may be regulated to emit red, green, and/or blue light. In some embodiments, the quantum dots may be configured to emit white light by combining light of one or more suitable colors.

In the light emitting elements ED of one or more embodiments, as shown in FIG. 3 to FIG. 6, the electron transport region ETR may be provided on the light-emitting layer EML. The electron transport region ETR may include at least one of an electron blocking layer HBL, an electron transport layer ETL, or an electron injection layer EIL. However, embodiments of the present disclosure are not limited thereto.

The electron transport region ETR may have a single layer formed utilizing a single material, a single layer formed utilizing multiple different materials, or a multilayer structure having multiple layers formed utilizing multiple different materials.

For example, in one or more embodiments, the electron transport region ETR may have a single layer structure of an electron injection layer EIL or an electron transport layer ETL, or a single layer structure formed utilizing an electron injection material and/or an electron transport material. Further, in some embodiments, the electron transport region ETR may have a single layer structure formed utilizing multiple different materials, or a structure stacked from the light-emitting layer EML of electron transport layer ETL/electron injection layer EIL, or hole blocking layer HBL/electron transport layer ETL/electron injection layer EIL, without limitation. A thickness of the electron transport region ETR may be, for example, from about 1,000 Å to about 1,500 Å.

The electron transport region ETR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2.

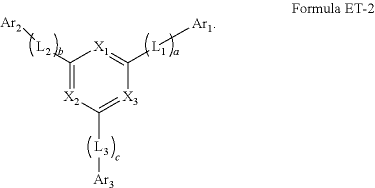

In Formula ET-2, at least one selected from among X₁ to X₃ may be N, and the remainder are CRₐ. Rₐ may be hydrogen, deuterium, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” to “c” are each independently an integer of 2 or more, L₁ to L_{3 may each independently be a substituted or unsubstituted arylene group of} 6 to 30 ring-forming carbon atoms or a substituted or unsubstituted heteroarylene group of 2 to ring-forming carbon atoms.

In one or more embodiments, the electron transport region ETR may include an anthracene-based compound. However, embodiments of the present disclosure are not limited thereto, for example, in some embodiments, the electron transport region ETR may include, for example, at least one selected from among tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N 1,O8)-(1,1′-biphenyl-4-olato)aluminum (BAIq), berylliumbis(benzoquinolin-10-olate (Bebq₂), 9,10-di(naphthalen-2-yl)anthracene (ADN), 1,3-bis[3,5-di(pyridin-3-yl)phenyl]benzene (BmPyPhB), CNNPTRZ(4′-(4-(4-(4,6-diphenyl-1,3,5-triazin-2-yl)phenyl)naphthalen-1-yl)-[1,1′-biphenyl]-4-carbonitrile), and mixtures thereof, without limitation.

In one or more embodiments, the electron transport region ETR may include at least one selected from among Compounds ET1 to ET36.

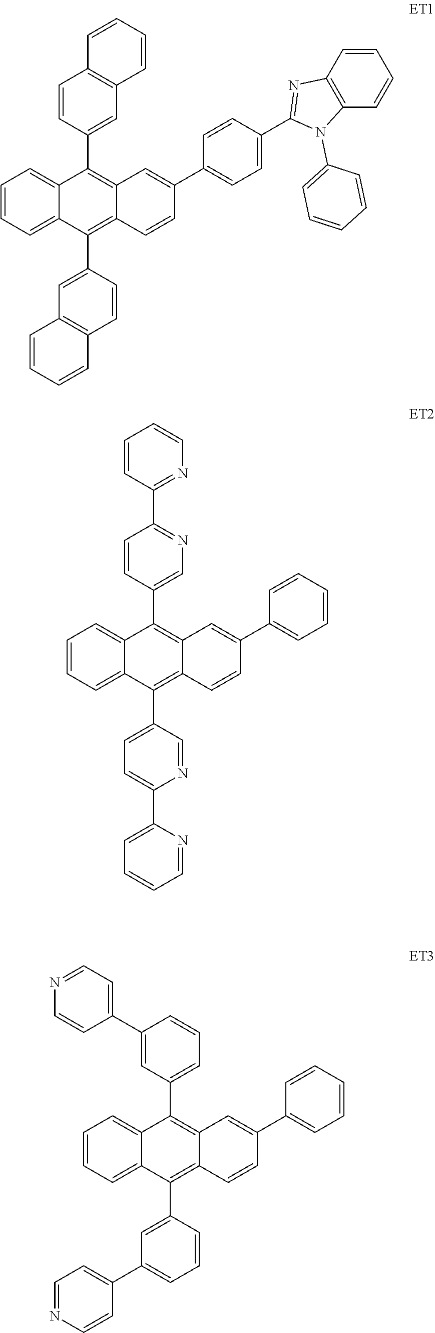

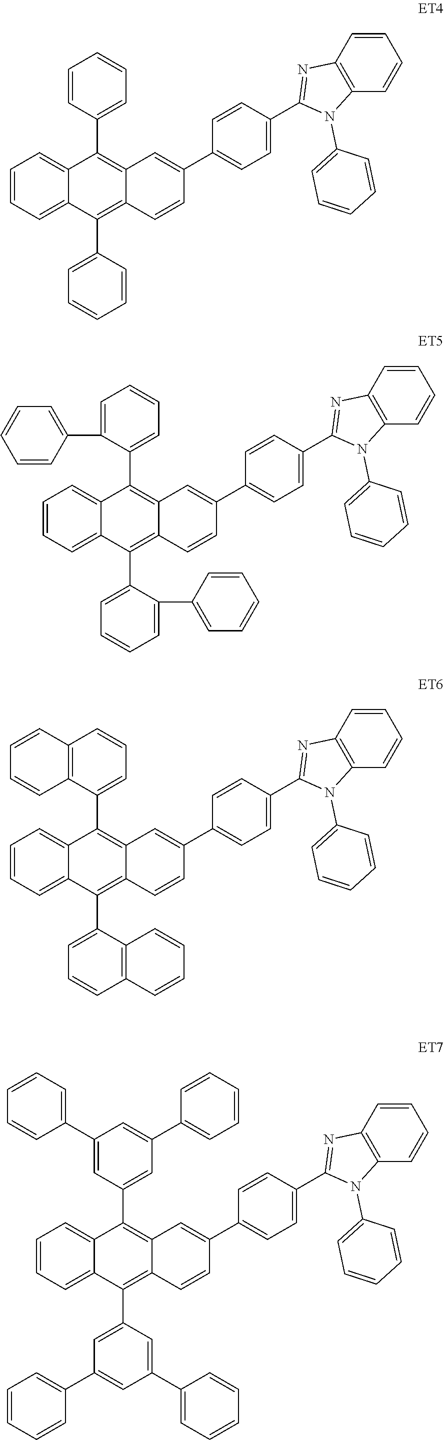 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

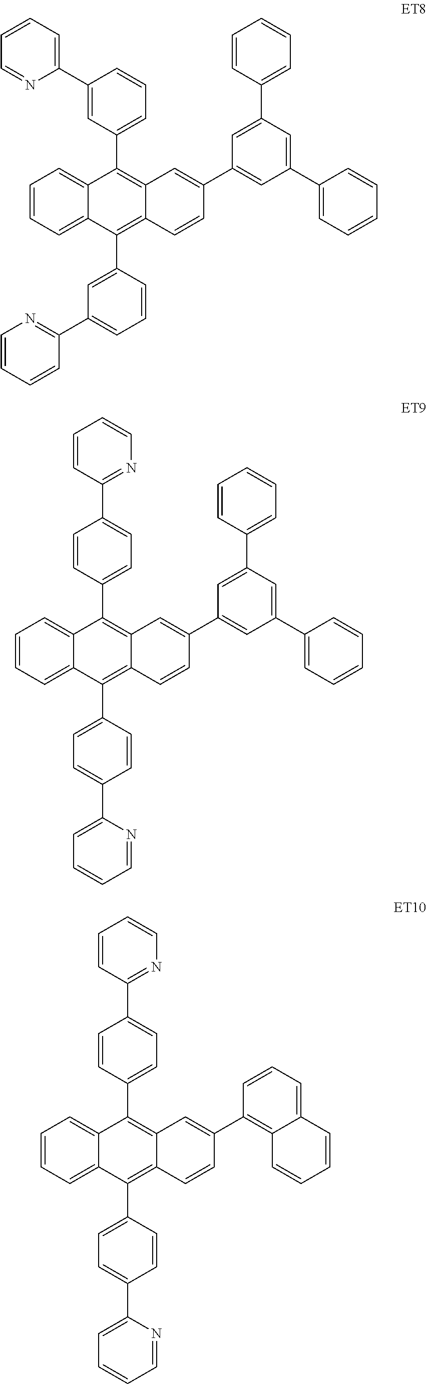

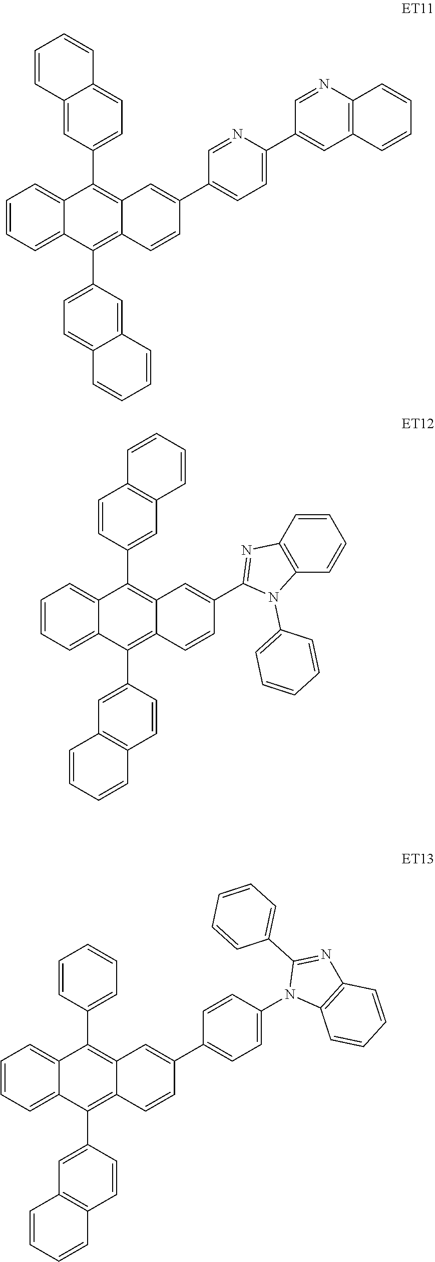 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

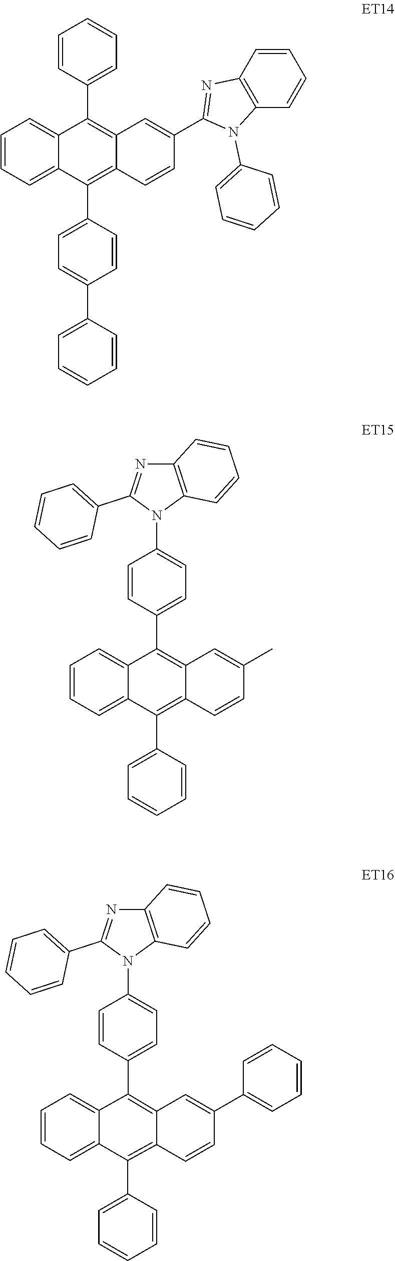 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

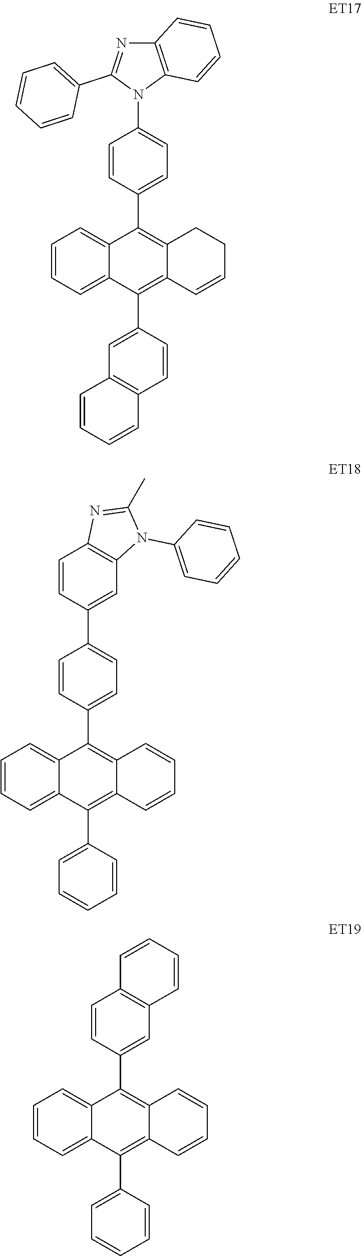

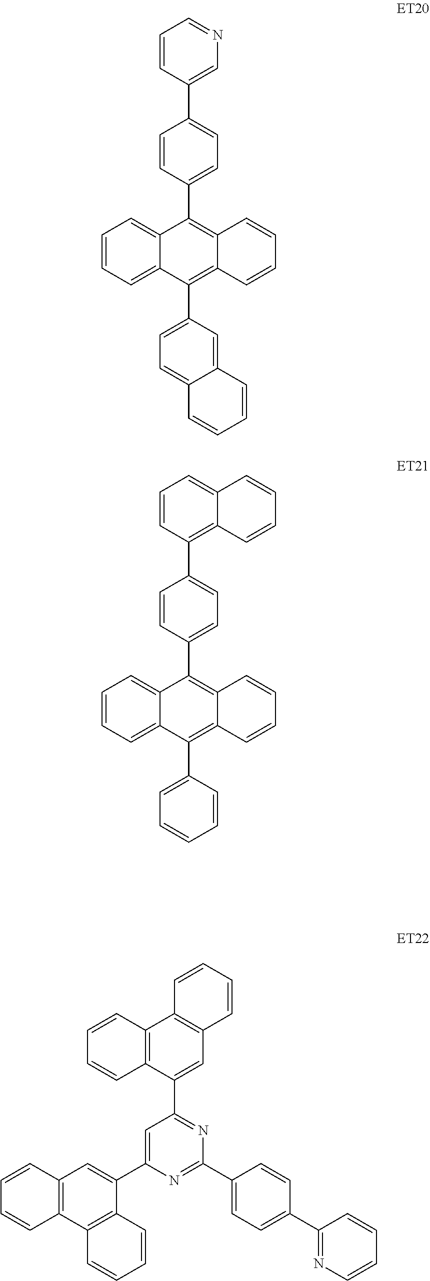

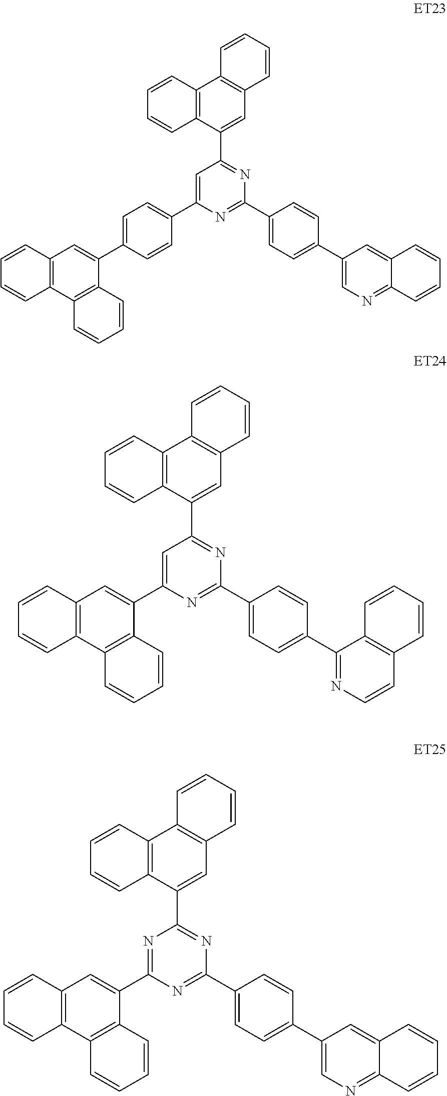 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

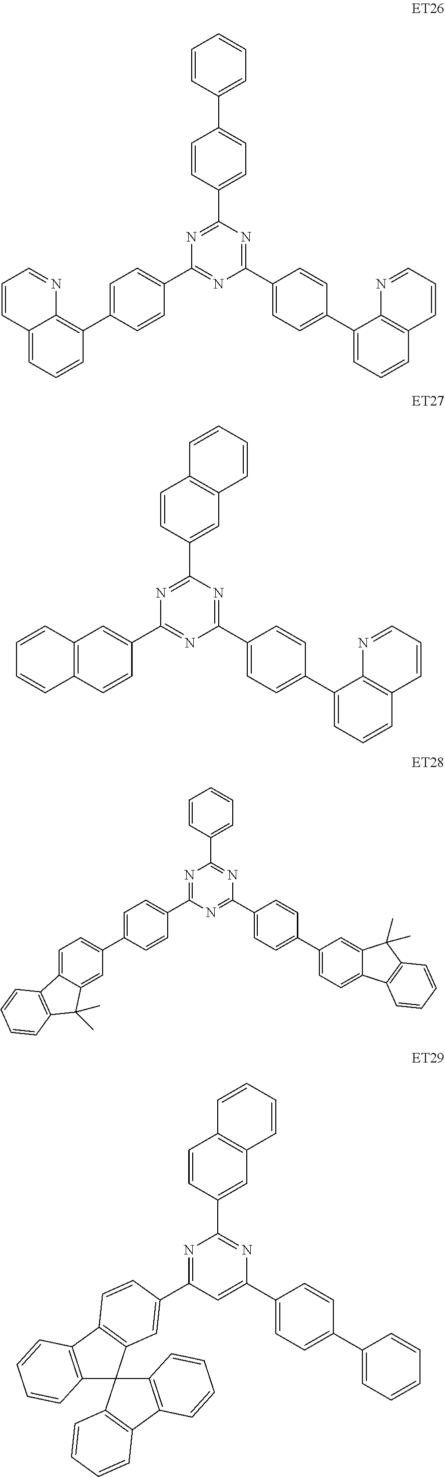 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

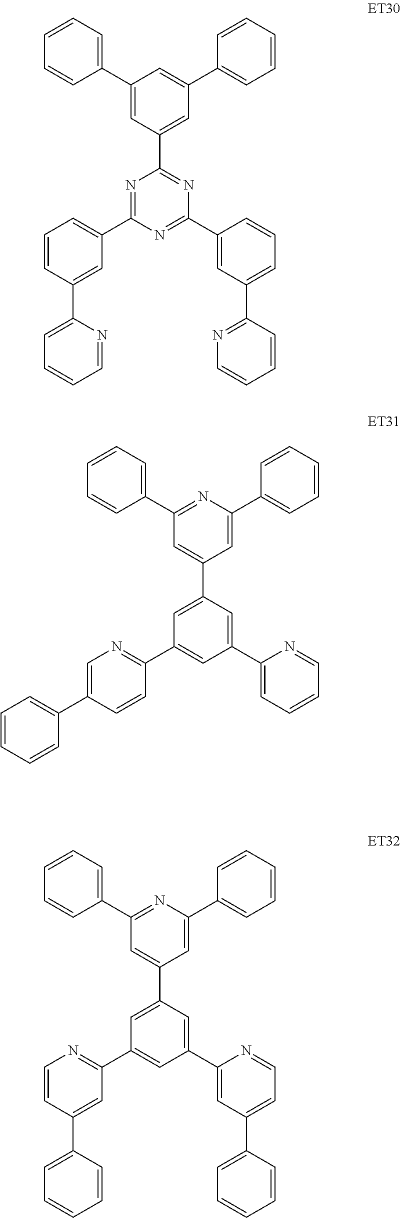 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

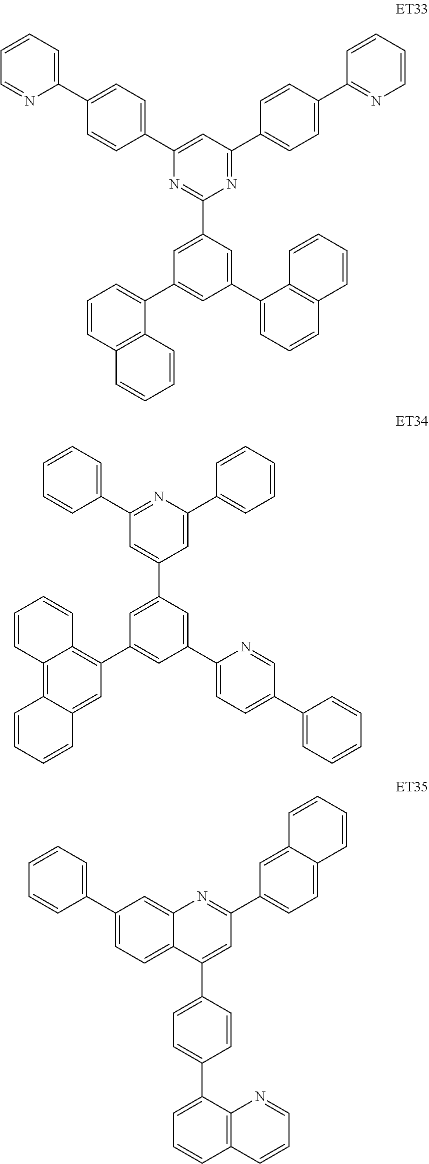

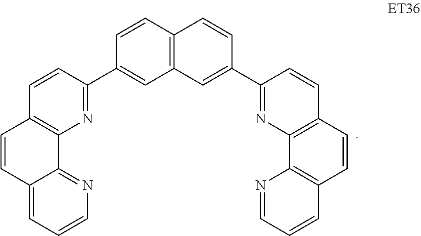 [SMILES: c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and/or KI, a lanthanide metal such as Yb, or a co-depositing material of the metal halide and the lanthanide metal. For example, in some embodiments, the electron transport region ETR may include KI:Yb, RbI:Yb, LiF:Yb, etc., as the co-depositing material. In some embodiments, the electron transport region ETR may utilize a metal oxide such as Li₂O and/or BaO, or 8-hydroxy-lithium quinolate (Liq). However, embodiments of the present disclosure are not limited thereto. In some embodiments, the electron transport region ETR may also be formed utilizing a mixture material of an electron transport material and an insulating organo metal salt. The organo metal salt may be a material having an energy band gap of about 4 eV or more. For example, the organo metal salt may include, for example, metal acetates, metal benzoates, metal acetoacetates, metal acetylacetonates, and/or metal stearates.

In one or more embodiments, the electron transport region ETR may include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the aforementioned materials. However, embodiments of the present disclosure are not limited thereto.

The electron transport region ETR may include the compounds of the electron transport region in at least one selected from among an electron injection layer EIL, an electron transport layer ETL, and a hole blocking layer HBL.

When the electron transport region ETR includes an electron transport layer ETL, a thickness of the electron transport layer ETL may be from about 100 Å to about 1,000 Å, for example, from about 150 Å to about 500 Å. When the thickness of the electron transport layer ETL satisfies the above-described range, satisfactory electron transport properties may be obtained without a substantial increase in driving voltage.

When the electron transport region ETR includes an electron injection layer EIL, a thickness of the electron injection layer EIL may be from about 1 Å to about 100 Å, or from about 3 Å to about 90 Å. When the thickness of the electron injection layer EIL satisfies the above described range, satisfactory electron injection properties may be obtained without inducing a substantial increase in driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, when the first electrode EL1 is an anode, the second cathode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, then the second electrode EL2 may include a transparent metal oxide, for example, ITO, IZO, ZnO, ITZO, etc.

When the second electrode EL2 is the transflective electrode or the reflective electrode, then the second electrode EL2 may include Ag, Mg, Cu, Al, Pt, Pd, Au, Ni, Nd, Ir, Cr, Li, Ca, LiF/Ca, LiF/Al, Mo, Ti, Yb, W, one or more compounds thereof, or one or more mixtures thereof (for example, AgMg, AgYb, or MgYb). In some embodiments, the second electrode EL2 may have a multilayered structure including a reflective layer or a transflective layer formed utilizing one or more selected from among the above-described materials and a transparent conductive layer formed utilizing ITO, IZO, ZnO, ITZO, etc. For example, the second electrode EL2 may include one or more selected from among the aforementioned metal materials, any combination of two or more metal materials selected from the aforementioned metal materials, or any oxide of the aforementioned metal materials.

In some embodiments, the second electrode EL2 may be connected with an auxiliary electrode. When the second electrode EL2 is connected with the auxiliary electrode, then the resistance of the second electrode EL2 may decrease.

In some embodiments, on the second electrode EL2 in the light emitting element ED, a capping layer CPL may be further disposed. The capping layer CPL may include a multilayer or a single layer.

In one or more embodiments, the capping layer CPL may be an organic layer or an inorganic layer. For example, in some embodiments, when the capping layer CPL includes an inorganic material, the inorganic material may include an alkali metal compound such as LiF, an alkaline earth metal compound such as MgF₂, SiON, SiNₓ, SiO_{y}, etc.

For example, in some embodiments, when the capping layer CPL includes an organic material, the organic material may include 2,2′-dimethyl-N,N′-di-[(1-naphthyl)-N,N′-diphenyl]-1,1′-biphenyl-4,4′-diamine(α-NPD), NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., or may include an epoxy resin, and/or acrylate such as methacrylate. In some embodiments, a capping layer CPL may include at least one selected from among Compounds P1 to P5, but embodiments of the present disclosure are not limited thereto.

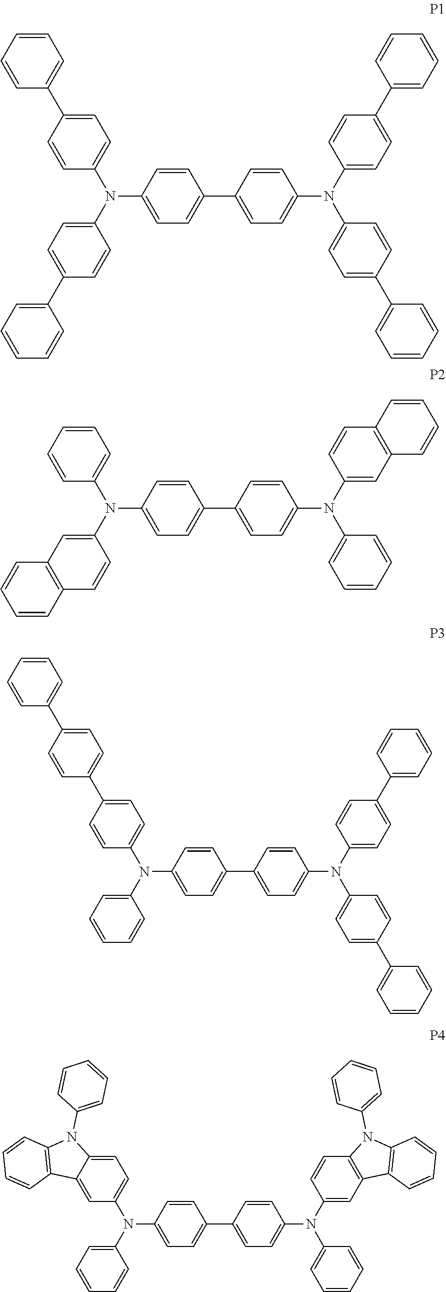 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

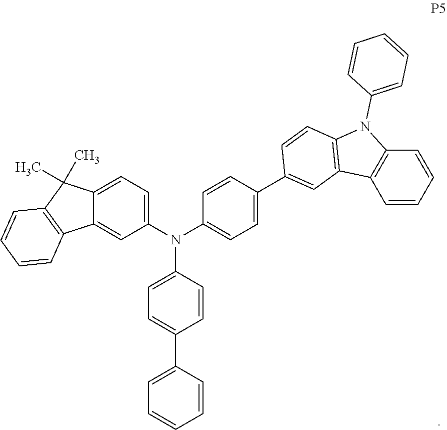 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, in one or more embodiments, the refractive index of the capping layer CPL with respect to light in a wavelength range of about 550 nm to about 660 nm may be about 1.6 or more.

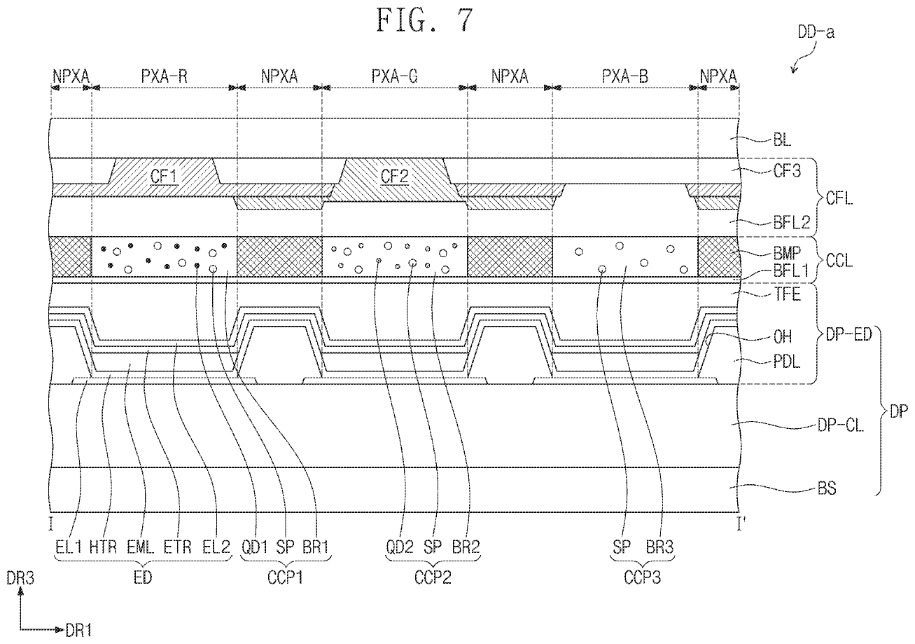

FIG. 7 to FIG. 10 are each a cross-sectional view on a display apparatus according to one or more embodiments of the present disclosure. In the explanation on the display apparatuses of embodiments by referring to FIG. 7 to FIG. 10, the overlapping parts with the explanation on FIG. 1 to FIG. 6 will not be explained/described again for conciseness, and only different features will be explained chiefly and mainly.

Referring to FIG. 7, the display apparatus DD-a according to one or more embodiments may include a display panel DP including a display device layer DP-ED, a light controlling layer CCL on the display panel DP, and a color filter layer CFL.

In one or more embodiments shown in FIG. 7, the display panel DP may include a base layer BS, a circuit layer DP-CL provided on the base layer BS, and a display device layer DP-ED, and the display device layer DP-ED may include a light emitting element ED.

The light emitting element ED may include a first electrode EL1, a hole transport region HTR on the first electrode EL1, a light-emitting layer EML on the hole transport region HTR, an electron transport region ETR on the light-emitting layer EML, and a second electrode EL2 on the electron transport region ETR. In one or more embodiments, the same structure as any one of the light emitting elements of FIG. 3 to FIG. 6 may be applied to the structure of the light emitting element ED shown in FIG. 7.

The hole transport region HTR of the light emitting element ED included in the display device DD-a according to one or more embodiments may include the amine compound of one or more embodiments described above.

Referring to FIG. 7, the light-emitting layer EML may be disposed in an opening part OH defined in a pixel definition layer PDL. For example, the light-emitting layer EML divided by the pixel definition layer PDL and correspondingly provided to each of luminous areas PXA-R, PXA-G, and PXA-B may be to emit light in substantially the same wavelength region. In the display apparatus DD-a of one or more embodiments, the light-emitting layer EML may be to emit blue light. In some embodiments, the light-emitting layer EML may be provided as a common layer for all luminous areas PXA-R, PXA-G, and PXA-B.

The light controlling layer CCL may be disposed on the display panel DP. The light controlling layer CCL may include a light converter. The light converter may be a quantum dot and/or a phosphor. The light converter may transform the wavelength of light provided and then emit. For example, the light controlling layer CCL may be a layer including a quantum dot or a layer including a phosphor.

The light controlling layer CCL may include multiple light controlling parts CCP1, CCP2, and CCP3. The light controlling parts CCP1, CCP2, and CCP3 may be separated and apart from one another.

Referring to FIG. 7, a partition pattern BMP may be disposed between the separated light controlling parts CCP1, CCP2, and CCP3, but embodiments of the present disclosure are not limited thereto. In FIG. 7, the partition pattern BMP is shown not to be overlapped with the light controlling parts CCP1, CCP2, and CCP3, but, in some embodiments, at least a portion of the edges of the light controlling parts CCP1, CCP2, and CCP3 may be overlapped with the partition pattern BMP.

The light controlling layer CCL may include a first light controlling part CCP1 including a first quantum dot QD1 converting first color light provided from the light emitting element ED into second color light, a second light controlling part CCP2 including a second quantum dot QD2 converting first color light into third color light, and a third light controlling part CCP3 transmitting first color light.

In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light emitting element ED. For example, in some embodiments, the first quantum dot QD1 may be a red quantum dot to emit red light, and the second quantum dot QD2 may be a green quantum dot to emit green light. On the quantum dots QD1 and QD2, the same content as those described above may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) any quantum dot but may include the scatterer SP.

The scatterer SP may be an inorganic particle. For example, the scatterer SP may include at least one selected from among TiO₂, ZnO, Al₂O₃, SiO₂, and hollow silica. In one or more embodiments, the scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

The first light controlling part CCP1, the second light controlling part CCP2, and the third light controlling part CCP3 may respectively include base resins BR1, BR2, and BR3 dispersing the quantum dots QD1 and QD2 and the scatterer SP. In one or more embodiments, the first light controlling part CCP1 may include the first quantum dot QD1 and the scatterer SP dispersed in a first base resin BR1, the second light controlling part CCP2 may include the second quantum dot QD2 and the scatterer SP dispersed in a second base resin BR2, and the third light controlling part CCP3 may include the scatterer particle SP dispersed in a third base resin BR3.

The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2, and BR3 may each independently be acrylic-based resins, urethane-based resins, silicone-based resins, epoxy-based resins, etc.

The base resins BR1, BR2, and BR3 may be transparent resins. In one or more embodiments, the first base resin BR1, the second base resin BR2, and the third base resin BR3 may be the same or different from each other.

In one or more embodiments, the light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may block or reduce the exposure of the light controlling parts CCP1, CCP2, and CCP3 to humidity/oxygen. In some embodiments, the barrier layer BFL1 may cover the light controlling parts CCP1, CCP2, and CCP3. In some embodiments, a color filter layer CFL, which will be explained later, may include a barrier layer BFL2 disposed on the light controlling parts CCP1, CCP2, and CCP3.

The barrier layers BFL1 and BFL2 may include at least one inorganic layer. For example, in some embodiments, the barrier layers BFL1 and BFL2 may be formed by including an inorganic material. For example, the barrier layers BFL1 and BFL2 may be formed by including one or more selected from among silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, and a metal thin film securing light transmittance. In some embodiments, the barrier layers BFL1 and BFL2 may further include an organic layer. The barrier layers BFL1 and BFL2 may be composed of a single layer or multiple layers.

In the display apparatus DD-a of one or more embodiments, the color filter layer CFL may be disposed on the light controlling layer CCL. For example, the color filter layer CFL may be disposed directly on the light controlling layer CCL. In these embodiments, the barrier layer BFL2 may not be provided.

The color filter layer CFL may include filters CF1, CF2, and CF3. Each of the first to third filters CF1, CF2, and CF3 may be disposed corresponding to a red luminous area PXA-R, a green luminous area PXA-G, and a blue luminous area PXA-B, respectively.

The color filter layer CFL may include a first filter CF1 transmitting the second color light, a second filter CF2 transmitting the third color light, and a third filter CF3 transmitting the first color light. For example, in some embodiments, the first filter CF1 may be a red filter, the second filter CF2 may be a green filter, and the third filter CF3 may be a blue filter. Each of the filters CF1, CF2, and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye.

In some embodiments, the third filter CF3 may not include (e.g., may exclude) any pigment and/or dye. The third filter CF3 may include a polymer photosensitive resin and not include any pigment and/or dye. The third filter CF3 may be transparent. The third filter CF3 may be formed utilizing a transparent photosensitive resin.

In some embodiments, the first filter CF1 and the second filter CF2 may each be yellow filters. The first filter CF1 and the second filter CF2 may be provided in one body without distinction.

In one or more embodiments, the color filter layer CFL may further include a light blocking part. The color filter layer CFL may include the light blocking part disposed to overlap the boundaries of neighboring filters CF1, CF2, and CF3. The light blocking part may be a black matrix. The light blocking part may be formed by including an organic light blocking material and/or an inorganic light blocking material including a black pigment and/or black dye. The light blocking part may prevent or reduce light leakage phenomenon and divide the boundaries among adjacent filters CF1, CF2, and CF3.

On the color filter layer CFL, a base substrate BL may be disposed/provided. The base substrate BL may be a member providing a base surface on which the color filter layer CFL, the light controlling layer CCL, etc. are disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may not be provided.

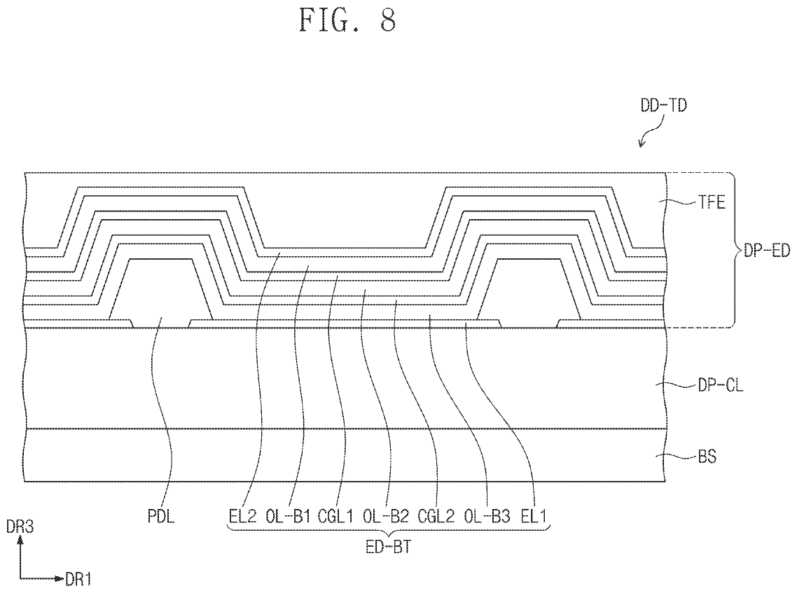

FIG. 8 is a cross-sectional view showing a portion of the display apparatus according to one or more embodiments. In a display apparatus DD-TD of one or more embodiments, the light emitting element ED-BT may include multiple light emitting structures OL-B1, OL-B2, and OL-B3. The light emitting element ED-BT may include oppositely disposed first electrode EL1 and second electrode EL2, and the multiple light emitting structures OL-B1, OL-B2, and OL-B3 stacked in order in a thickness direction and provided between the first electrode EL1 and the second electrode EL2. Each of the light emitting structures OL-B1, OL-B2, and OL-B3 may include an light-emitting layer EML (FIG. 7), and a hole transport region HTR and an electron transport region ETR disposed with the light-emitting layer EML (FIG. 7) therebetween.

For example, in some embodiments, the light emitting element ED-BT included in the display apparatus DD-TD may be a light emitting element of a tandem structure including multiple light-emitting layers.

In one or more embodiments shown in FIG. 8, light emitted from the light emitting structures OL-B1, OL-B2, and OL-B3 may be all blue light. However, embodiments of the present disclosure are not limited thereto, and the wavelength regions of light emitted from the light emitting structures OL-B1, OL-B2, and OL-B3 may be different from each other. For example, in one or more embodiments, the light emitting element ED-BT including the multiple light emitting structures OL-B1, OL-B2, and OL-B3 each emitting light in different wavelength regions may be to emit white light (e.g., combined white light).

Between neighboring light emitting structures OL-B1, OL-B2, and OL-B3, charge generating layers CGL1 and CGL2 may be disposed. The charge generating layers CGL1 and CGL2 may include a p-type or kind charge (e.g., P-charge) generating layer and/or an n-type or kind charge (e.g., N-charge) generating layer.

The above-described amine compound according to one or more embodiments may be contained in at least one selected from among the light emitting structures OL-B1, OL-B2, and OL-B3 included in a display device DD-TD according to one or more embodiments.

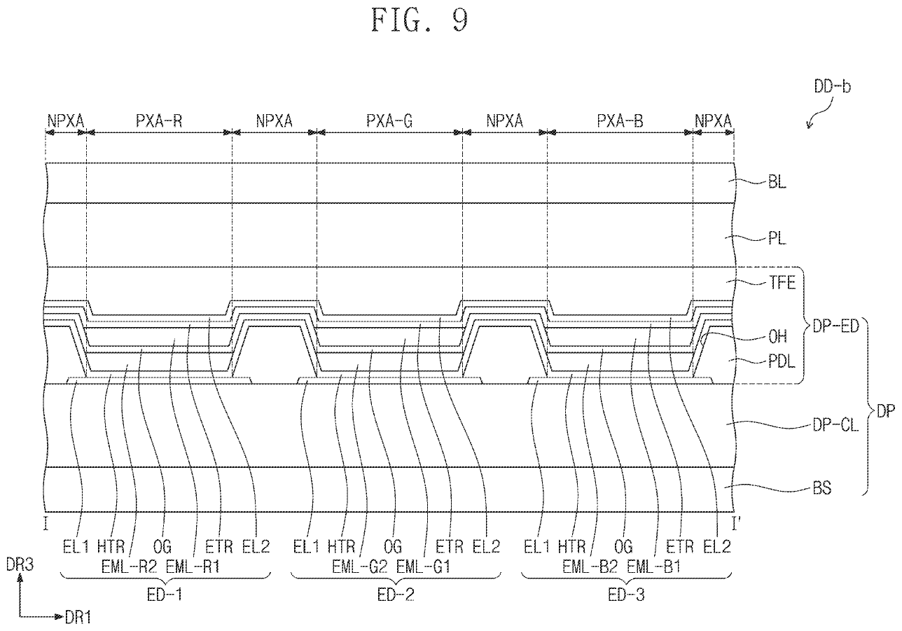

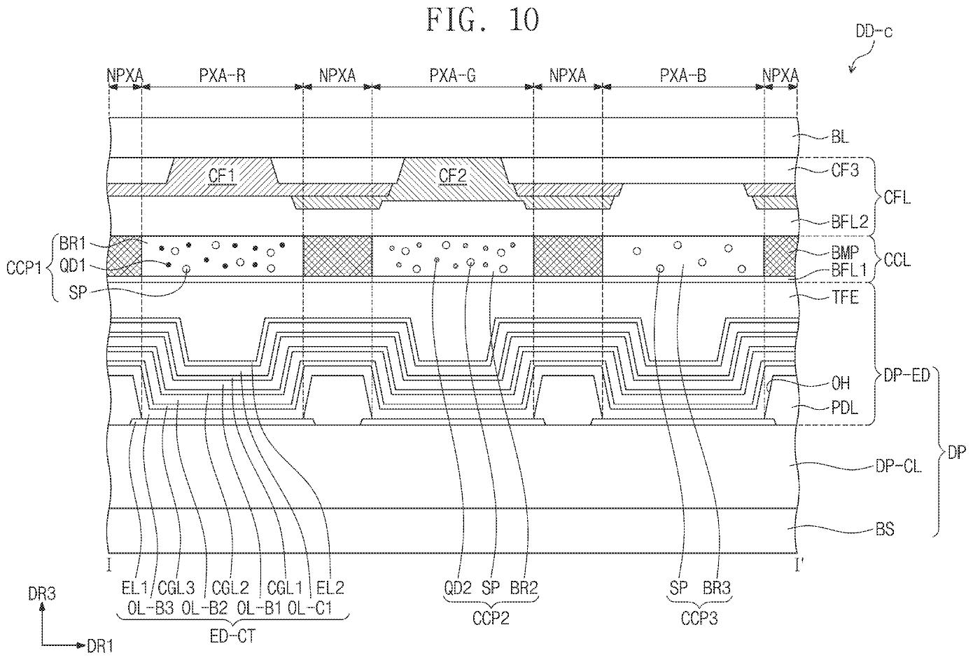

FIG. 9 is a cross-sectional view showing a display apparatus according to one or more embodiments of the present disclosure. FIG. 10 is a cross-sectional view showing a display apparatus according to one or more embodiments of the present disclosure.

Referring to FIG. 9, a display apparatus DD-b according to one or more embodiments may include light emitting elements ED-1, ED-2, and ED-3, each formed by stacking two light-emitting layers. Compared to the display apparatus DD shown in FIG. 2, the display apparatus DD-b shown in FIG. 9 is different in that first to third light emitting elements ED-1, ED-2, and ED-3 each include two light-emitting layers stacked in a thickness direction. In the first to third light emitting elements ED-1, ED-2, and ED-3, the two light-emitting layers may be to emit light in substantially the same wavelength region.

In one or more embodiments, the first light emitting element ED-1 may include a first red light-emitting layer EML-R1 and a second red light-emitting layer EML-R₂. The second light emitting element ED-2 may include a first green light-emitting layer EML-G1 and a second green light-emitting layer EML-G2. In addition, the third light emitting element ED-3 may include a first blue light-emitting layer EML-B1 and a second blue light-emitting layer EML-B2. Between the first red light-emitting layer EML-R1 and the second red light-emitting layer EML-R₂, between the first green light-emitting layer EML-G1 and the second green light-emitting layer EML-G2, and between the first blue light-emitting layer EML-B1 and the second blue light-emitting layer EML-B2, an emission auxiliary part OG may be disposed.

The emission auxiliary part OG may include a single layer or a multilayer. The emission auxiliary part OG may include a charge generating layer. For example, the emission auxiliary part OG may include an electron transport region, a charge generating layer, and a hole transport region stacked in order (e.g., in the stated order). The emission auxiliary part OG may be provided as a common layer in all of the first to third light emitting elements ED-1, ED-2, and ED-3. However, embodiments of the present disclosure are not limited thereto, and the emission auxiliary part OG may be patterned and provided in an opening part OH defined in a pixel definition layer PDL.

The first red light-emitting layer EML-R1, the first green light-emitting layer EML-G1, and the first blue light-emitting layer EML-B1 may each be disposed between the electron transport region ETR and the emission auxiliary part OG. The second red light-emitting layer EML-R2, the second green light-emitting layer EML-G2, and the second blue light-emitting layer EML-B2 may each be disposed between the emission auxiliary part OG and the hole transport region HTR.

For example, in one or more embodiments, the first light emitting element ED-1 may include a first electrode EL1, a hole transport region HTR, a second red light-emitting layer EML-R2, an emission auxiliary part OG, a first red light-emitting layer EML-R₁, an electron transport region ETR, and a second electrode EL2, stacked in order (e.g., in the stated order). The second light emitting element ED-2 may include a first electrode EL1, a hole transport region HTR, a second green light-emitting layer EML-G2, an emission auxiliary part OG, a first green light-emitting layer EML-G1, an electron transport region ETR, and a second electrode EL2, stacked in order (e.g., in the stated order). The third light emitting element ED-3 may include a first electrode EL1, a hole transport region HTR, a second blue light-emitting layer EML-B2, an emission auxiliary part OG, a first blue light-emitting layer EML-B1, an electron transport region ETR, and a second electrode EL2, stacked in order (e.g., in the stated order).

In one or more embodiments, an optical auxiliary layer PL may be disposed on a display device layer DP-ED. The optical auxiliary layer PL may include a polarization layer. The optical auxiliary layer PL may be disposed on a display panel DP and may control reflected light at the display panel DP by external light. In some embodiments, the optical auxiliary layer PL may not be provided from the display apparatus.

Different from FIG. 8 and FIG. 9, a display apparatus DD-c in FIG. 10 is shown to include four light emitting structures OL-B1, OL-B2, OL-B3, and OL-C1. A light emitting element ED-CT may include oppositely disposed first electrode EL1 and second electrode EL2, and first to fourth light emitting structures OL-B1, OL-B2, OL-B3, and OL-C1 stacked in a thickness direction between the first electrode EL1 and the second electrode EL2. For example, the third light emitting structures OL-B3, the second light emitting structures OL-B2, the first light emitting structures OL-B1, and the fourth light emitting structures OL-C1 are stacked in order (e.g., in the stated order) in a thickness direction. Between the first to fourth light emitting structures OL-B1, OL-B2, OL-B3, and OL-C1, charge generating layers CGL1, CGL2, and CGL3 may be disposed. For example, A first charge generating layer CGL1 is disposed between the first light emitting structures OL-B1 and the fourth light emitting structures OL-C1. A second charge generating layer CGL2 is disposed between the first light emitting structures OL-B1 and the second light emitting structures OL-B2. A third charge generating layer CGL3 is disposed between the second light emitting structures OL-B2 and the third light emitting structures OL-B3.

Among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3, and OL-C1 may be to emit different wavelengths of light.

The above-described amine compound according to one or more embodiments may be contained in at least one selected from among the light emitting structures OL-B1, OL-B2, OL-B3, and OL-C1 included in a display device DD-c according to one or more embodiments.

The light-emitting element ED according to one or more embodiments of the present disclosure may contain the above-described amine compound according to one or more embodiments in at least one of functional layers disposed between the first electrode EL1 and the second electrode EL2, and thus may exhibit improved luminous efficiency and improved lifespan characteristics. The light-emitting element ED according to one or more embodiments may contain the above-described amine compound according to one or more embodiments in at least one selected from among a hole transport region HTR, a light-emitting layer EML, and an electron transport region ETR, each disposed between the first electrode EL1 and the second electrode EL2, or also in a capping layer CPL. For example, the amine compound according to one or more embodiments may be contained in the hole transport region HTR of the light-emitting element ED according to one or more embodiments, and the light-emitting element ED according to one or more embodiments may exhibit high efficiency and long-lifespan characteristics.

The above-described amine compound according to one or more embodiments includes a first amine group, a second amine group, a first linker connecting the first amine group and the second amine group, and a first substituent connected to the first amine group, the first substituent including a naphthyl moiety and a first sub-substituent, and thus stability of materials may increase and hole transport ability may be improved. Therefore, the light-emitting element containing the amine compound according to one or more embodiments may have improved lifespan and efficiency. In some embodiments, the light-emitting element according to one or more embodiments may contain the amine compound according to one or more embodiments in the hole transport layer, thereby exhibiting the improved efficiency and lifespan characteristics.

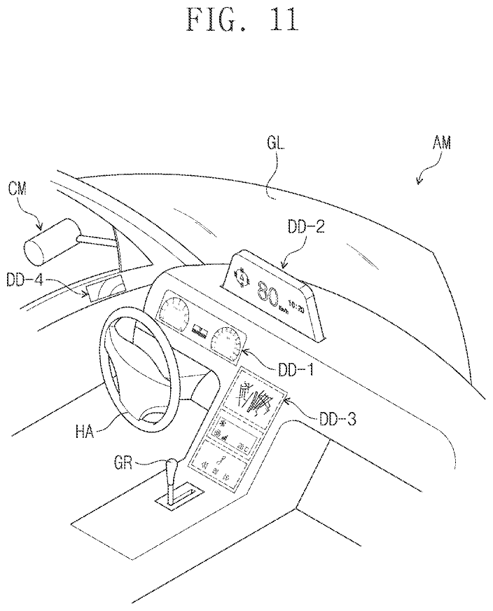

FIG. 11 is a diagram showing an automobile AM in which first to fourth display apparatuses DD-1, DD-2, DD-3, and DD-4 are disposed. At least one selected from among the first to fourth display apparatuses DD-1, DD-2, DD-3, and DD-4 may include substantially the same configurations of the display apparatuses DD, DD-TD, DD-a, DD-b, and/or DD-c, explained referring to FIGS. 1, 2, and 7 to 10.

In FIG. 11, a vehicle is shown as an automobile AM, but this is a mere example. The first to fourth display apparatuses DD-1, DD-2, DD-3 and DD-4 may be disposed on other transport apparatuses such as bicycles, motorcycles, trains, ships, and/or airplanes. In some embodiments, at least one selected from among the first to fourth display apparatuses DD-1, DD-2, DD-3, and DD-4 including substantially the same configurations of the display apparatuses DD, DD-TD, DD-a, DD-b, and/or DD-c may be introduced in personal computers, laptop computers, personal digital terminals, game consoles, portable electronic devices, televisions, monitors, external billboards, and/or the like. These are suggested as mere examples, and the display apparatus may be introduced in other electronic devices as long as not deviated from the present disclosure.

In one or more embodiments, at least one selected from among the first to fourth display devices DD-1, DD-2, DD-3, and DD-4 may include the light emitting element ED of one or more embodiments described with reference to FIGS. 3 to 6.

The light emitting element ED according to one or more embodiments may include the amine compound of one or more embodiments. In one or more embodiments, at least one selected from among the first to fourth display devices DD-1, DD-2, DD-3, and DD-4 may include a light emitting element ED containing the amine compound of one or more embodiments, thereby increasing lifespan.

Referring to FIG. 11, an automobile AM may include a steering wheel HA for the operation of the automobile AM and a gear GR. In addition, the automobile AM may include a front window GL disposed to face a driver.

A first display apparatus DD-1 may be disposed in a first region overlapping with the steering wheel HA. For example, the first display apparatus DD-1 may be a digital cluster displaying first information of the automobile AM. The first information may include a first graduation showing the travel speed of the automobile AM, a second graduation showing the number of revolution of an engine (i.e., revolutions per minute (RPM)), and images showing a fuel state. The first graduation and second graduation may be represented by digital images.

A second display apparatus DD-2 may be disposed in a second region facing a driver seat and overlapping with the front window GL. The driver seat may be a seat where the steering wheel HA faces. For example, the second display apparatus DD-2 may be a head up display (HUD) showing second information of the automobile AM. The second display apparatus DD-2 may be optically clear. The second information may include digital numbers showing the travel speed of the automobile AM and may further include information including the current time. In some embodiments, the second information of the second display apparatus DD-2 may be projected and displayed on the front window GL.

A third display apparatus DD-3 may be disposed in a third region adjacent to the gear GR. For example, the third display apparatus DD-3 may be a center information display (CID) for the automobile, disposed between a driver seat and a passenger seat and showing third information. The passenger seat may be a seat separated from the driver seat with the gear GR therebetween. The third information may include information on road conditions (for example, navigation information), on playing music or radio, on playing a dynamic image (or image), on the temperature in the automobile AM, and/or the like.

A fourth display apparatus DD-4 may be disposed in a fourth region separated from the steering wheel HA and the gear GR and adjacent to a side of the automobile AM. For example, the fourth display apparatus DD-4 may be a digital wing mirror displaying fourth information. The fourth display apparatus DD-4 may display external images of the automobile AM, taken by a camera module CM disposed at the outside of the automobile AM. The fourth information may include the external image of the automobile AM.

The above-described first to fourth information are mere examples, and the first to fourth display apparatuses DD-1, DD-2, DD-3, and DD-4 may further display information on the inside and outside of the automobile. The first to fourth information may include different information from each other. However, embodiments of the present disclosure are not limited thereto, for example, in some embodiments, a portion of the first to fourth information may include the same information.

Hereinafter, with reference to Examples and Comparative Examples, an amine compound according to one or more embodiments of the present disclosure and a light-emitting element according to one or more embodiments will be described in more detail. In addition, Examples described are only shown for the understanding of the present disclosure, but the scope of the present disclosure is not limited thereto.

### EXAMPLES

### 1. Synthesis of Amine Compounds

First, synthetic methods of amine compounds according to one or more embodiments will be described in more detail by example synthetic methods of Compounds 64, 67, 73, 76, 81, 84, 88, 91, 92, 94, 96, 100, 121, 122, 127, 128, 130, 135, 140, 145, 147, 153, 155, 157, and 181. In some embodiments, the—described synthetic methods of the amine compounds are provided as examples, but the synthetic methods of the compounds according to embodiments of the present disclosure are not limited to Examples.

### (1) Synthesis of Compound 64

### Synthesis of Intermediate 64-1

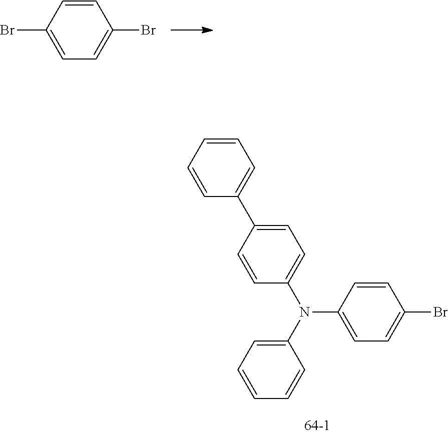 [SMILES: Brc1ccc(Br)cc1 ; Brc1ccc(N(c2ccccc2)c2ccc(-c3ccccc3)cc2)cc1]

1,4-dibromobenzene (26 mmol, 1.3 eq), N-phenyl-[1,1′-biphenyl]-4-amine (20 mmol, 1 eq), tris(dibenzylideneacetone)dipalladium(0) (Pd₂(dba)₃) (1 mmol, 0.05 eq), sodium tert-butoxide (t-BuONa) (40 mmol, 2 eq), 2,2′-bis(diphenylphosphino)-1,1′-binaphthyl (BINAP, 2 mmol, 0.1 eq), and 300 mL of toluene were put into a single-neck-round flask, and the mixture was stirred at about 110° C. for about 1 hour. After the reaction was completed, the resultant was worked up with ether/H₂, and then was purified by column chromatography utilizing dichloromethane/hexane=1/5 (v/v) as an eluent, to obtain about 12 mmol of Intermediate 64-1. (yield=60%)

### Synthesis of Compound 64

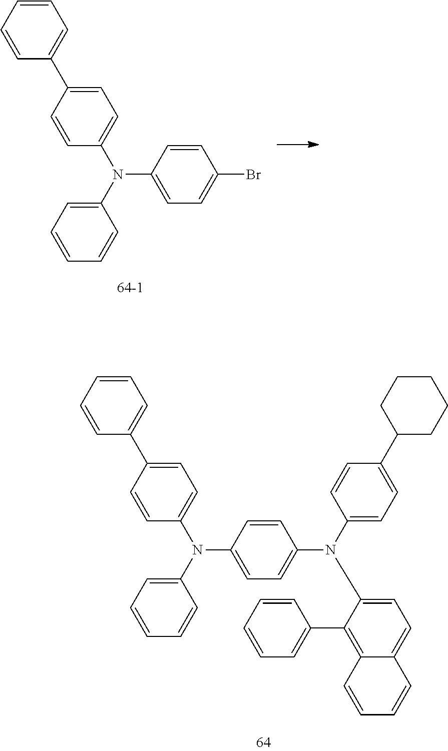 [SMILES: Brc1ccc(N(c2ccccc2)c2ccc(-c3ccccc3)cc2)cc1 ; c1ccc(-c2ccc(N(c3ccccc3)c3ccc(N(c4ccc(C5CCCCC5)cc4)c4ccc5ccccc5c4-c4ccccc4)cc3)cc2)cc1]

Intermediate 64-1 (12 mmol, 1.2 eq), N-(4-cyclohexylphenyl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.3 mmol of Compound 64. (yield=63%)

### (2) Synthesis of Compound 67

### Synthesis of Intermediate 67-1

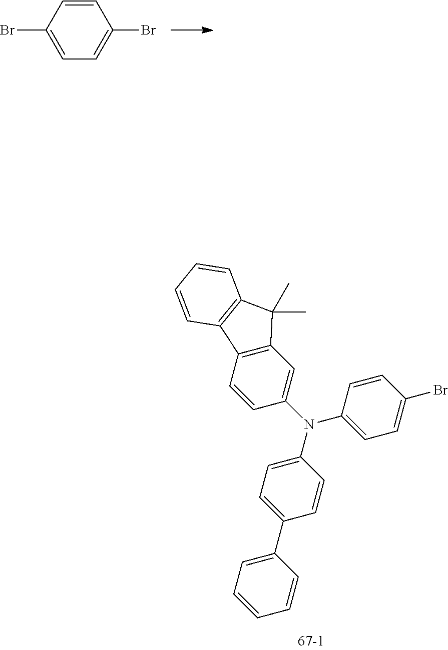 [SMILES: Brc1ccc(Br)cc1 ; CC1(C)c2ccccc2-c2ccc(N(c3ccc(Br)cc3)c3ccc(-c4ccccc4)cc3)cc21]

1,4-dibromobenzene (26 mmol, 1.3 eq), N-([1,1′-biphenyl]-4-yl)-9,9-dimethyl-9H-fluoren-2-amine (20 mmol, 1 eq), Pd₂(dba)₃ (1 mmol, 0.05 eq), t-BuONa (40 mmol, eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 13 mmol of Intermediate 67-1. (yield=65%)

### Synthesis of Compound 67

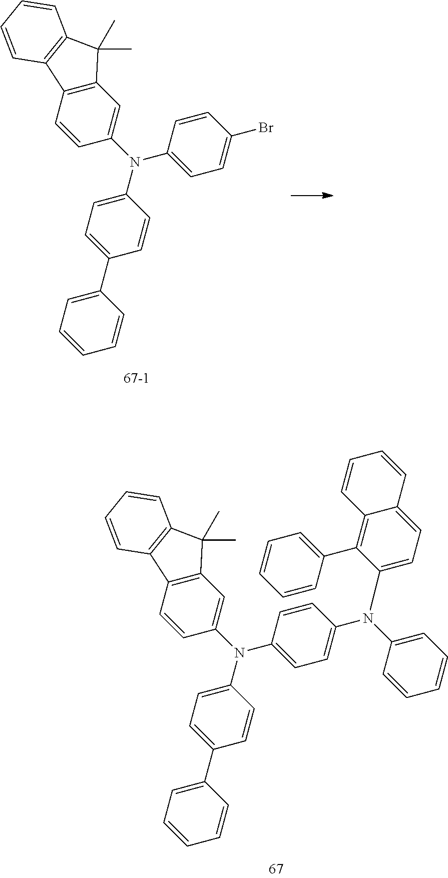 [SMILES: CC1(C)c2ccccc2-c2ccc(N(c3ccc(Br)cc3)c3ccc(-c4ccccc4)cc3)cc21 ; CC1(C)c2ccccc2-c2ccc(N(c3ccc(-c4ccccc4)cc3)c3ccc(N(c4ccccc4)c4ccc5ccccc5c4-c4ccccc4)cc3)cc21]

Intermediate 67-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as eluent, to obtain about 5.9 mmol of Compound 67. (yield=59%)

### (3) Synthesis of Compound 73

### Synthesis of Intermediate 73-1

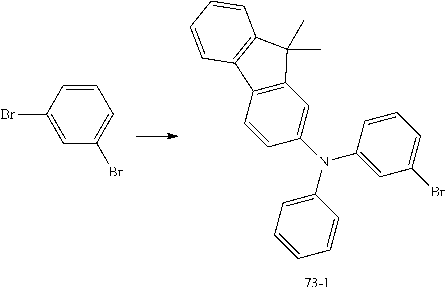 [SMILES: Brc1cccc(Br)c1 ; CC1(C)c2ccccc2-c2ccc(N(c3ccccc3)c3cccc(Br)c3)cc21]

1,3-dibromobenzene (26 mmol, 1.3 eq), 9,9-dimethyl-N-phenyl-9H-fluoren-2-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 13 mmol of Intermediate 73-1. (yield=65%)

### Synthesis of Compound 73

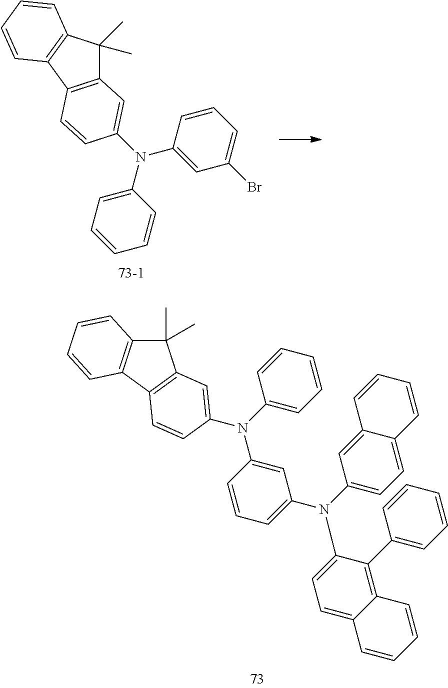 [SMILES: CC1(C)c2ccccc2-c2ccc(N(c3ccccc3)c3cccc(Br)c3)cc21 ; CC1(C)c2ccccc2-c2ccc(N(c3ccccc3)c3cccc(N(c4ccc5ccccc5c4)c4ccc5ccccc5c4-c4ccccc4)c3)cc21]

Intermediate 73-1 (12 mmol, 1.2 eq), N-(naphthalen-2-yl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.0 mmol of Compound 73. (yield=60%)

### (4) Synthesis of Compound 76

### Synthesis of Intermediate 76-1

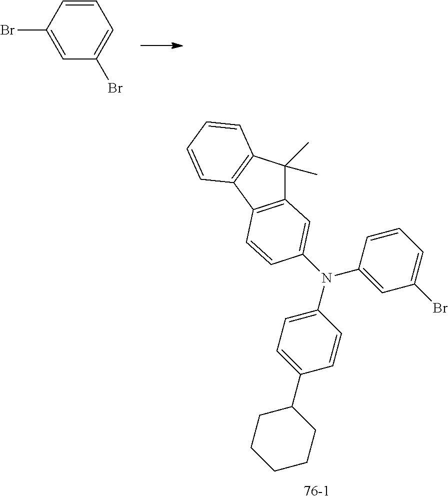 [SMILES: Brc1cccc(Br)c1 ; CC1(C)c2ccccc2-c2ccc(N(c3ccc(C4CCCCC4)cc3)c3cccc(Br)c3)cc21]

1,3-dibromobenzene (26 mmol, 1.3 eq), N-(4-cyclohexylphenyl)-9,9-dimethyl-9H-fluoren-2-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 76-1. (yield=60%)

### Synthesis of Compound 76

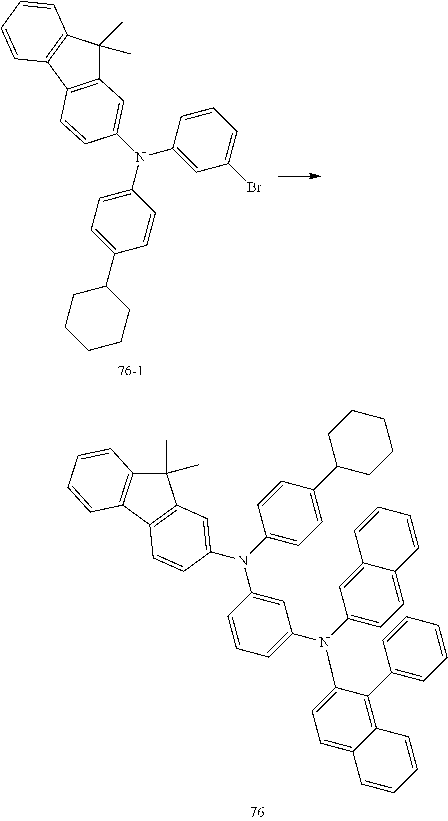 [SMILES: CC1(C)c2ccccc2-c2ccc(N(c3ccc(C4CCCCC4)cc3)c3cccc(Br)c3)cc21 ; CC1(C)c2ccccc2-c2ccc(N(c3ccc(C4CCCCC4)cc3)c3cccc(N(c4ccc5ccccc5c4)c4ccc5ccccc5c4-c4ccccc4)c3)cc21]

Intermediate 76-1 (12 mmol, 1.2 eq), N-(naphthalen-2-yl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 5.0 mmol of Compound 76. (yield=50%)

### (5) Synthesis of Compound 81

### Synthesis of Intermediate 81-1

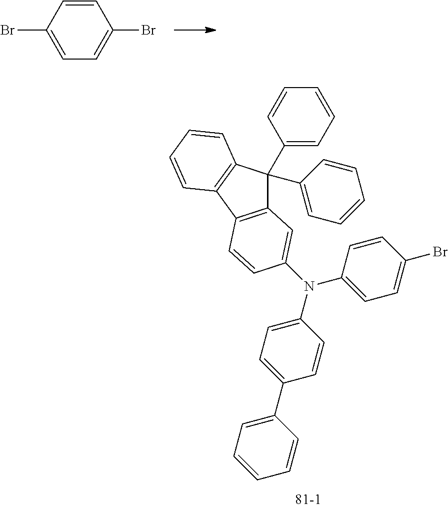 [SMILES: Brc1ccc(Br)cc1 ; Brc1ccc(N(c2ccc(-c3ccccc3)cc2)c2ccc3c(c2)C(c2ccccc2)(c2ccccc2)c2ccccc2-3)cc1]

1,4-dibromobenzene (26 mmol, 1.3 eq), N-([1,1′-biphenyl]-4-yl)-9,9-diphenyl-9H-fluoren-2-amine (20 mmol, 1 eq), Pd₂(dba)₃ (1 mmol, 0.05 eq), t-BuONa (40 mmol, eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 15 mmol of Intermediate 81-1. (yield=75%)

### Synthesis of Compound 81

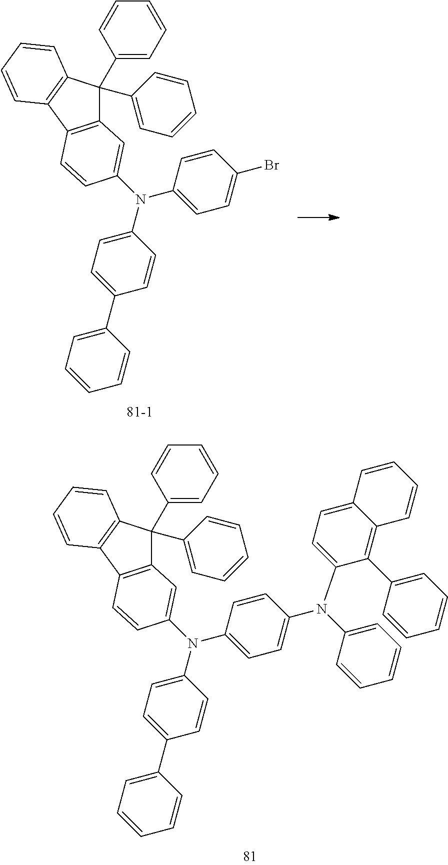 [SMILES: Brc1ccc(N(c2ccc(-c3ccccc3)cc2)c2ccc3c(c2)C(c2ccccc2)(c2ccccc2)c2ccccc2-3)cc1 ; c1ccc(-c2ccc(N(c3ccc(N(c4ccccc4)c4ccc5ccccc5c4-c4ccccc4)cc3)c3ccc4c(c3)C(c3ccccc3)(c3ccccc3)c3ccccc3-4)cc2)cc1]

Intermediate 81-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 7.0 mmol of Compound 81. (yield=70%)

### (6) Synthesis of Compound 84

### Synthesis of Intermediate 84-1

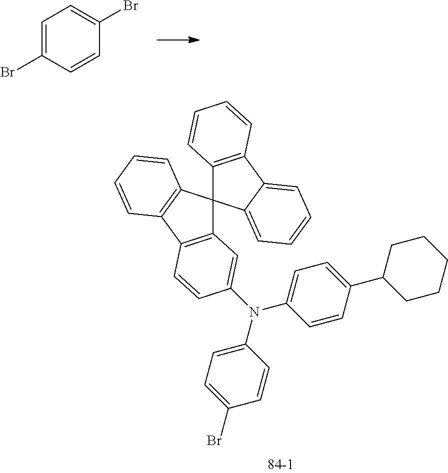 [SMILES: Brc1ccc(Br)cc1 ; Brc1ccc(N(c2ccc(C3CCCCC3)cc2)c2ccc3c(c2)C2(c4ccccc4-c4ccccc42)c2ccccc2-3)cc1]

1,4-dibromobenzene (26 mmol, 1.3 eq), N-(4-cyclohexylphenyl)-9,9′-spirobi[fluoren]-2-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 14 mmol of Intermediate 84-1. (yield=70%)

### Synthesis of Compound 84

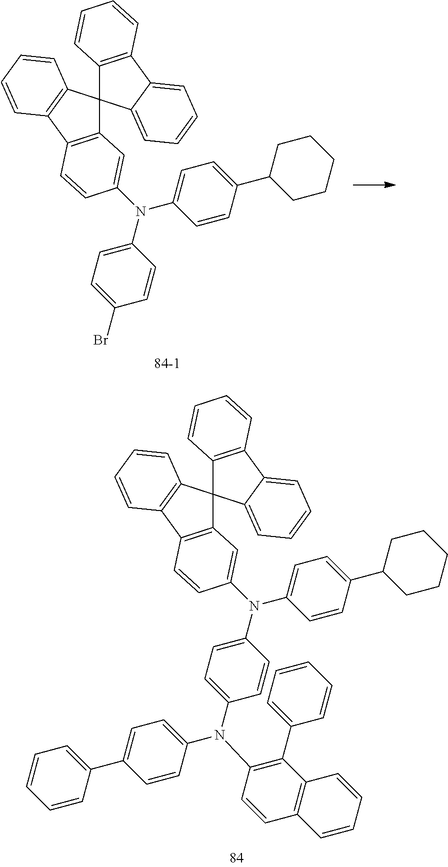 [SMILES: Brc1ccc(N(c2ccc(C3CCCCC3)cc2)c2ccc3c(c2)C2(c4ccccc4-c4ccccc42)c2ccccc2-3)cc1 ; c1ccc(-c2ccc(N(c3ccc(N(c4ccc(C5CCCCC5)cc4)c4ccc5c(c4)C4(c6ccccc6-c6ccccc64)c4ccccc4-5)cc3)c3ccc4ccccc4c3-c3ccccc3)cc2)cc1]

Intermediate 84-1 (12 mmol, 1.2 eq), N-([1,1′-biphenyl]-4-yl)-1-phenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 7.0 mmol of Compound 84. (yield=70%)

### (7) Synthesis of Compound 88

### Synthesis of Intermediate 88-1

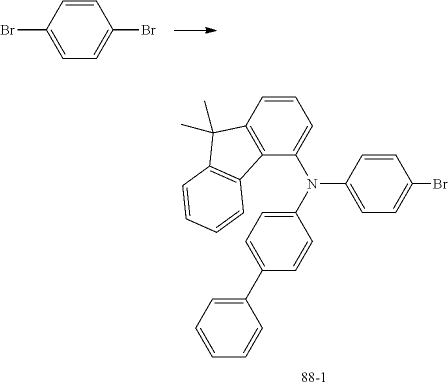 [SMILES: Brc1ccc(Br)cc1 ; CC1(C)c2ccccc2-c2c(N(c3ccc(Br)cc3)c3ccc(-c4ccccc4)cc3)cccc21]

1,4-dibromobenzene (26 mmol, 1.3 eq), N-([1,1′-biphenyl]-4-yl)-9,9-dimethyl-9H-fluoren-4-amine (20 mmol, 1 eq), Pd₂(dba)₃ (1 mmol, 0.05 eq), t-BuONa (40 mmol, eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 13 mmol of Intermediate 88-1. (yield=65%)

### Synthesis of Compound 88

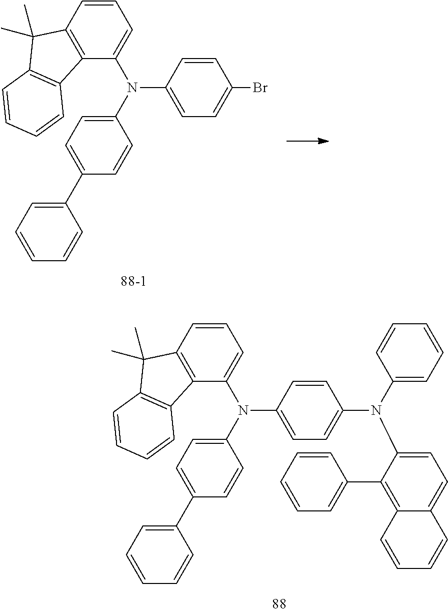 [SMILES: CC1(C)c2ccccc2-c2c(N(c3ccc(Br)cc3)c3ccc(-c4ccccc4)cc3)cccc21 ; CC1(C)c2ccccc2-c2c(N(c3ccc(-c4ccccc4)cc3)c3ccc(N(c4ccccc4)c4ccc5ccccc5c4-c4ccccc4)cc3)cccc21]

Intermediate 88-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 5.9 mmol of Compound 88. (yield=59%)

### (8) Synthesis of Compound 91

### Synthesis of Intermediate 91-1

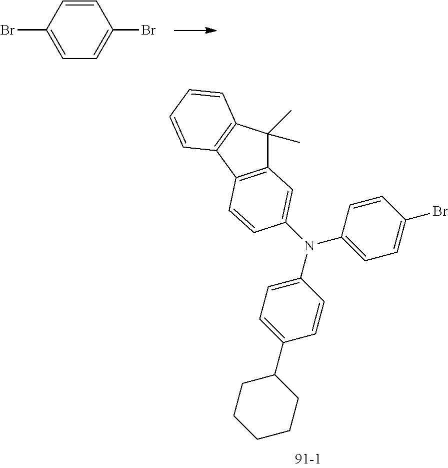 [SMILES: Brc1ccc(Br)cc1 ; CC1(C)c2ccccc2-c2ccc(N(c3ccc(Br)cc3)c3ccc(C4CCCCC4)cc3)cc21]

1,4-dibromobenzene (26 mmol, 1.3 eq), N-(4-cyclohexylphenyl)-9,9-dimethyl-9H-fluoren-2-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 91-1. (yield=60%)

### Synthesis of Compound 91

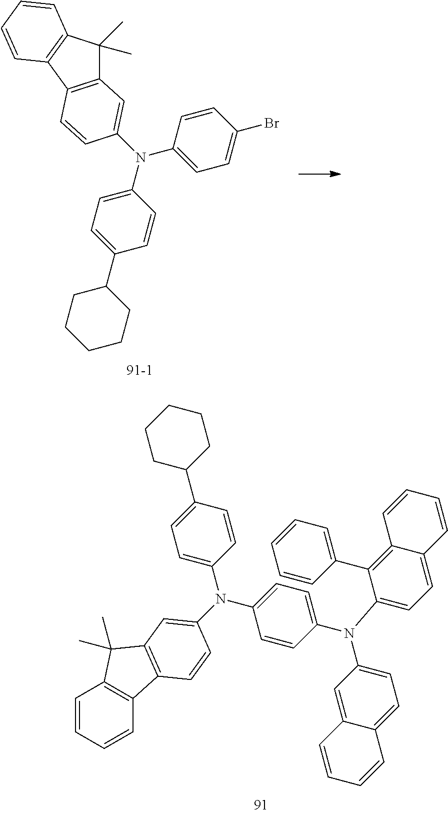 [SMILES: CC1(C)c2ccccc2-c2ccc(N(c3ccc(Br)cc3)c3ccc(C4CCCCC4)cc3)cc21 ; CC1(C)c2ccccc2-c2ccc(N(c3ccc(C4CCCCC4)cc3)c3ccc(N(c4ccc5ccccc5c4)c4ccc5ccccc5c4-c4ccccc4)cc3)cc21]

Intermediate 91-1 (12 mmol, 1.2 eq), N-(naphthalen-2-yl)-1-phenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 5.0 mmol of Compound 91. (yield=50%)

### (9) Synthesis of Compound 92

### Synthesis of Intermediate 92-1

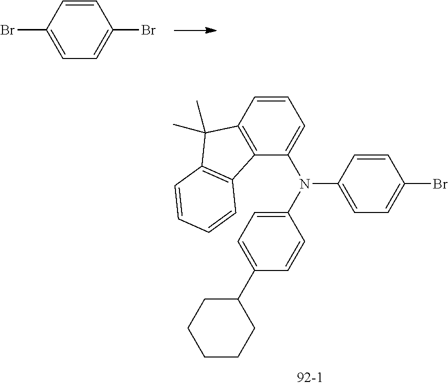 [SMILES: Brc1ccc(Br)cc1 ; CC1(C)c2ccccc2-c2c(N(c3ccc(Br)cc3)c3ccc(C4CCCCC4)cc3)cccc21]

1,4-dibromobenzene (26 mmol, 1.3 eq), N-(4-cyclohexylphenyl)-9,9-dimethyl-9H-fluoren-4-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 13 mmol of Intermediate 92-1. (yield=65%)

### Synthesis of Compound 92

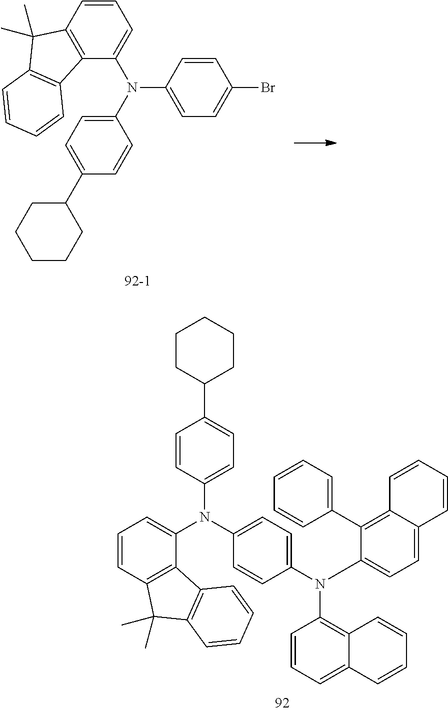 [SMILES: CC1(C)c2ccccc2-c2c(N(c3ccc(Br)cc3)c3ccc(C4CCCCC4)cc3)cccc21 ; CC1(C)c2ccccc2-c2c(N(c3ccc(C4CCCCC4)cc3)c3ccc(N(c4ccc5ccccc5c4-c4ccccc4)c4cccc5ccccc45)cc3)cccc21]

Intermediate 92-1 (12 mmol, 1.2 eq), N-(naphthalen-1-yl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 4.0 mmol of Compound 92. (yield=40%)

### (10) Synthesis of Compound 94

### Synthesis of Intermediate 94-1

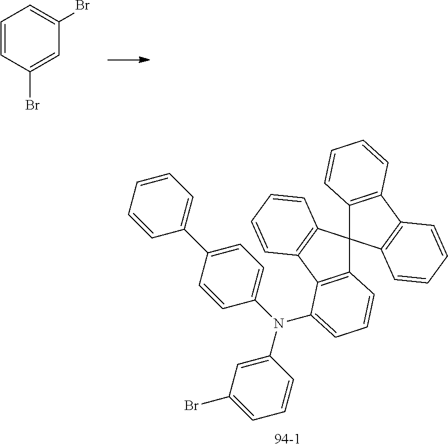 [SMILES: Brc1cccc(Br)c1 ; Brc1cccc(N(c2ccc(-c3ccccc3)cc2)c2cccc3c2-c2ccccc2C32c3ccccc3-c3ccccc32)c1]

1,3-dibromobenzene (26 mmol, 1.3 eq), N-([1,1′-biphenyl]-4-yl)-9,9′-spirobi[fluoren]-4-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 15 mmol of Intermediate 94-1. (yield=75%)

### Synthesis of Compound 94

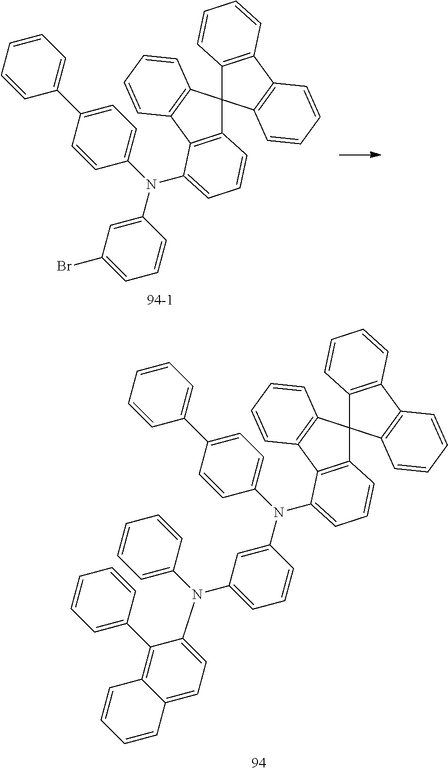 [SMILES: Brc1cccc(N(c2ccc(-c3ccccc3)cc2)c2cccc3c2-c2ccccc2C32c3ccccc3-c3ccccc32)c1 ; c1ccc(-c2ccc(N(c3cccc(N(c4ccccc4)c4ccc5ccccc5c4-c4ccccc4)c3)c3cccc4c3-c3ccccc3C43c4ccccc4-c4ccccc43)cc2)cc1]

Intermediate 94-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.8 mmol of Compound 94. (yield=68%)

### (11) Synthesis of Compound 96

### Synthesis of Intermediate 96-1

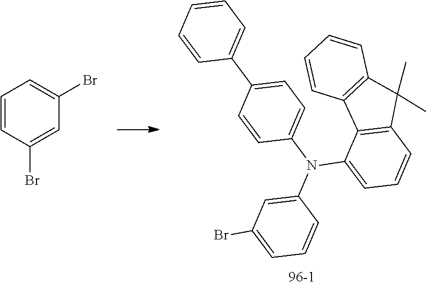 [SMILES: Brc1cccc(Br)c1 ; CC1(C)c2ccccc2-c2c(N(c3ccc(-c4ccccc4)cc3)c3cccc(Br)c3)cccc21]

1,3-dibromobenzene (26 mmol, 1.3 eq), N-([1,1′-biphenyl]-4-yl)-9,9′-dimethyl-9H-fluoren-4-amine (20 mmol, 1 eq), Pd₂(dba)₃ (1 mmol, 0.05 eq), t-BuONa (40 mmol, eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 16 mmol of Intermediate 96-1. (yield=80%)

### Synthesis of Compound 96

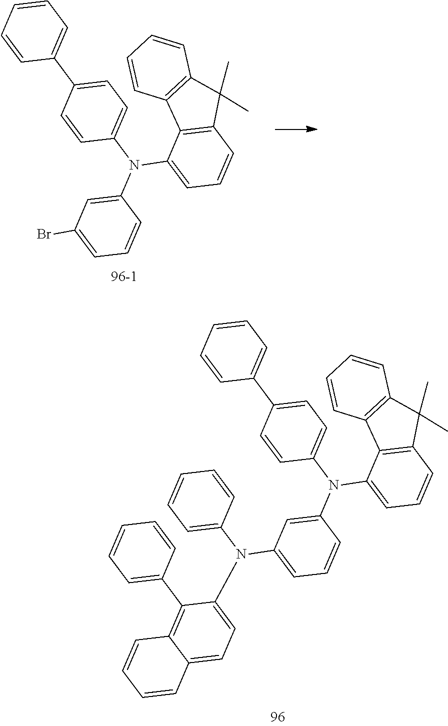 [SMILES: CC1(C)c2ccccc2-c2c(N(c3ccc(-c4ccccc4)cc3)c3cccc(Br)c3)cccc21 ; CC1(C)c2ccccc2-c2c(N(c3ccc(-c4ccccc4)cc3)c3cccc(N(c4ccccc4)c4ccc5ccccc5c4-c4ccccc4)c3)cccc21]

Intermediate 96-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.4 mmol of Compound 96. (yield=64%)

### (12) Synthesis of Compound 100

### Synthesis of Intermediate 100-1

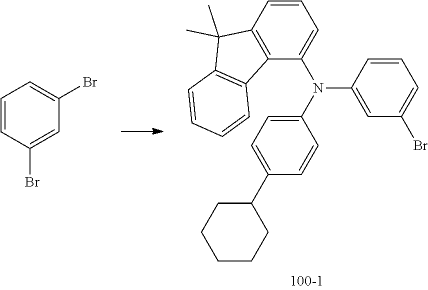 [SMILES: Brc1cccc(Br)c1 ; CC1(C)c2ccccc2-c2c(N(c3ccc(C4CCCCC4)cc3)c3cccc(Br)c3)cccc21]

1,3-dibromobenzene (26 mmol, 1.3 eq), N-(4-cyclohexylphenyl)-9,9-dimethyl-9H-fluoren-4-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 13 mmol of Intermediate 100-1. (yield=65%)

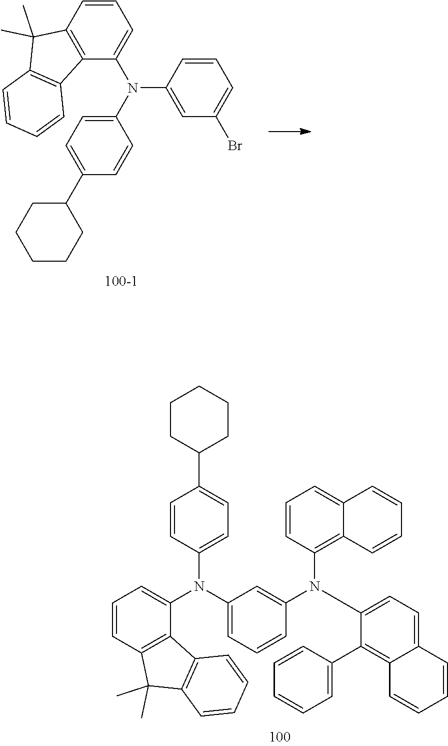 [SMILES: CC1(C)c2ccccc2-c2c(N(c3ccc(C4CCCCC4)cc3)c3cccc(Br)c3)cccc21 ; CC1(C)c2ccccc2-c2c(N(c3ccc(C4CCCCC4)cc3)c3cccc(N(c4ccc5ccccc5c4-c4ccccc4)c4cccc5ccccc45)c3)cccc21]

### Synthesis of Compound 100

Intermediate 100-1 (12 mmol, 1.2 eq), N-(naphthalen-1-yl)-1-phenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 4.2 mmol of Compound 100. (yield=42%)

### (13) Synthesis of Compound 121

### Synthesis of Intermediate 121-1

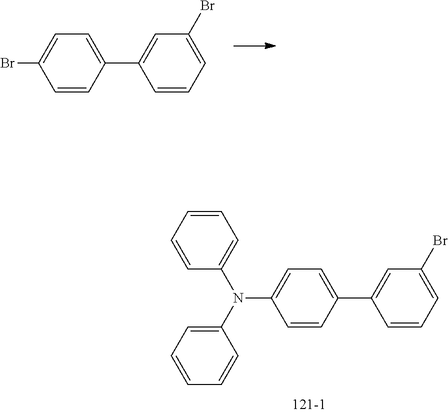 [SMILES: Brc1ccc(-c2cccc(Br)c2)cc1 ; Brc1cccc(-c2ccc(N(c3ccccc3)c3ccccc3)cc2)c1]

3,4′-dibromo-1,1′-biphenyl (26 mmol, 1.3 eq), diphenylamine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 15 mmol of Intermediate 121-1. (yield=75%)

### Synthesis of Compound 121

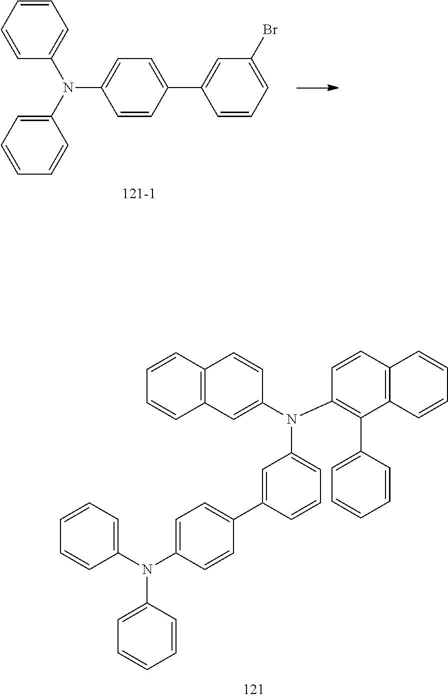 [SMILES: Brc1cccc(-c2ccc(N(c3ccccc3)c3ccccc3)cc2)c1 ; c1ccc(-c2c(N(c3cccc(-c4ccc(N(c5ccccc5)c5ccccc5)cc4)c3)c3ccc4ccccc4c3)ccc3ccccc23)cc1]

Intermediate 121-1 (12 mmol, 1.2 eq), N-(naphthalen-2-yl)-1-phenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 5.0 mmol of Compound 121. (yield=50%)

### (14) Synthesis of Compound 122

### Synthesis of Intermediate 122-1

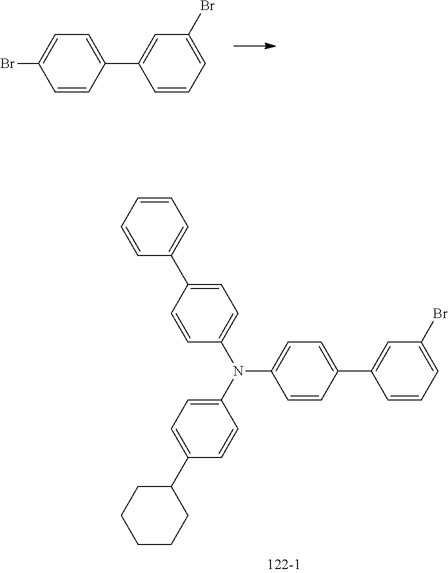 [SMILES: Brc1ccc(-c2cccc(Br)c2)cc1 ; Brc1cccc(-c2ccc(N(c3ccc(-c4ccccc4)cc3)c3ccc(C4CCCCC4)cc3)cc2)c1]

3,4′-dibromo-1,1′-biphenyl (26 mmol, 1.3 eq), N-(4-cyclohexylphenyl)-[1,1′-biphenyl]-4-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 122-1. (yield=60%)

### Synthesis of Compound 122

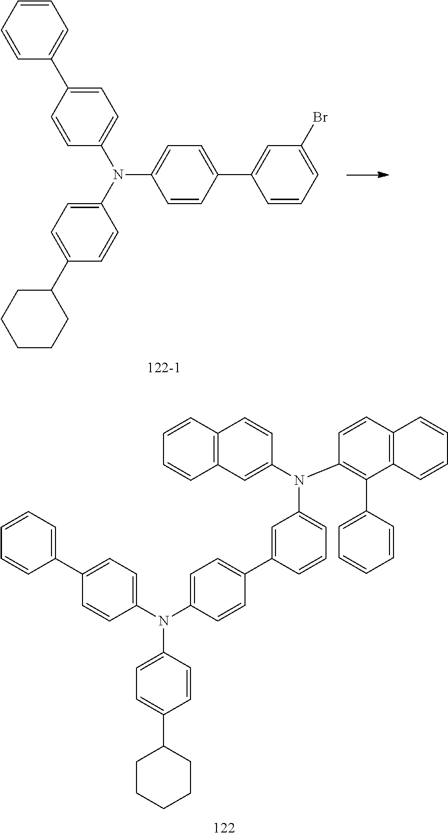 [SMILES: Brc1cccc(-c2ccc(N(c3ccc(-c4ccccc4)cc3)c3ccc(C4CCCCC4)cc3)cc2)c1 ; c1ccc(-c2ccc(N(c3ccc(-c4cccc(N(c5ccc6ccccc6c5)c5ccc6ccccc6c5-c5ccccc5)c4)cc3)c3ccc(C4CCCCC4)cc3)cc2)cc1]

Intermediate 122-1 (12 mmol, 1.2 eq), N-(naphthalen-2-yl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 4.8 mmol of Compound 122. (yield=48%)

### (15) Synthesis of Compound 127

### Synthesis of Intermediate 127-1

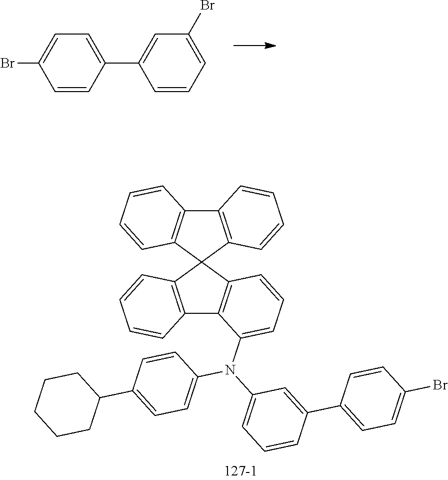 [SMILES: Brc1ccc(-c2cccc(Br)c2)cc1 ; Brc1ccc(-c2cccc(N(c3ccc(C4CCCCC4)cc3)c3cccc4c3-c3ccccc3C43c4ccccc4-c4ccccc43)c2)cc1]

3,4′-dibromo-1,1′-biphenyl (26 mmol, 1.3 eq), N-(4-cyclohexylphenyl)-9,9′-spirobi[fluoren]-4-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 127-1. (yield=60%)

### Synthesis of Compound 127

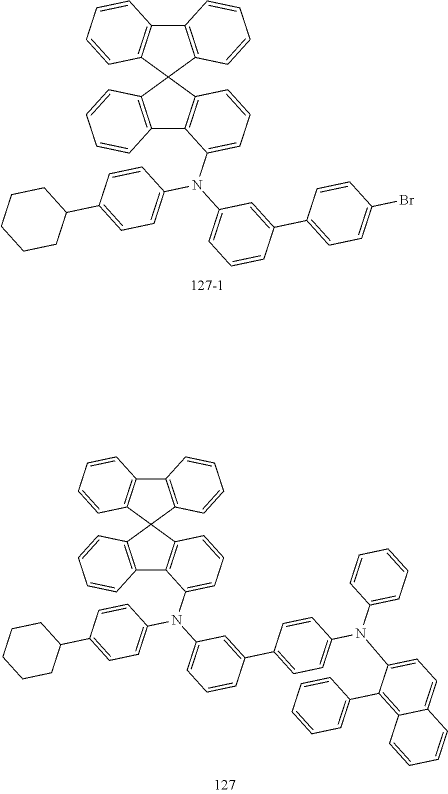 [SMILES: Brc1ccc(-c2cccc(N(c3ccc(C4CCCCC4)cc3)c3cccc4c3-c3ccccc3C43c4ccccc4-c4ccccc43)c2)cc1 ; c1ccc(-c2c(N(c3ccccc3)c3ccc(-c4cccc(N(c5ccc(C6CCCCC6)cc5)c5cccc6c5-c5ccccc5C65c6ccccc6-c6ccccc65)c4)cc3)ccc3ccccc23)cc1]

Intermediate 127-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 5.8 mmol of Compound 127. (yield=58%)

### (16) Synthesis of Compound 128

### Synthesis of Intermediate 128-1

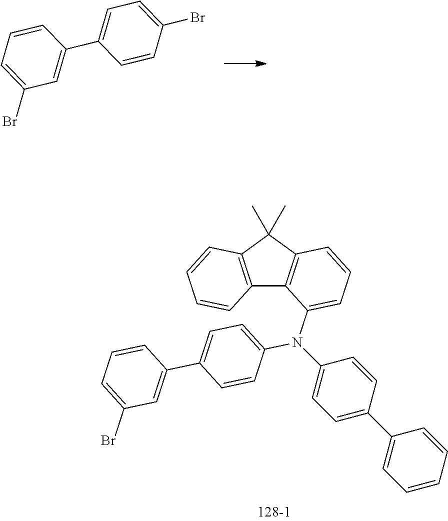 [SMILES: Brc1ccc(-c2cccc(Br)c2)cc1 ; CC1(C)c2ccccc2-c2c(N(c3ccc(-c4ccccc4)cc3)c3ccc(-c4cccc(Br)c4)cc3)cccc21]

3,4′-dibromo-1,1′-biphenyl (26 mmol, 1.3 eq), N-([1,1′-biphenyl]-4-yl)-9,9-dimethyl-9H-fluoren-4-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 13 mmol of Intermediate 128-1. (yield=65%)

### Synthesis of Compound 128

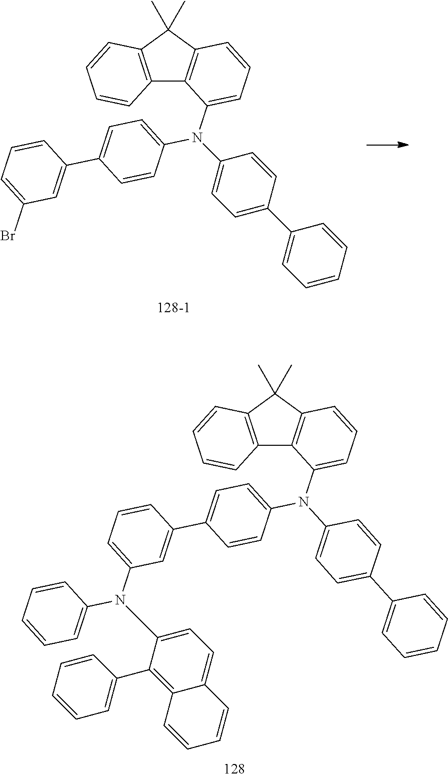 [SMILES: CC1(C)c2ccccc2-c2c(N(c3ccc(-c4ccccc4)cc3)c3ccc(-c4cccc(Br)c4)cc3)cccc21 ; CC1(C)c2ccccc2-c2c(N(c3ccc(-c4ccccc4)cc3)c3ccc(-c4cccc(N(c5ccccc5)c5ccc6ccccc6c5-c5ccccc5)c4)cc3)cccc21]

Intermediate 128-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.2 mmol of Compound 128. (yield=62%)

### (17) Synthesis of Compound 130

### Synthesis of Intermediate 130-1

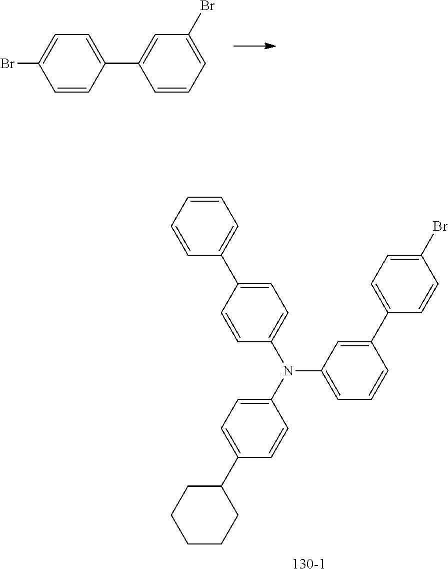 [SMILES: Brc1ccc(-c2cccc(Br)c2)cc1 ; Brc1ccc(-c2cccc(N(c3ccc(-c4ccccc4)cc3)c3ccc(C4CCCCC4)cc3)c2)cc1]

3,4′-dibromo-1,1′-biphenyl (26 mmol, 1.3 eq), N-(4-cyclohexylphenyl)-[1,1′-biphenyl]-4-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 15 mmol of Intermediate 130-1. (yield=65%)

### Synthesis of Compound 130

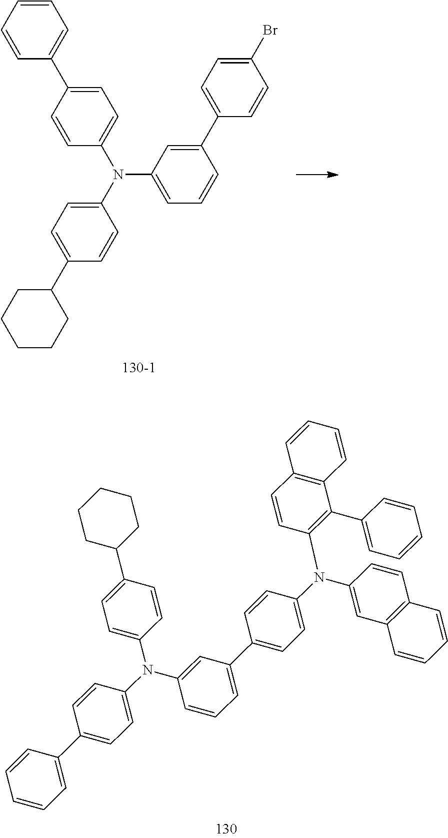 [SMILES: Brc1ccc(-c2cccc(N(c3ccc(-c4ccccc4)cc3)c3ccc(C4CCCCC4)cc3)c2)cc1 ; c1ccc(-c2ccc(N(c3ccc(C4CCCCC4)cc3)c3cccc(-c4ccc(N(c5ccc6ccccc6c5)c5ccc6ccccc6c5-c5ccccc5)cc4)c3)cc2)cc1]

Intermediate 130-1 (12 mmol, 1.2 eq), N-(naphthalen-2-yl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.1 mmol of Compound 130. (yield=61%)

### (18) Synthesis of Compound 135

### Synthesis of Intermediate 135-1

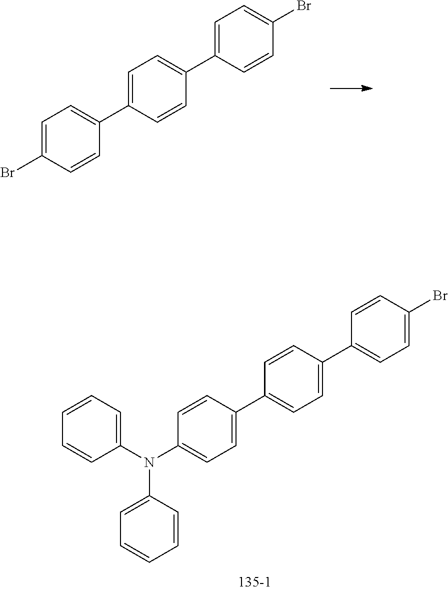 [SMILES: Brc1ccc(-c2ccc(-c3ccc(Br)cc3)cc2)cc1 ; Brc1ccc(-c2ccc(-c3ccc(N(c4ccccc4)c4ccccc4)cc3)cc2)cc1]

4,4″-dibromo-1,1′:4′,1″-terphenyl (26 mmol, 1.3 eq), diphenylamine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 135-1. (yield=60%)

### Synthesis of Compound 135

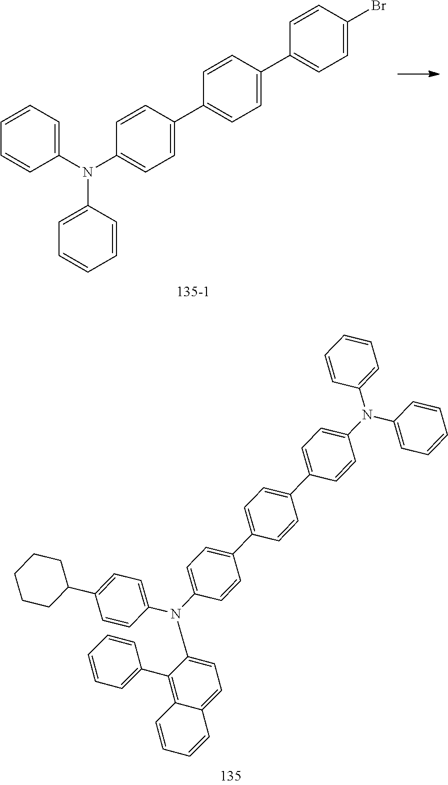 [SMILES: Brc1ccc(-c2ccc(-c3ccc(N(c4ccccc4)c4ccccc4)cc3)cc2)cc1 ; c1ccc(-c2c(N(c3ccc(-c4ccc(-c5ccc(N(c6ccccc6)c6ccccc6)cc5)cc4)cc3)c3ccc(C4CCCCC4)cc3)ccc3ccccc23)cc1]

Intermediate 135-1 (12 mmol, 1.2 eq), N-(4-cyclohexylphenyl)-1-phenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.9 mmol of Compound 135. (yield=69%)

### (19) Synthesis of Compound 140

### Synthesis of Intermediate 140-1

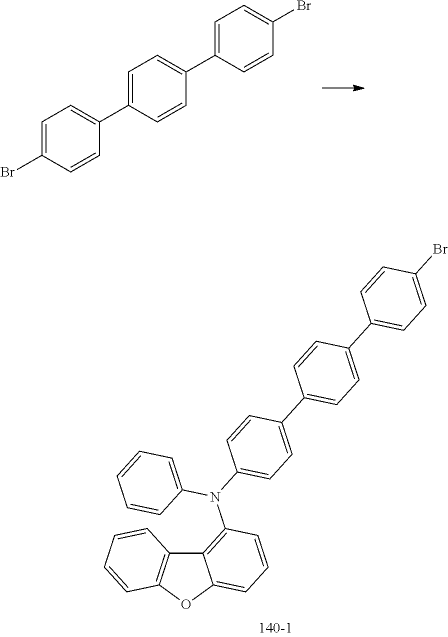 [SMILES: Brc1ccc(-c2ccc(-c3ccc(Br)cc3)cc2)cc1 ; Brc1ccc(-c2ccc(-c3ccc(N(c4ccccc4)c4cccc5oc6ccccc6c45)cc3)cc2)cc1]

4,4″-dibromo-1,1′:4′,1″-terphenyl (26 mmol, 1.3 eq), N-phenyldibenzo[b,d]furan-1-amine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 14 mmol of Intermediate 140-1. (yield=70%)

### Synthesis of Compound 140

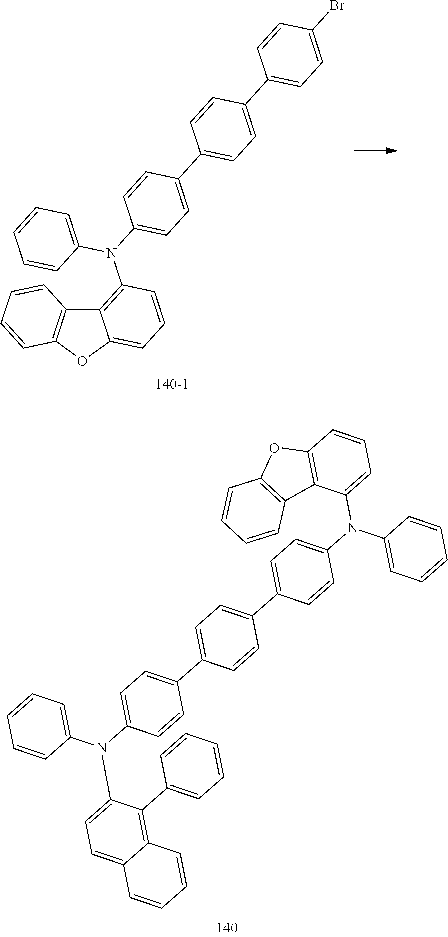 [SMILES: Brc1ccc(-c2ccc(-c3ccc(N(c4ccccc4)c4cccc5oc6ccccc6c45)cc3)cc2)cc1 ; c1ccc(-c2c(N(c3ccccc3)c3ccc(-c4ccc(-c5ccc(N(c6ccccc6)c6cccc7oc8ccccc8c67)cc5)cc4)cc3)ccc3ccccc23)cc1]

Intermediate 140-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.0 mmol of Compound 140. (yield=60%)

### (20) Synthesis of Compound 145

### Synthesis of Intermediate 145-1

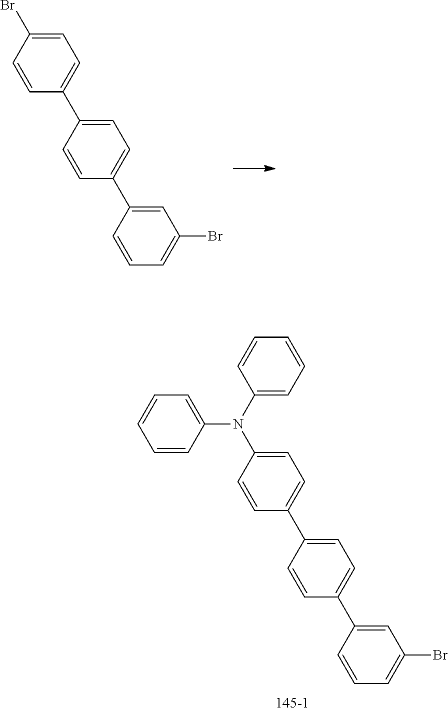 [SMILES: Brc1ccc(-c2ccc(-c3cccc(Br)c3)cc2)cc1 ; Brc1cccc(-c2ccc(-c3ccc(N(c4ccccc4)c4ccccc4)cc3)cc2)c1]

3,4″-dibromo-1,1′:4′,1″-terphenyl (26 mmol, 1.3 eq), diphenylamine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 145-1. (yield=60%)

### Synthesis of Compound 145

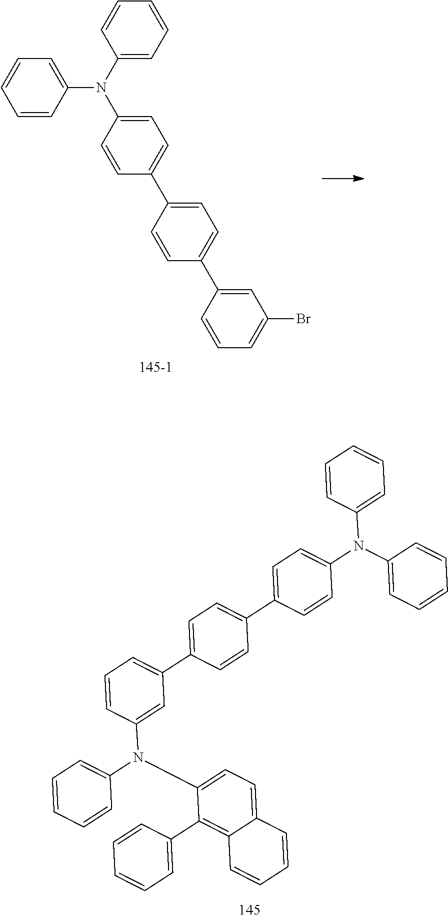 [SMILES: Brc1cccc(-c2ccc(-c3ccc(N(c4ccccc4)c4ccccc4)cc3)cc2)c1 ; c1ccc(-c2c(N(c3ccccc3)c3cccc(-c4ccc(-c5ccc(N(c6ccccc6)c6ccccc6)cc5)cc4)c3)ccc3ccccc23)cc1]

Intermediate 145-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 5.9 mmol of Compound 145. (yield=59%)

### (21) Synthesis of Compound 147

### Synthesis of Intermediate 147-1

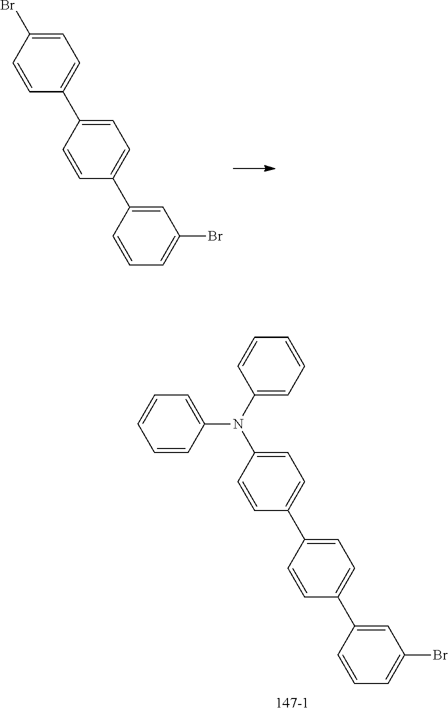 [SMILES: Brc1ccc(-c2ccc(-c3cccc(Br)c3)cc2)cc1 ; Brc1cccc(-c2ccc(-c3ccc(N(c4ccccc4)c4ccccc4)cc3)cc2)c1]

3,4″-dibromo-1,1′:4′,1″-terphenyl (26 mmol, 1.3 eq), diphenylamine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 14 mmol of Intermediate 147-1. (yield=70%)

### Synthesis of Compound 147

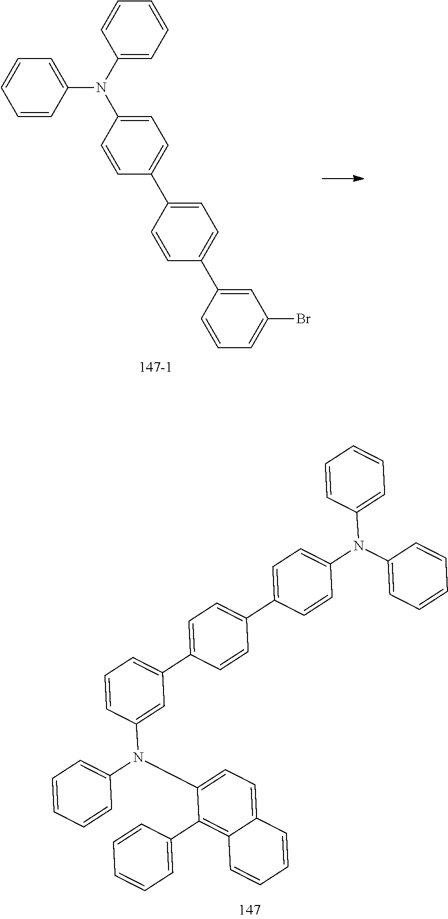 [SMILES: Brc1cccc(-c2ccc(-c3ccc(N(c4ccccc4)c4ccccc4)cc3)cc2)c1 ; c1ccc(-c2c(N(c3ccccc3)c3cccc(-c4ccc(-c5ccc(N(c6ccccc6)c6ccccc6)cc5)cc4)c3)ccc3ccccc23)cc1]

Intermediate 147-1 (12 mmol, 1.2 eq), N-(4-cyclohexylphenyl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 6.0 mmol of Compound 147. (yield=60%)

### (22) Synthesis of Compound 153

### Synthesis of Intermediate 153-1

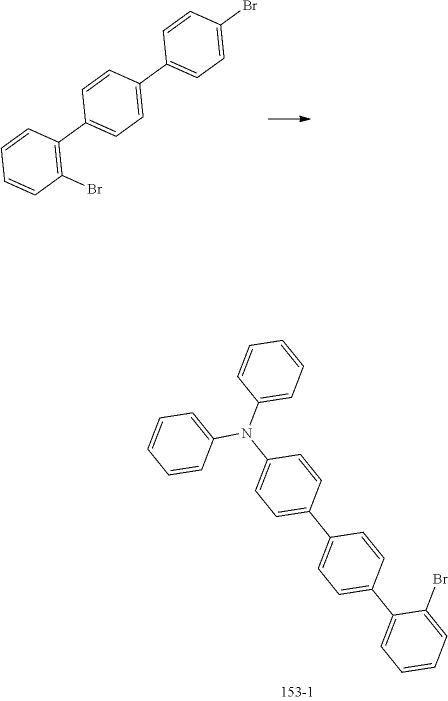 [SMILES: Brc1ccc(-c2ccc(-c3ccccc3Br)cc2)cc1 ; Brc1ccccc1-c1ccc(-c2ccc(N(c3ccccc3)c3ccccc3)cc2)cc1]

2,4″-dibromo-1,1′:4′,1″-terphenyl (26 mmol, 1.3 eq), diphenylamine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 153-1. (yield=60%)

### Synthesis of Compound 153

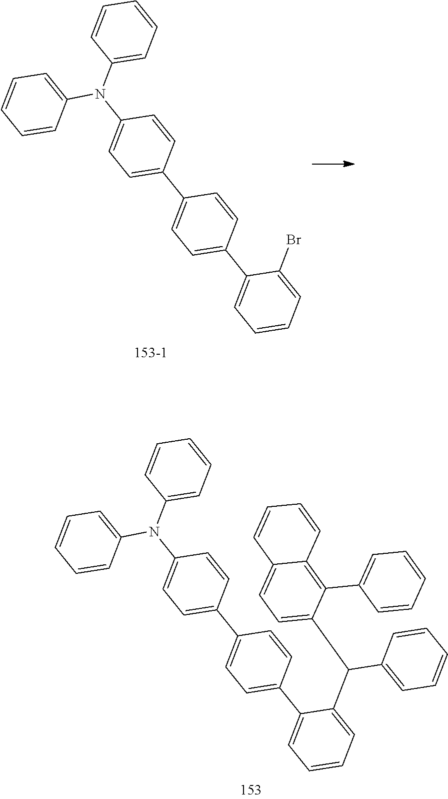 [SMILES: Brc1ccccc1-c1ccc(-c2ccc(N(c3ccccc3)c3ccccc3)cc2)cc1 ; c1ccc(-c2c(C(c3ccccc3)c3ccccc3-c3ccc(-c4ccc(N(c5ccccc5)c5ccccc5)cc4)cc3)ccc3ccccc23)cc1]

Intermediate 153-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 4.9 mmol of Compound 153. (yield=49%)

### (23) Synthesis of Compound 155

### Synthesis of Intermediate 155-1

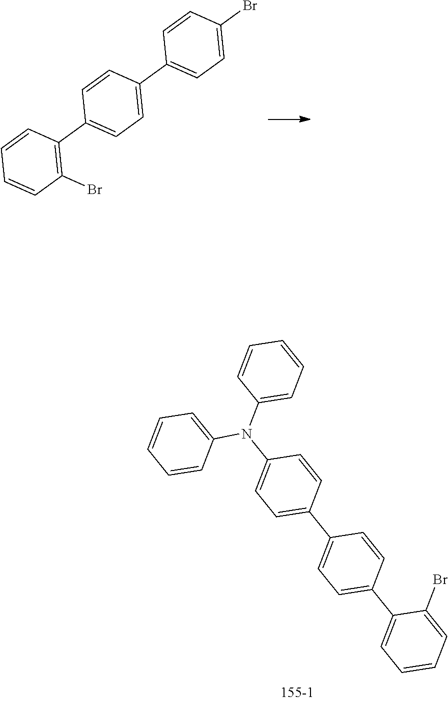 [SMILES: Brc1ccc(-c2ccc(-c3ccccc3Br)cc2)cc1 ; Brc1ccccc1-c1ccc(-c2ccc(N(c3ccccc3)c3ccccc3)cc2)cc1]

2,4″-dibromo-1,1′:4′,1″-terphenyl (26 mmol, 1.3 eq), diphenylamine (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 155-1. (yield=60%)

### Synthesis of Compound 155

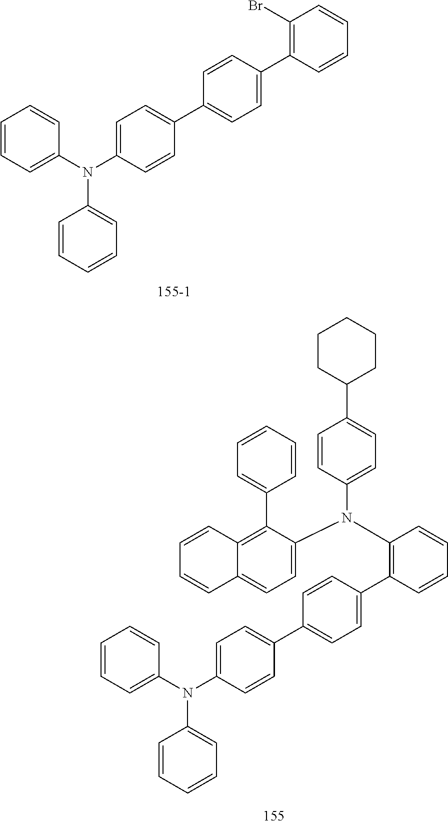 [SMILES: Brc1ccccc1-c1ccc(-c2ccc(N(c3ccccc3)c3ccccc3)cc2)cc1 ; c1ccc(-c2c(N(c3ccc(C4CCCCC4)cc3)c3ccccc3-c3ccc(-c4ccc(N(c5ccccc5)c5ccccc5)cc4)cc3)ccc3ccccc23)cc1]

Intermediate 155-1 (12 mmol, 1.2 eq), N-(4-cyclohexylphenyl)-1-phenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 4.3 mmol of Compound 155. (yield=43%)

### (24) Synthesis of Compound 157

### Synthesis of Intermediate 157-1

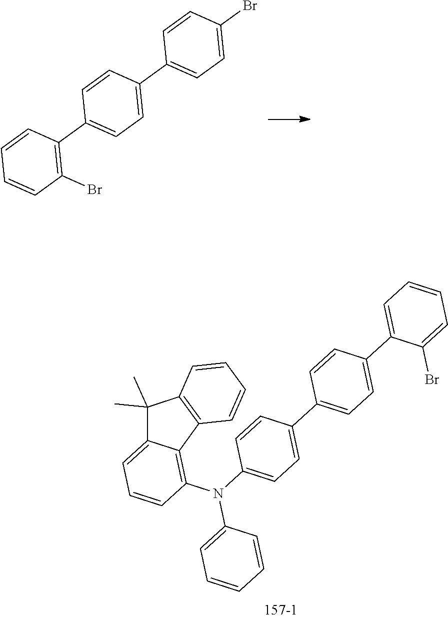 [SMILES: Brc1ccc(-c2ccc(-c3ccccc3Br)cc2)cc1 ; CC1(C)c2ccccc2-c2c(N(c3ccccc3)c3ccc(-c4ccc(-c5ccccc5Br)cc4)cc3)cccc21]

2,4″-dibromo-1,1′:4′,1″-terphenyl (26 mmol, 1.3 eq), N-phenyl-9,9-dimethyl-9H-fluoren-4-amine (20 mmol, 1 eq), Pd₂(dba)₃ (1 mmol, 0.05 eq), t-BuONa (40 mmol, eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 12 mmol of Intermediate 157-1. (yield=60%)

### Synthesis of Compound 157

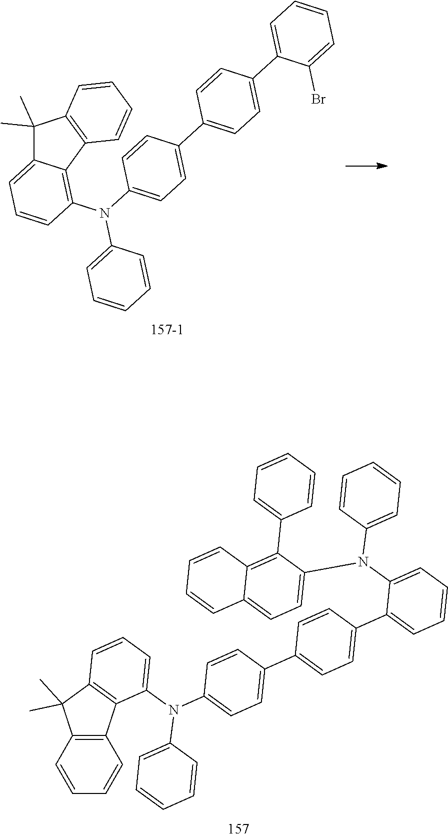 [SMILES: CC1(C)c2ccccc2-c2c(N(c3ccccc3)c3ccc(-c4ccc(-c5ccccc5Br)cc4)cc3)cccc21 ; CC1(C)c2ccccc2-c2c(N(c3ccccc3)c3ccc(-c4ccc(-c5ccccc5N(c5ccccc5)c5ccc6ccccc6c5-c5ccccc5)cc4)cc3)cccc21]

Intermediate 157-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-BusP (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 4.5 mmol of Compound 157. (yield=45%)

### (25) Synthesis of Compound 181

### Synthesis of Intermediate 181-1

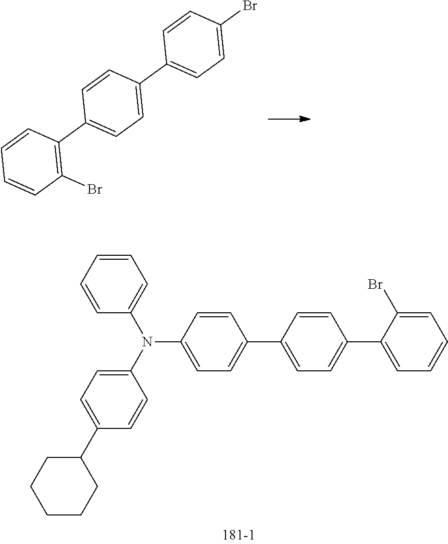 [SMILES: Brc1ccc(-c2ccc(-c3ccccc3Br)cc2)cc1 ; Brc1ccccc1-c1ccc(-c2ccc(N(c3ccccc3)c3ccc(C4CCCCC4)cc3)cc2)cc1]

2,4″-dibromo-1,1′:4′,1″-terphenyl (26 mmol, 1.3 eq), N-(4-cyclohexylphenyl)aniline (20 mmol, 1 eq), Pd₂(dba)₃ (1 mmol, 0.05 eq), t-BuONa (40 mmol, 2 eq), BINAP (2 mmol, 0.1 eq), and 300 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/5 (v/v) as an eluent, to obtain about 13 mmol of Intermediate 181-1. (yield=65%)

### Synthesis of Compound 181

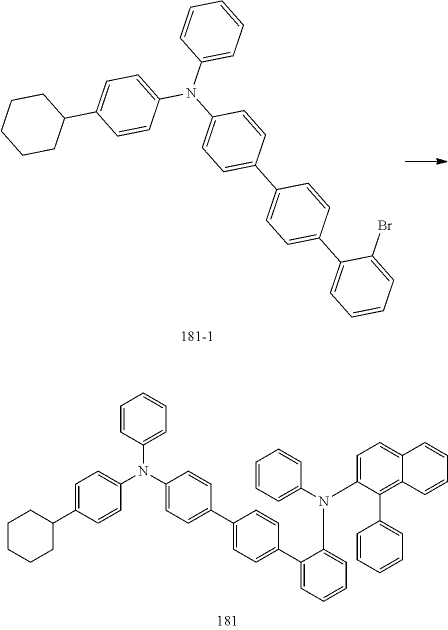 [SMILES: Brc1ccccc1-c1ccc(-c2ccc(N(c3ccccc3)c3ccc(C4CCCCC4)cc3)cc2)cc1 ; c1ccc(-c2c(N(c3ccccc3)c3ccccc3-c3ccc(-c4ccc(N(c5ccccc5)c5ccc(C6CCCCC6)cc5)cc4)cc3)ccc3ccccc23)cc1]

Intermediate 181-1 (12 mmol, 1.2 eq), N,1-diphenylnaphthalen-2-amine (10 mmol, 1 eq), Pd₂(dba)₃ (0.5 mol, 0.05 eq), t-BuONa (20 mmol, 2 eq), t-Bu₃P (0.1 mmol, 0.1 eq), and 150 mL of toluene were put into a single-neck-round flask, and the mixture was stirred at about 110° C. for about 1 hour. After the reaction was completed, the resultant was worked up with ether/H₂O, and then was purified by column chromatography utilizing dichloromethane/hexane=1/8 (v/v) as an eluent, to obtain about 4.0 mmol of Compound 181. (yield=40%)

### 2. Manufacture and Evaluation of Light-Emitting Elements

Light-emitting elements containing the amine compounds according to Examples were each manufactured as follows. Amine Compounds 64, 67, 73, 76, 81, 84, 88, 91, 92, 94, 96, 100, 121, 122, 127, 128, 130, 135, 140, 145, 147, 153, 155, 157, and 181, which are the above-described Example Compounds, were each utilized as a hole transport layer material to manufacture the light-emitting elements according to Examples 1 to 25, respectively. Light-emitting elements according to Comparative Examples 1 to 5 correspond to light-emitting elements which were each manufactured utilizing Comparative Example Compounds C1 to C5 as a hole transport layer material, respectively.

### Example Compounds

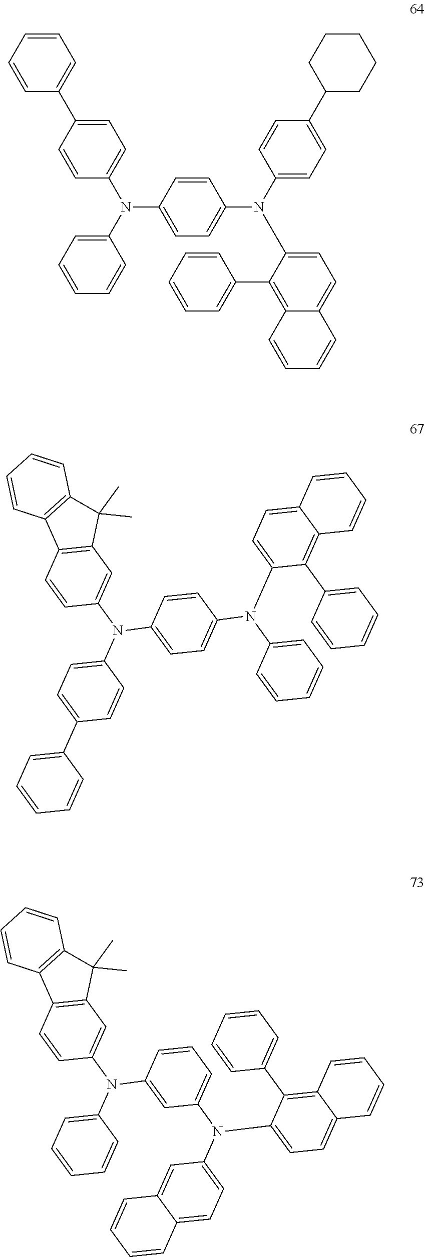 [SMILES: c1ccc(-c2ccc(N(c3ccccc3)c3ccc(N(c4ccc(C5CCCCC5)cc4)c4ccc5ccccc5c4-c4ccccc4)cc3)cc2)cc1 ; CC1(C)c2ccccc2-c2ccc(N(c3ccc(-c4ccccc4)cc3)c3ccc(N(c4ccccc4)c4ccc5ccccc5c4-c4ccccc4)cc3)cc21 ; CC1(C)c2ccccc2-c2ccc(N(c3ccccc3)c3cccc(N(c4ccc5ccccc5c4)c4ccc5ccccc5c4-c4ccccc4)c3)cc21]

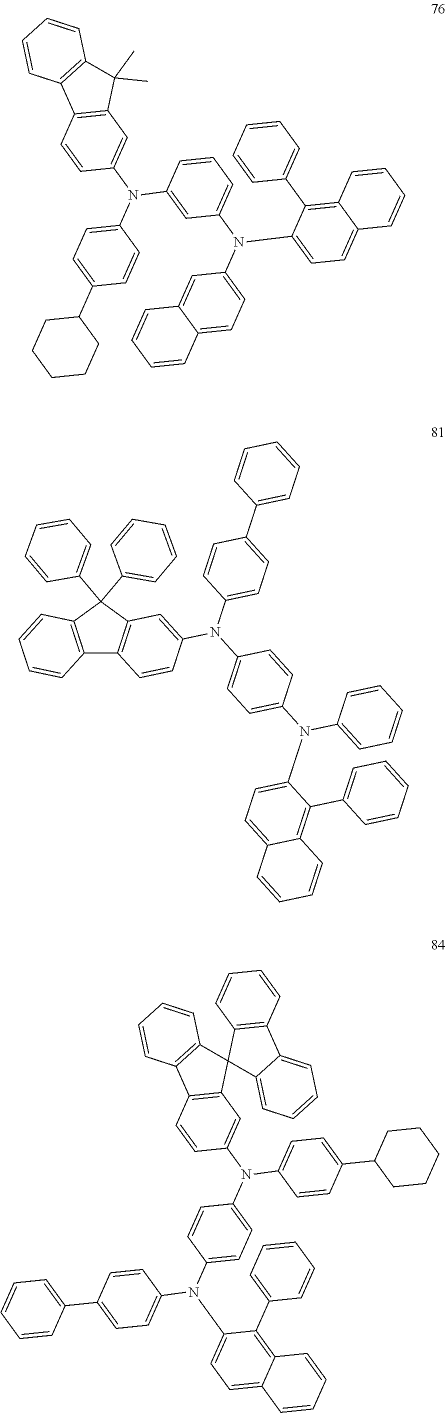 [SMILES: CC1(C)c2ccccc2-c2ccc(N(c3ccc(C4CCCCC4)cc3)c3cccc(N(c4ccc5ccccc5c4)c4ccc5ccccc5c4-c4ccccc4)c3)cc21 ; c1ccc(-c2ccc(N(c3ccc(N(c4ccccc4)c4ccc5ccccc5c4-c4ccccc4)cc3)c3ccc4c(c3)C(c3ccccc3)(c3ccccc3)c3ccccc3-4)cc2)cc1 ; c1ccc(-c2ccc(N(c3ccc(N(c4ccc(C5CCCCC5)cc4)c4ccc5c(c4)C4(c6ccccc6-c6ccccc64)c4ccccc4-5)cc3)c3ccc4ccccc4c3-c3ccccc3)cc2)cc1]

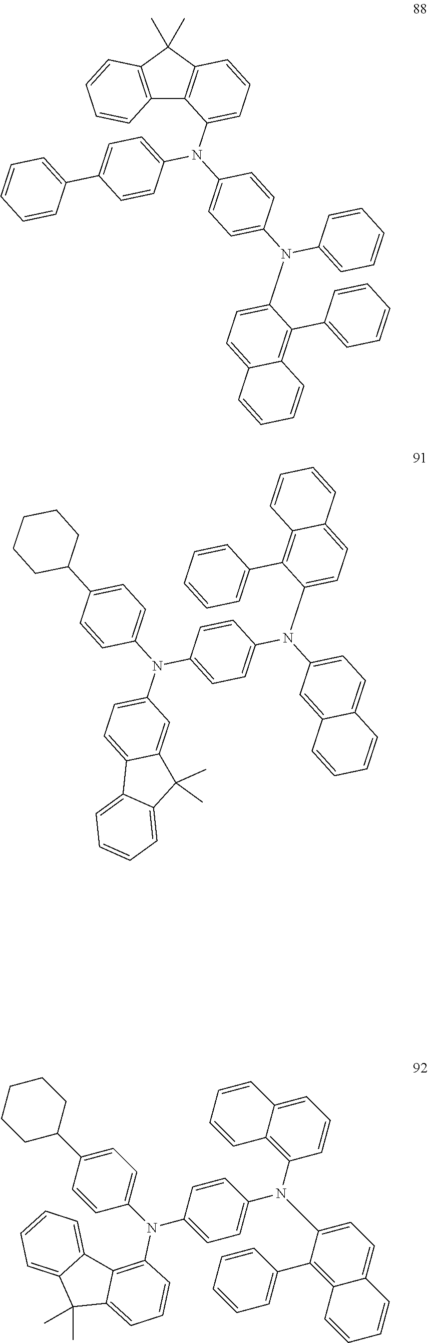 [SMILES: CC1(C)c2ccccc2-c2c(N(c3ccc(-c4ccccc4)cc3)c3ccc(N(c4ccccc4)c4ccc5ccccc5c4-c4ccccc4)cc3)cccc21 ; CC1(C)c2ccccc2-c2ccc(N(c3ccc(C4CCCCC4)cc3)c3ccc(N(c4ccc5ccccc5c4)c4ccc5ccccc5c4-c4ccccc4)cc3)cc21 ; CC1(C)c2ccccc2-c2c(N(c3ccc(C4CCCCC4)cc3)c3ccc(N(c4ccc5ccccc5c4-c4ccccc4)c4cccc5ccccc45)cc3)cccc21]

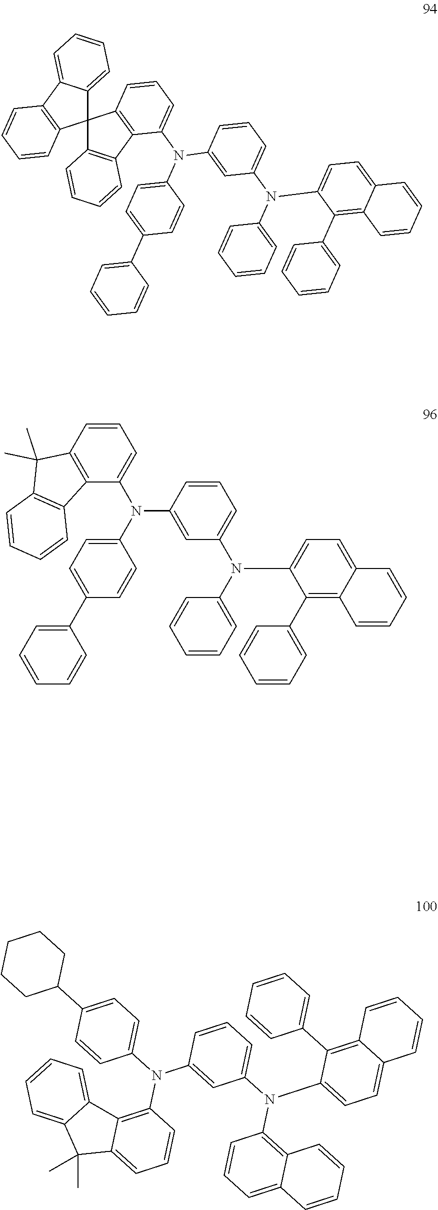 [SMILES: c1ccc(-c2ccc(N(c3cccc(N(c4ccccc4)c4ccc5ccccc5c4-c4ccccc4)c3)c3cccc4c3-c3ccccc3C43c4ccccc4-c4ccccc43)cc2)cc1 ; CC1(C)c2ccccc2-c2c(N(c3ccc(-c4ccccc4)cc3)c3cccc(N(c4ccccc4)c4ccc5ccccc5c4-c4ccccc4)c3)cccc21 ; CC1(C)c2ccccc2-c2c(N(c3ccc(C4CCCCC4)cc3)c3cccc(N(c4ccc5ccccc5c4-c4ccccc4)c4cccc5ccccc45)c3)cccc21]

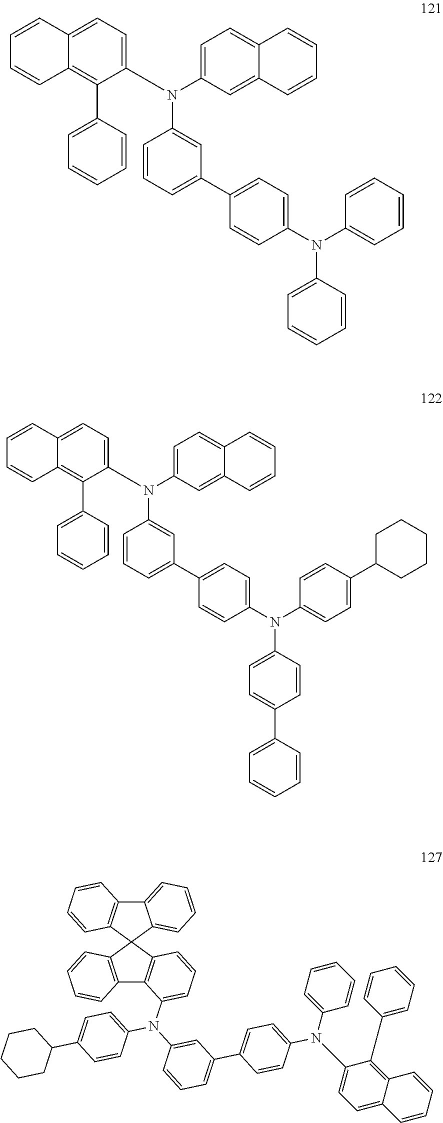 [SMILES: c1ccc(-c2c(N(c3cccc(-c4ccc(N(c5ccccc5)c5ccccc5)cc4)c3)c3ccc4ccccc4c3)ccc3ccccc23)cc1 ; c1ccc(-c2ccc(N(c3ccc(-c4cccc(N(c5ccc6ccccc6c5)c5ccc6ccccc6c5-c5ccccc5)c4)cc3)c3ccc(C4CCCCC4)cc3)cc2)cc1 ; c1ccc(-c2c(N(c3ccccc3)c3ccc(-c4cccc(N(c5ccc(C6CCCCC6)cc5)c5cccc6c5-c5ccccc5C65c6ccccc6-c6ccccc65)c4)cc3)ccc3ccccc23)cc1]

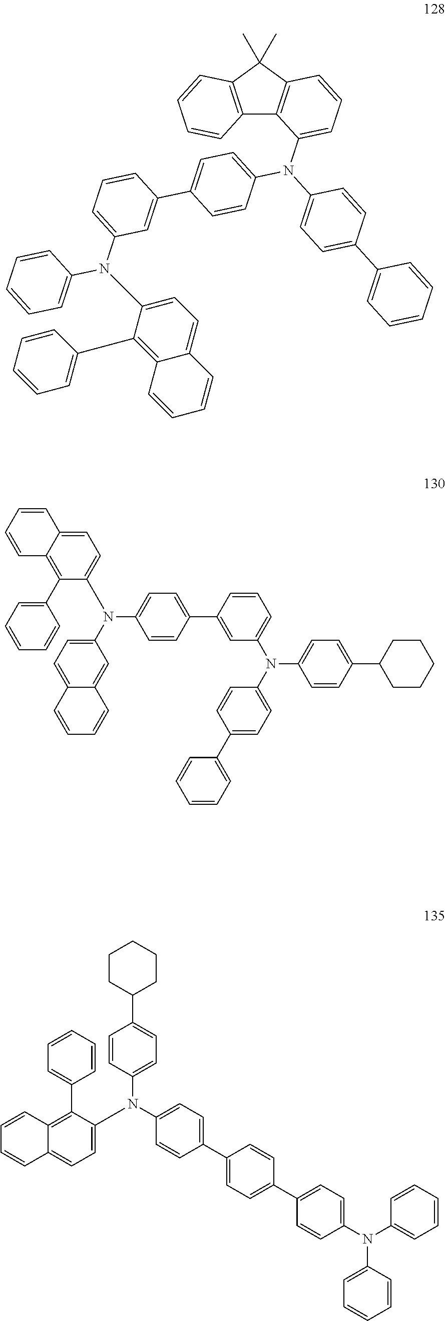 [SMILES: CC1(C)c2ccccc2-c2c(N(c3ccc(-c4ccccc4)cc3)c3ccc(-c4cccc(N(c5ccccc5)c5ccc6ccccc6c5-c5ccccc5)c4)cc3)cccc21 ; c1ccc(-c2ccc(N(c3ccc(C4CCCCC4)cc3)c3cccc(-c4ccc(N(c5ccc6ccccc6c5)c5ccc6ccccc6c5-c5ccccc5)cc4)c3)cc2)cc1 ; c1ccc(-c2c(N(c3ccc(-c4ccc(-c5ccc(N(c6ccccc6)c6ccccc6)cc5)cc4)cc3)c3ccc(C4CCCCC4)cc3)ccc3ccccc23)cc1]

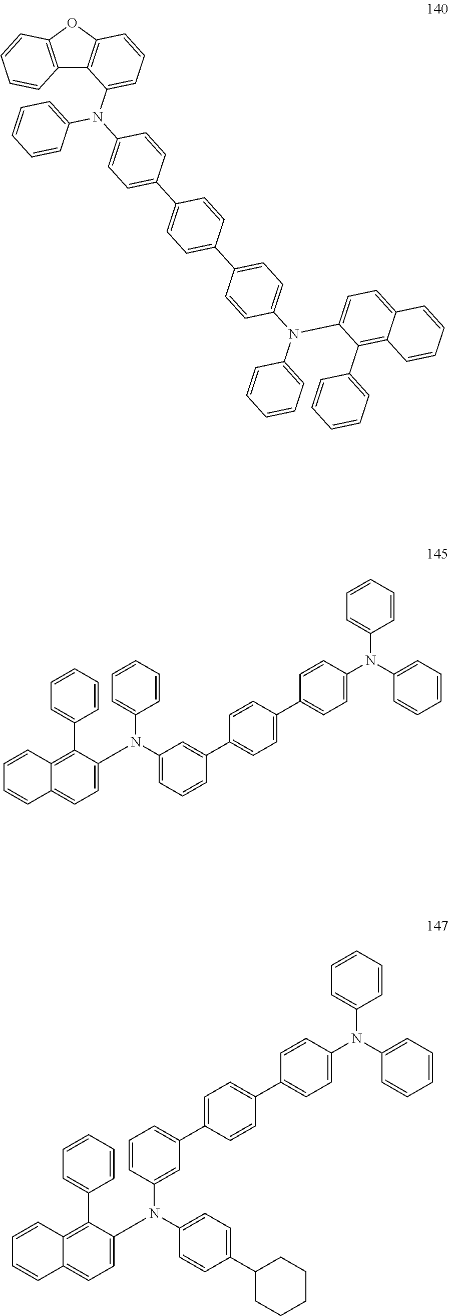 [SMILES: c1ccc(-c2c(N(c3ccccc3)c3ccc(-c4ccc(-c5ccc(N(c6ccccc6)c6cccc7oc8ccccc8c67)cc5)cc4)cc3)ccc3ccccc23)cc1 ; c1ccc(-c2c(N(c3ccccc3)c3cccc(-c4ccc(-c5ccc(N(c6ccccc6)c6ccccc6)cc5)cc4)c3)ccc3ccccc23)cc1 ; c1ccc(-c2c(N(c3ccc(C4CCCCC4)cc3)c3cccc(-c4ccc(-c5ccc(N(c6ccccc6)c6ccccc6)cc5)cc4)c3)ccc3ccccc23)cc1]

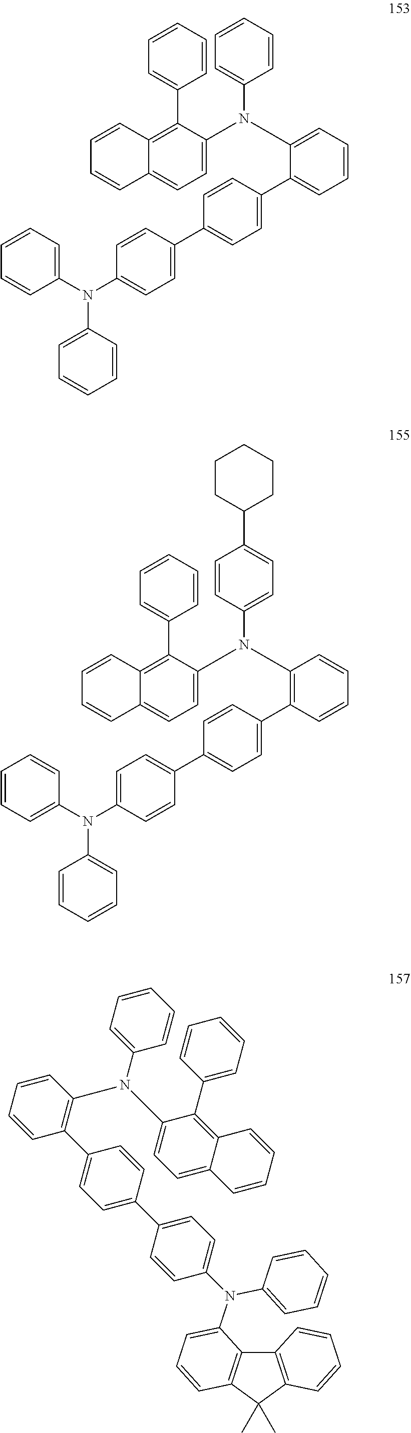 [SMILES: c1ccc(-c2c(N(c3ccccc3)c3ccccc3-c3ccc(-c4ccc(N(c5ccccc5)c5ccccc5)cc4)cc3)ccc3ccccc23)cc1 ; c1ccc(-c2c(N(c3ccc(C4CCCCC4)cc3)c3ccccc3-c3ccc(-c4ccc(N(c5ccccc5)c5ccccc5)cc4)cc3)ccc3ccccc23)cc1 ; CC1(C)c2ccccc2-c2c(N(c3ccccc3)c3ccc(-c4ccc(-c5ccccc5N(c5ccccc5)c5ccc6ccccc6c5-c5ccccc5)cc4)cc3)cccc21]

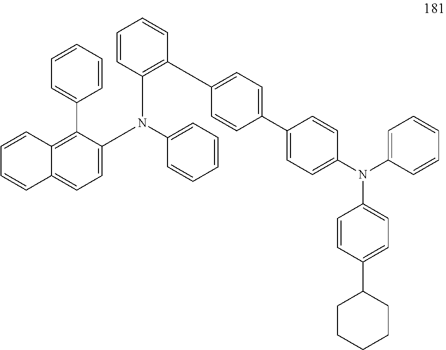 [SMILES: c1ccc(-c2c(N(c3ccccc3)c3ccccc3-c3ccc(-c4ccc(N(c5ccccc5)c5ccc(C6CCCCC6)cc5)cc4)cc3)ccc3ccccc23)cc1]

### Comparative Example Compounds

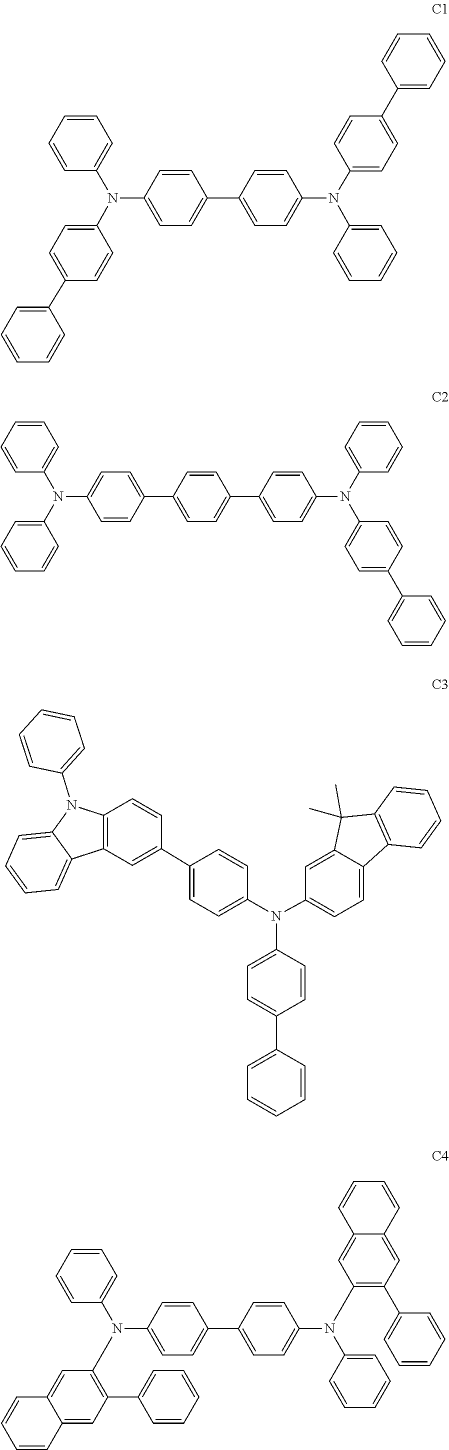 [SMILES: c1ccc(-c2ccc(N(c3ccccc3)c3ccc(-c4ccc(N(c5ccccc5)c5ccc(-c6ccccc6)cc5)cc4)cc3)cc2)cc1 ; c1ccc(-c2ccc(N(c3ccccc3)c3ccc(-c4ccc(-c5ccc(N(c6ccccc6)c6ccccc6)cc5)cc4)cc3)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2cc3ccccc3cc2N(c2ccccc2)c2ccc(-c3ccc(N(c4ccccc4)c4cc5ccccc5cc4-c4ccccc4)cc3)cc2)cc1]

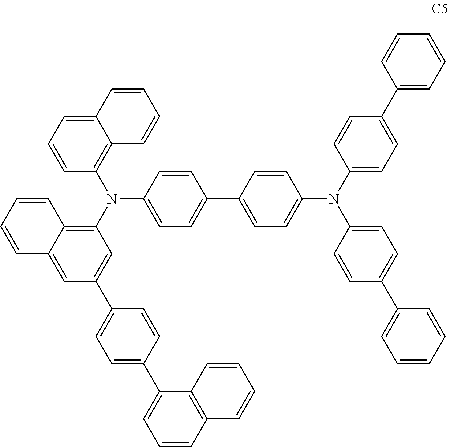 [SMILES: c1ccc(-c2ccc(N(c3ccc(-c4ccccc4)cc3)c3ccc(-c4ccc(N(c5cccc6ccccc56)c5cc(-c6ccc(-c7cccc8ccccc78)cc6)cc6ccccc56)cc4)cc3)cc2)cc1]

### Manufacture of Light-Emitting Elements

An 15 Ω/cm² (1,500 Å) of ITO glass substrate made by Corning Co. Ltd, was cut to a size of about 50 mm×50 mm×0.7 mm, washed with isopropyl alcohol and pure water, cleansed by ultrasonic waves for about five minutes each, and then irradiated with UV rays for about 30 minutes. Thereafter, ozone treatment was performed. Thereafter, NPD was vacuum deposited to form a hole injection layer having a thickness of about 300 Å, and then, Example Compound or Comparative Example Compound was vacuum deposited to form a hole transport layer having a thickness of about 200 Å. CzSi was vacuum deposited on the hole transport layer to form a light-emitting auxiliary layer having a thickness of about 300 Å.

HT3, EHT66, AD-38, and D1 were co-deposited on the light-emitting auxiliary layer at a weight ratio of about 42:42:15:1 to form a light-emitting layer having a thickness of about 200 Å, TSPO1 was deposited on the light-emitting layer to form a hole-blocking layer having a thickness of about 200 Å, and then TPBi was deposited on the hole-blocking layer to form an electron transport layer having a thickness of about 300 Å. Then, LiF was deposited on the electron transport layer to form an electron injection layer having a thickness of about 10 Å. Al was deposited on the electron injection layer to form a cathode having a thickness of about 3000 Å.

In addition, compounds of each functional layer utilized in the manufacture of the light-emitting elements were as follows.

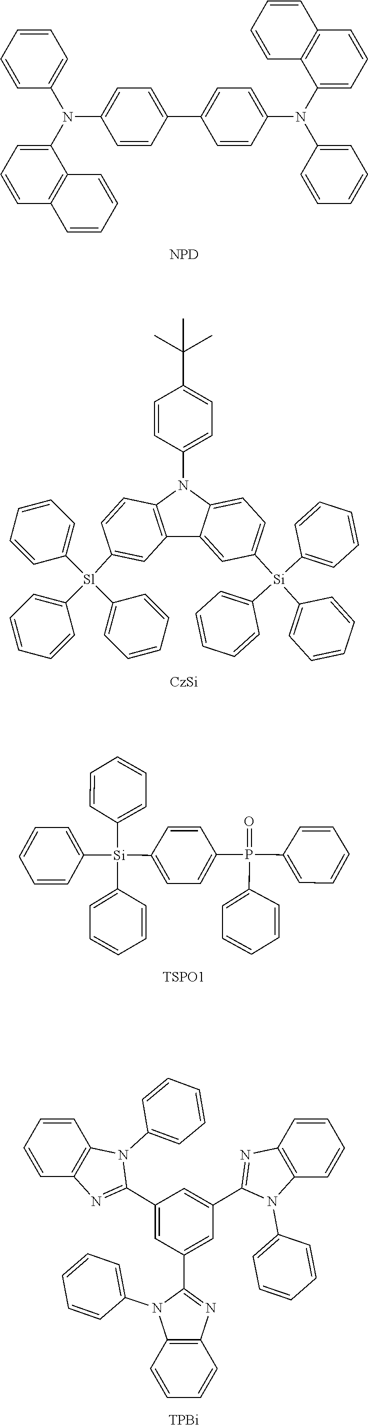

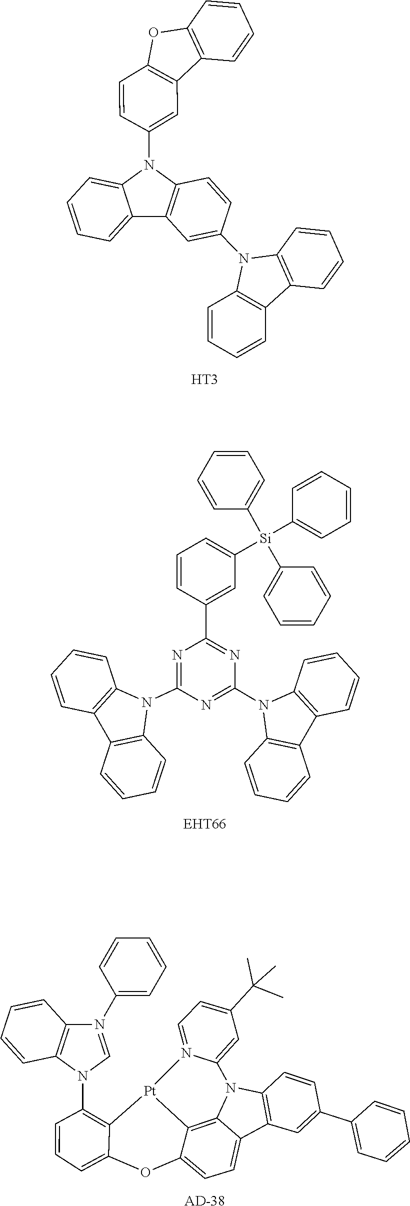

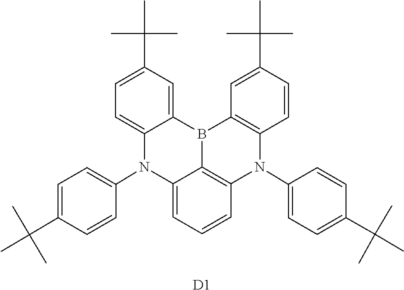 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; "D1"]

### Evaluation of Light-Emitting Elements

The light-emitting elements according to Examples 1 to 25, and Comparative Examples 1 to 5 were each evaluated, and the results are listed in Table 1. A driving voltage, luminous efficiency, emission wavelength, and half-lifespan of each the manufactured light-emitting elements are listed in Table 1.

In the characteristic evaluation results of Examples and Comparative Examples in Table 1, the values of the driving voltage (V) and the current density were each measured by utilizing V7000 OLED IVL Test System, (Polaronix). For the evaluation of characteristics of each of the light-emitting elements manufactured according to Examples 1 to 25, and Comparative Example 1 to 5, the driving voltage, and the luminous efficiency were measured at a current density of about 50 mA/cm², and the time taken to decrease to 95% of an initial luminance during substantially continuous driving at the current density of about 100 mA/cm² was measured as a half-lifespan.

TABLE 1

| | | | | Light | |
|---|---|---|---|---|---|
| | Hole | Driving | Effi- | emitting | Life- |
| | transport | voltage | ciency | wavelength | span |
| | material | (V) | (cd/A) | (nm) | (T95) |
| Example 1 | Compound 64 | 3.71 | 30.5 | 460 | 320 |
| Example 2 | Compound 67 | 3.61 | 30.2 | 460 | 350 |
| Example 3 | Compound 73 | 3.65 | 32.2 | 460 | 360 |
| Example 4 | Compound 76 | 3.65 | 31.4 | 462 | 340 |
| Example 5 | Compound 81 | 3.57 | 30.6 | 459 | 360 |
| Example 6 | Compound 84 | 3.53 | 30.4 | 459 | 370 |
| Example 7 | Compound 88 | 3.54 | 30.5 | 459 | 360 |
| Example 8 | Compound 91 | 3.61 | 31.5 | 460 | 360 |
| Example 9 | Compound 92 | 3.64 | 31.6 | 460 | 350 |
| Example 10 | Compound 94 | 3.59 | 33.0 | 461 | 350 |
| Example 11 | Compound 96 | 3.48 | 32.8 | 460 | 330 |
| Example 12 | Compound 100 | 3.52 | 32.7 | 460 | 345 |
| Example 13 | Compound 121 | 3.55 | 31.7 | 462 | 310 |
| Example 14 | Compound 122 | 3.65 | 30.6 | 462 | 340 |
| Example 15 | Compound 127 | 3.57 | 31.3 | 460 | 360 |
| Example 16 | Compound 128 | 3.53 | 31.4 | 460 | 370 |
| Example 17 | Compound 130 | 3.54 | 31.5 | 460 | 360 |
| Example 18 | Compound 135 | 3.61 | 32.0 | 459 | 360 |
| Example 19 | Compound 140 | 3.79 | 30.9 | 460 | 350 |
| Example 20 | Compound 145 | 3.59 | 31.2 | 460 | 350 |
| Example 21 | Compound 147 | 3.48 | 30.8 | 462 | 330 |
| Example 22 | Compound 153 | 3.52 | 30.9 | 462 | 345 |
| Example 23 | Compound 155 | 3.52 | 33.0 | 461 | 330 |
| Example 24 | Compound 157 | 3.55 | 33.0 | 461 | 310 |
| Example 25 | Compound 181 | 3.48 | 33.0 | 461 | 395 |
| Comparative | Comparative | 4.4 | 26.3 | 462 | 220 |
| Example 1 | Example | | | | |
| | Compound C1 | | | | |
| Comparative | Comparative | 4.18 | 27.5 | 461 | 240 |
| Example 2 | Example | | | | |
| | Compound C2 | | | | |
| Comparative | Comparative | 4.20 | 25.0 | 460 | 200 |
| Example 3 | Example | | | | |
| | Compound C3 | | | | |
| Comparative | Comparative | 4.15 | 24.5 | 461 | 230 |
| Example 4 | Example | | | | |
| | Compound C4 | | | | |
| Comparative | Comparative | 4.48 | 27.0 | 461 | 180 |
| Example 5 | Example | | | | |
| | Compound C5 | | | | |

Referring to Table 1, it can be seen that the light-emitting elements according to Examples utilizing the amine compound according to one or more embodiments of the present disclosure as a hole transport layer material each exhibit relatively low driving voltage, relatively high luminous efficiency, and relatively long-lifespan than those according to Comparative Examples. In the embodiments of Comparative Example Compounds, compared to Example Compounds, it can be confirmed that when the Comparative Example Compounds are each applied to the light-emitting element, the driving voltage is high, the luminous efficiency decreases, and the lifespan decreases. For example, referring to Table 1, it can be confirmed that the light-emitting elements utilizing the amine compound according to one or more embodiments of the present disclosure each exhibit more improved element characteristics in the luminous efficiency or element lifespan than the light-emitting elements according to Comparative Examples.

The amine compounds according to Examples may have a structure in which two amine groups are connected via a first linker, and a first substituent is connected to at least one among the two amine groups. The first substituent may include a naphthyl moiety and a first sub-substituent connected to the naphthyl moiety. Because each of the amine compounds according to Examples includes a structure in which the first substituent is connected to the at least one among the two amine groups, a hole transport ability and stability of a radical cation state may be improved. Therefore, the light emitting elements according to Examples, which contain such amine compounds according to embodiments as the hole transport layer materials, may be expected to exhibit high luminous efficiency and long element lifespan.

Comparative Example compounds C1 to C3 utilized in Comparative Examples 1 to 3, respectively, are each a diamine compound including two amine groups but include no first substituent that is suggested in the present disclosure. Therefore, the hole transport characteristics of the light-emitting elements according to Comparative Examples may deteriorate compared to those according to Examples. It is thought, due to such results, that the light-emitting element according to Comparative Examples 1-3 may have lower luminous efficiency and lifespan than those according to Examples.

When Comparative Examples 4 and 5 are compared with Examples 13 to 17, Comparative Example Compounds C4 and C5 utilized in Comparative Examples 4 and 5, respectively, correspond to embodiments in which the linker that connects two amine groups is p,p-biphenylene. For example, Comparative Example Compounds C4 and C5 are each a compound having a structure in which two amine groups are connected via p,p-biphenylene, which corresponds to Formula A-1, and may thus have increased planarity compared with Example Compounds 121, 122, 127, 128, and 130, which each have m,p-biphenylene. Therefore, charge mobility may decrease.

In addition, it can be confirmed that, when Comparative Examples 4 and 5 were compared with Examples 18 and 19, the light-emitting elements according to Examples 18 and 19 each have a lower driving voltage and longer element lifespan, and higher efficiency than those according to Comparative Examples 4 and 5. Compounds 135 and 140 utilized in Examples 18 and 19, respectively, are compounds which each contain terphenylene as a linker connecting two amine groups. It can be seen that, compared with Comparative Compounds C4 and C5, even though all three benzene structures included in the linker of Compounds 135 and 140 are connected in a para-relation, a distance that charges may move is secured by an increase in the number of phenyls of the linker, and thus the charge mobility is recovered again, resulting in improvements in the lifespan and efficiency of the light emitting element. For example, in the amine compound according to one or more embodiments, when the phenyl structure included in the linker contains three or more benzene structures, a movement distance of charges in molecule may be sufficiently secured, and thus even though all three or more benzene structures are connected in a para-relation, the charge mobility may be improved compared with an amine compound having, as a linker, p,p-biphenyl in which two benzene structures are connected in a para-relation.

A light-emitting element according to one or more embodiments may exhibit characteristics of high efficiency and long-lifespan by containing the amine compound according to one or more embodiments.

When the amine compound according to one or more embodiments is applied to the light-emitting element, high efficiency and long-lifespan characteristics may be exhibited.

An electronic device according to one or more embodiments may exhibit excellent or suitable display quality.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

In present disclosure, “not include a or any ‘component’” “exclude a or any ‘component”, “‘component’-free”, and/or the like refers to that the “component” not being added, selected, or utilized as a component in a compound/composition, but the “component” of less than a suitable amount may still be included due to other impurities and/or external factors in a composition.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

In the present disclosure, when particles are spherical, “size” indicates a particle diameter or an average particle diameter, and when the particles are non-spherical, the “size” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The light-emitting element, the display device, the electronic apparatus, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Hitherto, although the embodiments of the present disclosure have been described, it is understood that the present disclosure should not be limited to these embodiments but one or more suitable changes and modifications can be made by one ordinary skilled in the art within the spirit and scope of the present disclosure as hereinafter claimed. Accordingly, the technical scope of the present disclosure is not intended to be limited to the content set forth in the detailed description of the present disclosure, but is intended to be defined by the appended claims and equivalents thereof.

What is claimed is:

1. A light-emitting element comprising:
a first electrode;
a second electrode on the first electrode; and
at least one functional layer between the first electrode and the second electrode and comprising an amine compound represented by Formula 1: 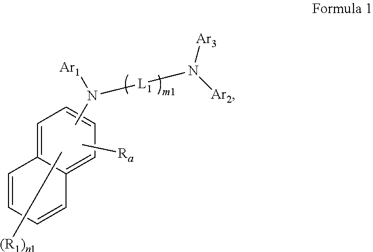
wherein, in Formula 1,
Ar₁ to Ar₃ are each independently a substituted or unsubstituted oxy group, a substituted or unsubstituted silyl group, a substituted or unsubstituted alkyl group having 1 to 20 carbon atoms, a substituted or unsubstituted alkenyl group having 2 to carbon atoms, a substituted or unsubstituted alkynyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
a case where Ar₁ comprises a fluorene group, a dibenzofuran group, or a dibenzothiophene group is excluded,
L₁ is a substituted or unsubstituted phenylene group,
Rₐ is a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
R₁ is hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1 is an integer of 0 to 6,
m1 is an integer of 1 to 4, and
when m1 is 2, then a case where L₁ is represented by Formula A-1 is excluded: 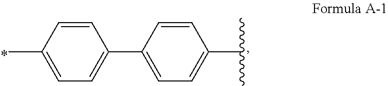
wherein, in Formula A-1, [symbol]
is a position at which a nitrogen atom, to which Ar₁ is connected in Formula 1 is connected, and 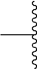
is a position at which a nitrogen atom, to which Ar₂ and Ar₃ are connected in Formula 1, is connected.

2. The light-emitting element of claim 1, wherein the at least one functional layer comprises:
a light-emitting layer;
a hole transport region between the first electrode and the light-emitting layer; and
an electron transport region between the light-emitting layer and the second electrode, and
the hole transport region comprises the amine compound represented by Formula 1.

3. The light-emitting element of claim 2, wherein the hole transport region comprises:
a hole injection layer on the first electrode; and
a hole transport layer on the hole injection layer, and
the hole transport layer comprises the amine compound represented by Formula 1.

4. The light-emitting element of claim 2, wherein a layer adjacent to the light-emitting layer selected from among a plurality of layers included in the hole transport region comprises the amine compound represented by Formula 1.

5. The light-emitting element of claim 1, wherein the amine compound represented by Formula 1 is a diamine compound.

6. The light-emitting element of claim 1,
wherein the amine compound represented by Formula 1 is represented by Formula 2: 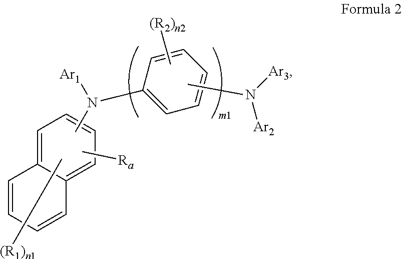
wherein, in Formula 2,
R₂ is hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n2 is an integer of 0 to 4, and
Ar₁ to Ar₃, Rₐ, R₁, n1, and m1 are each the same as defined in Formula 1.

7. The light-emitting element of claim 1,
wherein the amine compound represented by Formula 1 is represented by Formula 3: 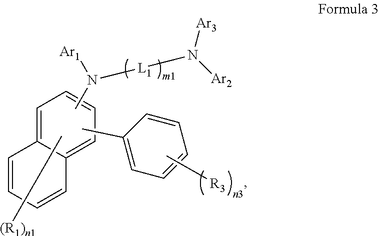
wherein, in Formula 3,
R₃ is hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n3 is an integer of 0 to 5, and
Ar₁ to Ar₃, L₁, R₁, n1, and m1 are each the same as defined in Formula 1.

8. The light-emitting element of claim 7,
wherein the amine compound represented by Formula 3 is represented by Formula 4-1 or Formula 4-2: 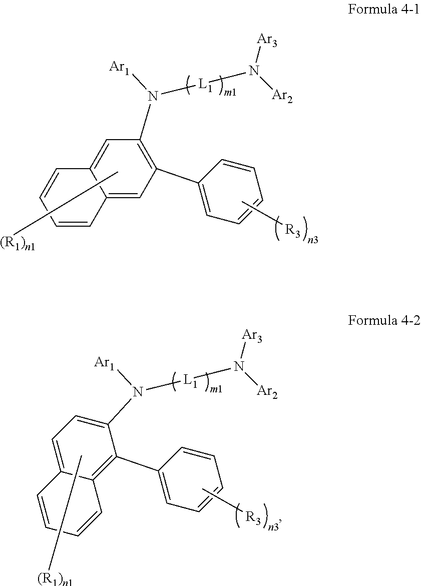
wherein, in Formulas 4-1 and 4-2,
Ar₁ to Ar₃, L₁, R₁, R₃, n1, n3, and m1 are each the same as defined in Formulas and 3.

9. The light-emitting element of claim 1,
wherein the amine compound represented by Formula 1 is represented by any one selected from among Formulas 5-1 to 5-5: 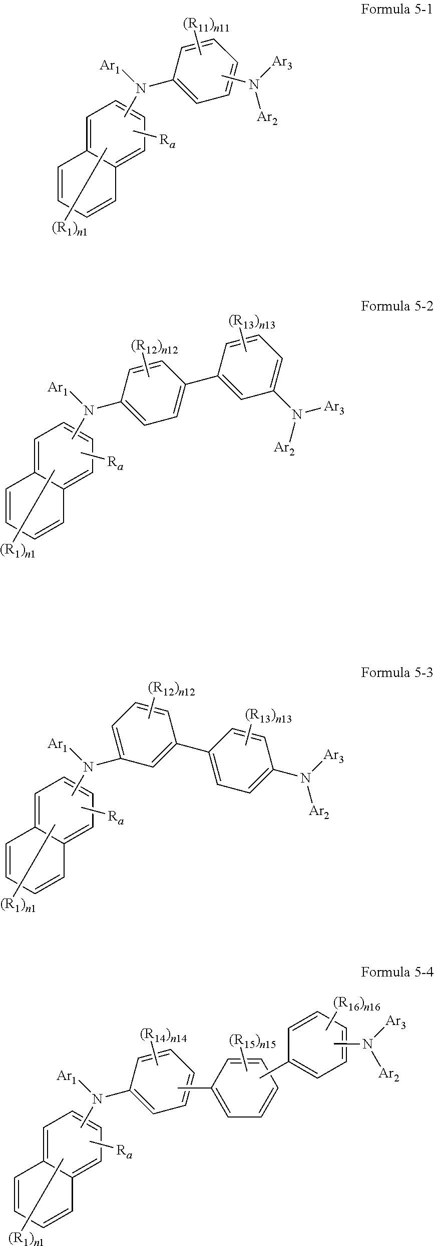 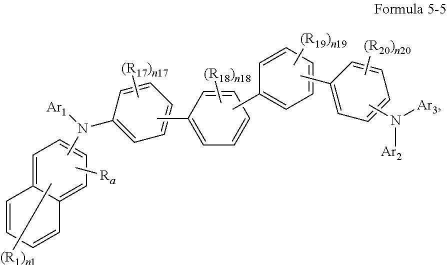
wherein, in Formulas 5-1 to 5-5,
R₁₁ to R₂₀ are each independently hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n11 to n20 are each independently an integer of 0 to 4, and
Ar₁ and Ar₃, Rₐ, R₁, and n1 are each the same as defined in Formula 1.

10. The light-emitting element of claim 1,
wherein L₁ is represented by any one selected from among Formulas L-1 to L-22: 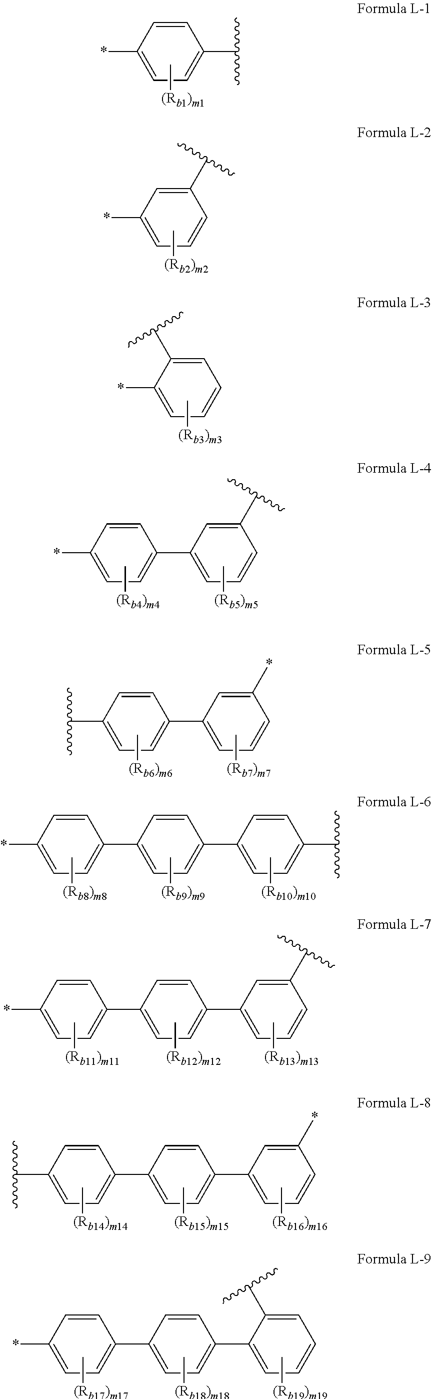 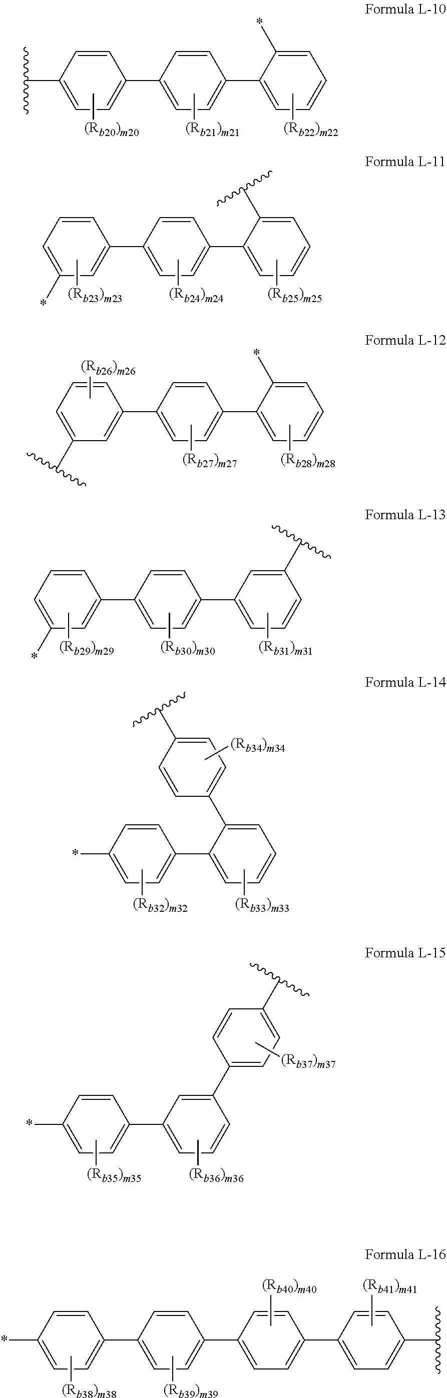 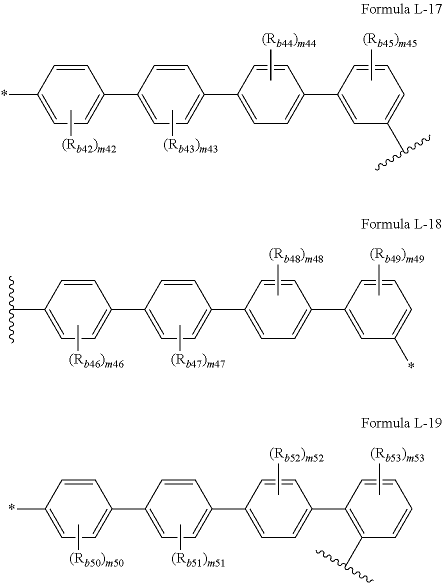 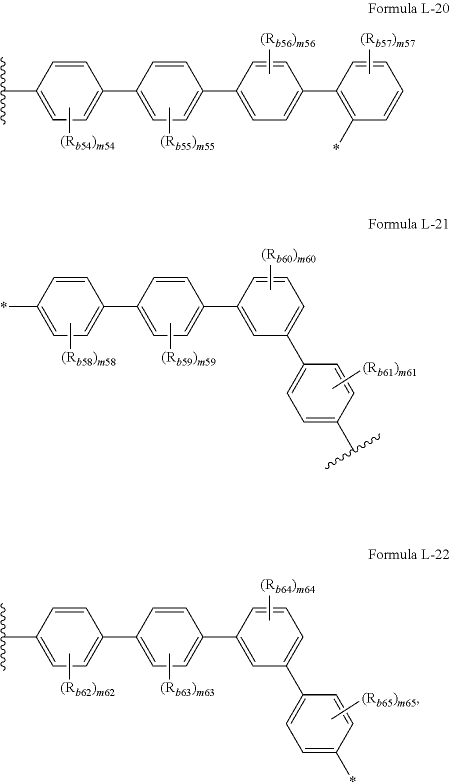
wherein, in Formulas L-1 to L-22,
R_{b1} to R_{b65} are each independently hydrogen, deuterium, a halogen, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and
m1 to m65 are each independently an integer of 0 to 4.

11. The light-emitting element of claim 1, wherein Ar₁ is a substituted or unsubstituted phenyl group, a substituted or unsubstituted biphenyl group, or a substituted or unsubstituted naphthyl group, and Ar₂ and Ar₃ are each independently a substituted or unsubstituted phenyl group, a substituted or unsubstituted biphenyl group, a substituted or unsubstituted naphthyl group, or a substituted or unsubstituted fluorenyl group.

12. The light-emitting element of claim 1,
wherein Ar₁ is represented by any one selected from among Formulas B-1 to B-4, and Ar₂ and Ar₃ are each independently represented by any one selected from among Formulas B-1 to B-7: 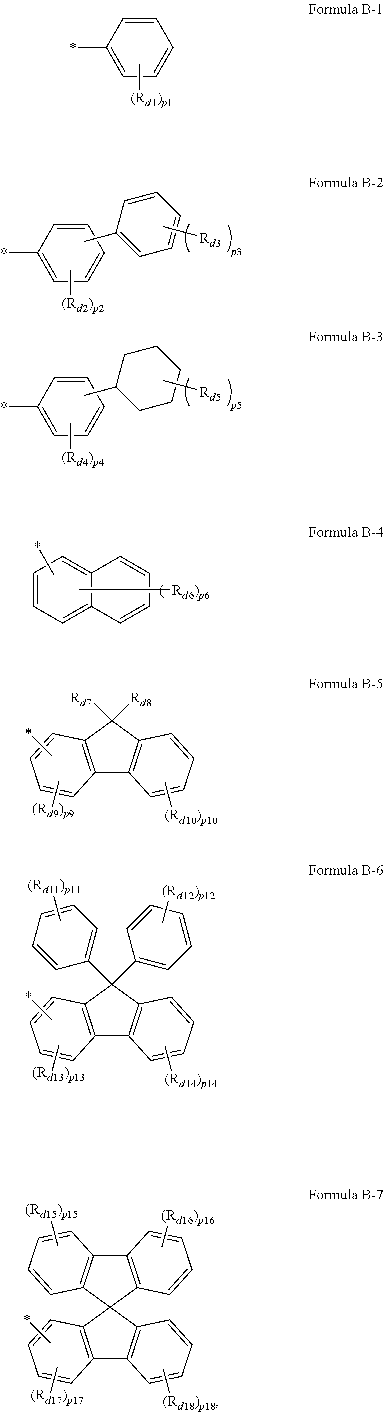
wherein, in Formulas B-1 to B-7,
R_{d1} to R_{d18} are each independently hydrogen, deuterium, a halogen, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
p1, p3, p11, and p12 are each independently an integer of 0 to 5,
p2, p4, p0, p14, p15, p16, and p18 are each independently an integer of 0 to 4,
p5 is an integer of 0 to 11,
p6 is an integer of 0 to 7, and
p9, p13, and p17 are each independently an integer of 0 to 3.

13. The light-emitting element of claim 1,
wherein the amine compound represented by Formula 1 is represented by Formula 6-1 or Formula 6-2: 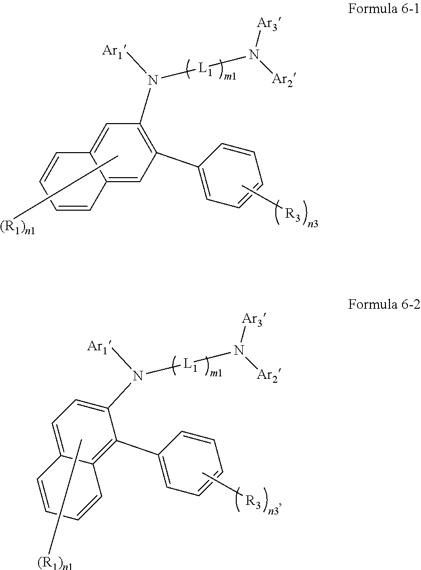
wherein, in Formulas 6-1 and 6-2,
R₃ is hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n3 is an integer of 0 to 5, and
Ar₁′ to Ar₃′ are each independently selected from Substituent Group A, provided that Ar₁′ is not any one selected from among a36 to a47: 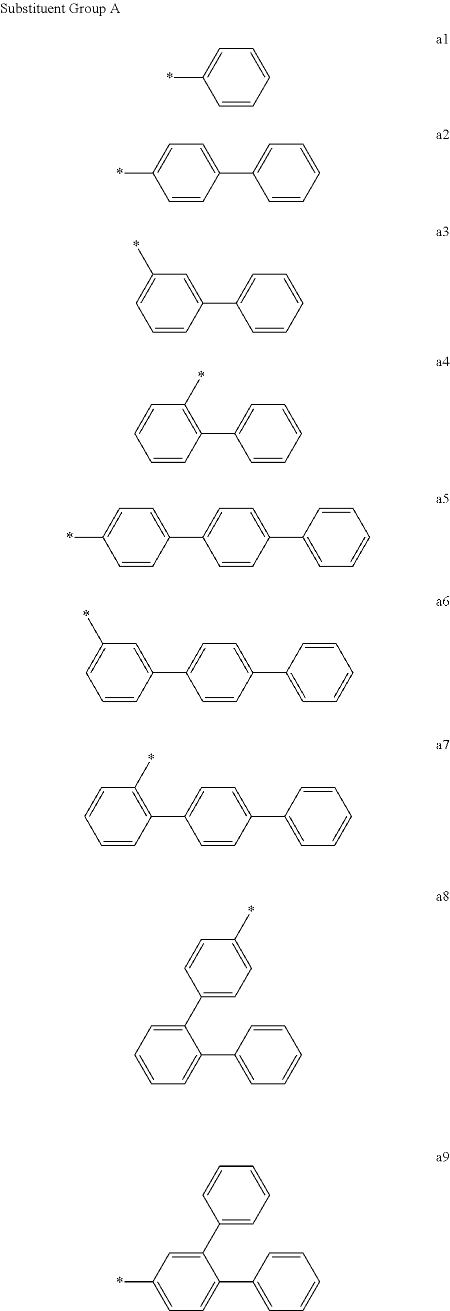 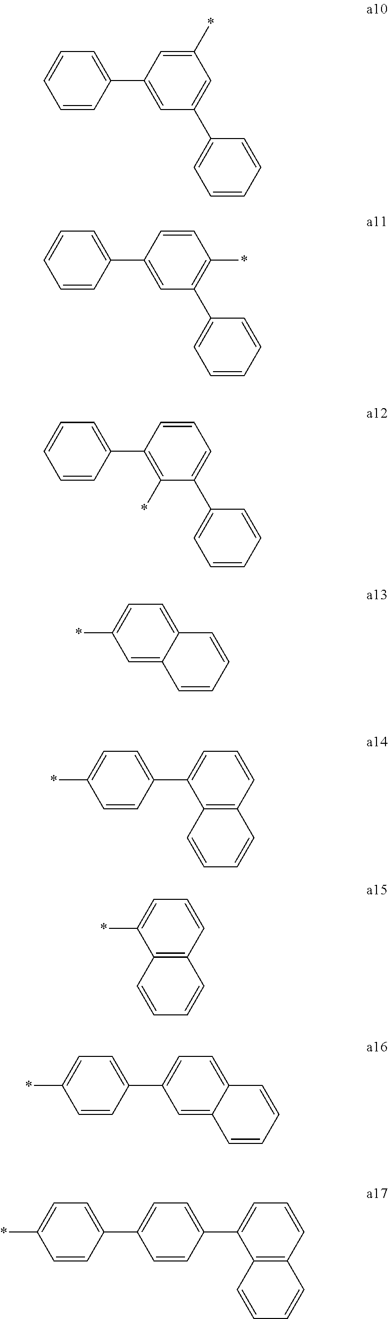 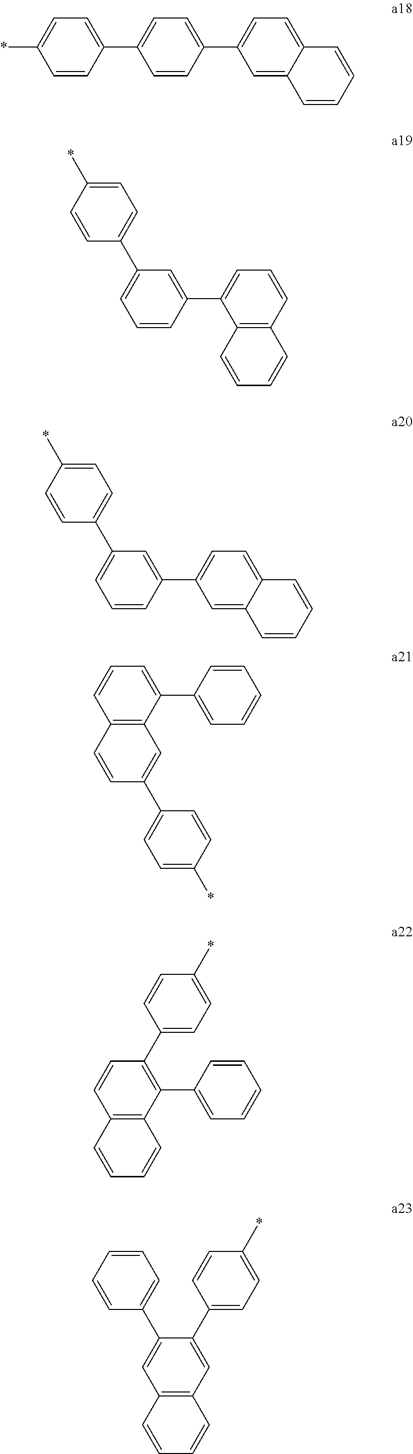 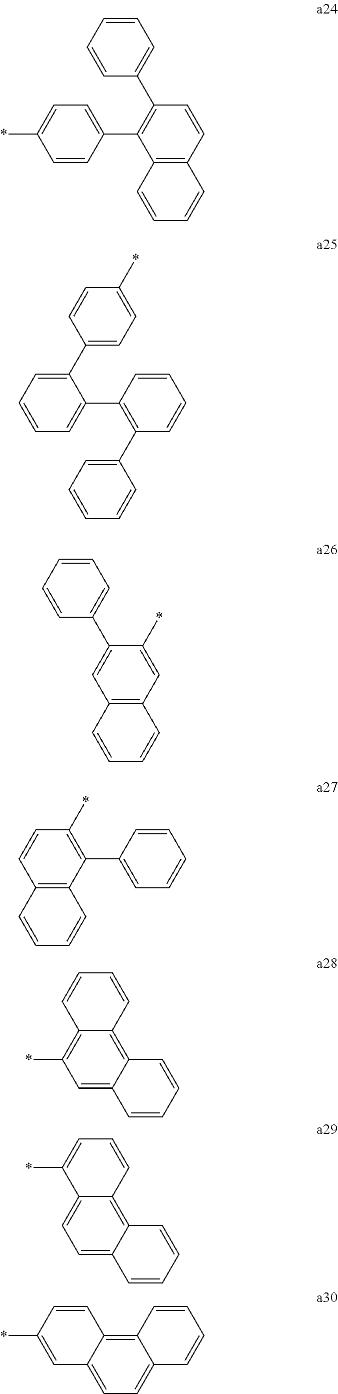 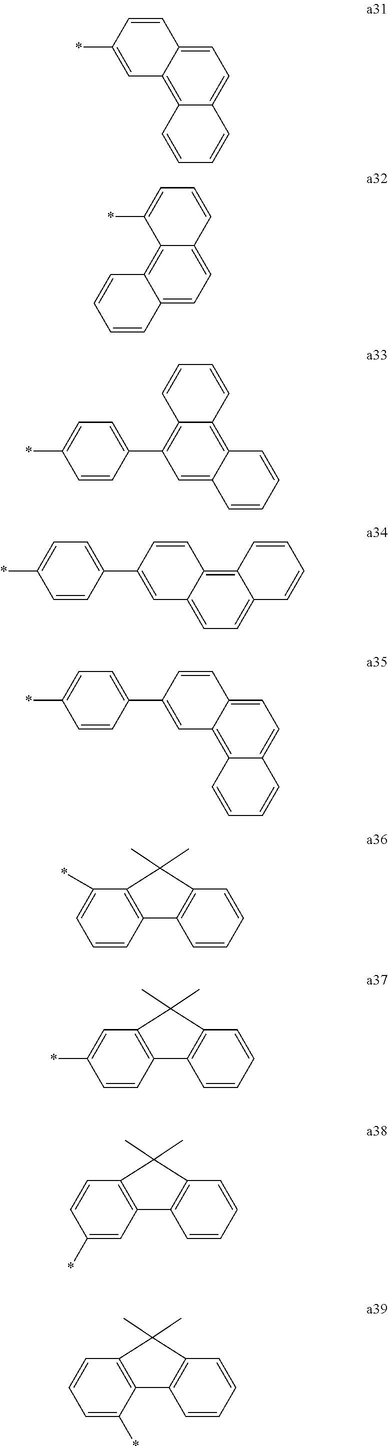 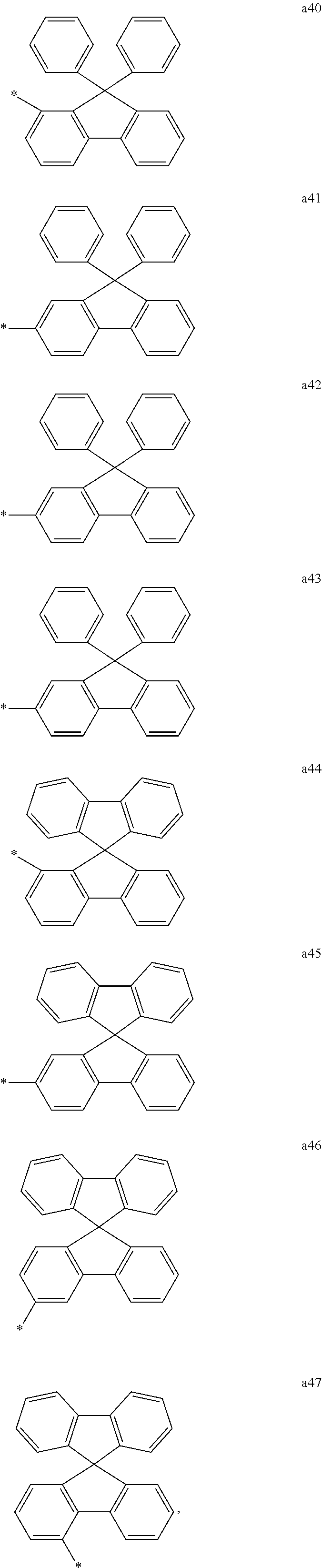
wherein, in Formulas 6-1 and 6-2,
L₁, R₁, n1, and m1 are each the same as defined in Formula 1.

14. The light-emitting element of claim 1,
wherein the amine compound represented by Formula 1 is any one selected from among compounds in Compound Group 1:

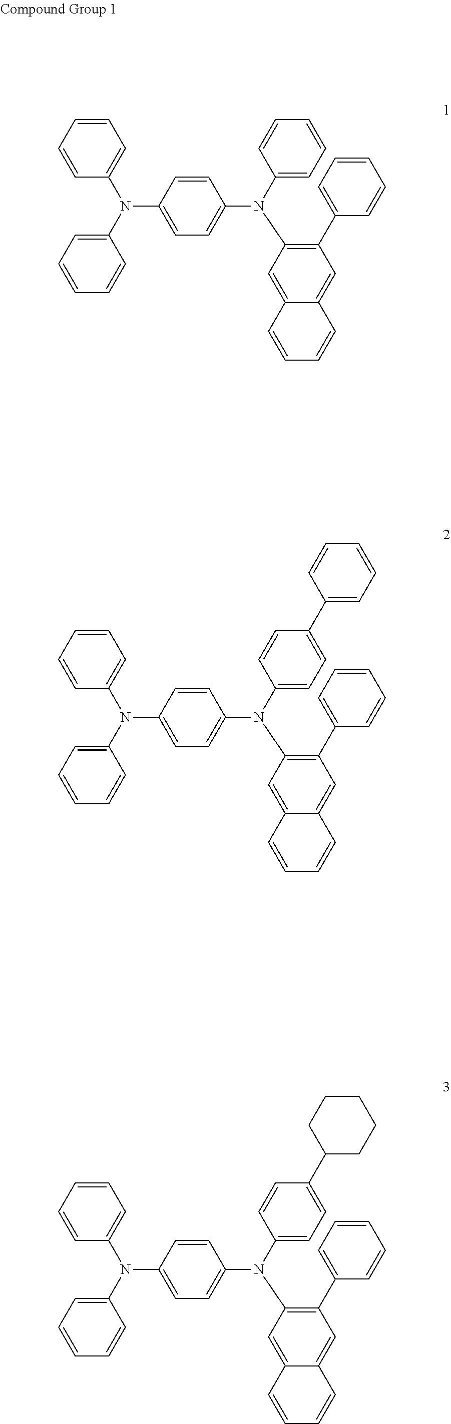 [SMILES: c1ccc(-c2cc3ccccc3cc2N(c2ccccc2)c2ccc(N(c3ccccc3)c3ccccc3)cc2)cc1 ; c1ccc(-c2ccc(N(c3ccc(N(c4ccccc4)c4ccccc4)cc3)c3cc4ccccc4cc3-c3ccccc3)cc2)cc1 ; c1ccc(-c2cc3ccccc3cc2N(c2ccc(C3CCCCC3)cc2)c2ccc(N(c3ccccc3)c3ccccc3)cc2)cc1]

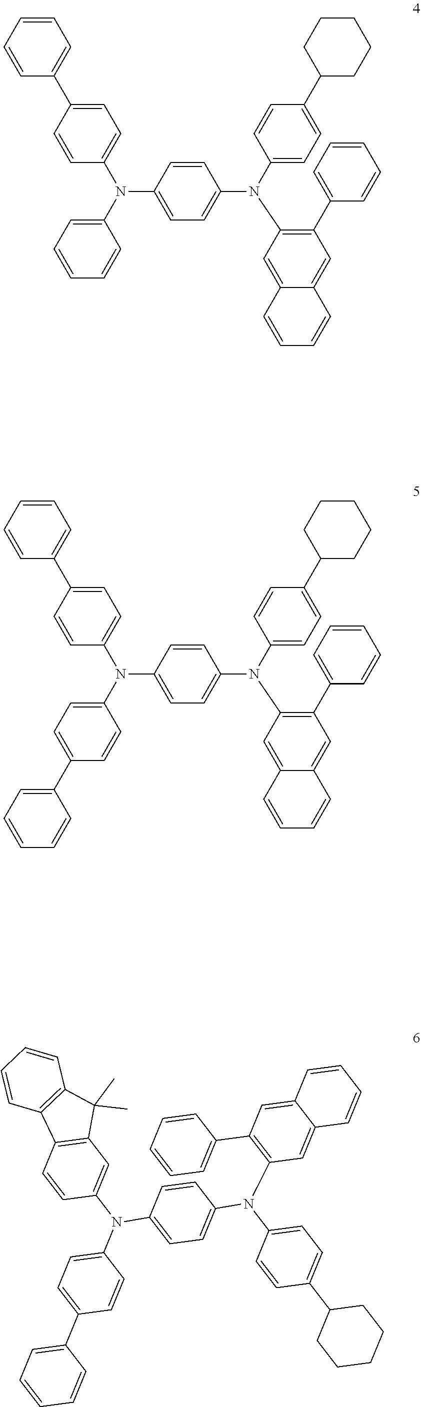 [SMILES: c1ccc(-c2ccc(N(c3ccccc3)c3ccc(N(c4ccc(C5CCCCC5)cc4)c4cc5ccccc5cc4-c4ccccc4)cc3)cc2)cc1 ; c1ccc(-c2ccc(N(c3ccc(-c4ccccc4)cc3)c3ccc(N(c4ccc(C5CCCCC5)cc4)c4cc5ccccc5cc4-c4ccccc4)cc3)cc2)cc1 ; CC1(C)c2ccccc2-c2ccc(N(c3ccc(-c4ccccc4)cc3)c3ccc(N(c4ccc(C5CCCCC5)cc4)c4cc5ccccc5cc4-c4ccccc4)cc3)cc21]

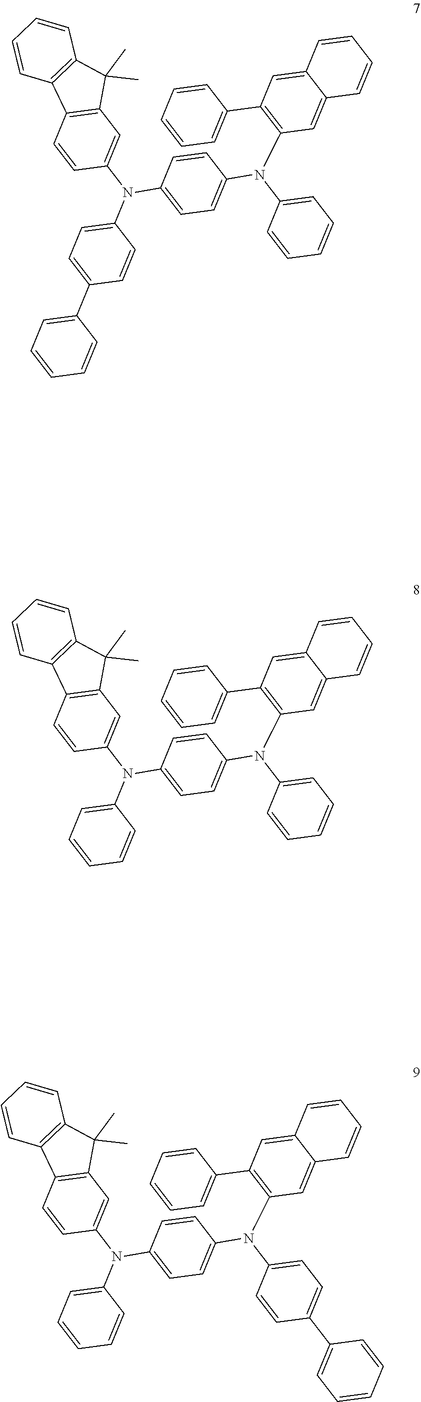 [SMILES: CC1(C)c2ccccc2-c2ccc(N(c3ccc(-c4ccccc4)cc3)c3ccc(N(c4ccccc4)c4cc5ccccc5cc4-c4ccccc4)cc3)cc21 ; CC1(C)c2ccccc2-c2ccc(N(c3ccccc3)c3ccc(N(c4ccccc4)c4cc5ccccc5cc4-c4ccccc4)cc3)cc21 ; CC1(C)c2ccccc2-c2ccc(N(c3ccccc3)c3ccc(N(c4ccc(-c5ccccc5)cc4)c4cc5ccccc5cc4-c4ccccc4)cc3)cc21]

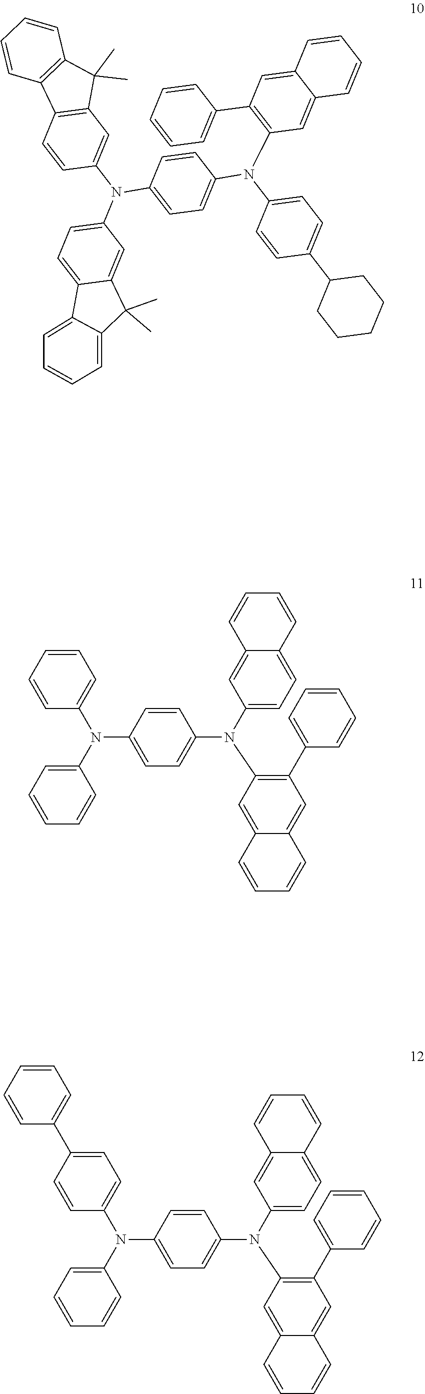 [SMILES: CC1(C)c2ccccc2-c2ccc(N(c3ccc(N(c4ccc(C5CCCCC5)cc4)c4cc5ccccc5cc4-c4ccccc4)cc3)c3ccc4c(c3)C(C)(C)c3ccccc3-4)cc21 ; c1ccc(-c2cc3ccccc3cc2N(c2ccc(N(c3ccccc3)c3ccccc3)cc2)c2ccc3ccccc3c2)cc1 ; c1ccc(-c2ccc(N(c3ccccc3)c3ccc(N(c4ccc5ccccc5c4)c4cc5ccccc5cc4-c4ccccc4)cc3)cc2)cc1]

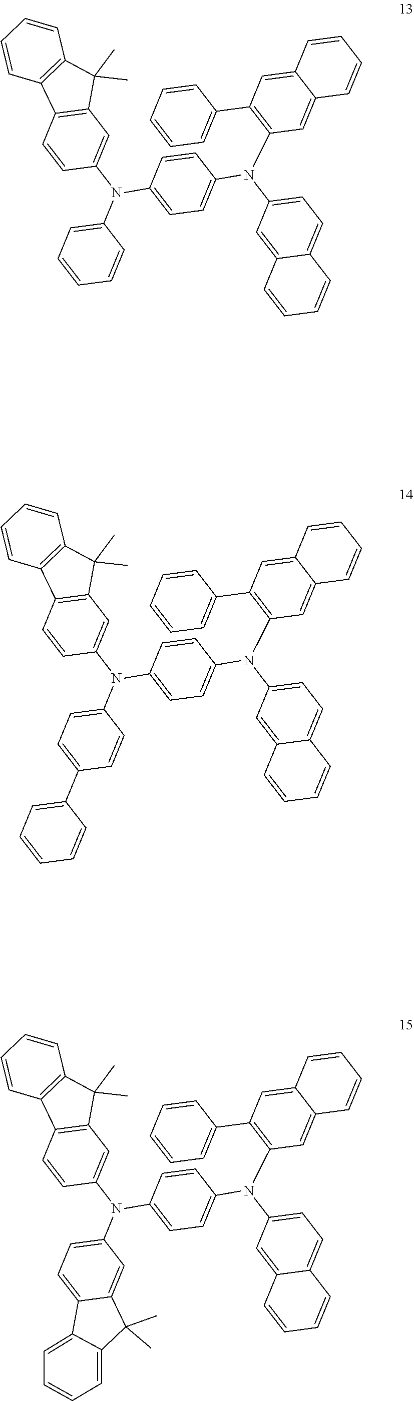 [SMILES: CC1(C)c2ccccc2-c2ccc(N(c3ccccc3)c3ccc(N(c4ccc5ccccc5c4)c4cc5ccccc5cc4-c4ccccc4)cc3)cc21 ; CC1(C)c2ccccc2-c2ccc(N(c3ccc(-c4ccccc4)cc3)c3ccc(N(c4ccc5ccccc5c4)c4cc5ccccc5cc4-c4ccccc4)cc3)cc21 ; CC1(C)c2ccccc2-c2ccc(N(c3ccc(N(c4ccc5ccccc5c4)c4cc5ccccc5cc4-c4ccccc4)cc3)c3ccc4c(c3)C(C)(C)c3ccccc3-4)cc21]

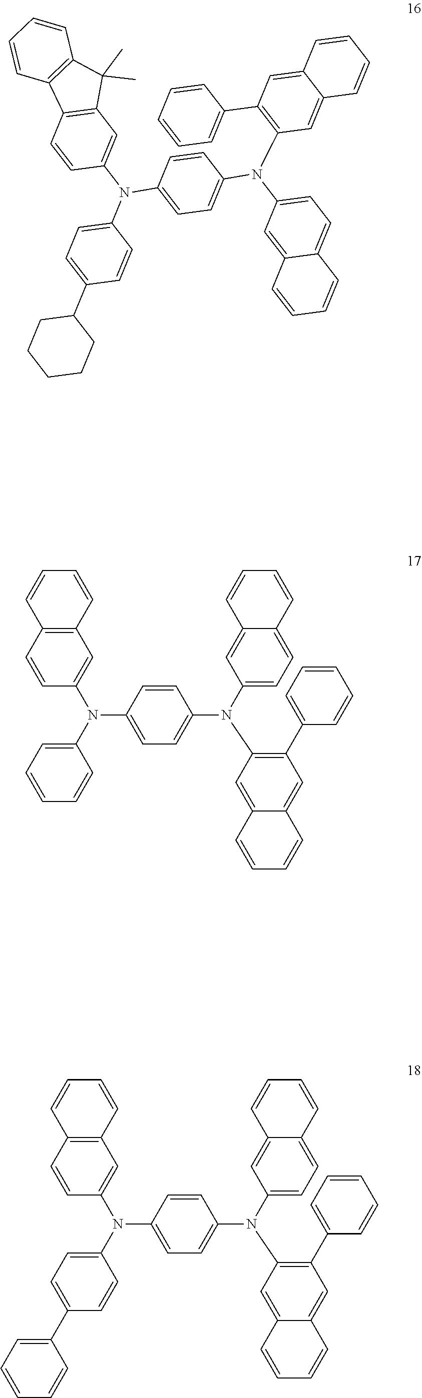 [SMILES: CC1(C)c2ccccc2-c2ccc(N(c3ccc(C4CCCCC4)cc3)c3ccc(N(c4ccc5ccccc5c4)c4cc5ccccc5cc4-c4ccccc4)cc3)cc21 ; c1ccc(-c2cc3ccccc3cc2N(c2ccc(N(c3ccccc3)c3ccc4ccccc4c3)cc2)c2ccc3ccccc3c2)cc1 ; c1ccc(-c2ccc(N(c3ccc(N(c4ccc5ccccc5c4)c4cc5ccccc5cc4-c4ccccc4)cc3)c3ccc4ccccc4c3)cc2)cc1]

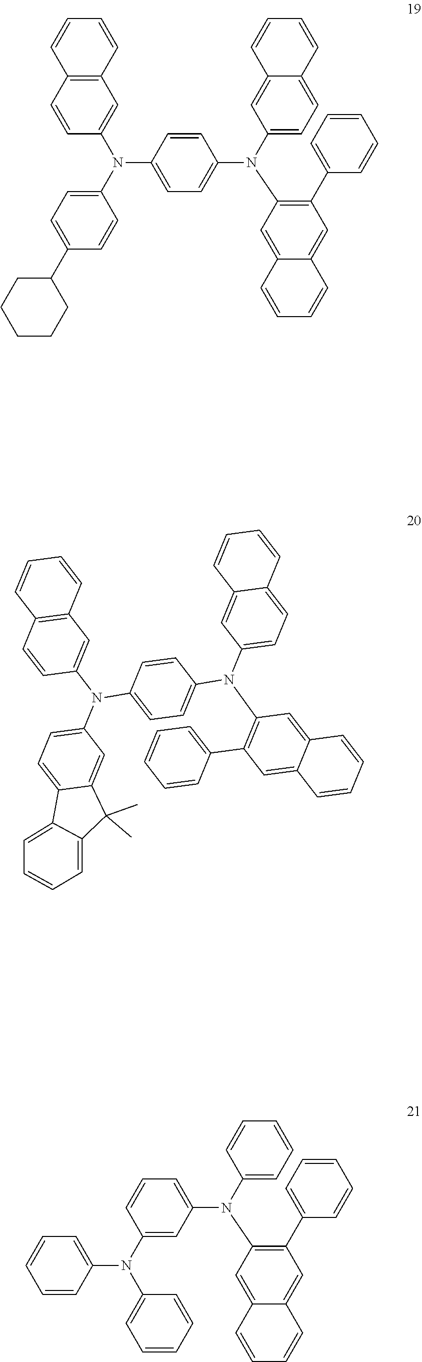 [SMILES: c1ccc(-c2cc3ccccc3cc2N(c2ccc(N(c3ccc(C4CCCCC4)cc3)c3ccc4ccccc4c3)cc2)c2ccc3ccccc3c2)cc1 ; CC1(C)c2ccccc2-c2ccc(N(c3ccc(N(c4ccc5ccccc5c4)c4cc5ccccc5cc4-c4ccccc4)cc3)c3ccc4ccccc4c3)cc21 ; c1ccc(-c2cc3ccccc3cc2N(c2ccccc2)c2cccc(N(c3ccccc3)c3ccccc3)c2)cc1]

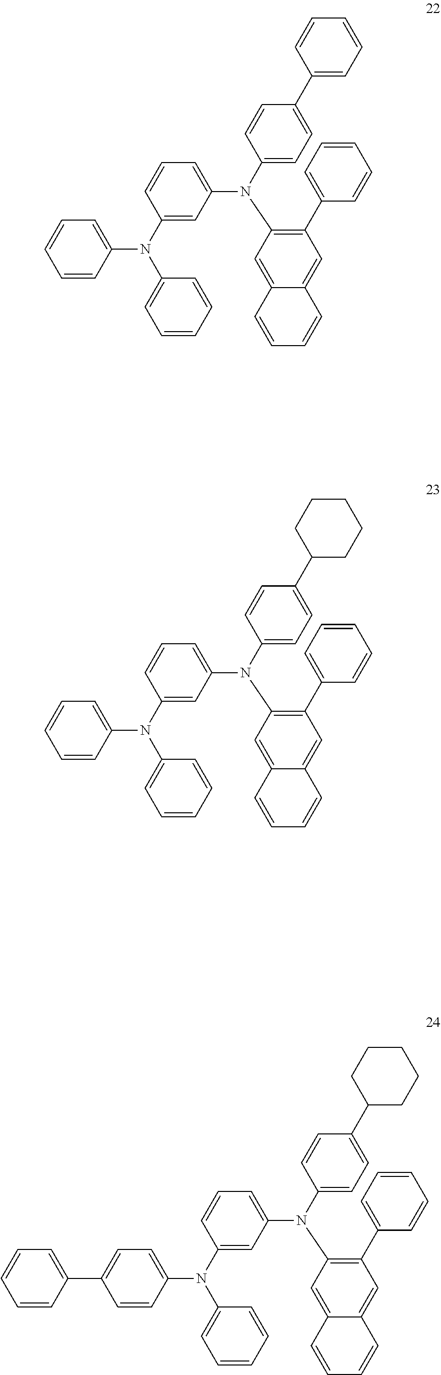 [SMILES: c1ccc(-c2ccc(N(c3cccc(N(c4ccccc4)c4ccccc4)c3)c3cc4ccccc4cc3-c3ccccc3)cc2)cc1 ; c1ccc(-c2cc3ccccc3cc2N(c2ccc(C3CCCCC3)cc2)c2cccc(N(c3ccccc3)c3ccccc3)c2)cc1 ; c1ccc(-c2ccc(N(c3ccccc3)c3cccc(N(c4ccc(C5CCCCC5)cc4)c4cc5ccccc5cc4-c4ccccc4)c3)cc2)cc1]

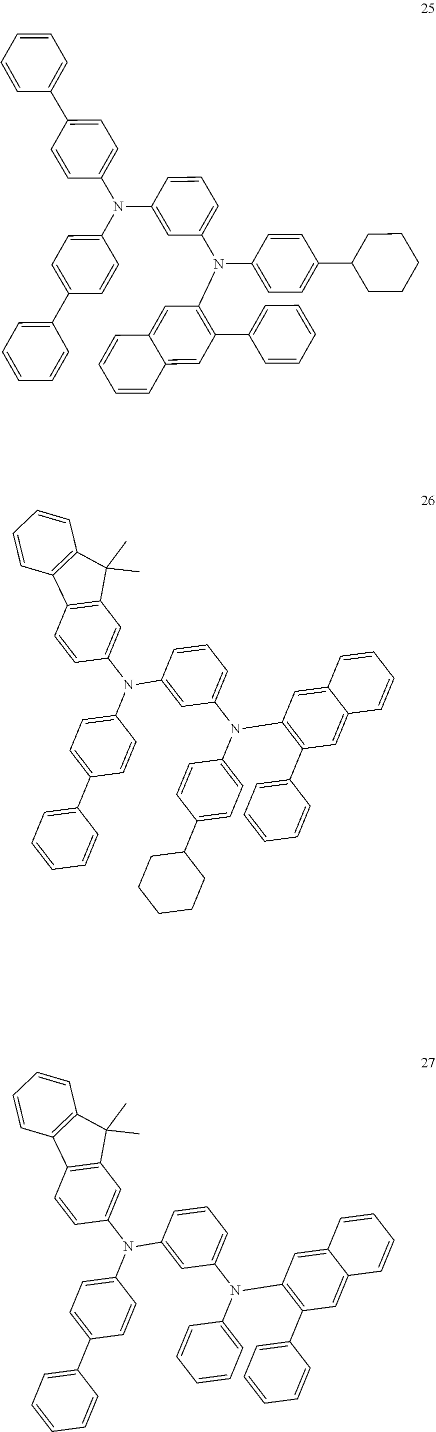 [SMILES: c1ccc(-c2ccc(N(c3ccc(-c4ccccc4)cc3)c3cccc(N(c4ccc(C5CCCCC5)cc4)c4cc5ccccc5cc4-c4ccccc4)c3)cc2)cc1 ; CC1(C)c2ccccc2-c2ccc(N(c3ccc(-c4ccccc4)cc3)c3cccc(N(c4ccc(C5CCCCC5)cc4)c4cc5ccccc5cc4-c4ccccc4)c3)cc21 ; CC1(C)c2ccccc2-c2ccc(N(c3ccc(-c4ccccc4)cc3)c3cccc(N(c4ccccc4)c4cc5ccccc5cc4-c4ccccc4)c3)cc21]

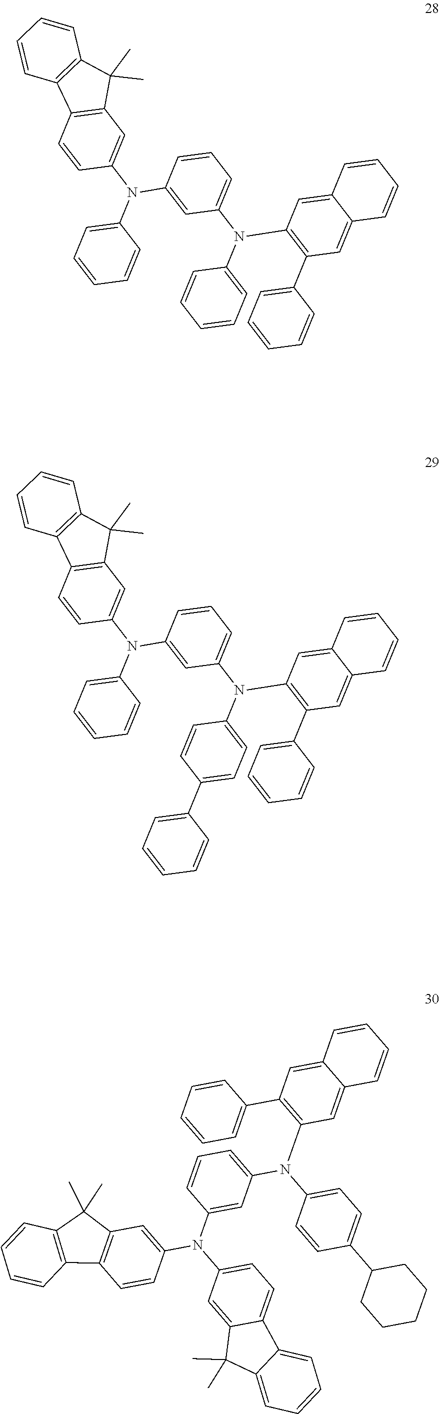 [SMILES: CC1(C)c2ccccc2-c2ccc(N(c3ccccc3)c3cccc(N(c4ccccc4)c4cc5ccccc5cc4-c4ccccc4)c3)cc21 ; CC1(C)c2ccccc2-c2ccc(N(c3ccccc3)c3cccc(N(c4ccc(-c5ccccc5)cc4)c4cc5ccccc5cc4-c4ccccc4)c3)cc21 ; CC1(C)c2ccccc2-c2ccc(N(c3cccc(N(c4ccc(C5CCCCC5)cc4)c4cc5ccccc5cc4-c4ccccc4)c3)c3ccc4c(c3)C(C)(C)c3ccccc3-4)cc21]

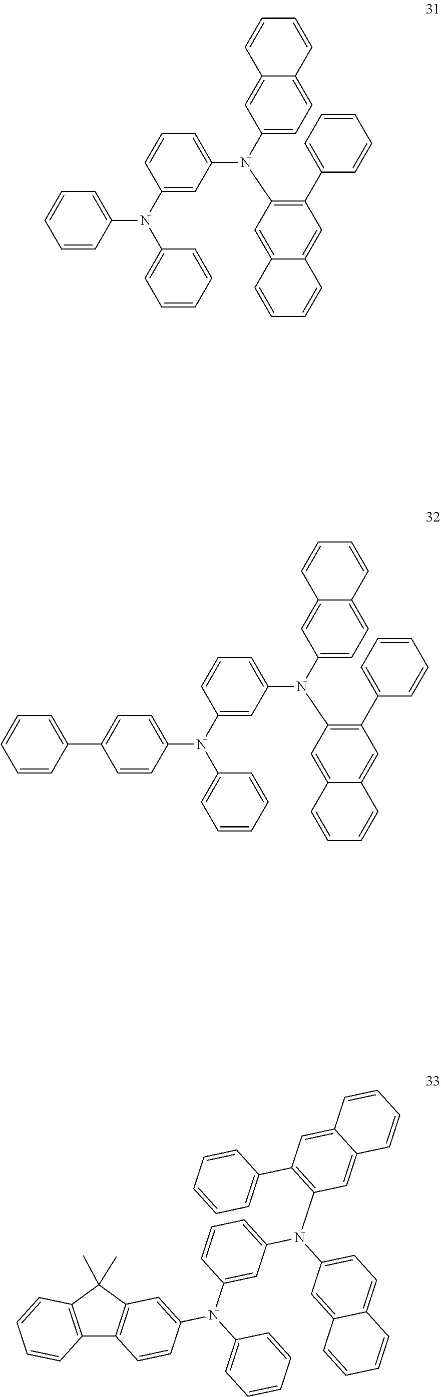 [SMILES: c1ccc(-c2cc3ccccc3cc2N(c2cccc(N(c3ccccc3)c3ccccc3)c2)c2ccc3ccccc3c2)cc1 ; c1ccc(-c2ccc(N(c3ccccc3)c3cccc(N(c4ccc5ccccc5c4)c4cc5ccccc5cc4-c4ccccc4)c3)cc2)cc1 ; CC1(C)c2ccccc2-c2ccc(N(c3ccccc3)c3cccc(N(c4ccc5ccccc5c4)c4cc5ccccc5cc4-c4ccccc4)c3)cc21]

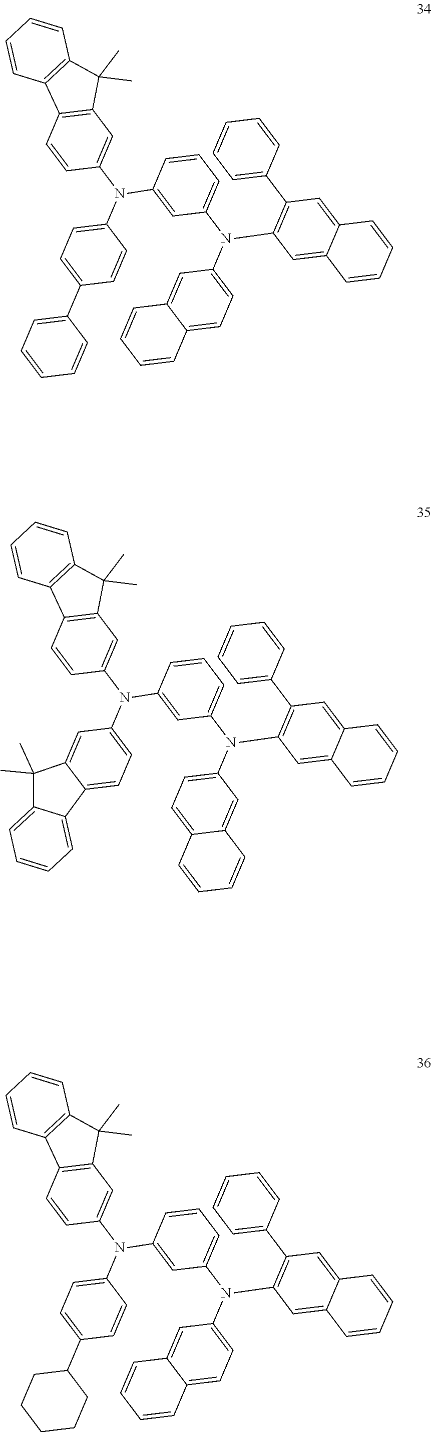 [SMILES: CC1(C)c2ccccc2-c2ccc(N(c3ccc(-c4ccccc4)cc3)c3cccc(N(c4ccc5ccccc5c4)c4cc5ccccc5cc4-c4ccccc4)c3)cc21 ; CC1(C)c2ccccc2-c2ccc(N(c3cccc(N(c4ccc5ccccc5c4)c4cc5ccccc5cc4-c4ccccc4)c3)c3ccc4c(c3)C(C)(C)c3ccccc3-4)cc21 ; CC1(C)c2ccccc2-c2ccc(N(c3ccc(C4CCCCC4)cc3)c3cccc(N(c4ccc5ccccc5c4)c4cc5ccccc5cc4-c4ccccc4)c3)cc21]

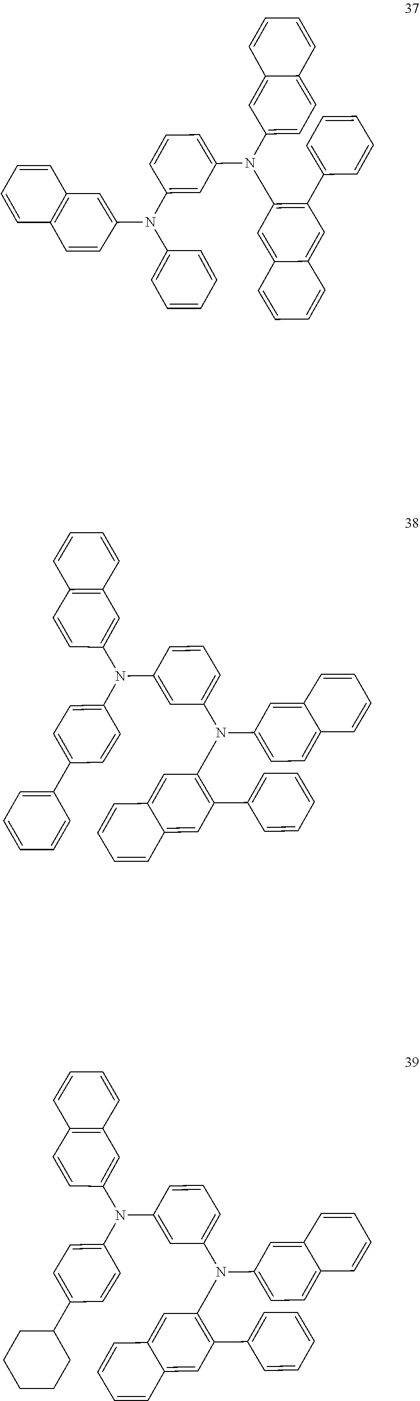 [SMILES: c1ccc(-c2cc3ccccc3cc2N(c2cccc(N(c3ccccc3)c3ccc4ccccc4c3)c2)c2ccc3ccccc3c2)cc1 ; c1ccc(-c2ccc(N(c3cccc(N(c4ccc5ccccc5c4)c4cc5ccccc5cc4-c4ccccc4)c3)c3ccc4ccccc4c3)cc2)cc1 ; c1ccc(-c2cc3ccccc3cc2N(c2cccc(N(c3ccc(C4CCCCC4)cc3)c3ccc4ccccc4c3)c2)c2ccc3ccccc3c2)cc1]

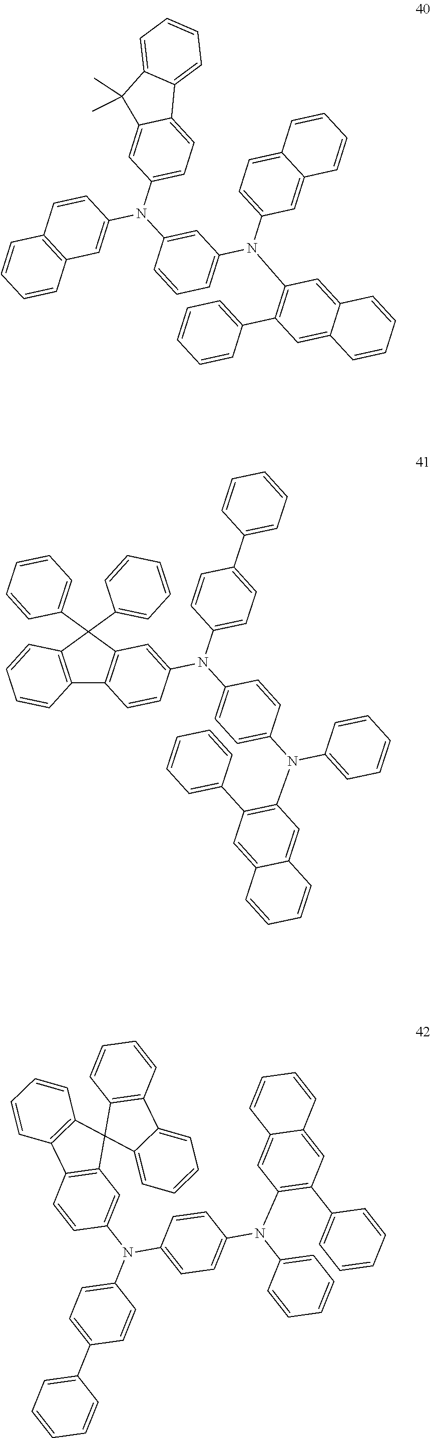 [SMILES: CC1(C)c2ccccc2-c2ccc(N(c3cccc(N(c4ccc5ccccc5c4)c4cc5ccccc5cc4-c4ccccc4)c3)c3ccc4ccccc4c3)cc21 ; c1ccc(-c2ccc(N(c3ccc(N(c4ccccc4)c4cc5ccccc5cc4-c4ccccc4)cc3)c3ccc4c(c3)C(c3ccccc3)(c3ccccc3)c3ccccc3-4)cc2)cc1 ; c1ccc(-c2ccc(N(c3ccc(N(c4ccccc4)c4cc5ccccc5cc4-c4ccccc4)cc3)c3ccc4c(c3)C3(c5ccccc5-c5ccccc53)c3ccccc3-4)cc2)cc1]

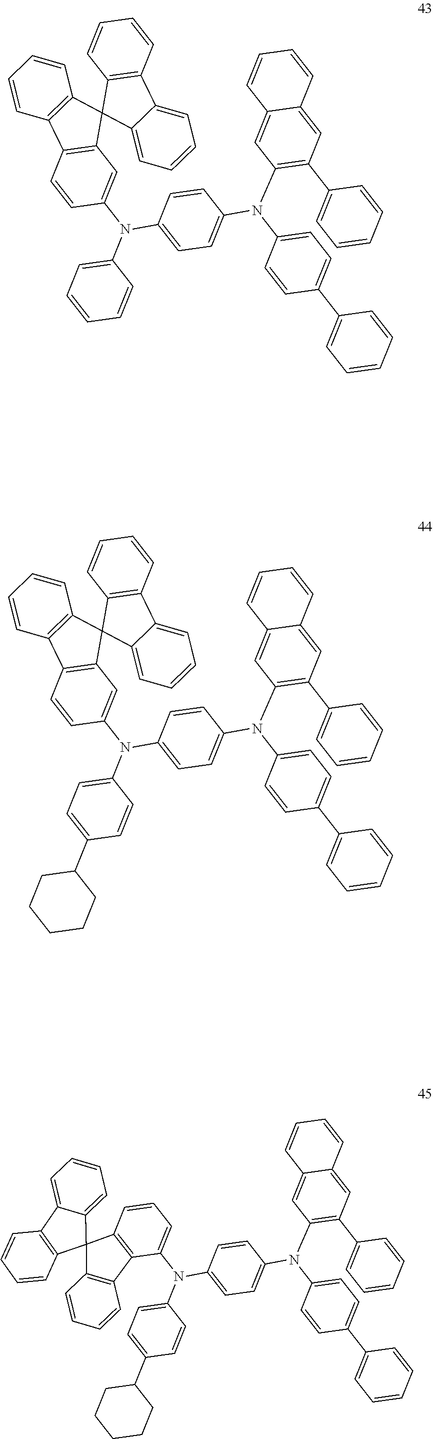 [SMILES: c1ccc(-c2ccc(N(c3ccc(N(c4ccccc4)c4ccc5c(c4)C4(c6ccccc6-c6ccccc64)c4ccccc4-5)cc3)c3cc4ccccc4cc3-c3ccccc3)cc2)cc1 ; c1ccc(-c2ccc(N(c3ccc(N(c4ccc(C5CCCCC5)cc4)c4ccc5c(c4)C4(c6ccccc6-c6ccccc64)c4ccccc4-5)cc3)c3cc4ccccc4cc3-c3ccccc3)cc2)cc1 ; c1ccc(-c2ccc(N(c3ccc(N(c4ccc(C5CCCCC5)cc4)c4cccc5c4-c4ccccc4C54c5ccccc5-c5ccccc54)cc3)c3cc4ccccc4cc3-c3ccccc3)cc2)cc1]

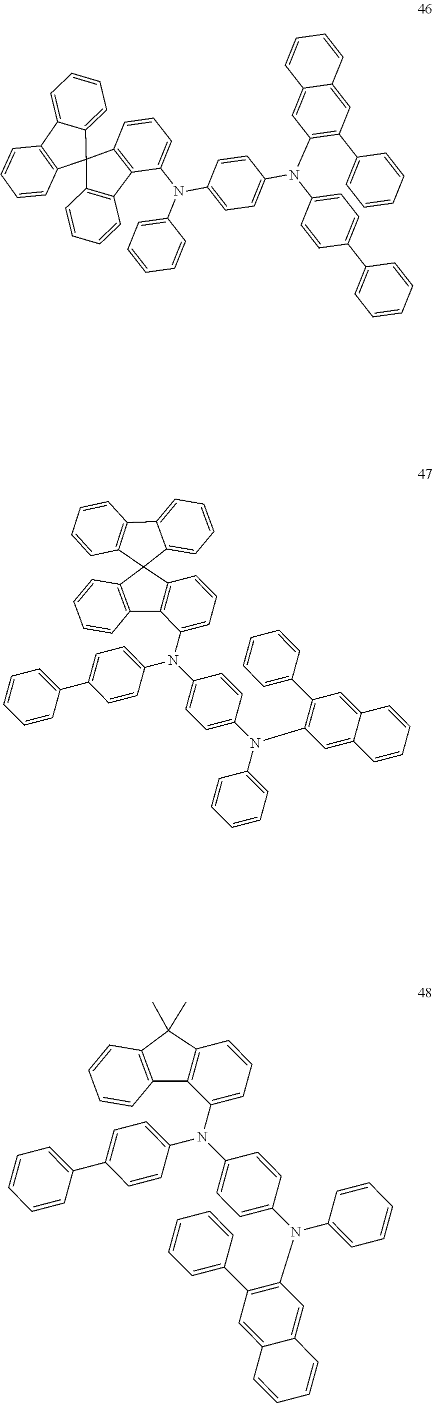 [SMILES: c1ccc(-c2ccc(N(c3ccc(N(c4ccccc4)c4cccc5c4-c4ccccc4C54c5ccccc5-c5ccccc54)cc3)c3cc4ccccc4cc3-c3ccccc3)cc2)cc1 ; c1ccc(-c2ccc(N(c3ccc(N(c4ccccc4)c4cc5ccccc5cc4-c4ccccc4)cc3)c3cccc4c3-c3ccccc3C43c4ccccc4-c4ccccc43)cc2)cc1 ; CC1(C)c2ccccc2-c2c(N(c3ccc(-c4ccccc4)cc3)c3ccc(N(c4ccccc4)c4cc5ccccc5cc4-c4ccccc4)cc3)cccc21]

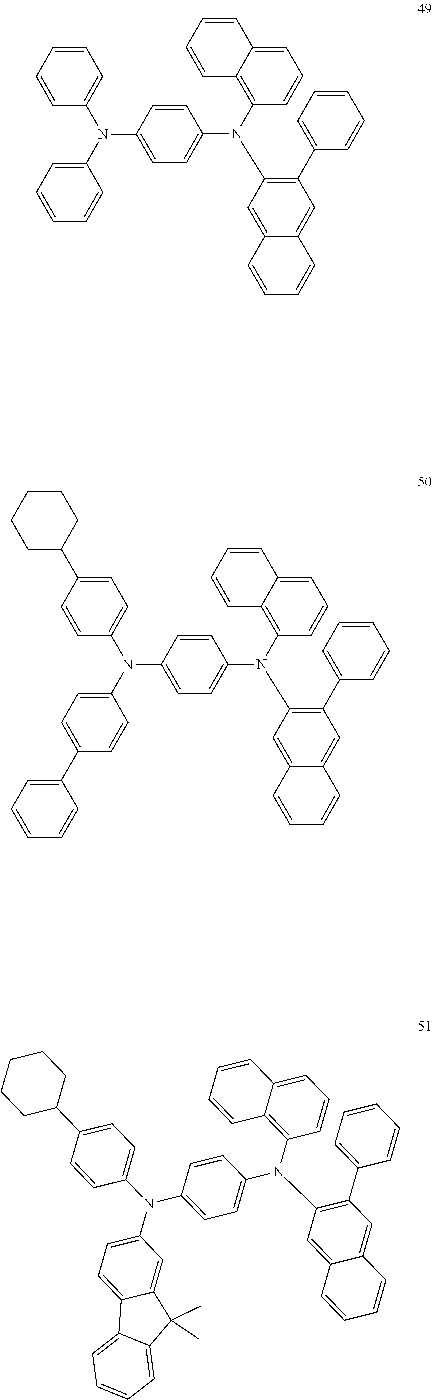 [SMILES: c1ccc(-c2cc3ccccc3cc2N(c2ccc(N(c3ccccc3)c3ccccc3)cc2)c2cccc3ccccc23)cc1 ; c1ccc(-c2ccc(N(c3ccc(C4CCCCC4)cc3)c3ccc(N(c4cc5ccccc5cc4-c4ccccc4)c4cccc5ccccc45)cc3)cc2)cc1 ; CC1(C)c2ccccc2-c2ccc(N(c3ccc(C4CCCCC4)cc3)c3ccc(N(c4cc5ccccc5cc4-c4ccccc4)c4cccc5ccccc45)cc3)cc21]

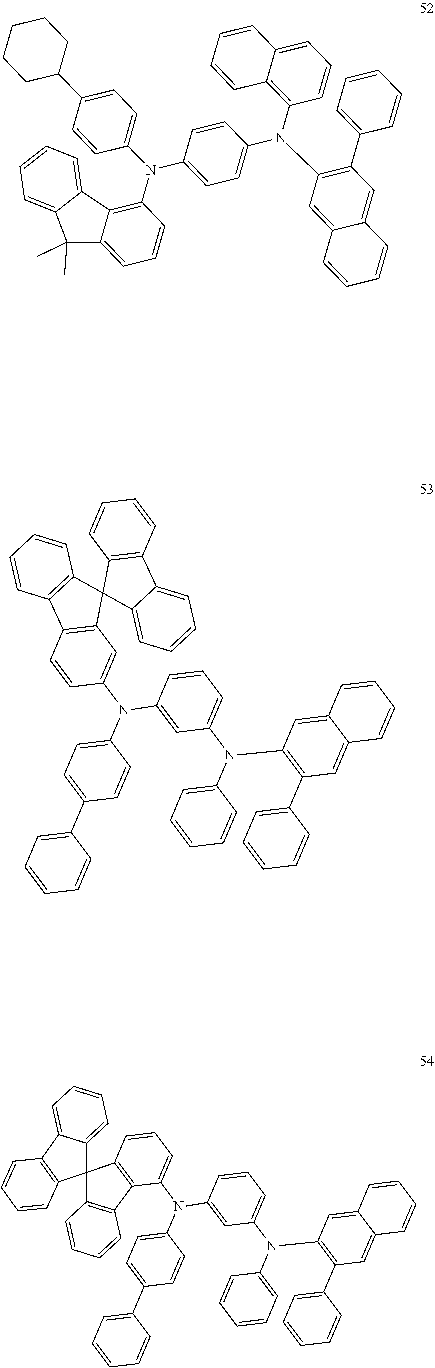

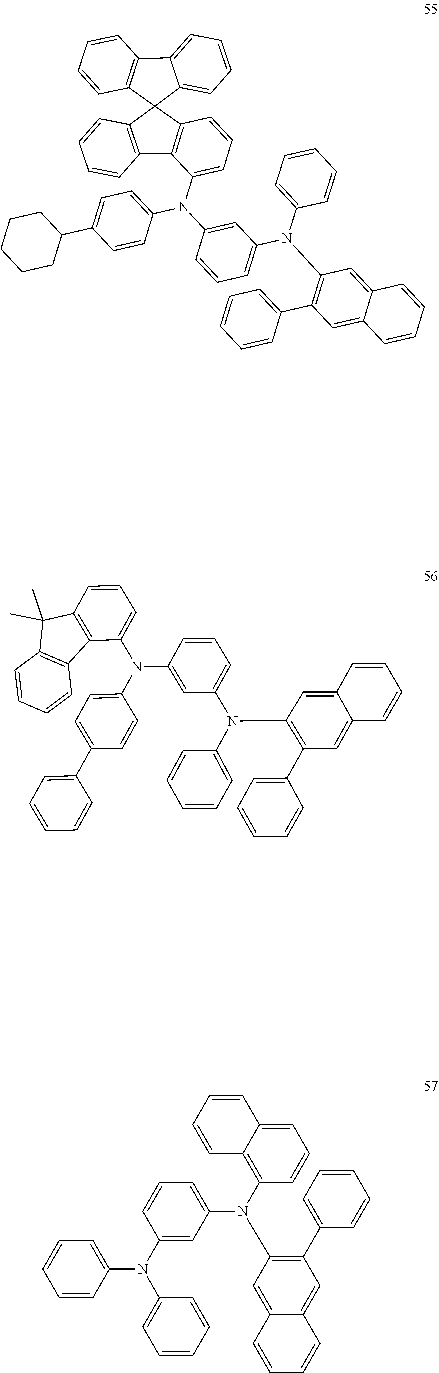 [SMILES: c1ccc(-c2cc3ccccc3cc2N(c2ccccc2)c2cccc(N(c3ccc(C4CCCCC4)cc3)c3cccc4c3-c3ccccc3C43c4ccccc4-c4ccccc43)c2)cc1 ; CC1(C)c2ccccc2-c2c(N(c3ccc(-c4ccccc4)cc3)c3cccc(N(c4ccccc4)c4cc5ccccc5cc4-c4ccccc4)c3)cccc21 ; c1ccc(-c2cc3ccccc3cc2N(c2cccc(N(c3ccccc3)c3ccccc3)c2)c2cccc3ccccc23)cc1]

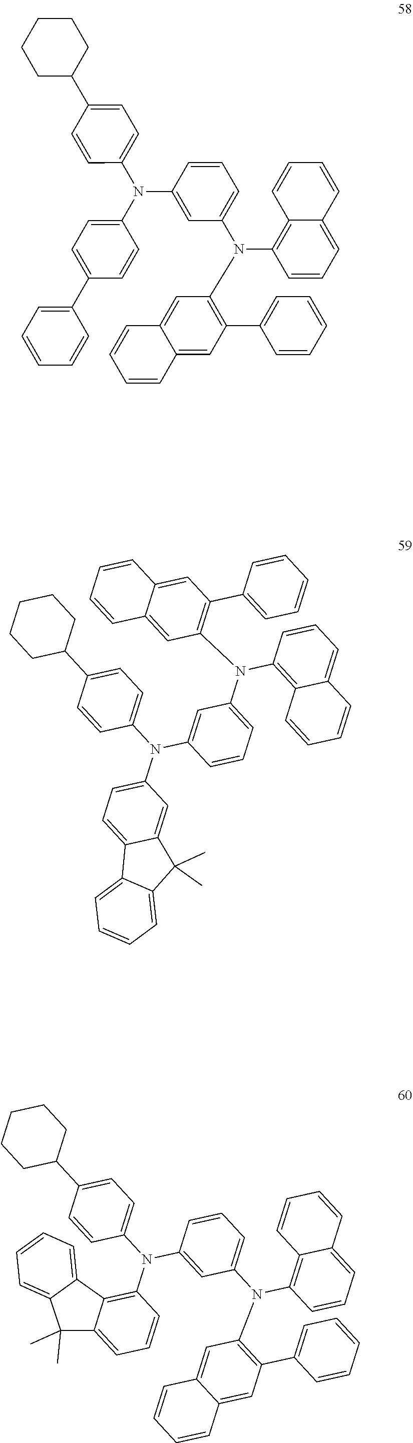 [SMILES: c1ccc(-c2ccc(N(c3ccc(C4CCCCC4)cc3)c3cccc(N(c4cc5ccccc5cc4-c4ccccc4)c4cccc5ccccc45)c3)cc2)cc1 ; CC1(C)c2ccccc2-c2ccc(N(c3ccc(C4CCCCC4)cc3)c3cccc(N(c4cc5ccccc5cc4-c4ccccc4)c4cccc5ccccc45)c3)cc21 ; CC1(C)c2ccccc2-c2c(N(c3ccc(C4CCCCC4)cc3)c3cccc(N(c4cc5ccccc5cc4-c4ccccc4)c4cccc5ccccc45)c3)cccc21]

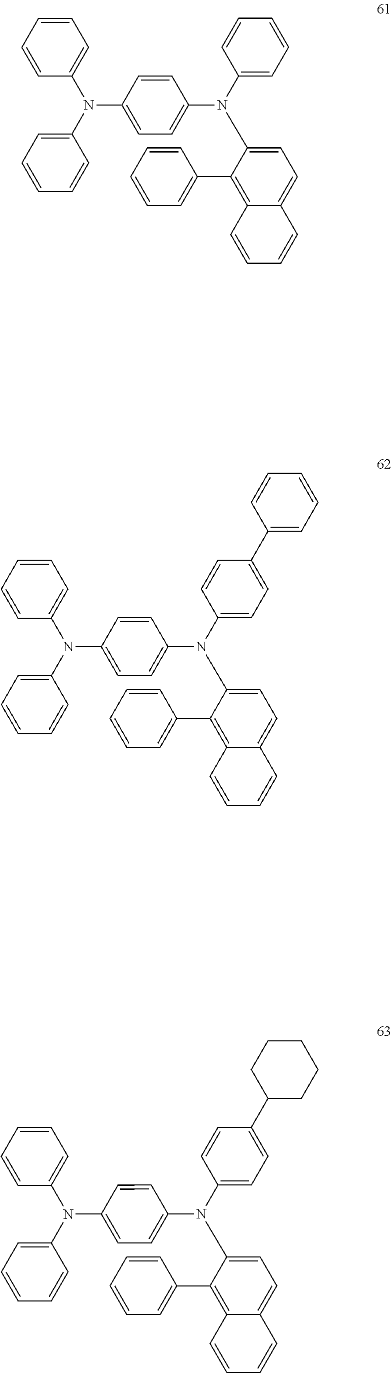 [SMILES: c1ccc(-c2c(N(c3ccccc3)c3ccc(N(c4ccccc4)c4ccccc4)cc3)ccc3ccccc23)cc1 ; c1ccc(-c2ccc(N(c3ccc(N(c4ccccc4)c4ccccc4)cc3)c3ccc4ccccc4c3-c3ccccc3)cc2)cc1 ; c1ccc(-c2c(N(c3ccc(C4CCCCC4)cc3)c3ccc(N(c4ccccc4)c4ccccc4)cc3)ccc3ccccc23)cc1]

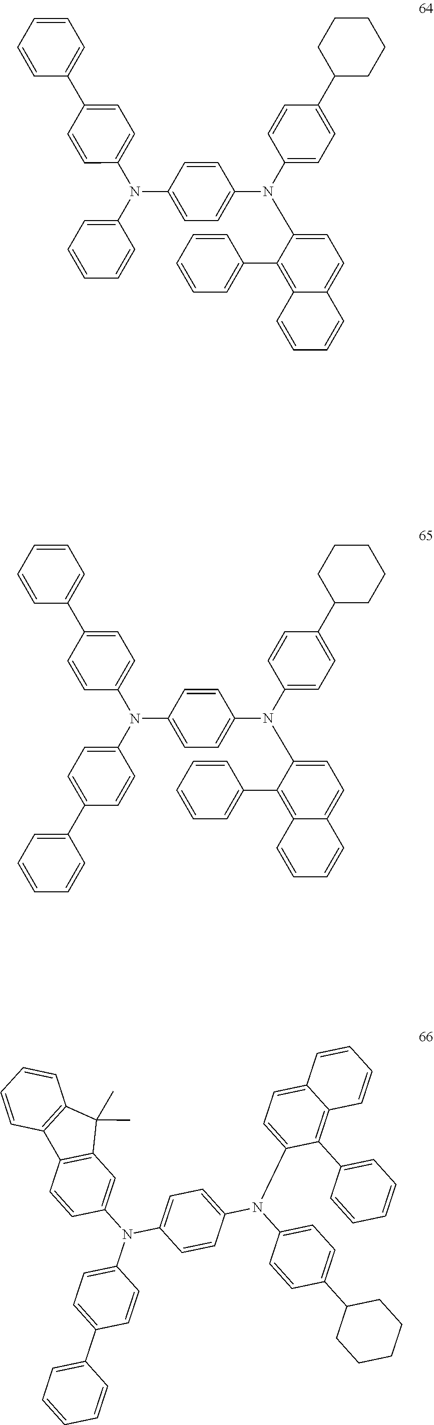 [SMILES: c1ccc(-c2ccc(N(c3ccccc3)c3ccc(N(c4ccc(C5CCCCC5)cc4)c4ccc5ccccc5c4-c4ccccc4)cc3)cc2)cc1 ; c1ccc(-c2ccc(N(c3ccc(-c4ccccc4)cc3)c3ccc(N(c4ccc(C5CCCCC5)cc4)c4ccc5ccccc5c4-c4ccccc4)cc3)cc2)cc1 ; CC1(C)c2ccccc2-c2ccc(N(c3ccc(-c4ccccc4)cc3)c3ccc(N(c4ccc(C5CCCCC5)cc4)c4ccc5ccccc5c4-c4ccccc4)cc3)cc21]

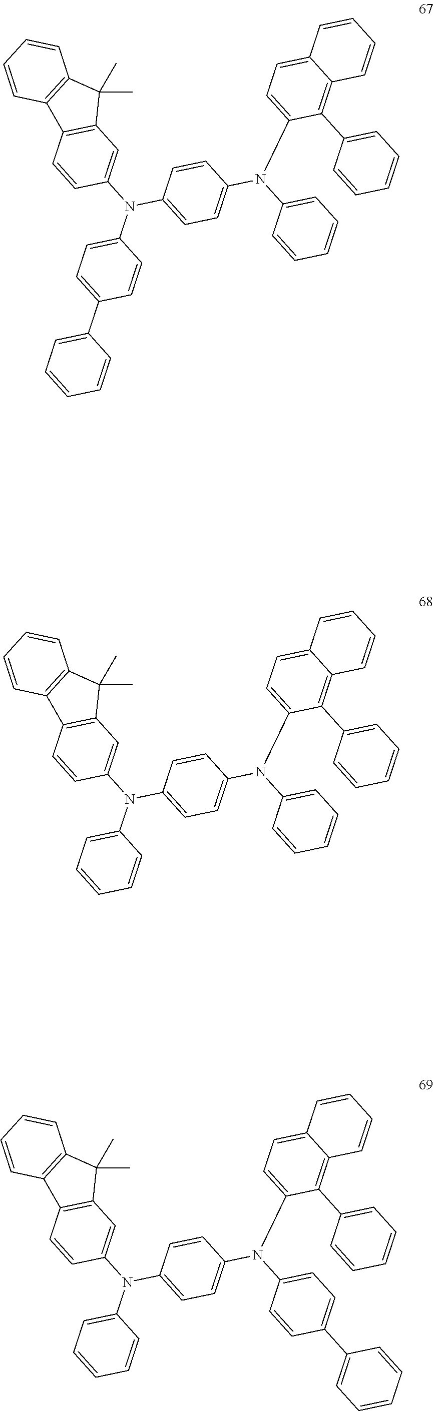 [SMILES: CC1(C)c2ccccc2-c2ccc(N(c3ccc(-c4ccccc4)cc3)c3ccc(N(c4ccccc4)c4ccc5ccccc5c4-c4ccccc4)cc3)cc21 ; CC1(C)c2ccccc2-c2ccc(N(c3ccccc3)c3ccc(N(c4ccccc4)c4ccc5ccccc5c4-c4ccccc4)cc3)cc21 ; CC1(C)c2ccccc2-c2ccc(N(c3ccccc3)c3ccc(N(c4ccc(-c5ccccc5)cc4)c4ccc5ccccc5c4-c4ccccc4)cc3)cc21]

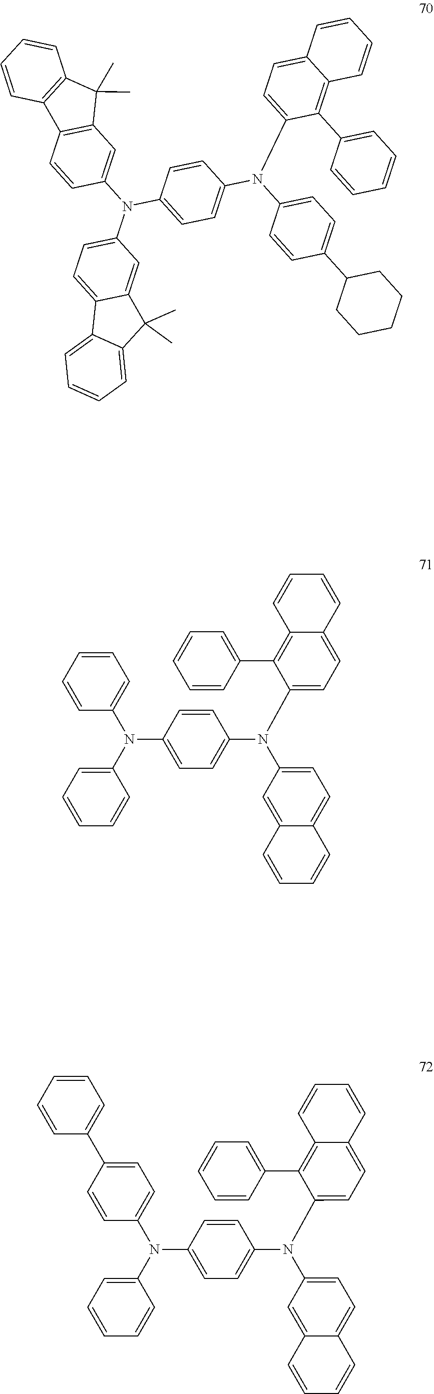 [SMILES: CC1(C)c2ccccc2-c2ccc(N(c3ccc(N(c4ccc(C5CCCCC5)cc4)c4ccc5ccccc5c4-c4ccccc4)cc3)c3ccc4c(c3)C(C)(C)c3ccccc3-4)cc21 ; c1ccc(-c2c(N(c3ccc(N(c4ccccc4)c4ccccc4)cc3)c3ccc4ccccc4c3)ccc3ccccc23)cc1 ; c1ccc(-c2ccc(N(c3ccccc3)c3ccc(N(c4ccc5ccccc5c4)c4ccc5ccccc5c4-c4ccccc4)cc3)cc2)cc1]

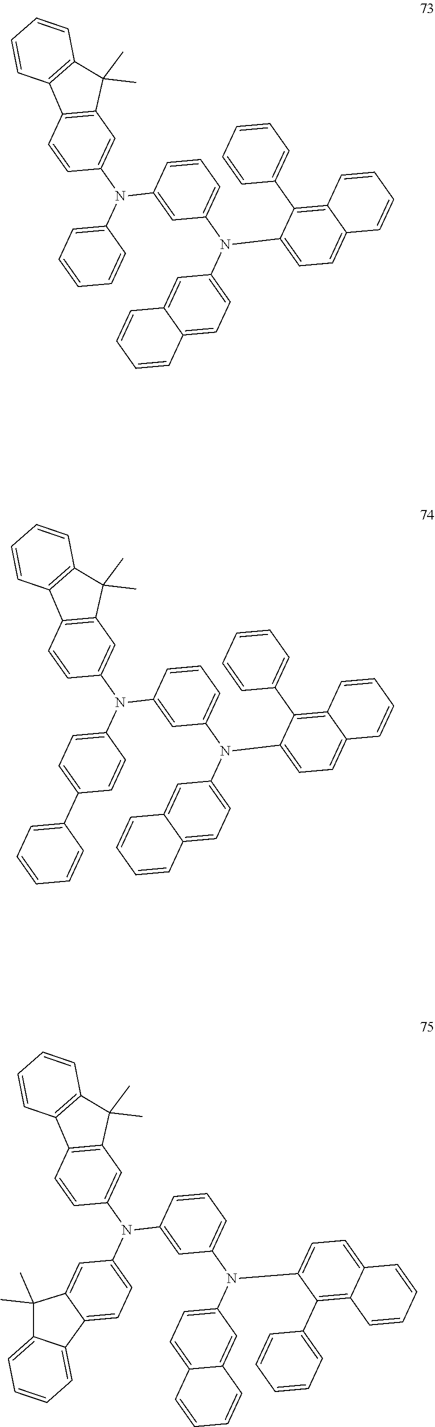 [SMILES: CC1(C)c2ccccc2-c2ccc(N(c3ccccc3)c3cccc(N(c4ccc5ccccc5c4)c4ccc5ccccc5c4-c4ccccc4)c3)cc21 ; CC1(C)c2ccccc2-c2ccc(N(c3ccc(-c4ccccc4)cc3)c3cccc(N(c4ccc5ccccc5c4)c4ccc5ccccc5c4-c4ccccc4)c3)cc21 ; CC1(C)c2ccccc2-c2ccc(N(c3cccc(N(c4ccc5ccccc5c4)c4ccc5ccccc5c4-c4ccccc4)c3)c3ccc4c(c3)C(C)(C)c3ccccc3-4)cc21]

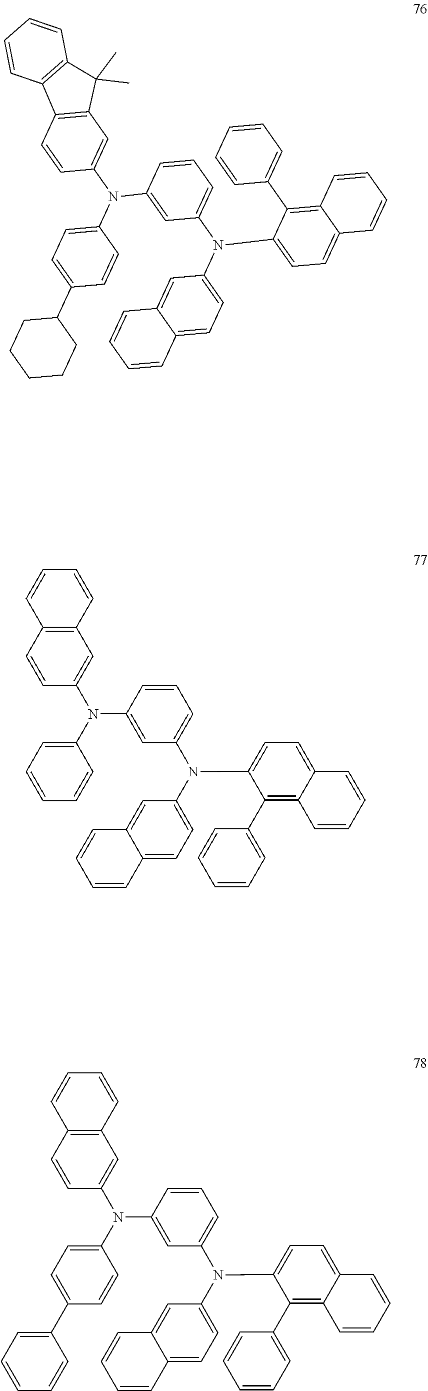 [SMILES: CC1(C)c2ccccc2-c2ccc(N(c3ccc(C4CCCCC4)cc3)c3cccc(N(c4ccc5ccccc5c4)c4ccc5ccccc5c4-c4ccccc4)c3)cc21 ; c1ccc(-c2c(N(c3cccc(N(c4ccccc4)c4ccc5ccccc5c4)c3)c3ccc4ccccc4c3)ccc3ccccc23)cc1 ; c1ccc(-c2ccc(N(c3cccc(N(c4ccc5ccccc5c4)c4ccc5ccccc5c4-c4ccccc4)c3)c3ccc4ccccc4c3)cc2)cc1]

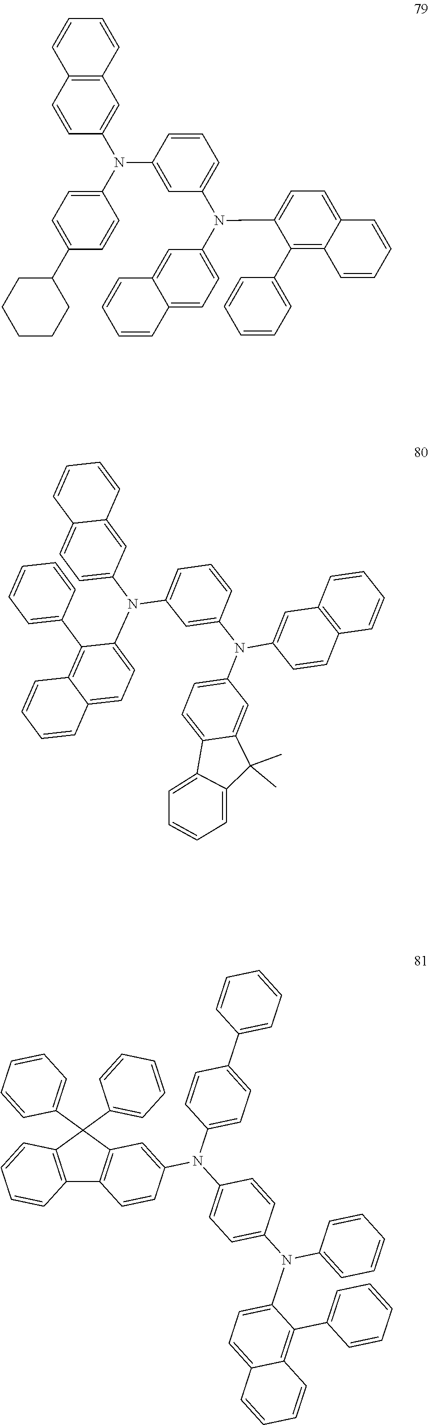 [SMILES: c1ccc(-c2c(N(c3cccc(N(c4ccc(C5CCCCC5)cc4)c4ccc5ccccc5c4)c3)c3ccc4ccccc4c3)ccc3ccccc23)cc1 ; CC1(C)c2ccccc2-c2ccc(N(c3cccc(N(c4ccc5ccccc5c4)c4ccc5ccccc5c4-c4ccccc4)c3)c3ccc4ccccc4c3)cc21 ; c1ccc(-c2ccc(N(c3ccc(N(c4ccccc4)c4ccc5ccccc5c4-c4ccccc4)cc3)c3ccc4c(c3)C(c3ccccc3)(c3ccccc3)c3ccccc3-4)cc2)cc1]

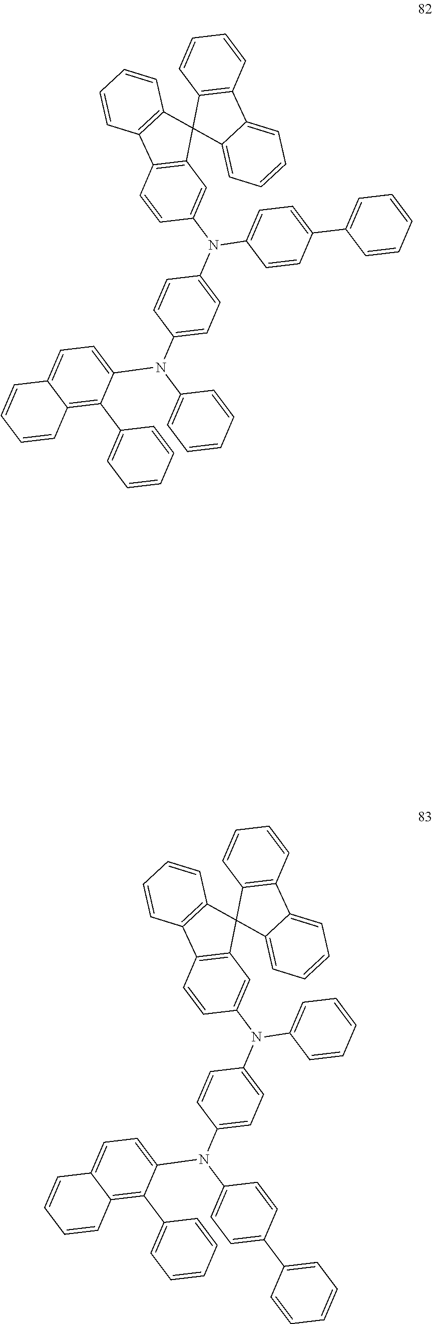 [SMILES: c1ccc(-c2ccc(N(c3ccc(N(c4ccccc4)c4ccc5ccccc5c4-c4ccccc4)cc3)c3ccc4c(c3)C3(c5ccccc5-c5ccccc53)c3ccccc3-4)cc2)cc1 ; c1ccc(-c2ccc(N(c3ccc(N(c4ccccc4)c4ccc5c(c4)C4(c6ccccc6-c6ccccc64)c4ccccc4-5)cc3)c3ccc4ccccc4c3-c3ccccc3)cc2)cc1]

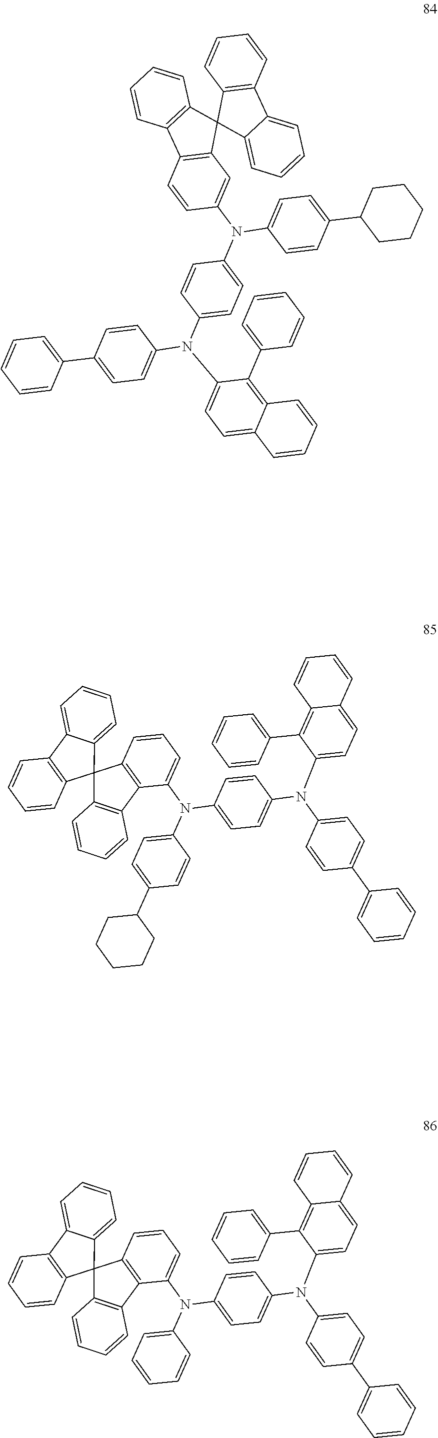 [SMILES: c1ccc(-c2ccc(N(c3ccc(N(c4ccc(C5CCCCC5)cc4)c4ccc5c(c4)C4(c6ccccc6-c6ccccc64)c4ccccc4-5)cc3)c3ccc4ccccc4c3-c3ccccc3)cc2)cc1 ; c1ccc(-c2ccc(N(c3ccc(N(c4ccc(C5CCCCC5)cc4)c4cccc5c4-c4ccccc4C54c5ccccc5-c5ccccc54)cc3)c3ccc4ccccc4c3-c3ccccc3)cc2)cc1 ; c1ccc(-c2ccc(N(c3ccc(N(c4ccccc4)c4cccc5c4-c4ccccc4C54c5ccccc5-c5ccccc54)cc3)c3ccc4ccccc4c3-c3ccccc3)cc2)cc1]

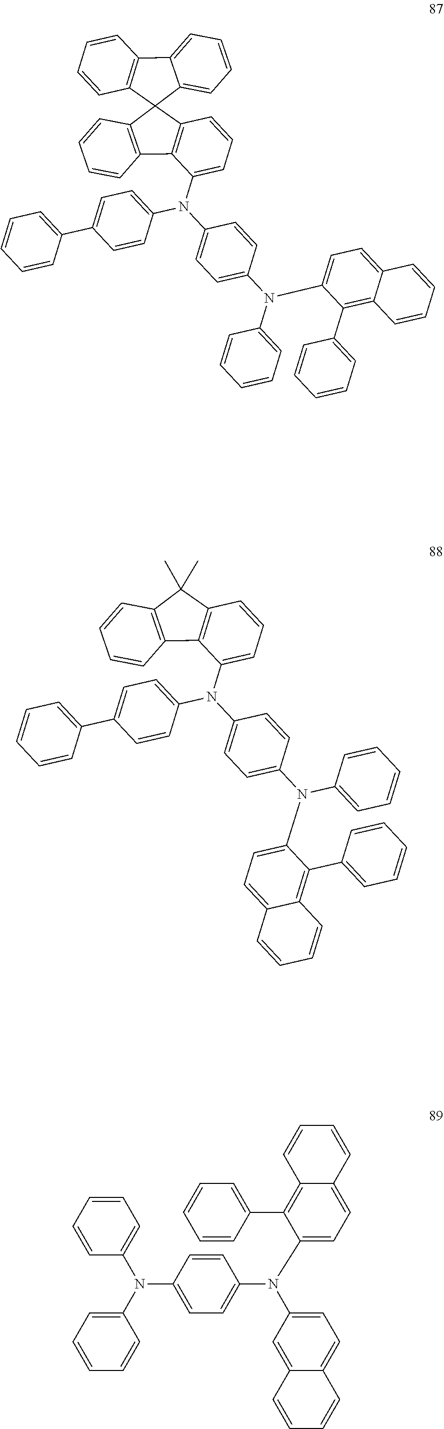 [SMILES: c1ccc(-c2ccc(N(c3ccc(N(c4ccccc4)c4ccc5ccccc5c4-c4ccccc4)cc3)c3cccc4c3-c3ccccc3C43c4ccccc4-c4ccccc43)cc2)cc1 ; CC1(C)c2ccccc2-c2c(N(c3ccc(-c4ccccc4)cc3)c3ccc(N(c4ccccc4)c4ccc5ccccc5c4-c4ccccc4)cc3)cccc21 ; c1ccc(-c2c(N(c3ccc(N(c4ccccc4)c4ccccc4)cc3)c3ccc4ccccc4c3)ccc3ccccc23)cc1]

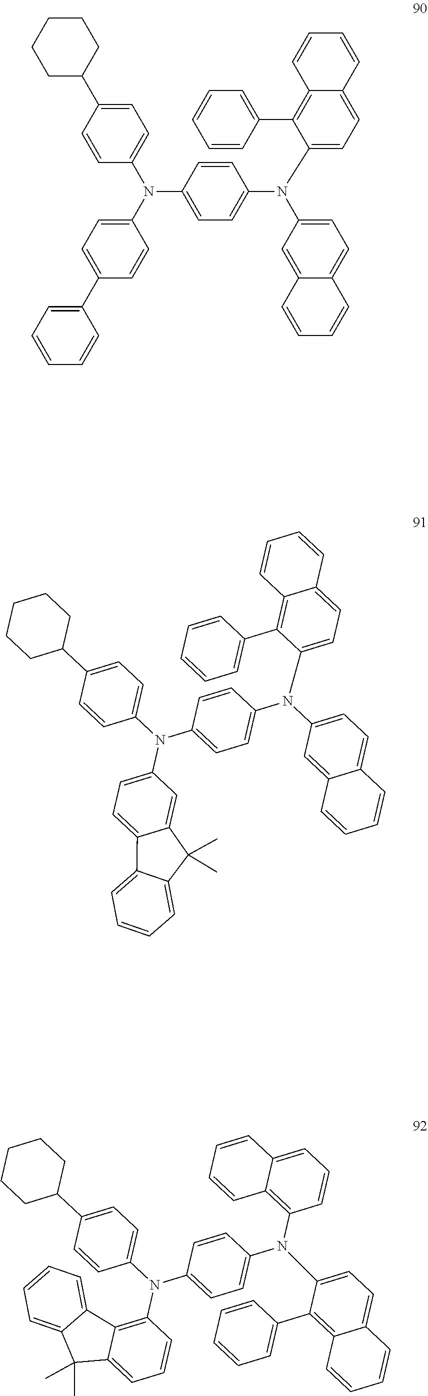 [SMILES: c1ccc(-c2ccc(N(c3ccc(C4CCCCC4)cc3)c3ccc(N(c4ccc5ccccc5c4)c4ccc5ccccc5c4-c4ccccc4)cc3)cc2)cc1 ; CC1(C)c2ccccc2-c2ccc(N(c3ccc(C4CCCCC4)cc3)c3ccc(N(c4ccc5ccccc5c4)c4ccc5ccccc5c4-c4ccccc4)cc3)cc21 ; CC1(C)c2ccccc2-c2c(N(c3ccc(C4CCCCC4)cc3)c3ccc(N(c4ccc5ccccc5c4-c4ccccc4)c4cccc5ccccc45)cc3)cccc21]

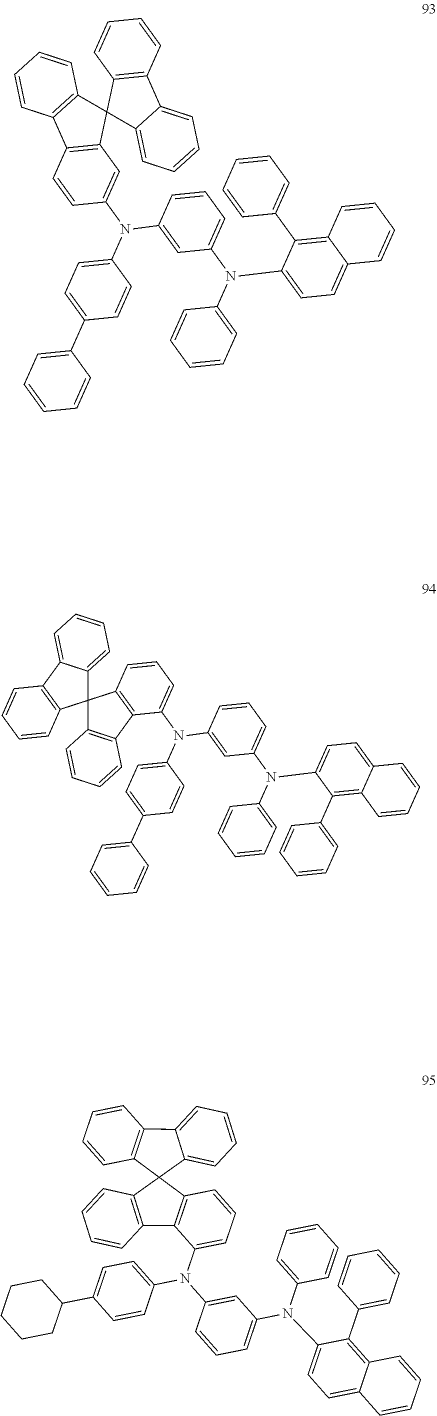 [SMILES: c1ccc(-c2ccc(N(c3cccc(N(c4ccccc4)c4ccc5ccccc5c4-c4ccccc4)c3)c3ccc4c(c3)C3(c5ccccc5-c5ccccc53)c3ccccc3-4)cc2)cc1 ; c1ccc(-c2ccc(N(c3cccc(N(c4ccccc4)c4ccc5ccccc5c4-c4ccccc4)c3)c3cccc4c3-c3ccccc3C43c4ccccc4-c4ccccc43)cc2)cc1 ; c1ccc(-c2c(N(c3ccccc3)c3cccc(N(c4ccc(C5CCCCC5)cc4)c4cccc5c4-c4ccccc4C54c5ccccc5-c5ccccc54)c3)ccc3ccccc23)cc1]

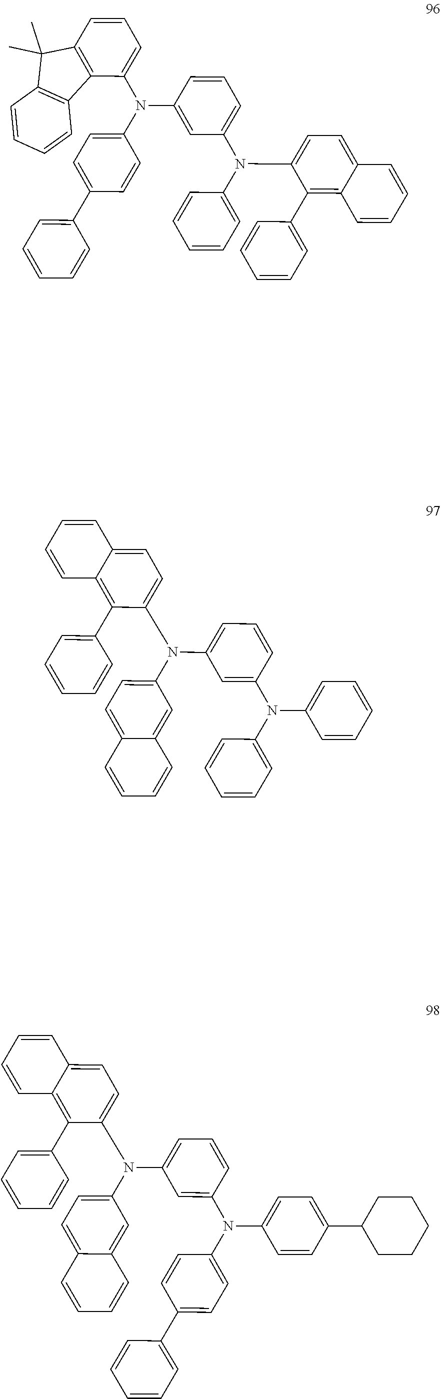 [SMILES: CC1(C)c2ccccc2-c2c(N(c3ccc(-c4ccccc4)cc3)c3cccc(N(c4ccccc4)c4ccc5ccccc5c4-c4ccccc4)c3)cccc21 ; c1ccc(-c2c(N(c3cccc(N(c4ccccc4)c4ccccc4)c3)c3ccc4ccccc4c3)ccc3ccccc23)cc1 ; c1ccc(-c2ccc(N(c3ccc(C4CCCCC4)cc3)c3cccc(N(c4ccc5ccccc5c4)c4ccc5ccccc5c4-c4ccccc4)c3)cc2)cc1]

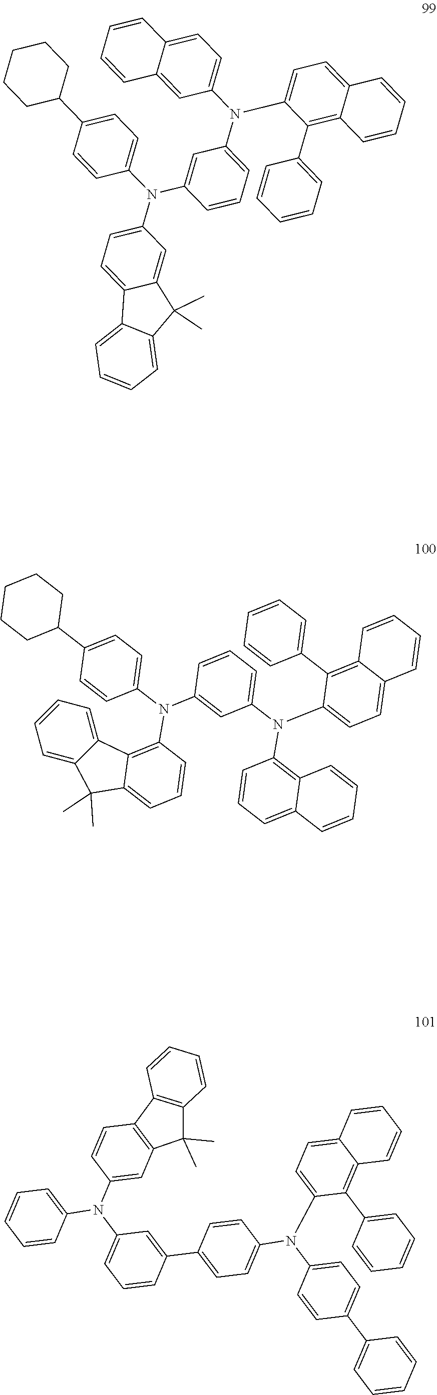

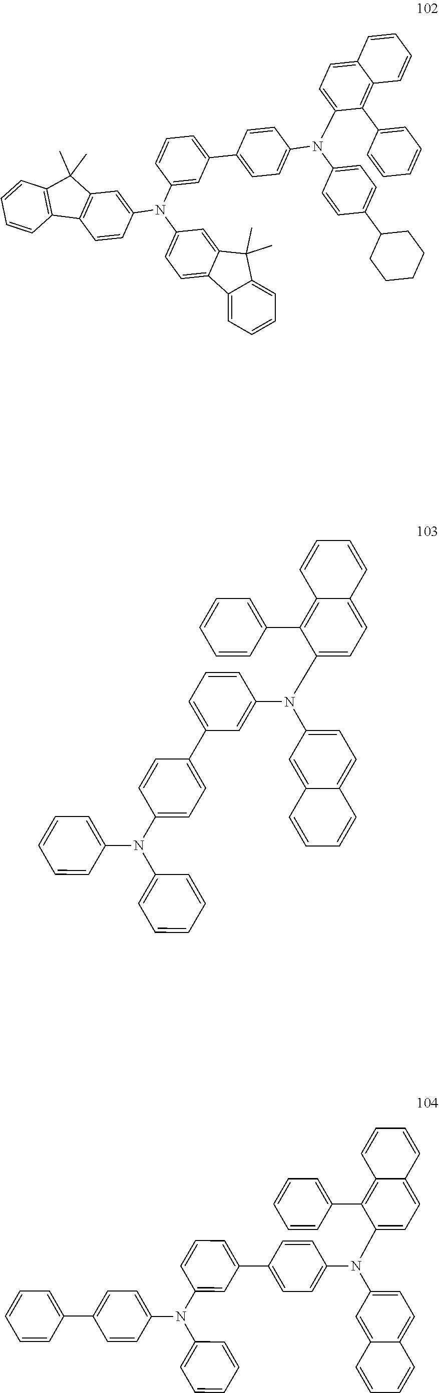

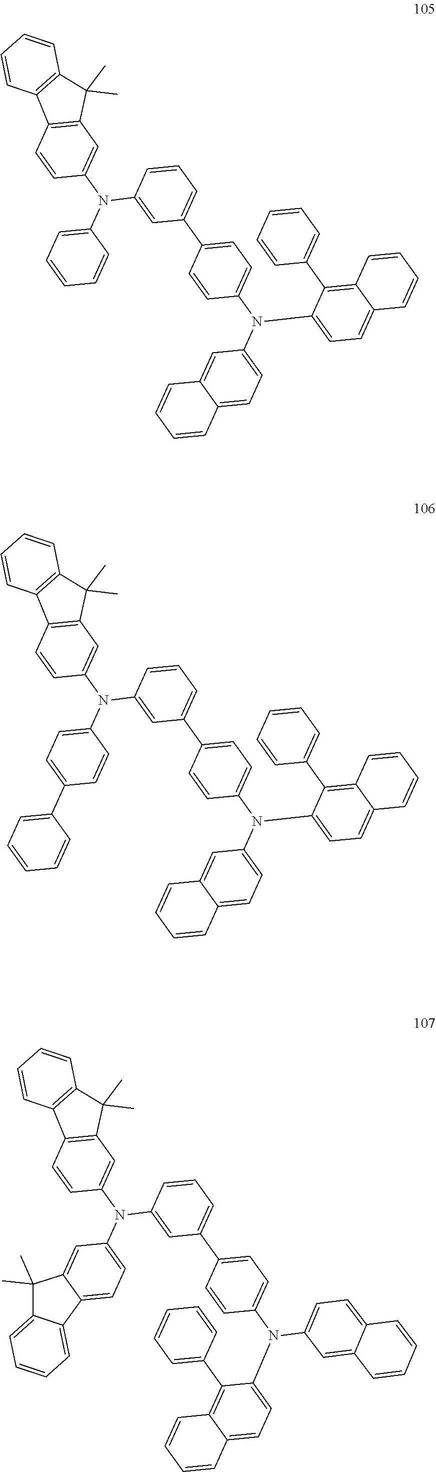

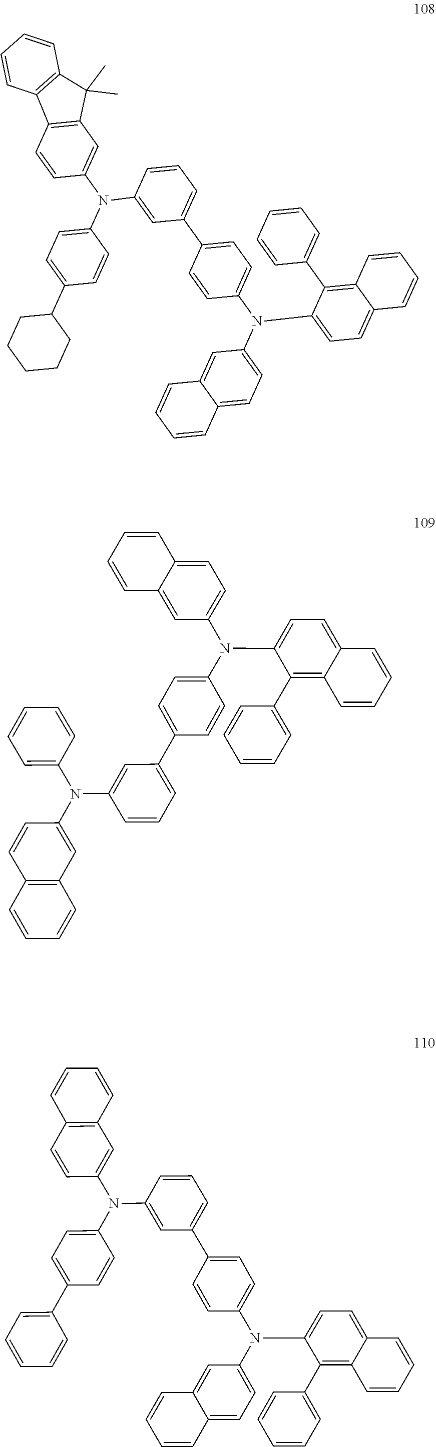 [SMILES: CC1(C)c2ccccc2-c2ccc(N(c3ccc(C4CCCCC4)cc3)c3cccc(-c4ccc(N(c5ccc6ccccc6c5)c5ccc6ccccc6c5-c5ccccc5)cc4)c3)cc21 ; c1ccc(-c2c(N(c3ccc(-c4cccc(N(c5ccccc5)c5ccc6ccccc6c5)c4)cc3)c3ccc4ccccc4c3)ccc3ccccc23)cc1 ; c1ccc(-c2ccc(N(c3cccc(-c4ccc(N(c5ccc6ccccc6c5)c5ccc6ccccc6c5-c5ccccc5)cc4)c3)c3ccc4ccccc4c3)cc2)cc1]

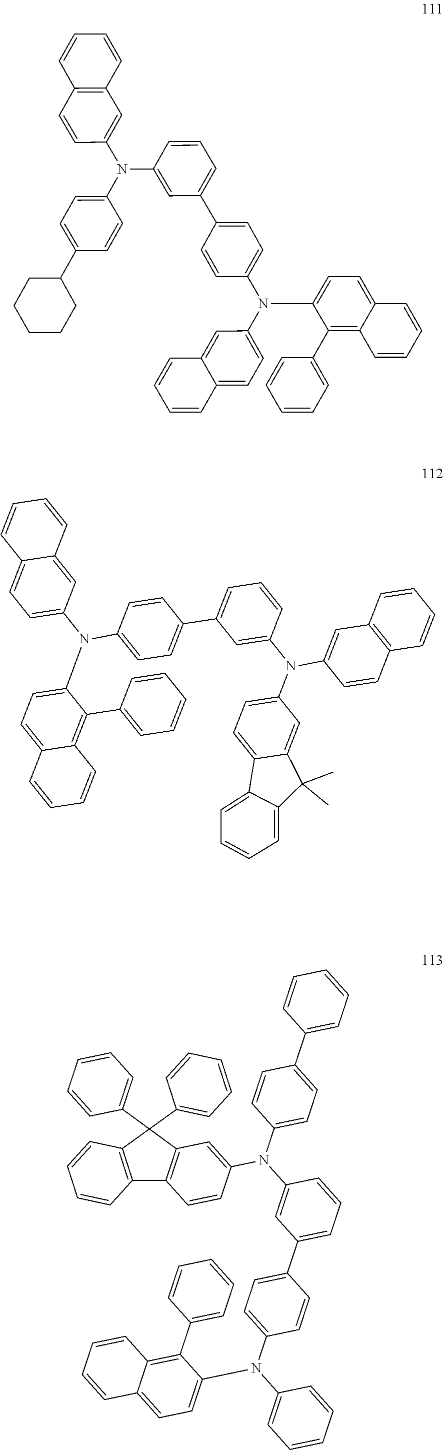 [SMILES: c1ccc(-c2c(N(c3ccc(-c4cccc(N(c5ccc(C6CCCCC6)cc5)c5ccc6ccccc6c5)c4)cc3)c3ccc4ccccc4c3)ccc3ccccc23)cc1 ; CC1(C)c2ccccc2-c2ccc(N(c3cccc(-c4ccc(N(c5ccc6ccccc6c5)c5ccc6ccccc6c5-c5ccccc5)cc4)c3)c3ccc4ccccc4c3)cc21 ; c1ccc(-c2ccc(N(c3cccc(-c4ccc(N(c5ccccc5)c5ccc6ccccc6c5-c5ccccc5)cc4)c3)c3ccc4c(c3)C(c3ccccc3)(c3ccccc3)c3ccccc3-4)cc2)cc1]

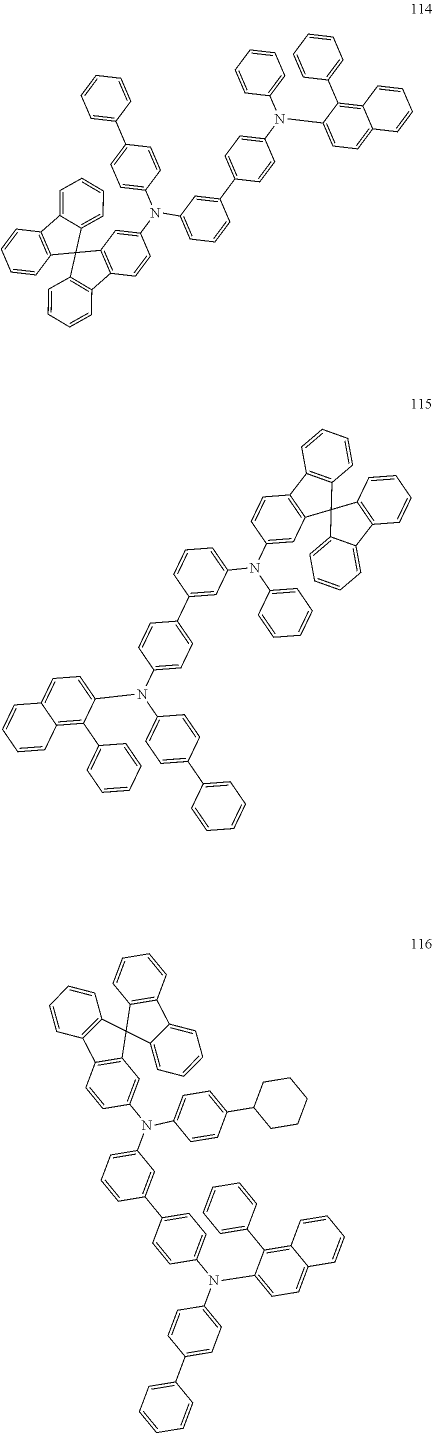 [SMILES: c1ccc(-c2ccc(N(c3cccc(-c4ccc(N(c5ccccc5)c5ccc6ccccc6c5-c5ccccc5)cc4)c3)c3ccc4c(c3)C3(c5ccccc5-c5ccccc53)c3ccccc3-4)cc2)cc1 ; c1ccc(-c2ccc(N(c3ccc(-c4cccc(N(c5ccccc5)c5ccc6c(c5)C5(c7ccccc7-c7ccccc75)c5ccccc5-6)c4)cc3)c3ccc4ccccc4c3-c3ccccc3)cc2)cc1 ; c1ccc(-c2ccc(N(c3ccc(-c4cccc(N(c5ccc(C6CCCCC6)cc5)c5ccc6c(c5)C5(c7ccccc7-c7ccccc75)c5ccccc5-6)c4)cc3)c3ccc4ccccc4c3-c3ccccc3)cc2)cc1]

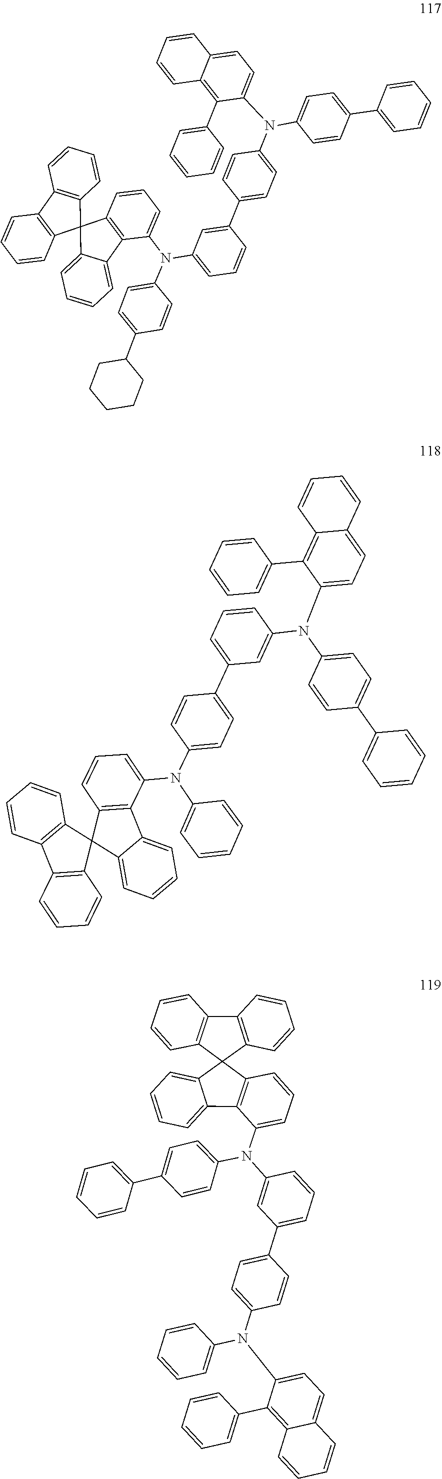 [SMILES: c1ccc(-c2ccc(N(c3ccc(-c4cccc(N(c5ccc(C6CCCCC6)cc5)c5cccc6c5-c5ccccc5C65c6ccccc6-c6ccccc65)c4)cc3)c3ccc4ccccc4c3-c3ccccc3)cc2)cc1 ; c1ccc(-c2ccc(N(c3cccc(-c4ccc(N(c5ccccc5)c5cccc6c5-c5ccccc5C65c6ccccc6-c6ccccc65)cc4)c3)c3ccc4ccccc4c3-c3ccccc3)cc2)cc1 ; c1ccc(-c2ccc(N(c3cccc(-c4ccc(N(c5ccccc5)c5ccc6ccccc6c5-c5ccccc5)cc4)c3)c3cccc4c3-c3ccccc3C43c4ccccc4-c4ccccc43)cc2)cc1]

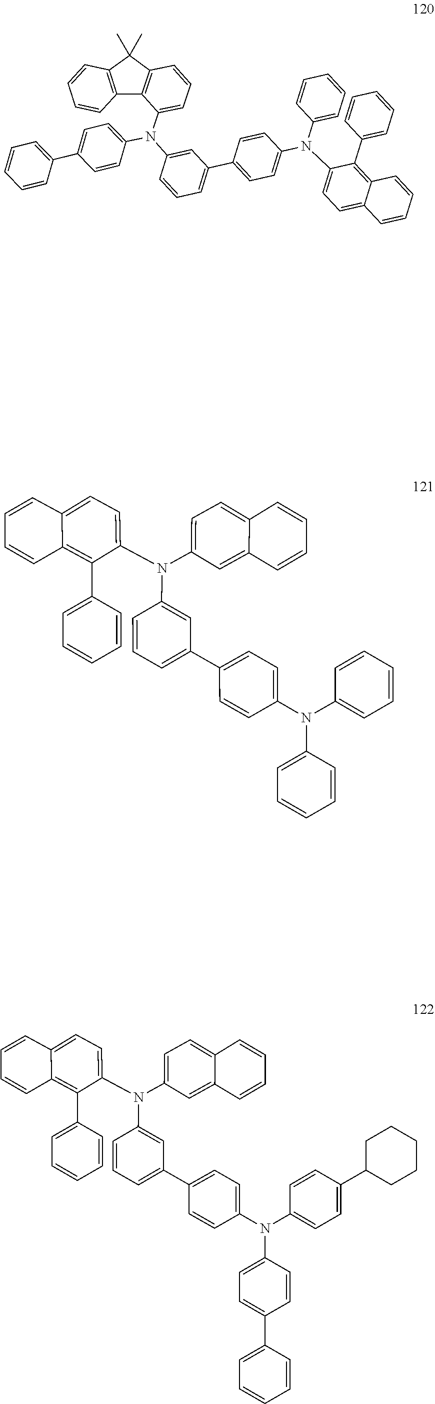 [SMILES: CC1(C)c2ccccc2-c2c(N(c3ccc(-c4ccccc4)cc3)c3cccc(-c4ccc(N(c5ccccc5)c5ccc6ccccc6c5-c5ccccc5)cc4)c3)cccc21 ; c1ccc(-c2c(N(c3cccc(-c4ccc(N(c5ccccc5)c5ccccc5)cc4)c3)c3ccc4ccccc4c3)ccc3ccccc23)cc1 ; c1ccc(-c2ccc(N(c3ccc(-c4cccc(N(c5ccc6ccccc6c5)c5ccc6ccccc6c5-c5ccccc5)c4)cc3)c3ccc(C4CCCCC4)cc3)cc2)cc1]

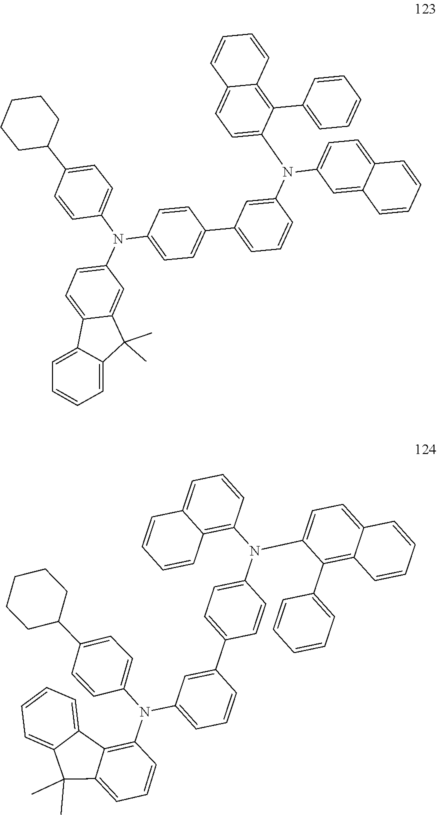 [SMILES: CC1(C)c2ccccc2-c2ccc(N(c3ccc(-c4cccc(N(c5ccc6ccccc6c5)c5ccc6ccccc6c5-c5ccccc5)c4)cc3)c3ccc(C4CCCCC4)cc3)cc21 ; CC1(C)c2ccccc2-c2c(N(c3ccc(C4CCCCC4)cc3)c3cccc(-c4ccc(N(c5ccc6ccccc6c5-c5ccccc5)c5cccc6ccccc56)cc4)c3)cccc21]

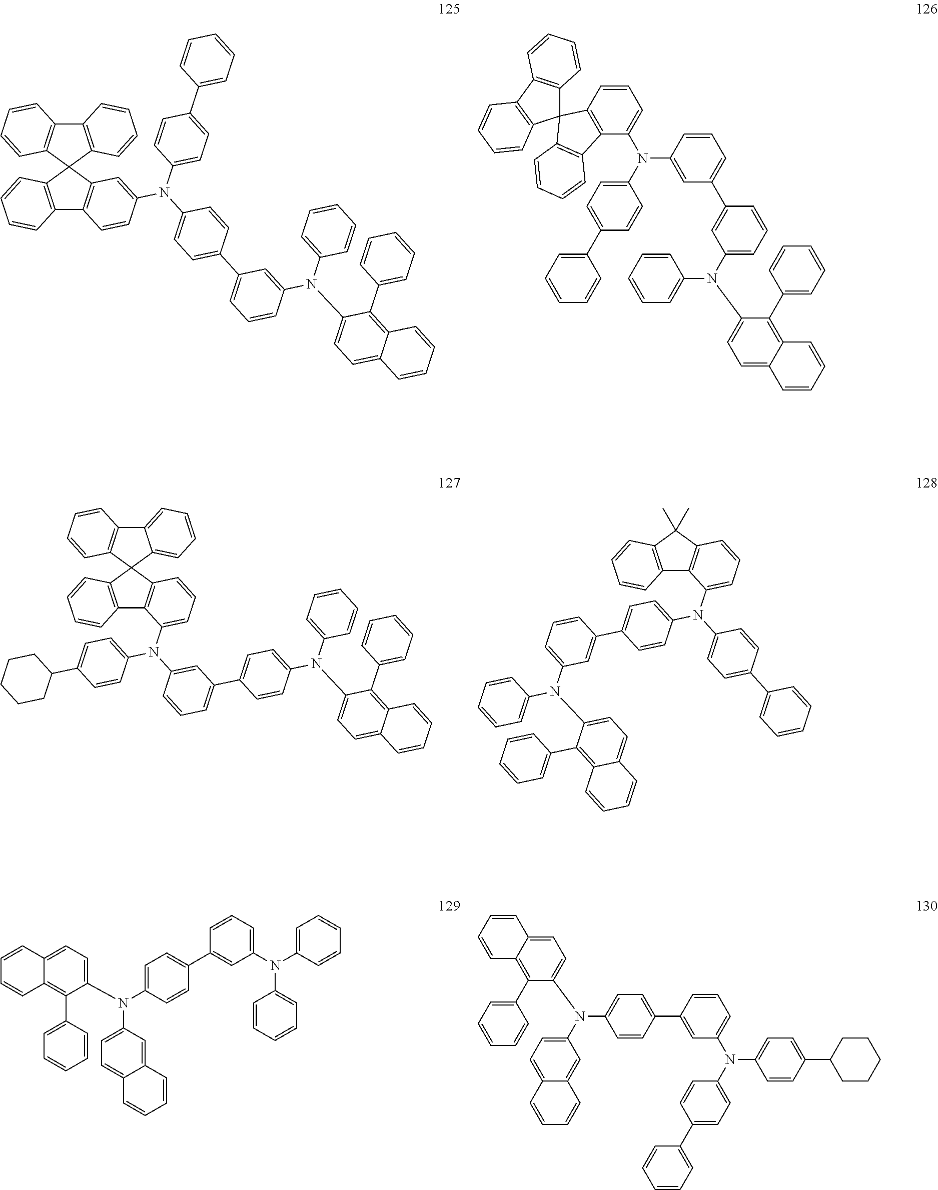

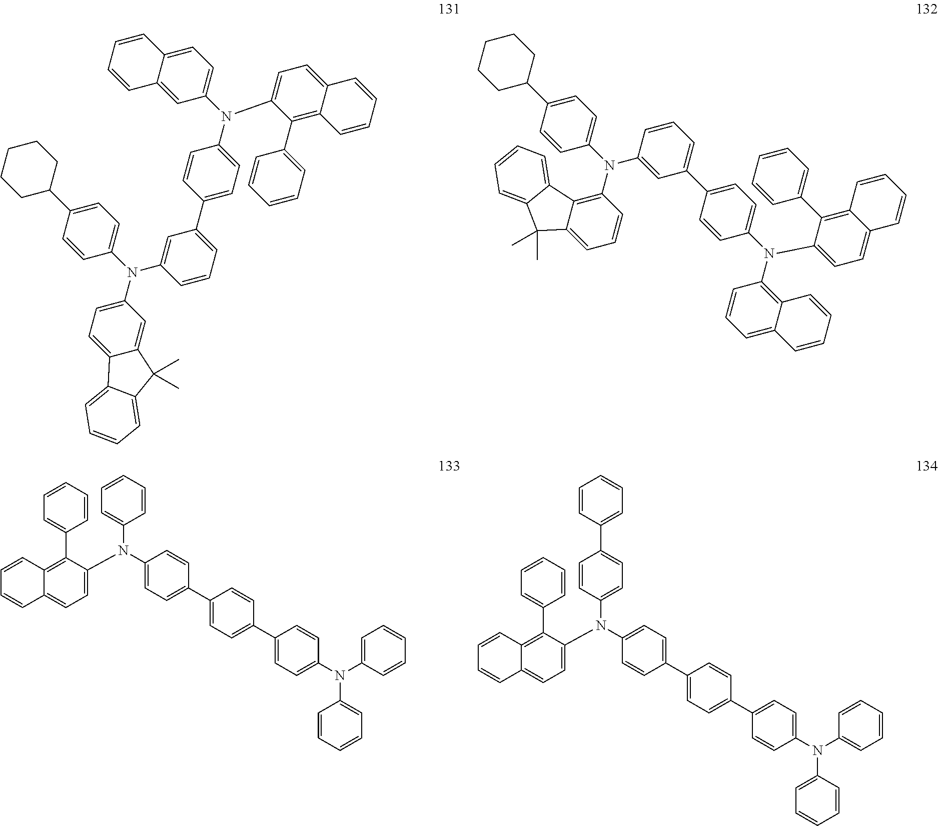

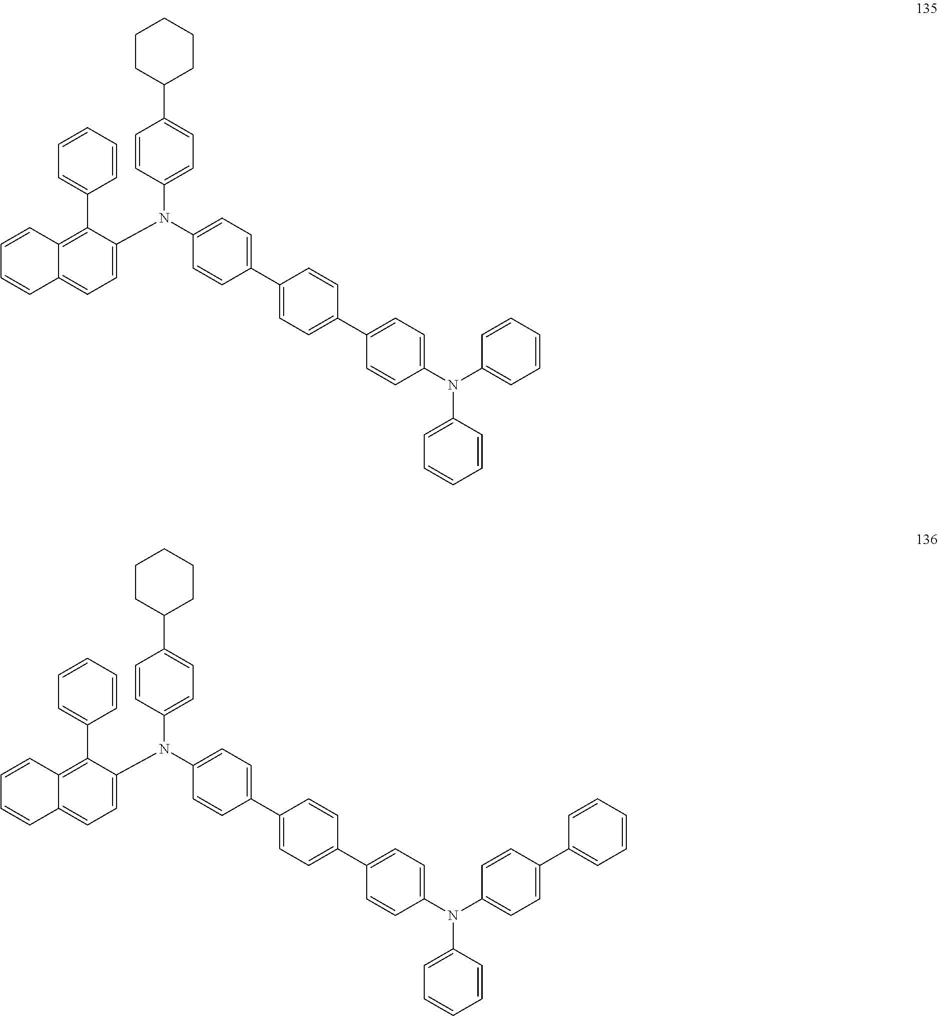 [SMILES: c1ccc(-c2c(N(c3ccc(-c4ccc(-c5ccc(N(c6ccccc6)c6ccccc6)cc5)cc4)cc3)c3ccc(C4CCCCC4)cc3)ccc3ccccc23)cc1 ; c1ccc(-c2ccc(N(c3ccccc3)c3ccc(-c4ccc(-c5ccc(N(c6ccc(C7CCCCC7)cc6)c6ccc7ccccc7c6-c6ccccc6)cc5)cc4)cc3)cc2)cc1]

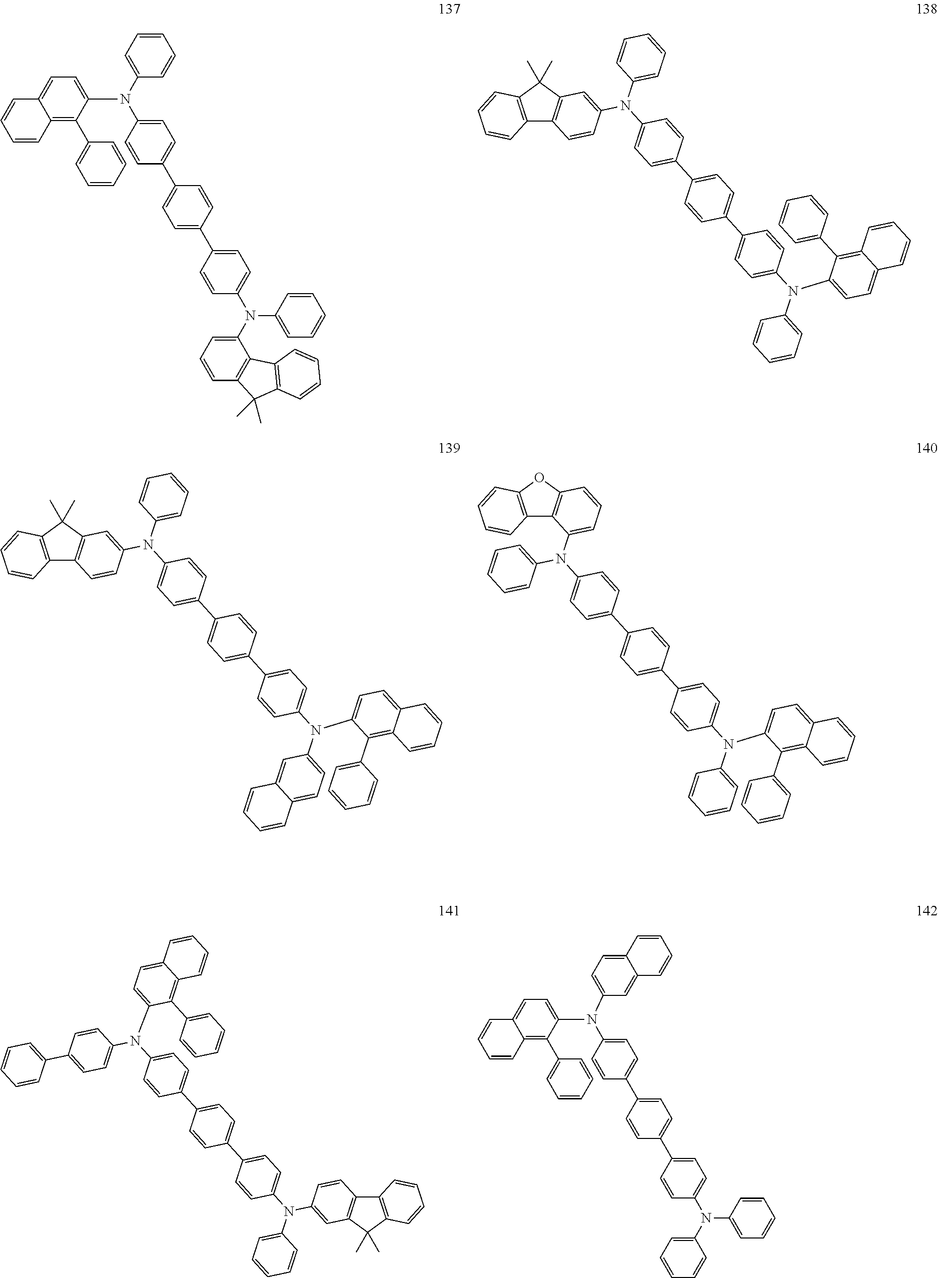

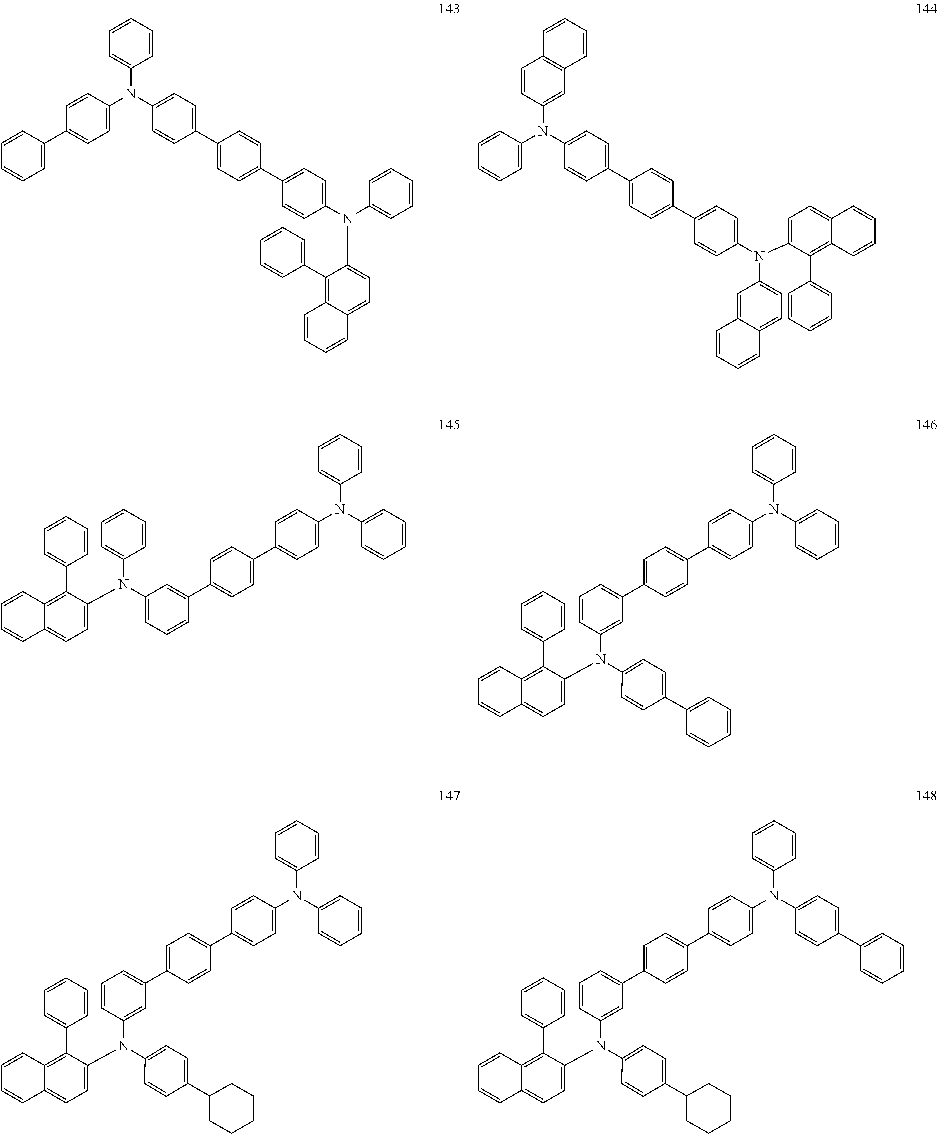

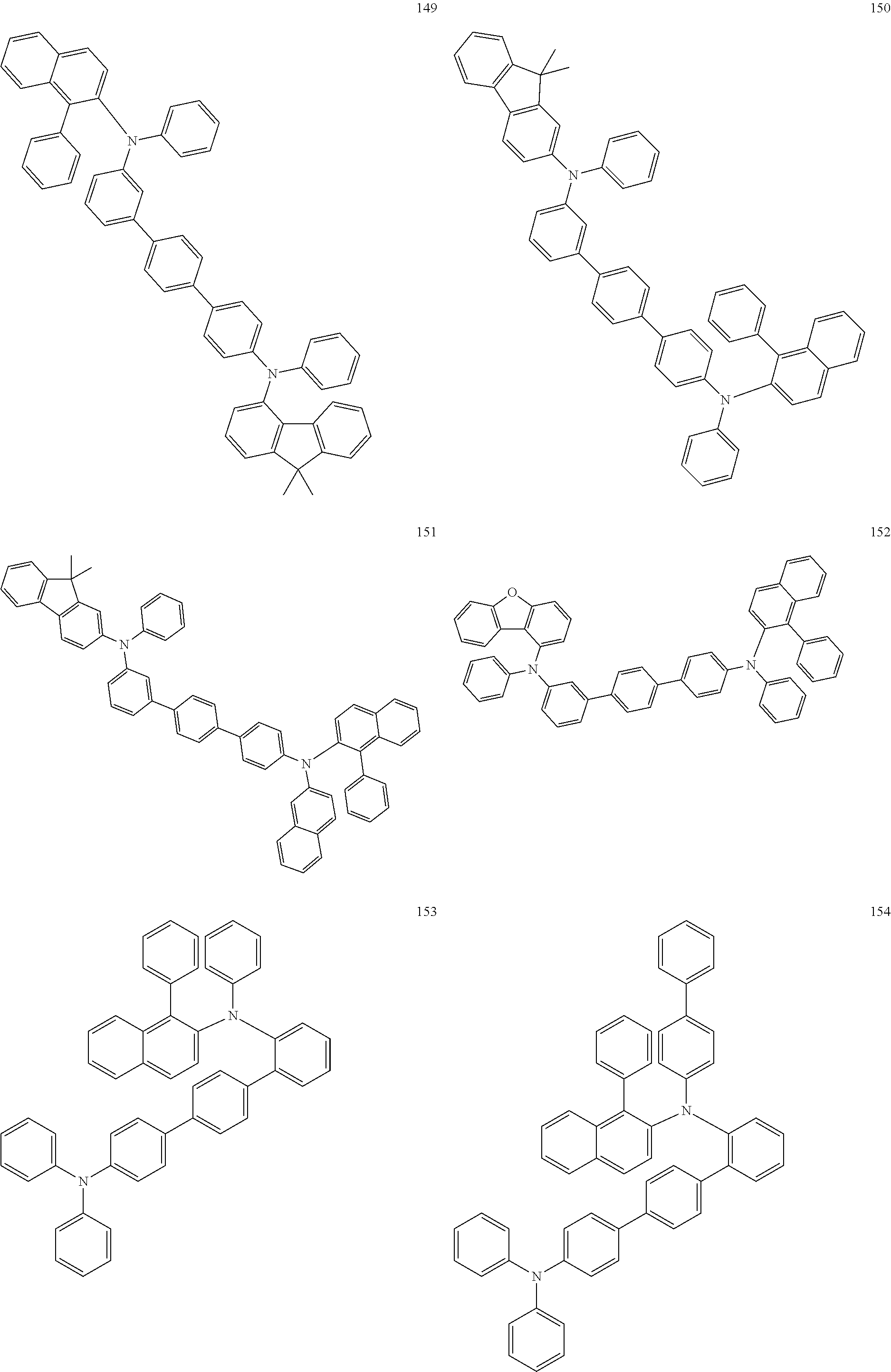

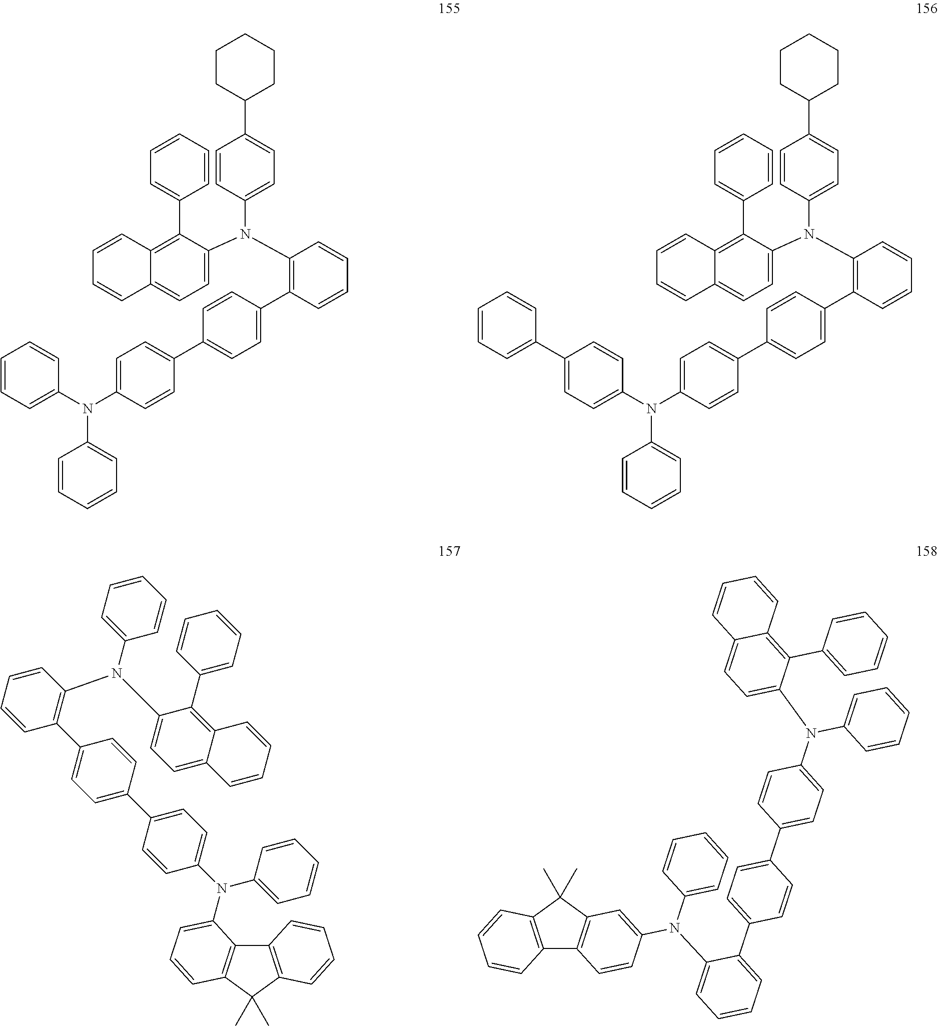 [SMILES: c1ccc(-c2c(N(c3ccc(C4CCCCC4)cc3)c3ccccc3-c3ccc(-c4ccc(N(c5ccccc5)c5ccccc5)cc4)cc3)ccc3ccccc23)cc1 ; c1ccc(-c2ccc(N(c3ccccc3)c3ccc(-c4ccc(-c5ccccc5N(c5ccc(C6CCCCC6)cc5)c5ccc6ccccc6c5-c5ccccc5)cc4)cc3)cc2)cc1 ; CC1(C)c2ccccc2-c2c(N(c3ccccc3)c3ccc(-c4ccc(-c5ccccc5N(c5ccccc5)c5ccc6ccccc6c5-c5ccccc5)cc4)cc3)cccc21 ; CC1(C)c2ccccc2-c2ccc(N(c3ccccc3)c3ccccc3-c3ccc(-c4ccc(N(c5ccccc5)c5ccc6ccccc6c5-c5ccccc5)cc4)cc3)cc21]

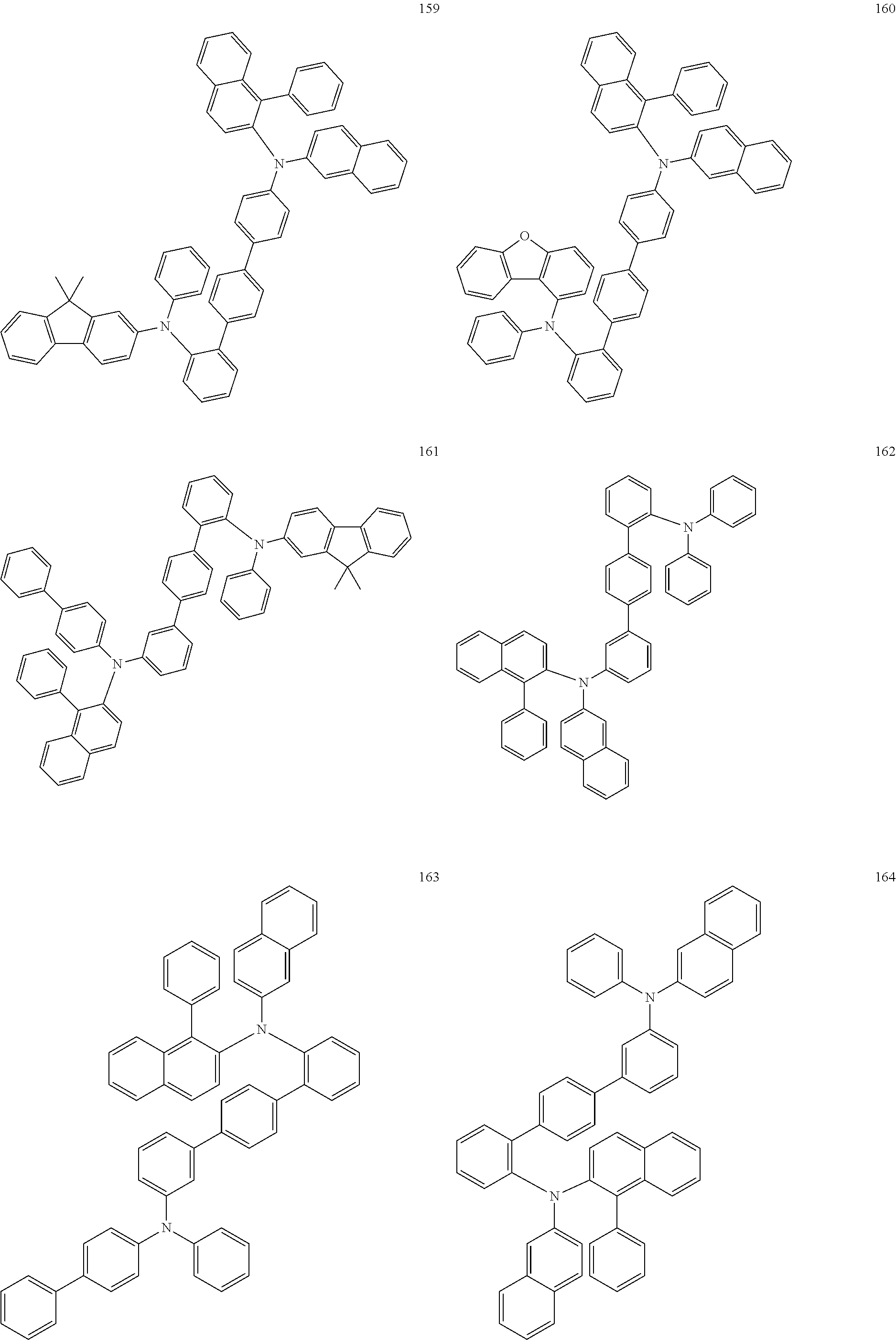

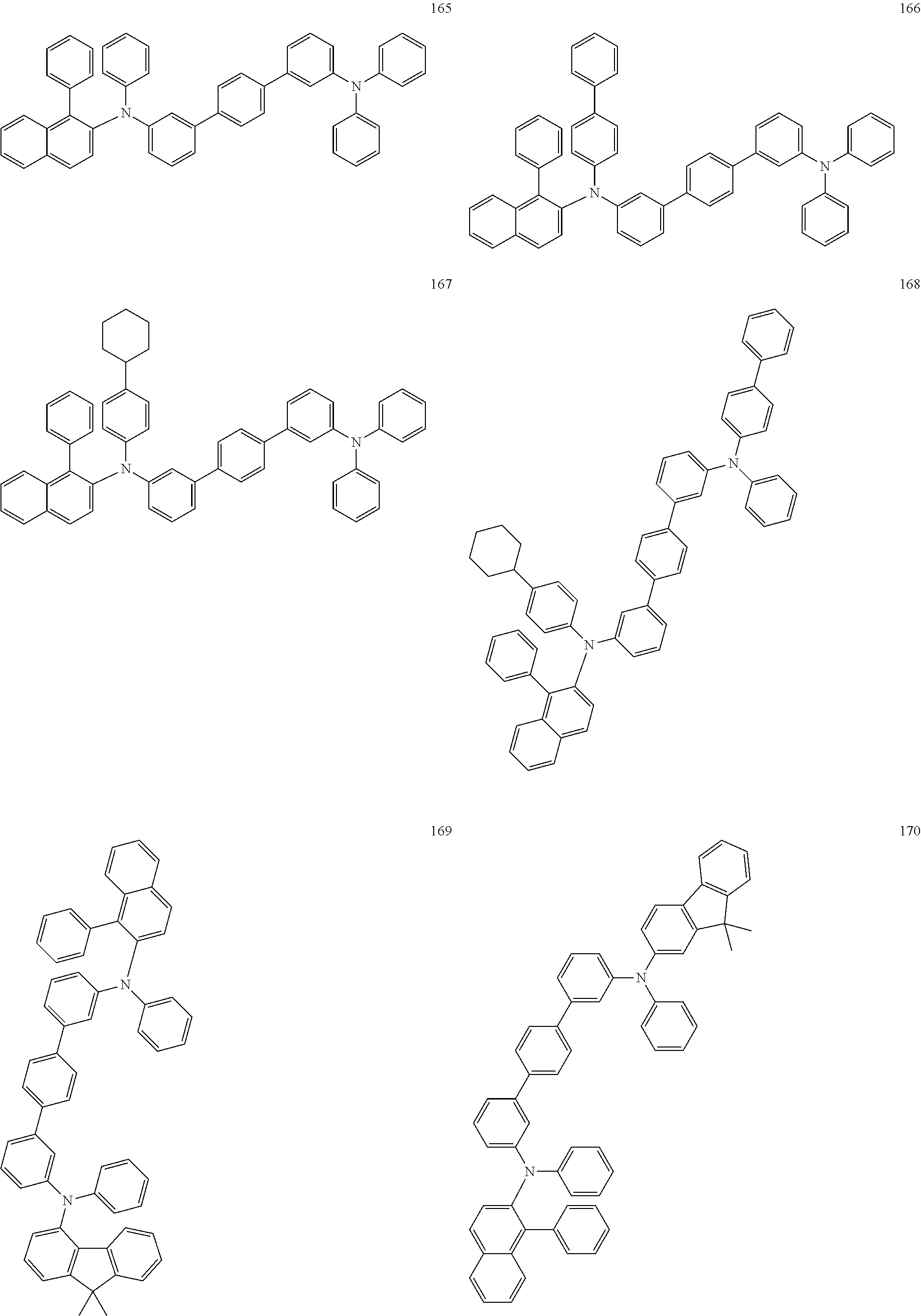

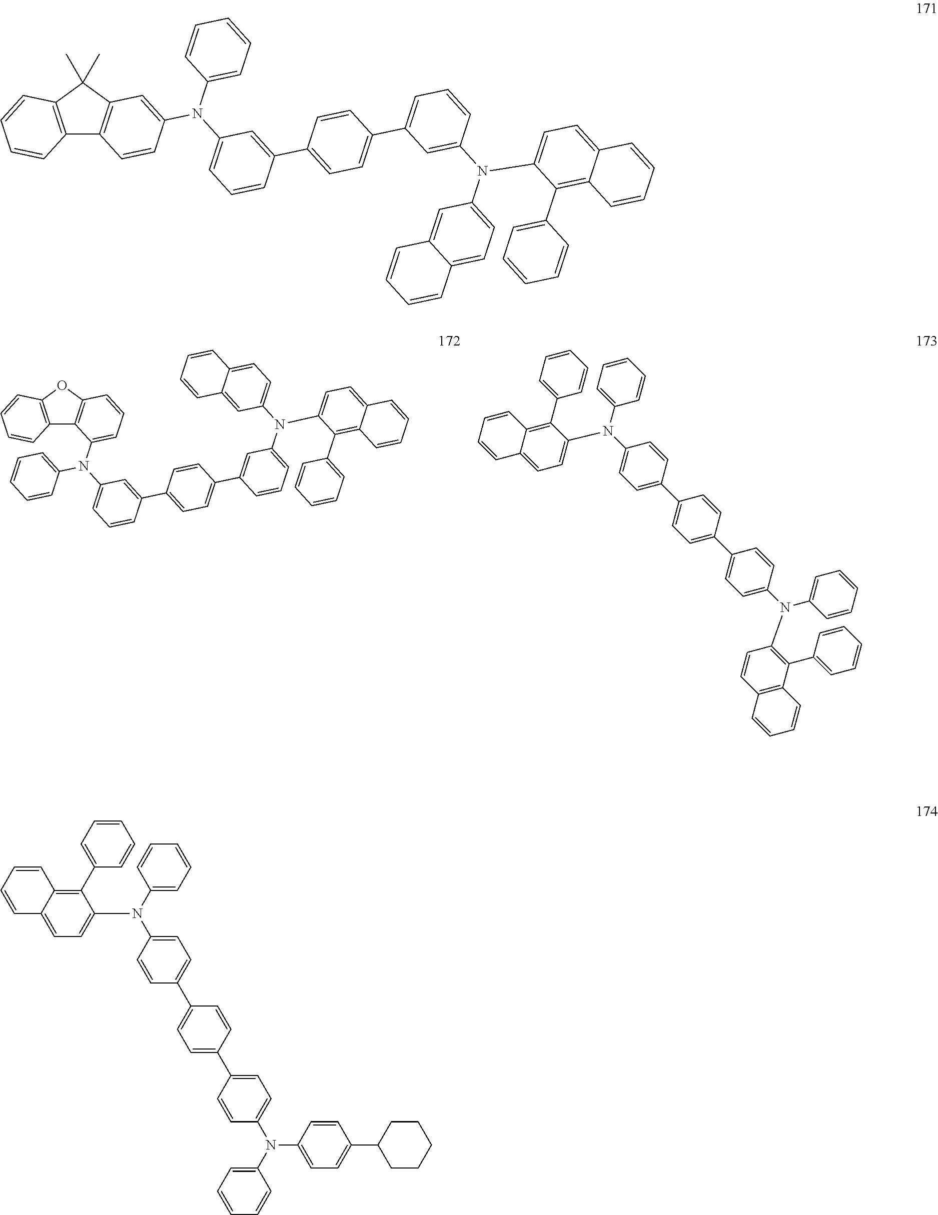 [SMILES: CC1(C)c2ccccc2-c2ccc(N(c3ccccc3)c3cccc(-c4ccc(-c5cccc(N(c6ccc7ccccc7c6)c6ccc7ccccc7c6-c6ccccc6)c5)cc4)c3)cc21 ; c1ccc(-c2c(N(c3cccc(-c4ccc(-c5cccc(N(c6ccccc6)c6cccc7oc8ccccc8c67)c5)cc4)c3)c3ccc4ccccc4c3)ccc3ccccc23)cc1 ; c1ccc(-c2c(N(c3ccccc3)c3ccc(-c4ccc(-c5ccc(N(c6ccccc6)c6ccc7ccccc7c6-c6ccccc6)cc5)cc4)cc3)ccc3ccccc23)cc1 ; c1ccc(-c2c(N(c3ccccc3)c3ccc(-c4ccc(-c5ccc(N(c6ccccc6)c6ccc(C7CCCCC7)cc6)cc5)cc4)cc3)ccc3ccccc23)cc1]

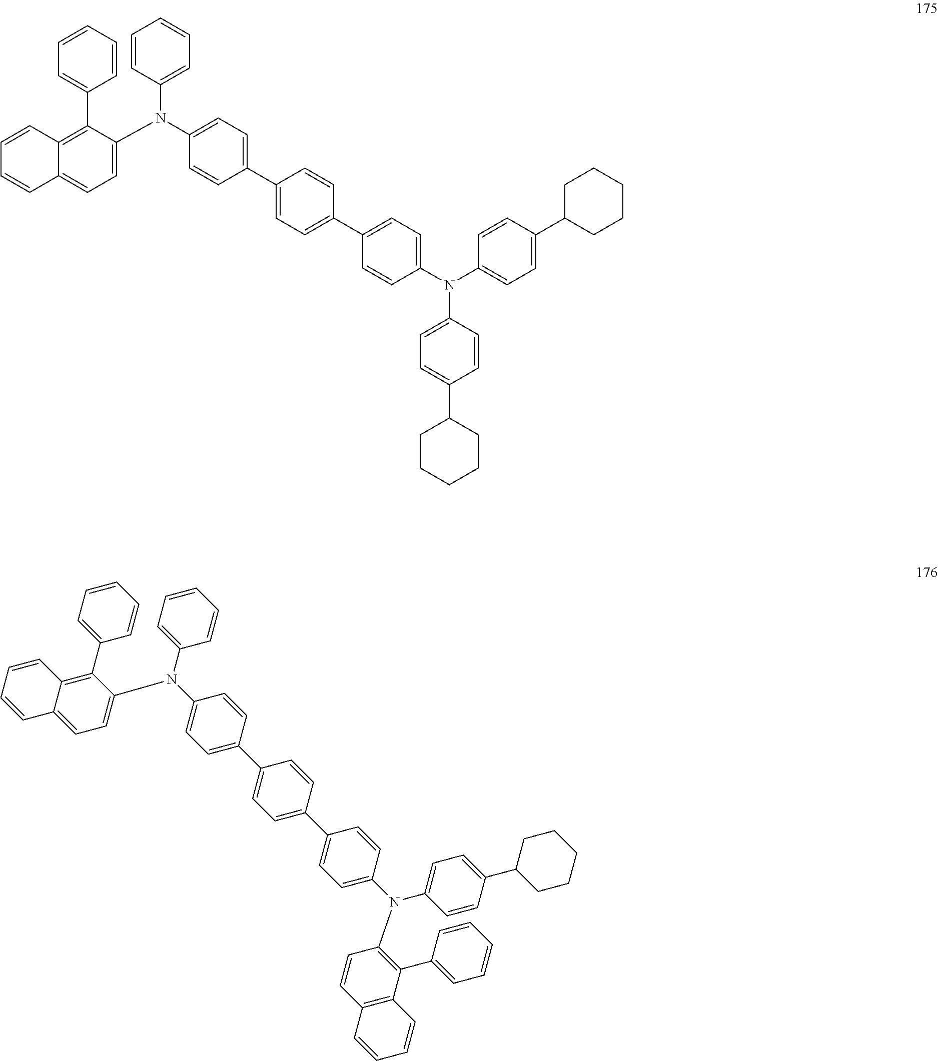 [SMILES: c1ccc(-c2c(N(c3ccccc3)c3ccc(-c4ccc(-c5ccc(N(c6ccc(C7CCCCC7)cc6)c6ccc(C7CCCCC7)cc6)cc5)cc4)cc3)ccc3ccccc23)cc1 ; c1ccc(-c2c(N(c3ccccc3)c3ccc(-c4ccc(-c5ccc(N(c6ccc(C7CCCCC7)cc6)c6ccc7ccccc7c6-c6ccccc6)cc5)cc4)cc3)ccc3ccccc23)cc1]

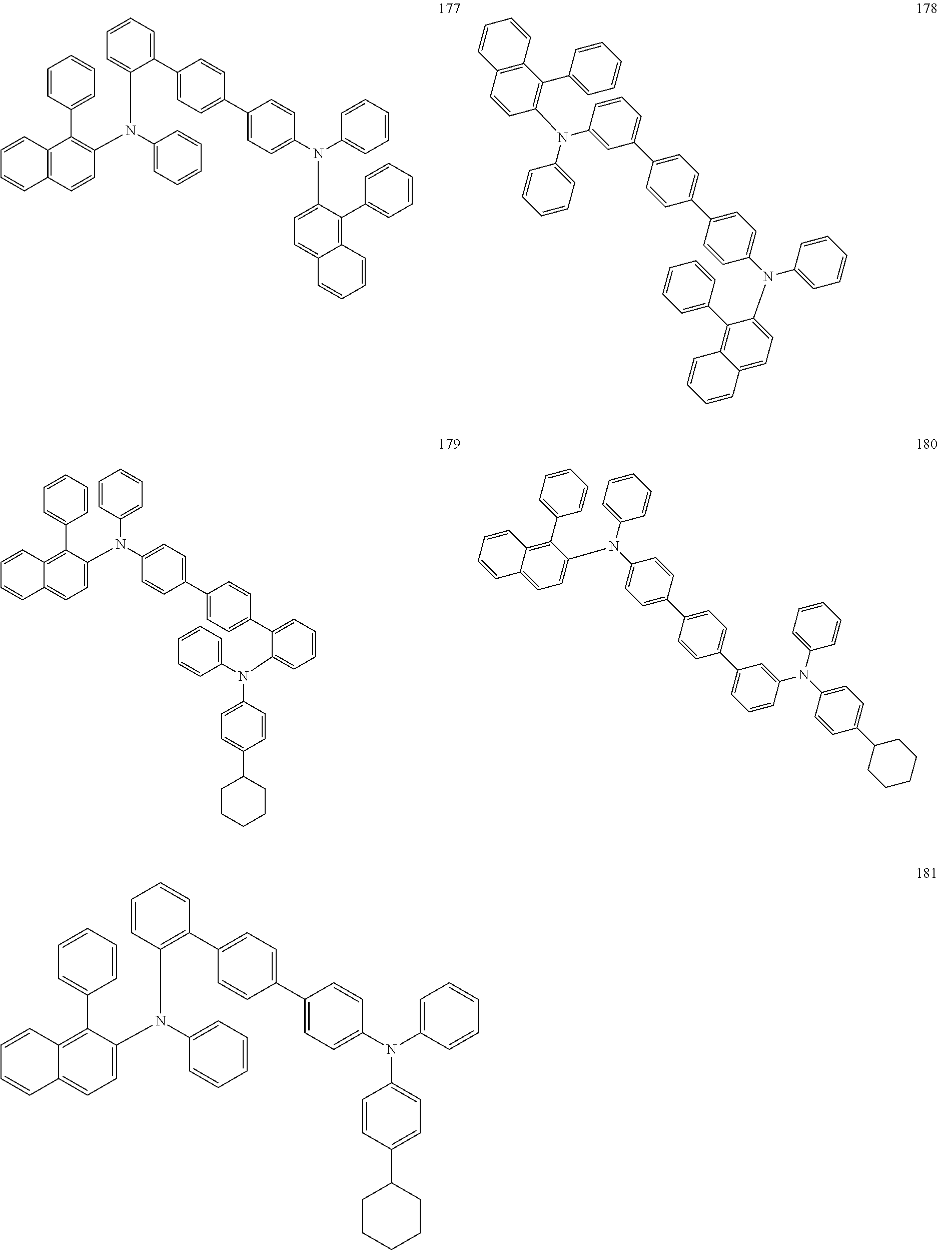

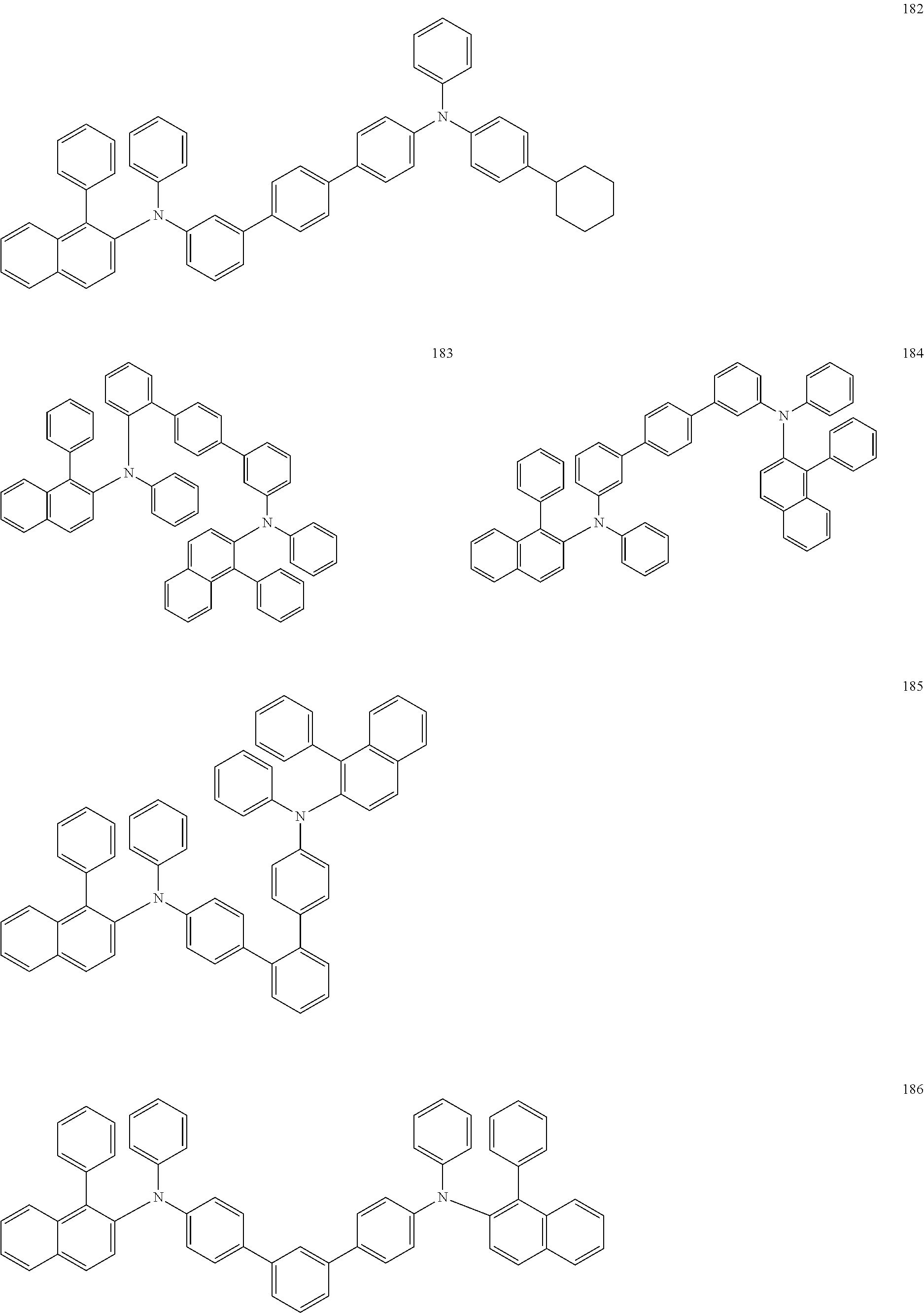

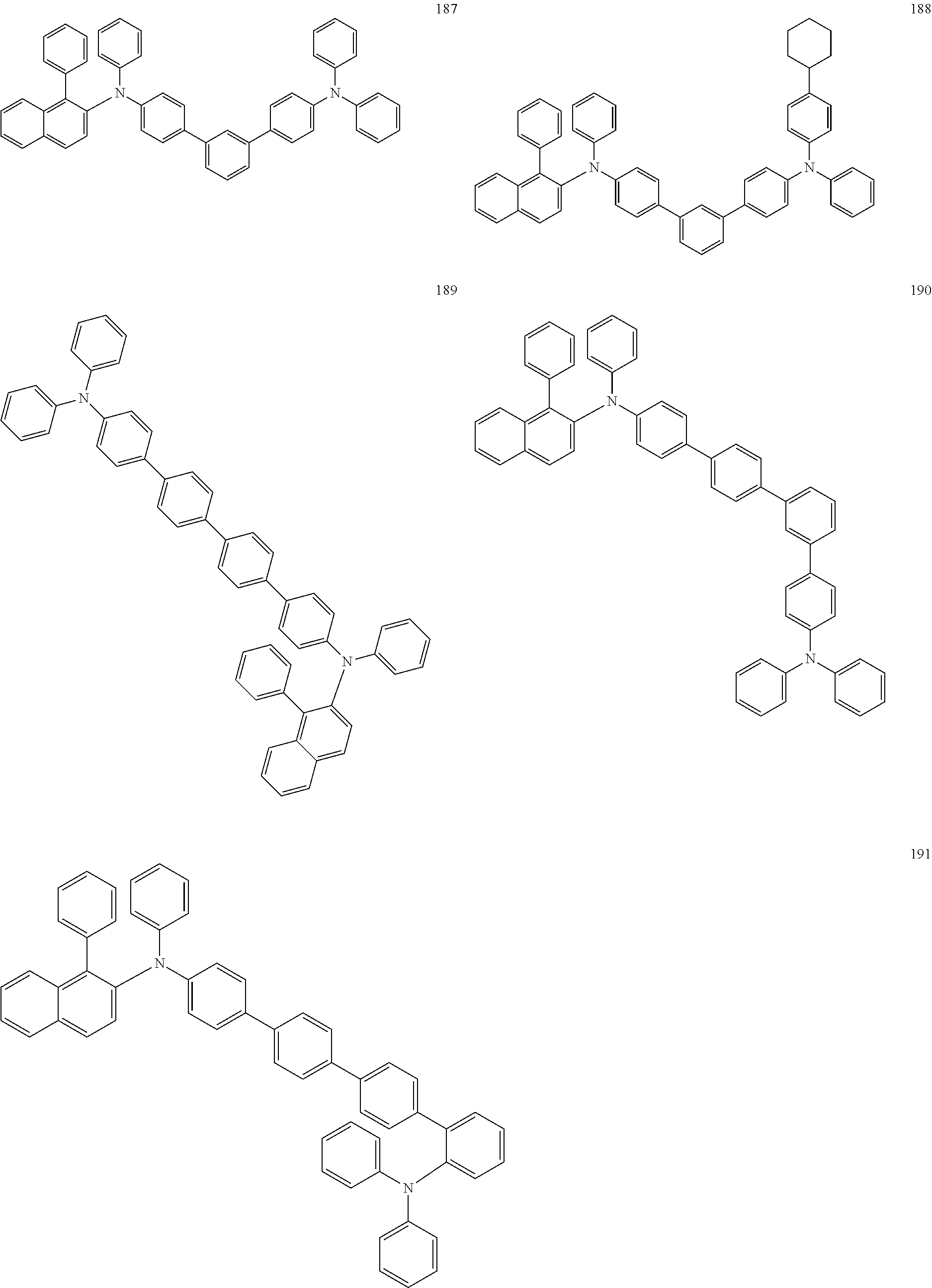 [SMILES: c1ccc(-c2c(N(c3ccccc3)c3ccc(-c4cccc(-c5ccc(N(c6ccccc6)c6ccccc6)cc5)c4)cc3)ccc3ccccc23)cc1 ; c1ccc(-c2c(N(c3ccccc3)c3ccc(-c4cccc(-c5ccc(N(c6ccccc6)c6ccc(C7CCCCC7)cc6)cc5)c4)cc3)ccc3ccccc23)cc1 ; c1ccc(-c2c(N(c3ccccc3)c3ccc(-c4ccc(-c5ccc(-c6ccc(N(c7ccccc7)c7ccccc7)cc6)cc5)cc4)cc3)ccc3ccccc23)cc1 ; c1ccc(-c2c(N(c3ccccc3)c3ccc(-c4ccc(-c5cccc(-c6ccc(N(c7ccccc7)c7ccccc7)cc6)c5)cc4)cc3)ccc3ccccc23)cc1 ; c1ccc(-c2c(N(c3ccccc3)c3ccc(-c4ccc(-c5ccc(-c6ccccc6N(c6ccccc6)c6ccccc6)cc5)cc4)cc3)ccc3ccccc23)cc1]

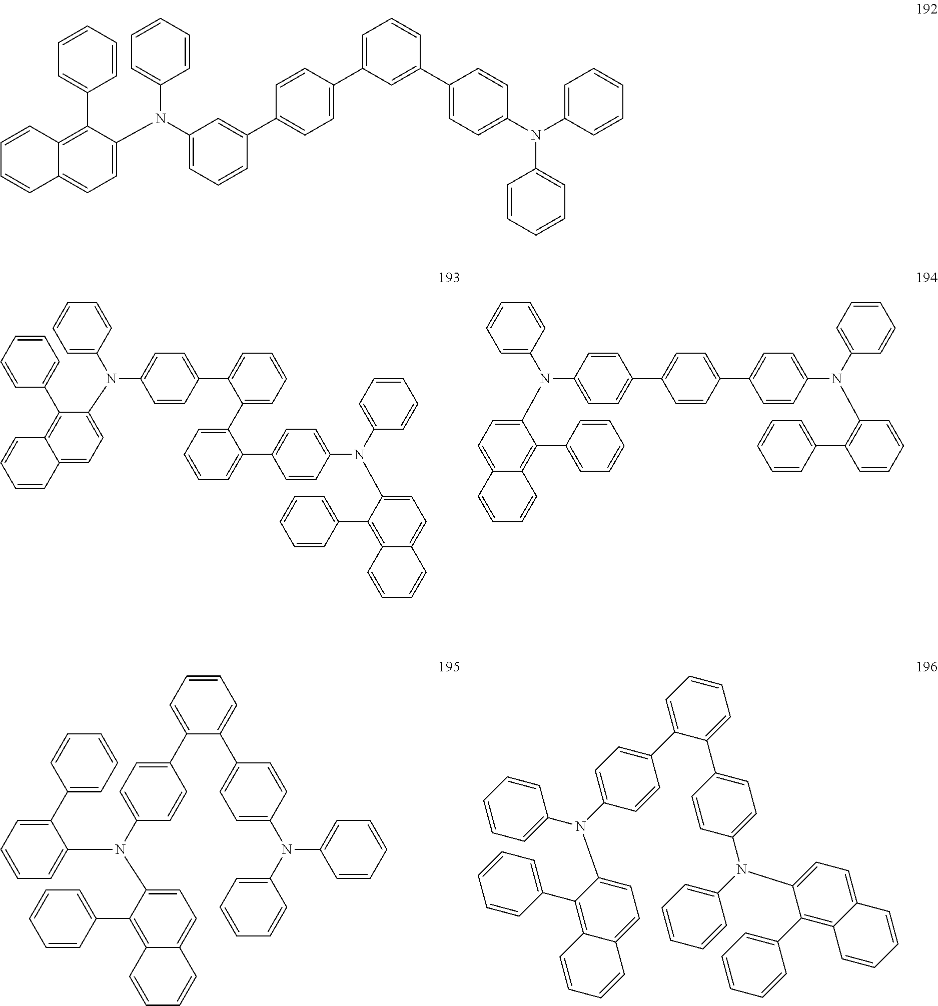

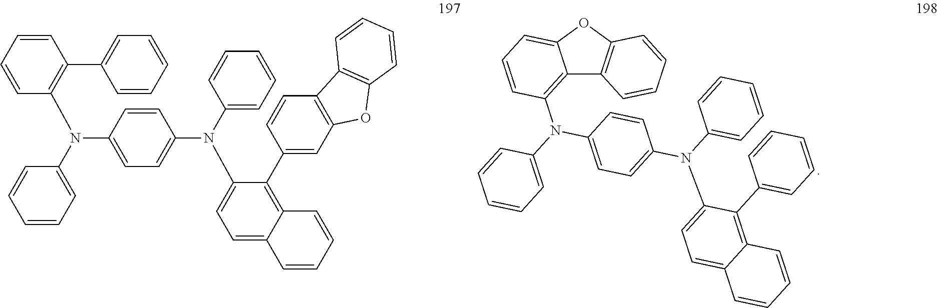 [SMILES: c1ccc(-c2ccccc2N(c2ccccc2)c2ccc(N(c3ccccc3)c3ccc4ccccc4c3-c3ccc4c(c3)oc3ccccc34)cc2)cc1 ; c1ccc(-c2c(N(c3ccccc3)c3ccc(N(c4ccccc4)c4cccc5oc6ccccc6c45)cc3)ccc3ccccc23)cc1]

15. An electronic device, the electronic device selected from among a television, a monitor, an outside billboard, a personal computer, a laptop computer, a personal digital assistant, a display device for a vehicle, a game console, a portable electronic device, and a camera, and comprising at least one light-emitting element,
wherein the at least one light-emitting element comprises an amine compound represented by Formula 1: 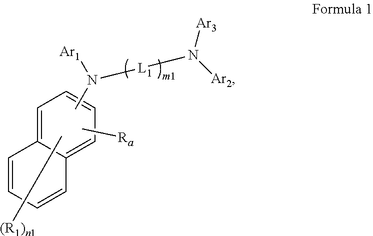
wherein, in Formula 1,
Ar₁ to Ar₃ are each independently a substituted or unsubstituted oxy group, a substituted or unsubstituted silyl group, a substituted or unsubstituted alkyl group having 1 to 20 carbon atoms, a substituted or unsubstituted alkenyl group having 2 to carbon atoms, a substituted or unsubstituted alkynyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
a case where Ar₁ comprises a fluorene group, a dibenzofuran group, or a dibenzothiophene group is excluded,
L₁ is a substituted or unsubstituted phenylene group,
Rₐ is a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
R₁ is hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1 is an integer of 0 to 6,
m1 is an integer of 1 to 4, and
when m1 is 2, then a case where L₁ is represented by Formula A-1 is excluded: 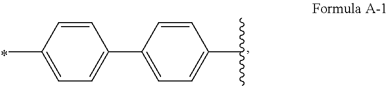
wherein, in Formula A-1, [symbol]
is a position at which a nitrogen atom, to which Ar₁ is connected in Formula 1 is connected, and 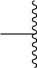
is a position at which a nitrogen atom, to which Ar₂ and Ar₃ are connected in Formula 1, is connected.

16. The electronic device of claim 15, wherein the electronic device comprises:
a base layer;
a circuit layer on the base layer;
a display element layer on the circuit layer and comprising the light-emitting element; and
an encapsulation layer on the display element layer, and
wherein the light-emitting element comprises:
a first electrode;
a second electrode on the first electrode; and
at least one functional layer between the first electrode and the second electrode and comprising the amine compound represented by Formula 1.

17. The electronic device of claim 16, wherein:
the light-emitting element further comprises a capping layer on the second electrode, the capping layer having a refractive index of at least about 1.6 for light in a wavelength range of about 550 nm to about 660 nm.

18. The electronic device of claim 16, further comprising a light control layer on the encapsulation layer,
wherein the light-emitting element is to emit a first color light, and
the light control layer comprises:
a first light control part comprising a first quantum dot which converts the first color light into a second color light with a longer wavelength than the first color light;
a second light control part comprising a second quantum dot which converts the first color light into a third color light with a longer wavelength than the first color light and the second color light; and
a third light control part which transmits the first color light.

19. The electronic device of claim 18, further comprising a color filter layer on the light control layer,
wherein the color filter layer comprises:
a first filter that transmits the second color light;
a second filter that transmits the third color light; and
a third filter that transmits the first color light.

20. An amine compound represented by Formula 1: 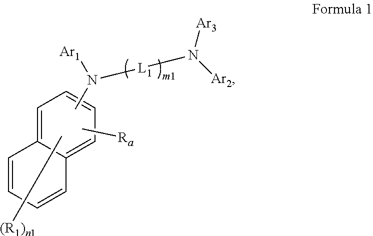
wherein, in Formula 1,
Ar₁ to Ar₃ are each independently a substituted or unsubstituted oxy group, a substituted or unsubstituted silyl group, a substituted or unsubstituted alkyl group having 1 to 20 carbon atoms, a substituted or unsubstituted alkenyl group having 2 to carbon atoms, a substituted or unsubstituted alkynyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
a case where Ar₁ comprises a fluorene group, a dibenzofuran group, a dibenzothiophene group is excluded,
L₁ is a substituted or unsubstituted phenylene group,
Rₐ is a substituted or unsubstituted oxy group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
R₁ is hydrogen, deuterium, a halogen, a substituted or unsubstituted oxy group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1 is an integer of 0 to 6,
m1 is an integer of 1 to 4, and
when m1 is 2, then a case where L₁ is represented by A-1 is excluded: 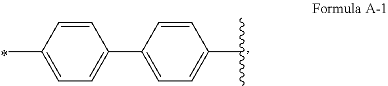
wherein, in Formula A-1, [symbol]
is a position at which a nitrogen atom, to which Ar₁ is connected in Formula 1, is connected, and 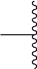
is a position at which a nitrogen atom, to which Ar₂ and Ar₃ are connected in Formula 1, is connected.
